(19) United States
(12) Patent Application Publication
(10) Pub. No.: US 2012/0322069 A1
(43) Pub. Date: Dec. 20, 2012

(54) Diagnositic Methods of Tumor Susceptibility With Nucleotide Polymorphisms Inside MicroRNA Target Sites

(75) Inventors: Milena Nicoloso, Aviano (IT); George Calin, Pearland, TX (US); Hao Sun, Shatin (HK); Ramana Davuluri, Philadelphia, PA (US)

(73) Assignees: The Ohio State Unversity, Columbus, OH (US); Board of Regents, The University of Texas System, Austin, TX (US)

(21) Appl. No.: 13/581,214

(22) PCT Filed: Feb. 23, 2011
(86) PCT No.: PCT/US11/25826

§ 371 Date: Aug. 24, 2012

Related U.S. Application Data
(60) Provisional application No. 61/307,898, filed on Feb. 25, 2010.

(51) Int. Cl. C12Q 1/68

(52) U.S. Cl. 435/6.11

(57) ABSTRACT

Methods of diagnosing tumor susceptibility or cancer including the step of determining whether a patient has one or more SNP-miRNA expression pattern combinations described herein. Each SNP-miRNA expression pattern combination is supported by data that shows that the SNP is associated with tumor susceptibility because of its ability to affect miRNA binding sites and/or miRNA:mRNA gene regulation.

### CROSS REFERENCE TO RELATED APPLICATIONS

None.

### STATEMENT REGARDING FEDERALLY SPONSORED RESEARCH OR DEVELOPMENT

None.

### THE NAMES OF PARTIES TO A JOINT RESEARCH AGREEMENT

None.

### INCORPORATION-BY-REFERENCE OF MATERIAL SUBMITTED ON COMPACT DISC

None.

### BACKGROUND OF THE INVENTION

Single nucleotide polymorphisms (SNPs) associated with polygenetic disorders, such as breast cancer (“BC”), can create, destroy or modify microRNA (“miRNA”) binding sites. SNPs known to be associated with tumor susceptibility (sometimes referred to herein as target SNPs), in silico and in vitro, have the ability to affect miRNA binding sites and consequently messenger RNA and protein regulation. However, to the extent SNPs interfere with miRNA gene regulation and affect cancer susceptibility remains largely unknown.

### SUMMARY OF THE INVENTION

Methods of diagnosing cancer presented herein. Such methodologies include the step of determining whether a patient has one or more SNP-miRNA expression patterns described herein. Each SNP-miRNA expression pattern combination is supported by data that shows that the SNP is associated with tumor susceptibility because of its ability to affect miRNA binding sites and/or miRNA:mRNA gene regulation. In addition, the methods of diagnosing or screening for breast cancer in patients presented herein may include an additional first step of testing the patient for known BRCA1 or the BRACA2 mutations identified with breast cancer, and, if the test is negative (or otherwise classified as non-cancerous via use of blood, mucal swabs, leukocytes etc. . . . ), then the patient is tested for one or more of the SNP-miRNA expression combinations presented herein to determine his or her tumor susceptibility. Kits useful in cancer diagnosis are also provided.

### BRIEF DESCRIPTION OF THE DRAWINGS

FIGS. 1A, 1B, 1C and 1D are real-time analyses of endogenous miRNA levels in breast samples and cell lines. miRNAs were tested for their interaction with breast cancer associated SNPs are expressed in breast samples (normal and tumor) from an independent set of patients. Samples 3, 6, and 9 are paired (normal and tumor), MM231 and MBMDA231.

FIGS. 2A, 2B and 2C show the minimum free energy (“MFE”) change distribution for certain SNPs located either in 3′ UTRs, CDS or 5′ UTRs. The ±8% threshold in MFE change is marked, corresponding for all three distributions to the peaks of the bimodal distribution and to the 20 and 80 percentiles.

FIG. 3 provides the results of a Luciferase reporter assay for pGL3-rs28382751-XIAP showing miRNA::mRNA duplex for rs28382751-XIAP::miR-542-5p interaction, with the active allele or the non-active allele. This figure also shows the results from the luciferase reporter assay for pGL3-rs28382751 co-transfected either with scrambled negative control and miR-542-5p. Luciferase activity is expressed relative to scrambled negative control (=1); values represent the average +/−standard deviation of 3 independent experiments performed in six replicates.

FIGS. 4A, 4B, 4C and 4D show the effects on luciferase activity and protein expression of a conserved miR-638 target inside BRCA1 3′ UTR. FIG. 4A shows that targets prediction for a miR-638 conserved target site inside BRCA1 3′ UTR. FIG. 4B shows the results of a Luciferase reporter assay for pGL3-BRCA1-3′ UTR co-transfected either with scrambled negative control or miR-638. Luciferase activity is expressed relative to scrambled negative control (=1); values represent the average +/−standard deviation of 3 independent experiments performed in six replicates. miR-638 does not repress the activity of the luciferase construct containing BRCA1 3′ UTR. FIG. 4 C shows the overexpression of miR-638 was validated by realtime PCR. FIG. 4D shows the WB for BRCA1 in MCF7 cells co-transfected with scrambled negative control or miR-638 together with a pCMV empty vector or pCMV BRCA1 expressing vector without its 3′ UTR BRCA1 CDS was mutagenized (QuikChange Site-Directed Mutagenesis Kit, Stratagene) to obtain two different pCMV-BRCA1 vectors encoding the rs777917 [C] and [T] alleles. Suppression of BRCA1 protein levels was achieved by miR-638 in the absence of BRCA1 3′ UTR and was greater with rs777917 [C] active allele.

FIG. 5 shows the general steps taken for miRNA::target SNP identification.

FIGS. 6A, 6B, 6C and 6D provide data that shows for approximately 15% of the investigated transcribed SNPs, association with breast cancer can be biologically due to interference with miRNA binding and therefore miRNA gene regulation

FIGS. 7A1, 7A2, 7A3, 7A4, 7B1, 7B2, and 7C1, 7C2 are illustrative of the effect of the two target SNPs rs799917-BRCA1 and rs334348-TGFBR1 on endogenous protein levels.

### DETAILED DESCRIPTION

Methods of determining tumor susceptibility and/or cancer susceptibility (risk) in a patient are provided herein. The methods include the step of determining the presence of at least one SNP allelic variant that disrupts an miRNA::mRNA interaction in a sample taken from the patient, wherein the presence of the SNP allelic variant and the miRNA expression (sometimes referred to as the “SNP-miRNA expression combination”) in the sample indicates an increase susceptibility of cancer in the patient. This methodology may also include the first step of determining whether the patient has mutations in the BRCA1 gene or BRCA2 gene, and if neither is found, then the additional step of determining the presence of the SNP-miRNA expression combination is made.

microRNAs (“miRNAs”) control gene expression by base pairing with messenger RNA (mRNAs). Single nucleotide polymorphisms (“SNPs”) within mRNA-miRNA target sites perturb RNA duplex minimum free energy (“MFE”), miRNA binding, and consequently gene expression. Germline occurrence of target SNP inside cancer-relevant genes varies among populations with different predisposition to breast cancer. A disruption of miRNA gene regulation by SNPs can contribute to tumor susceptibility. As such, methods and kits for determining the predisposition of certain types of cancers often having a hereditary component are provided herein. These methods are directed to the detection of a specific type of germline mutation in genes involved or associated with certain types of hereditary cancers including BRCA1 and TGFBR1 genes. The role of a SNP located in interactor sites with miRNAs mediate the genesis of these mutations. Somatic mutations of this type, particularly in the BRCA1 gene in human breast and ovarian cancer, are useful in the diagnosis and prognosis of such cancers. The identification of the mutations may be useful as kits of molecular diagnosis in families with familial breast cancer and other cancers, and the diagnosis of tumor susceptibility in general.

Hence, the discoveries provided herein offer a novel approach to molecular diagnosis and provide a mechanism by which the location of SNP in messenger RNA can be useful in evaluating a predisposition to cancer. As taught herein and discussed below, screening for such predisposition can be done by first checking for mutations in the protein coding regions of few genes such as BCRA1 and BCRA2 and others. See, U.S. Pat. No. 5,747,282, Col. 28, 1. 16 through Col. 31, 1. 53, Claims 1-20, incorporated herein by reference; U.S. Pat. No. 5,837,492. Col. 5, 1. 62 through Col. 6, 1. 45, Col. 11. 1. 10 through Col. 15, 1. 67, and Claims 1 through 30, incorporated herein by reference; U.S. Pat. No. 5,693,473, Claims 1-14 incorporated herein by reference; U.S. Pat. No. 5,709,999, Claims 1 through 35, incorporated herein by reference; and U.S. Pat. No. 5,710,001, Claims 1-35, incorporated herein by reference.

As noted below, it has become widely accepted that certain breast cancer risk can be specified by the combinatorial product of multiple low-penetrance alleles. However, the multiplicity of identified variants alone cannot account for the approximate 80 percent of familial breast cancer cases unrelated to high-penetrance breast cancer susceptibility genes. Therefore, the methods described herein use a combinatorial model of breast cancer predisposition (and tumors in general) based on the polymorphic variants inside PCG transcript that may alter miRNA gene regulation.

miRNAs are a family of endogenous, short non-coding RNAs that modulate post-transcriptional gene regulation. They exert their regulatory role on protein-coding gene (PCG) expression by binding to either full or partial complementary sequences primarily in the 3′ untranslated region (UTR), but also inside the coding sequence (CDS) and the 5′ UTR, of the corresponding messenger RNAs (mRNAs). Eventually, this affects mRNA stability and translation. Filipowicz, W., et al., Mechanisms of Post-Transcriptional Regulation By MicroRNAs: Are The Answers In Sight? 9:102-114, Nat Rev Genet (2008); Miranda, K. C., et al., A Pattern-Based Method For The Identification of MicroRNA Binding Sites And Their Corresponding Heteroduplexes, 126:1203-1217, Cell (2006); Tay Y., et al., MicroRNAs to Nanog, Oct4 and Sox2 Coding Regions Modulate Embryonic Stem Cell Differentiation, 455:1124-1128, Nature (2008). The role of miRNAs in human cancer pathogenesis can be established by the identification of genetic alterations in miRNA loci, miRNA expression signatures that define different neoplastic phenotypes and numerous oncogenes and tumor suppressor genes as miRNA targets. Spizzo R, et al, SnapShot: MicroRNAs in Cancer, 137:586-586, 581, Cell (2009). miRNA expression can be identified by quantitative RT PCR, a sensitive and rapid method. Further, detection of SNP includes quantitative methods such as direct Sanger sequencing, a next generation deep sequencing. This together with the measurement of miRNAs by quantitative RT PCR is sometimes referred to as QRTPCR.

The discovery of germline variants that influence cancer susceptibility has been acknowledged by others. See e.g., Pharoah PD, et al., Association Studies for Finding Cancer-Susceptibility Genetic Variants, 4:850-860, Nat Rev Cancer (2004). The variations include single nucleotide polymorphisms (“SNPs”), which occur approximately once every 100 to 300 base pairs in the human genome. SNPs constitute a source of information for unraveling the heterogeneity of human cancer pathogenesis, clinical course and response to treatment. SNPs are located in genes, of which about 50,000-250,000 SNPs, mostly located in or around the 25,000 PCGs, have a putative biological effect. Risch, N. J., Searching for Genetic Determinants in the New Millennium, 405:847-856, Nature (2000).

SNPs can affect protein function by changing the amino acid sequences (non synonymous SNP) or by perturbing their regulation (e.g. affecting promoter activity, splicing process, and DNA and pre-mRNA conformation). Nuckel H, et al., Association of a novel regulatory polymorphism (−938C>A) in the BCL2 Gene Promoter with Disease Progression and Survival In Chronic Lymphocytic Leukemia, 109:290-297 Blood (2007); Krawczak M, et al., The Mutational Spectrum of Single Base-Pair Substitutions In mRNA Splice Junctions Of Human Genes: Causes and Consequences, 90:41-54, Hum Genet (1992). When SNPs occur in 3′ UTRs, they may interfere with mRNA stability and translation by altering polyadenylation, protein::mRNA, and miRNA::mRNA regulatory interactions.

Recent studies have found 3′ UTR SNPs that affect PCG expression via miRNA gene regulation in different diseases. See e.g., Abelson J F, et al., Sequence Variants in SLITRK1 are Associated With Tourette's Syndrome, 310:317-320 Science (2005); Sethupathy P, et al., Human microRNA-155 on Chromosome 21 Differentially Interacts with Its Polymorphic Target in the AGTR1 3′ Untranslated Region: A Mechanism for Functional Single-Nucleotide Polymorphisms Related to Phenotypes, 81:405-413 μm J Hum Genet 2007; Mishra P J, et al., A miR-24 microRNA Binding-site Polymorphism In Dihydrofolate Reductase Gene Leads To Methotrexate Resistance, Proc Natl Acad Sci USA 2007. He H, et al. The Role of microRNA Genes in Papillary Thyroid Carcinoma, 102:19075-19080, Proc Natl Acad Sci USA (2005); Jazdzewski K, et al., Common SNP in Pre-miR-146a Decreases Mature miR Expression and Predisposes to Papillary Thyroid Carcinoma, 105:7269-7274, Proc Natl Acad Sci USA (2008); Adams B D, et al., The Micro-Ribonucleic Acid (miRNA) miR-206 Targets the Human Estrogen Receptor-Alpha (ERalpha) and Represses ERalpha Messenger RNA and Protein Expression In Breast Cancer Cell Lines, 21:1132-1147, Mol Endocrinol (2007).

As described herein, the disruption of miRNA target binding by SNPs located in the 5′ UTRs, CDS or 3′ UTRs is likely to be a widespread mechanism leading to cancer susceptibility and initiation. For example, it has been recently shown that a kRAS variant within the let-7 target site increased the risk for non-small cell lung carcinoma among moderate smokers. Chin L. J., et al, A SNP in a let-7 microRNA Complementary Site in the KRAS 3′ Untranslated Region Increases Non-Small Cell Lung Cancer Risk, 68:8535-8540, Cancer Res (2008). Moreover, since the 3′ UTRs of PCGs have been insufficiently screened for mutations/polymorphisms, the extent of such abnormalities is likely to be much greater than initially predicted.

Breast Cancer (BC) is one of the most common female malignancies with more than one million new cases diagnosed every year. Parkin, D. M. International Variation, 23:6329-6340, Oncogene (2004). It is widely accepted that the majority of BC risk might be specified by the combined product of multiple low-penetrance alleles. See, Stratton M R, et al., The Emerging Landscape of Breast Cancer Susceptibility, 40:17-22, Nat Genet (2008). However, the multiplicity of variants identified so far alone cannot account for ˜80% of familial BC cases that are unrelated to high-penetrance BC susceptibility genes. The role of polymorphic variants located in BC relevant genes in tumor susceptibility has been extensively addressed. Johnson N, et al., Counting Potentially Functional Variants in BRCA1, BRCA2 and ATM Predicts Breast Cancer Susceptibility, 16:1051-1057, Hum Mol Genet (2007); Easton, D. F., et al, Genome-Wide Association Study Identifies Novel Breast Cancer Susceptibility Loci, 447:1087-1093 Nature (2007).

Table I immediately is a list of transcribed SNPs found to be associated with breast cancer in PubMed (from January 2006 to December 2008).

TABLE I

| SNP | Gene | Location |
|---|---|---|
| rs7539542 | ADIPOR1 | 3′ UTR |
| rs4148946 | CHST3 | 3′ UTR |
| rs4148949 | CHST3 | 3′ UTR |
| rs10046 | CYP19A1 | 3′ UTR |
| rs1061646 | ZNF276 | 3′ UTR |
| rs7759 | DPAGT1 | 3′ UTR |
| rs8551 | DPAGT1 | 3′ UTR |
| rs11575213 | IGFBP5 | 3′ UTR |
| rs2675 | ITGB5 | 3′ UTR |
| rs11902171 | ITGAV | 3′ UTR |
| rs7702523 | JMY | 3′ UTR |
| rs3180 | SPOCK2 | 3′ UTR |
| rs34330 | CDKN1B | 5′ UTR |
| rs1045642 | ABCB1 | CDS |
| rs876755 | ACAD9 | CDS |
| rs2241766 | ADIPOQ | CDS |
| rs1130409 | APEX1 | CDS |
| rs1801516 | ATM | CDS |
| rs28997576 | BARD1 | CDS |
| rs1045485 | CASP8 | CDS |
| rs700519 | CYP19A1 | CDS |
| rs13181 | ERCC2 | CDS |
| rs17655 | ERCC5 | CDS |
| rs572169 | GHSR | CDS |
| rs643788 | DPAGT1 | CDS |
| rs2069849 | IL6 | CDS |
| rs1940475 | MMP8 | CDS |
| rs1800566 | NQO1 | CDS |
| rs1042438 | EHHADH | CDS |
| rs1042838 | PGR | CDS |
| rs1982073 | TGFB1 | CDS |
| rs10735810 | VDR | CDS |
| rs731236 | VDR | CDS |
| rs4588 | GC | CDS |
| rs7041 | GC | CDS |
| rs1799782 | XRCC1 | CDS |
| rs25487 | XRCC1 | CDS |
| rs132788 | XRCC6 | CDS |

Notwithstanding, there has not been an investigation of the pathogenetic implications of genomic variation on breast cancer susceptibility that has evaluated the role of SNPs in miRNA::mRNA gene regulation and its effect in breast cancer (sometimes noted herein as “BC”) development.

We analyzed SNPs associated with BC susceptibility for their ability to affect miRNA binding sites and miRNA::mRNA gene regulation. We identified SNP dependent miRNA interactions that might explain the pathogenetic relevance of known BC-associated SNPs. We performed a genome-wide scan for SNPs inside PCG transcribed regions (3′ UTR, CDS and 5′ UTR) that could potentially alter miRNA binding (referred to as target SNPs). Finally, we illustrated the functional consequences of target SNPs on miRNA regulation of protein expression and their possible clinical implications due to differences in frequency distribution among familial BCs, sporadic BCs and controls.

As a result, we identify a new pathogenetic mechanism to explain the association of certain SNPs with BC susceptibility by using the latest knowledge on post-transcriptional gene regulation by miRNAs. Evidence of the effect of SNPs on miRNA binding derived from our integrative analysis delineates a novel role for SNPs in gene expression modulation. Supporting this theory, a recent work suggested that differences in SNP allele frequency among ethnic groups account for differences in gene expression. Spielman, R. S., et al., Common Genetic Variants Account for Differences in Gene Expression Among Ethnic Groups, 39:226-231. Nat Genet (2007). Two out of the 11 cis SNPs identified by Spielman et al. were actually transcribed SNPs located in 3′ UTRs. As shown in Table IX immediately below, we found that both of the SNPs were qualified to induce minimum free energy (“MFE”) absolute changes greater than 8% with multiple putative interacting miRNAs. miRNAs predicted by miRanda-target the cis SNP rs1065663 and re1061810 which determine the phenotypic differences in gene expression between distinct genetic populations.

TABLE IX

| | | | | Minimum | |
|---|---|---|---|---|---|
| | Allelic | | Active | free | MFE |
| SNP ID | Variants | miR ID | Allele | energy | change |
| rs1061810 | A/C | miR-769-3p | C | −19.85 | 78% |
| rs1061810 | A/C | miR-345 | C | −17.21 | 40% |
| rs1061810 | A/C | miR-372 | C | −16.15 | 22% |
| rs1061810 | A/C | miR-512-3p | C | −19.75 | 15% |
| rs1061810 | A/C | miR-520f | A | −16.2 | −13% |
| rs1065663 | A/G | miR-484 | G | −20.93 | 181% |
| rs1065663 | A/G | miR-662 | G | −16.58 | 43% |
| rs1065663 | A/G | miR-133a | G | −16.61 | 38% |
| rs1065663 | A/G | miR-485-5p | G | −18.39 | 38% |
| rs1065663 | A/G | miR-412 | G | −19.48 | 30% |
| rs1065663 | A/G | miR-372 | A | −18.01 | −29% |
| rs1065663 | A/G | miR-523 | G | −17.23 | 27% |
| rs1065663 | A/G | miR-92b | G | −24.09 | 18% |
| rs1065663 | A/G | miR-596 | G | −19.45 | 11% |
| rs1065663 | A/G | miR-492 | A | −19.22 | −10% |

As a result, we believe that cis SNPs modulate phenotypic gene expression diversities, at least in part, through alteration of miRNA target binding capability; ultimately, leading to differences in the susceptibility to complex genetic diseases, such as breast cancer (“BC”). Notably, among the target SNPs identified as differentially expressed in BC, in our research, we found rs334348 located in the 3′ UTR of TGFBR1. Likewise, the association of this SNP with germline allele specific expression of TGFBR1 was recently found, and it was shown to confer an increased risk of colorectal cancer. Valle L, et al., Germline Allele-Specific Expression of TGFBR1 Confers an Increased Risk Of Colorectal Cancer, 321:1361-1365, Science (2008). This phenotype could be explained, for example, by an altered miRNA interaction, and therefore strongly supports our belief that SNPs affecting miRNAs function contribute to allele specific protein expression and consequently play a role in tumor susceptibility. Similarly, a recent study based on transcriptome analyses of heterozygous mouse strains, showed that polymorphic miRNA target sites determine differences in gene expression, which further supports our hypothesis. Kim J, Bartel D P, Allelic Imbalance Sequencing Reveals that Single-Nucleotide Polymorphisms Frequently Alter MicroRNA-Directed Repression, 27:472-477, Nat Biotechnol (2009).

As to the effect of transcribed SNPs on miRNA::mRNA interactions and the regulation of gene expression by these SNPs with possible implications in tumorigenesis, we found that numerous target SNPs can effectively interfere with miRNA target recognition. Furthermore, the distribution of these SNPs significantly varies among BC and control populations, suggesting a role in BC susceptibility. For example, the BC-associated rs1982073-TGFB1 variant had been previously linked with low expression levels of TGFB1 during the initial phases of tumorigenesis, while higher expression levels were found in more advanced metastatic BC stages. Gonzalez-Zuloeta Ladd, A. M., et al., Transforming-Growth Factor betal Leu10Pro Polymorphism and Breast Cancer Morbidity, 43:371-374, Eur J Cancer (2007); Dunning A. M., et al. A Transforming Growth Factorbetal Signal Peptide Variant Increases Secretion in vitro and is Associated with Increased Incidence of Invasive Breast Cancer, 63:2610-2615, Cancer Res (2003). This switch in TFGB1 expression could be due to concomitant changes of miR-187 expression levels and could be favored by carrying the [T] variant. Additionally, this described model, together with TGFB1 pro- or anti-tumorigenic activities in different phases of tumorigenesis, could explain the conflicting results observed for rs1982073-TGFB1 association with breast cancer.

Another interesting observation is that the stronger binding occurring between miRNA and active allele does not unequivocally mean repression. In fact, as shown for rs1799782-XRCC1, miR-187 stabilizes XRCC1 expression when carrying the active allele. This finding is in line with other evidence showing that miRNAs can positively modulate the protein expression. Orom UA, et al., MicroRNA-10a Binds the 5′UTR of Ribosomal Protein mRNAs and Enhances Their Translation, 30:460-471, Mol Cell (2008); Vasudevan S, et al., Switching From Repression To Activation: microRNAs Can Up-Regulate Translation, 318:1931-1934 Science (2007). Furthermore, by searching the complete PCG transcripts (5′ UTR, CDS, 3′ UTR) for miRNA target SNPs, we have identified miR-638 as regulator of BRCA1 via binding to its target site inside CDS (to our knowledge the first miRNA regulating BRCA1 published so far). Although a conserved interaction site was predicted by TargetScan in the 3′ UTR of BRCA1, we found that it is not a functional site in the conditions we tested.

Recently, Tay and coworkers also proved the existence and abundance of miRNA targets located inside the CDS of PCGs, confirming the possibility that miR-638 regulates BRCA1 in a similar way. Tay Y, et al., MicroRNAs to Nanog, Oct4 and Sox2 Coding Regions Modulate Embryonic Stem Cell Differentiation, 455:1124-1128, Nature (2008). Although large case-control studies have not reached a unifying conclusion on the relevance to BC susceptibility of the miRNA target SNP rs799917 inside BRCA1 CDS, its association with tumor susceptibility has been recently highlighted in a SNP analysis of DNA repair genes in glioblastomas multiforme samples. Freedman M. L., et al., A Haplotype-Based Case-Control Study of BRCA1 and Sporadic Breast Cancer Risk, 65:7516-7522, Cancer Res (2005); Cox D G, et al., Haplotype Analysis Of Common Variants in the BRCA1 Gene and Risk of Sporadic Breast Cancer, 7:R171-175, Breast Cancer Res (2005); Chang J S, et al., Pathway Analysis of Single-Nucleotide Polymorphisms Potentially Associated with Glioblastoma Multiforme Susceptibility Using Random Forests, 17:1368-1373, Cancer Epidemiol Biomarkers Prey (2008).

Through our work, we found that rs799917 [T] allele is also associated with a weaker miR-638 dependent BRCA1 reduction, and is linked with BC. Moreover, we observed that low frequency CDS mutations (both synonymous and non synonymous, data not shown) might exert on miRNA gene regulation effects similar to target SNPs. Wood LD, et al. The Genomic Landscapes of Human Breast and Colorectal Cancers. 318:1108-1113, Science (2007). This provides an innovative insight on the importance of synonymous mutations, which in the future should be considered and investigated with renewed attention.

Supported by the data presented here, we maintain that both the genetic variants (allelic SNPs) and microRNA expression patterns can account for gene expression differences among distinct populations. Therefore, the newly recognized class of target SNPs contribute to cancer susceptibility not per se (or as tags for specific haplotypes), but in concert with miRNA expression patterns. Considering the multifactorial model of breast cancer susceptibility, transcribed allelic variants that can alter miRNA PCG regulation are provided. Such altered miRNA PCG regulations thus increasing miRNA abnormalities, and contributing to tumor susceptibility, and the general risk of tumors, by a mechanism of subtle gene regulation.

### Example 1

### Methods

Data Sources.

We obtained mature human miRNA sequences (total of 885) from NCBI build 36 database (version13.0). We downloaded SNP data from HapMap (Release 21A). We retrieved 5′ UTR, 3′ UTR, and CDS mRNA genomic locations from the UCSC known gene table (human genome assembly, NCBI build 36, hg18). http://genome.ucsc.edu/.

miRNA::SNP Interaction Analyses.

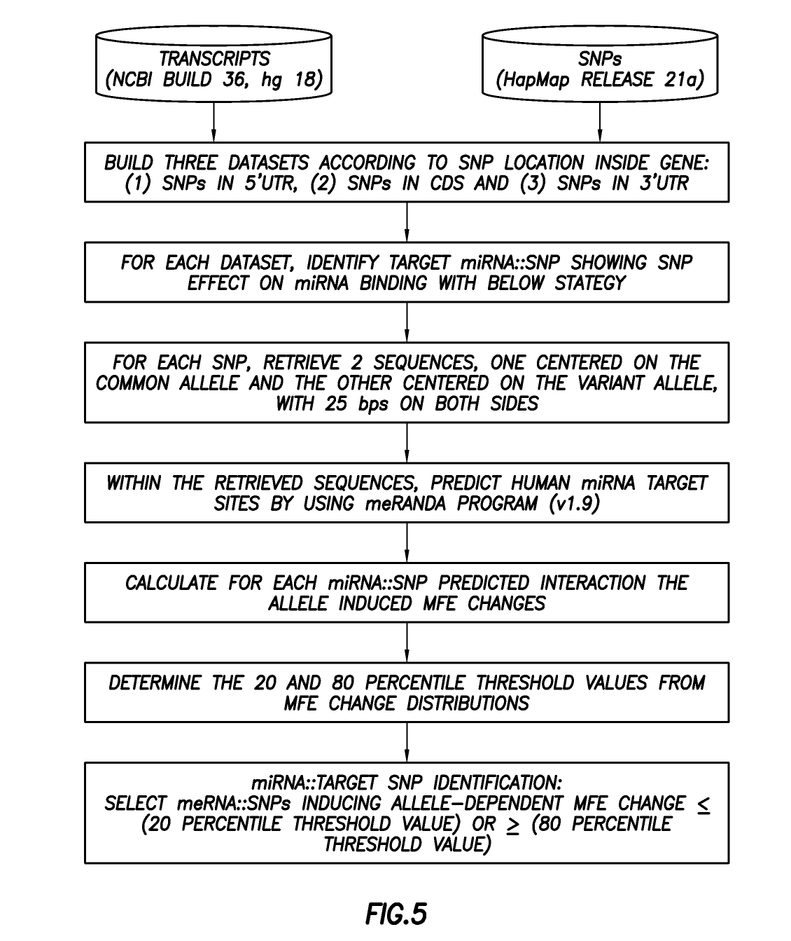

For each SNP, we retrieved 2 sequences, centered on each allele with 25 nucleotides flanking both sides. We used the miRNA target prediction program miRanda (2) with two different cut-offs (score≧80, MFE≦−16 Kcal/mol and score ≧50, MFE≦−5 Kcal/mol) to calculate minimum free energy (“MFE”) for all the possible miRNA::SNP centered sequences. For all the predicted interactions, we computed the allele dependent MFE changes. Based on the distribution of MFE changes, we identified the 20 and 80 percentile threshold values for miRNA::target SNP identification (FIG. 5). Based on these calculations, we termed SNP alleles as either active or non-active alleles, the active allele being the variant that induces a decrease in MFE and a stronger miRNA binding with the target.

Patient Sample Collection.

Blood samples from patients with familial and sporadic BC and from control subjects were collected at the Istituto Nazionale Tumori (INT), Milan Italy with their informed consent. Total genomic DNA was purified from peripheral blood leukocytes using the QIAamp® DNA mini kit (Qiagen).

Selection of SNPs Associated with Breast Cancer.

We performed a literature search by use of the MedLine/PubMed database covering the time between January 2006 and December 2008 in order to retrieve papers reporting association studies between SNPs and BC risk. We selected SNPs that were confirmed to be associated with BC risk in one or more papers and/or from large-scale genome wide association studies. Table 1 (above) shows the initial list of SNPs use for subsequent miRanda analysis.

DNA Sequencing and SNP Cloning.

PCR amplification of the transcribed SNP-containing region was performed on genomic DNA (Platinum Taq High Fidelity; Invitrogen). Primers are available upon request. Sequences were performed using the BigDye Terminator Reaction Chemistry v3.1 on Applied Biosystems 3730 DNA analyzers (Applied Biosystems). All sequence analyses and alignments were performed with the SeqmanPro program, Lasergene version 7.1 (DNASTAR). PCR amplified SNP-containing regions carrying either the active or NON-active alleles were XbaI cloned into the 3′ UTR of the pGL3-control vector (Promega).

Cell Cultures, Transfection and Immunoblotting.

All cell lines were obtained by ATCC. Cells were transfected using Lipofectamine 2000 (Invitrogen). Total protein extracts were prepared in 0.5% NP40 lysis buffer. For immunoblotting, protein extracts were separated in 4% to 20% SDS-PAGE (Criterion Precast Gel; Bio-Rad) and transferred to nitrocellulose membranes (Bio-Rad). Antibodies for TGFB, TGFBR1 and XRCC1 were from Cell Signaling; for BRCA1 from Calbiochem (clone MS110) and for Vinculin from Santa Cruz (clone N-19). Bands were quantified using GelDoc XR software (Biorad).

Luciferase Assay.

MCF7 cells (200×10⁵ cells per 24-well) were co-transfected with 0.4 ug of pGL3 (luciferase), 0.08 ug of pRLTK (renilla) (Promega) and 50 nM of precursor miRNA molecules or scrambled negative control (Ambion). Thirty hours after transfection, cells were lysed in 100 ul of passive lysis buffer according to the dual luciferase reporter assay protocol (Promega) and luciferase activity measured with Veritas luminometer (Turner BioSytems).

Statistical Analysis.

Fisher's exact test was used to determine the association of SNPs and patient disease status. Cochran-Armitage tend test was used to investigate whether there was a trend in binomial proportions of cancer patients (Familiar+Sporadic, Familiar, or Sporadic respectively) across levels of the different genotypes for each SNP. The univariate and multivariate logistic regression models were fit to evaluate the association of SNP genotype with patient state status.

Patient Information.

Cases were 335 female patients affected with invasive BC. Familial BC cases were ascertained through the Medical Genetics Unit of the INT Milan, as eligible for mutation testing in BRCA1 and BRCA2 genes, based on criteria including family history and age at cancer diagnosis (1). Mutation analysis was carried out as previously described Filipowicz, W. et al., Mechanisms of Post-Transcriptional Regulation by microRNAs: Are the Answers In Sight? 9:102-114, Nat Rev Genet (2008). Only individuals who tested negative for deleterious mutations in coding sequences of both genes were included in the study. This group included 169 women with BC (median age at diagnosis: 44; range: 21-77). Sporadic BC cases included 166 consecutive women at first diagnosis of BC, surgically treated at INT Milan between November 2004 and August 2005 and unselected for family history of cancer (median age at diagnosis: 56; range: 23-97). Controls were 186 Italian female blood donors recruited through the Immunohematology and Transfusion Medicine Service of INT Milan (median age: 56; range: 48-71).

Statistical Analysis.

For multivariate analysis, initially a full model including SNPs with p value less than 0.1 in the univariate analysis and age were fitted, then a backward selection procedure was used for model selection until all variables retained in the model were significant at the 0.05 significance level. The univariate and multivariate logistic regression analyses were performed for the following comparisons: Familiar+Sporadic vs. Control, Familiar vs. Control, Sporadic vs. control and Familiar vs. Sporadic.

RNA Extraction, Retrotranscription and Realtime PCR.

Cells total RNA was isolated from using TRIzol reagent (Invitrogen). For quantification of transfected and/or endogenous mature miRNA levels (data not shown) we used TaqMan® MicroRNA Reverse Transcription Kit, TaqMan® MicroRNA assays together with TaqMan® Universal PCR Master Mix, No AmpErase® UNG (Applied Biosystems). We employed the 2-Delta Ct method to calculate the relative abundance of microRNA compared with RNU6B expression (2). Realtime PCR reaction and analyses were carried out in 96-well optical reaction plates using iQ5 MultiColor Detection system (Biorad).

### Results

Analysis of Bc Susceptibility Alleles for their Ability to Affect miRNA Binding.

In order to test our hypothesis that SNPs can affect BC susceptibility by altering miRNA::mRNA binding, we took advantage of genetic variants that are already known to be associated with BC (see Methods and Table I above). For each of these SNPs, we retrieved two sequences 51 nucleotides long, one centered on the common allele and the other centered on the variant allele. We scanned the retrieved sequences with miRanda in search of miRNA::mRNA target sites and calculated the MFE changes induced by the allelic variants. Miranda KC, et al., A Pattern-Based Method for the Identification of MicroRNA Binding Sites and Their Corresponding Heteroduplexes, 126:1203-1217, Cell (2006).

TABLE II

| | | | | | | Score | MFE | |
|---|---|---|---|---|---|---|---|---|
| | | | | | | for | for | |
| | | | | | Active | miRNA: | miRNA: | |
| | Gene | | | Allelic | Allele | mRNA- | mRNA- | MFE |
| SNP ID | Symbol | Location | miRNA ID | Variants | (AA) | AA | AA | Change % |
| rs876755 | ACAD9 | CDS | hsa-let-7i* | C/T | C | 117 | C/T | −25.02% |
| rs876755 | ACAD9 | CDS | hsa-miR- | C/T | C | 142 | C/T | −17.17% |
| | | | 106b* | | | | | |
| rs876755 | ACAD9 | CDS | hsa-miR- | C/T | T | 90 | C/T | 9.96% |
| | | | 10a* | | | | | |
| rs876755 | ACAD9 | CDS | hsa-miR- | C/T | C | 104 | C/T | −8.43% |
| | | | 1180 | | | | | |
| rs876755 | ACAD9 | CDS | hsa-miR- | C/T | T | 108 | C/T | 12.02% |
| | | | 1203 | | | | | |
| rs876755 | ACAD9 | CDS | hsa-miR-122 | C/T | C | 126 | C/T | −12.02% |
| rs876755 | ACAD9 | CDS | hsa-miR- | C/T | C | 102 | C/T | −14.84% |
| | | | 1224-5p | | | | | |
| rs876755 | ACAD9 | CDS | hsa-miR- | C/T | C | 122 | C/T | −11.84% |
| | | | 1226* | | | | | |
| rs876755 | ACAD9 | CDS | hsa-miR- | C/T | T | 88 | C/T | 13.00% |
| | | | 1228* | | | | | |
| rs876755 | ACAD9 | CDS | hsa-miR- | C/T | C | 125 | C/T | −31.90% |
| | | | 1231 | | | | | |
| rs876755 | ACAD9 | CDS | hsa-miR- | C/T | C | 109 | C/T | −23.45% |
| | | | 1266 | | | | | |
| rs876755 | ACAD9 | CDS | hsa-miR- | C/T | C | 139 | C/T | −14.32% |
| | | | 1269 | | | | | |
| rs876755 | ACAD9 | CDS | hsa-miR- | C/T | C | 113 | C/T | −16.25% |
| | | | 1288 | | | | | |
| rs876755 | ACAD9 | CDS | hsa-miR-129- | C/T | C | 102 | C/T | −15.66% |
| | | | 5p | | | | | |
| rs876755 | ACAD9 | CDS | hsa-miR- | C/T | T | 111 | C/T | 107.76 |
| | | | 1296 | | | | | |
| rs876755 | ACAD9 | CDS | hsa-miR- | C/T | C | 85 | C/T | −16.88% |
| | | | 1303 | | | | | |
| rs876755 | ACAD9 | CDS | hsa-miR- | C/T | C | 128 | C/T | −12.00% |
| | | | 1323 | | | | | |
| rs876755 | ACAD9 | CDS | hsa-miR- | C/T | T | 110 | C/T | 22.41% |
| | | | 135a* | | | | | |
| rs876755 | ACAD9 | CDS | hsa-miR- | C/T | T | 128 | C/T | 17.18% |
| | | | 135b* | | | | | |
| rs876755 | ACAD9 | CDS | hsa-miR-138- | C/T | T | 93 | C/T | 12.82% |
| | | | 1* | | | | | |
| rs876755 | ACAD9 | CDS | hsa-miR-138- | C/T | T | 82 | C/T | 12.77% |
| | | | 2* | | | | | |
| rs876755 | ACAD9 | CDS | hsa-miR-139- | C/T | T | 107 | C/T | 92.09% |
| | | | 5p | | | | | |
| rs876755 | ACAD9 | CDS | hsa-miR-141 | C/T | C | 133 | C/T | −9.47% |
| rs876755 | ACAD9 | CDS | hsa-miR- | C/T | C | 101 | C/T | −14.22% |
| | | | 143* | | | | | |
| rs876755 | ACAD9 | CDS | hsa-miR- | C/T | T | 101 | C/T | 33.33% |
| | | | 1471 | | | | | |
| rs876755 | ACAD9 | CDS | hsa-miR- | C/T | C | 122 | C/T | −21.67% |
| | | | 150* | | | | | |
| rs876755 | ACAD9 | CDS | hsa-miR-151- | C/T | C | 131 | C/T | −8.15% |
| | | | 3p | | | | | |
| rs876755 | ACAD9 | CDS | hsa-miR-154 | C/T | T | 86 | C/T | 100.58 |
| rs876755 | ACAD9 | CDS | hsa-miR-17* | C/T | C | 96 | C/T | −32.31% |
| rs876755 | ACAD9 | CDS | hsa-miR- | C/T | C | 125 | C/T | −10.44% |
| | | | 181a-2* | | | | | |
| rs876755 | ACAD9 | CDS | hsa-miR- | C/T | C | 119 | C/T | −91.78% |
| | | | 185* | | | | | |
| rs876755 | ACAD9 | CDS | hsa-miR- | C/T | C | 91 | C/T | −11.95% |
| | | | 186* | | | | | |
| rs876755 | ACAD9 | CDS | hsa-miR- | C/T | C | 122 | C/T | −16.79% |
| | | | 1908 | | | | | |
| rs876755 | ACAD9 | CDS | hsa-miR- | C/T | C | 93 | C/T | −29.81% |
| | | | 1915 | | | | | |
| rs876755 | ACAD9 | CDS | hsa-miR-194 | C/T | T | 80 | C/T | 9.75% |
| rs876755 | ACAD9 | CDS | hsa-miR- | C/T | C | 108 | C/T | −27.75% |
| | | | 194* | | | | | |
| rs876755 | ACAD9 | CDS | hsa-miR- | C/T | C | 112 | C/T | −16.75% |
| | | | 195* | | | | | |
| rs876755 | ACAD9 | CDS | hsa-miR- | C/T | T | 86 | C/T | 37.86% |
| | | | 196a | | | | | |
| rs876755 | ACAD9 | CDS | hsa-miR- | C/T | T | 153 | C/T | 23.93% |
| | | | 1977 | | | | | |
| rs876755 | ACAD9 | CDS | hsa-miR-198 | C/T | C | 120 | C/T | −14.16% |
| rs876755 | ACAD9 | CDS | hsa-miR- | C/T | C | 83 | C/T | −22.91% |
| | | | 199a-3p | | | | | |
| rs876755 | ACAD9 | CDS | hsa-miR- | C/T | C | 83 | C/T | −22.91% |
| | | | 199b-3p | | | | | |
| rs876755 | ACAD9 | CDS | hsa-miR-206 | C/T | C | 104 | C/T | −16.38% |
| rs876755 | ACAD9 | CDS | hsa-miR-210 | C/T | T | 120 | C/T | 8.80% |
| rs876755 | ACAD9 | CDS | hsa-miR- | C/T | C | 112 | C/T | −15.13% |
| | | | 2110 | | | | | |
| rs876755 | ACAD9 | CDS | hsa-miR- | C/T | C | 140 | C/T | −8.85% |
| | | | 214* | | | | | |
| rs876755 | ACAD9 | CDS | hsa-miR-221 | C/T | C | 82 | C/T | −8.31% |
| rs876755 | ACAD9 | CDS | hsa-miR- | C/T | C | 88 | C/T | −19.77% |
| | | | 23a* | | | | | |
| rs876755 | ACAD9 | CDS | hsa-miR-25* | C/T | T | 89 | C/T | 38.35% |
| rs876755 | ACAD9 | CDS | hsa-miR-296- | C/T | C | 92 | C/T | −88.74% |
| | | | 3p | | | | | |
| rs876755 | ACAD9 | CDS | hsa-miR- | C/T | C | 100 | C/T | −45.32% |
| | | | 30b* | | | | | |
| rs876755 | ACAD9 | CDS | hsa-miR-324- | C/T | T | 95 | C/T | 31.53% |
| | | | 5p | | | | | |
| rs876755 | ACAD9 | CDS | hsa-miR-342- | C/T | C | 177 | C/T | −16.57% |
| | | | 5p | | | | | |
| rs876755 | ACAD9 | CDS | hsa-miR-34c- | C/T | C | 132 | C/T | −14.26% |
| | | | 5p | | | | | |
| rs876755 | ACAD9 | CDS | hsa-miR-362- | C/T | T | 87 | C/T | 92.59% |
| | | | 5p | | | | | |
| rs876755 | ACAD9 | CDS | hsa-miR-371- | C/T | C | 115 | C/T | −16.70% |
| | | | 5p | | | | | |
| rs876755 | ACAD9 | CDS | hsa-miR- | C/T | C | 107 | C/T | −13.51% |
| | | | 373* | | | | | |
| rs876755 | ACAD9 | CDS | hsa-miR-432 | C/T | T | 91 | C/T | 54.16% |
| rs876755 | ACAD9 | CDS | hsa-miR- | C/T | C | 103 | C/T | −30.45% |
| | | | 432* | | | | | |
| rs876755 | ACAD9 | CDS | hsa-miR- | C/T | C | 143 | C/T | −13.85% |
| | | | 449a | | | | | |
| rs876755 | ACAD9 | CDS | hsa-miR- | C/T | C | 139 | C/T | −15.71% |
| | | | 449b | | | | | |
| rs876755 | ACAD9 | CDS | hsa-miR-452 | C/T | C | 122 | C/T | −12.90% |
| rs876755 | ACAD9 | CDS | hsa-miR-485- | C/T | C | 124 | C/T | −18.96% |
| | | | 5p | | | | | |
| rs876755 | ACAD9 | CDS | hsa-miR-486- | C/T | C | 104 | C/T | −18.11% |
| | | | 3p | | | | | |
| rs876755 | ACAD9 | CDS | hsa-miR-490- | C/T | C | 116 | C/T | −16.07% |
| | | | 3p | | | | | |
| rs876755 | ACAD9 | CDS | hsa-miR-491- | C/T | C | 126 | C/T | −13.13% |
| | | | 5p | | | | | |
| rs876755 | ACAD9 | CDS | hsa-miR-498 | C/T | C | 103 | C/T | −9.61% |
| rs876755 | ACAD9 | CDS | hsa-miR- | C/T | C | 95 | C/T | −48.04% |
| | | | 518a-3p | | | | | |
| rs876755 | ACAD9 | CDS | hsa-miR- | C/T | T | 105 | C/T | 21.07% |
| | | | 518f* | | | | | |
| rs876755 | ACAD9 | CDS | hsa-miR-521 | C/T | T | 122 | C/T | 18.68% |
| rs876755 | ACAD9 | CDS | hsa-miR-523 | C/T | T | 101 | C/T | 8.95% |
| rs876755 | ACAD9 | CDS | hsa-miR-524- | C/T | C | 114 | C/T | −26.99% |
| | | | 3p | | | | | |
| rs876755 | ACAD9 | CDS | hsa-miR-557 | C/T | C | 127 | C/T | −14.68% |
| rs876755 | ACAD9 | CDS | hsa-miR-572 | C/T | C | 124 | C/T | −9.08% |
| rs876755 | ACAD9 | CDS | hsa-miR-593 | C/T | C | 106 | C/T | −17.03% |
| rs876755 | ACAD9 | CDS | hsa-miR- | C/T | T | 113 | C/T | 19.09% |
| | | | 593* | | | | | |
| rs876755 | ACAD9 | CDS | hsa-miR-604 | C/T | T | 119 | C/T | 27.34% |
| rs876755 | ACAD9 | CDS | hsa-miR-609 | C/T | T | 116 | C/T | 11.84% |
| rs876755 | ACAD9 | CDS | hsa-miR-611 | C/T | C | 108 | C/T | −12.59% |
| rs876755 | ACAD9 | CDS | hsa-miR-612 | C/T | C | 126 | C/T | −24.28% |
| rs876755 | ACAD9 | CDS | hsa-miR-615- | C/T | C | 112 | C/T | −10.22% |
| | | | 3p | | | | | |
| rs876755 | ACAD9 | CDS | hsa-miR-622 | C/T | C | 131 | C/T | −14.48% |
| rs876755 | ACAD9 | CDS | hsa-miR-625 | C/T | C | 115 | C/T | −9.68% |
| rs876755 | ACAD9 | CDS | hsa-miR-632 | C/T | C | 137 | C/T | −14.96% |
| rs876755 | ACAD9 | CDS | hsa-miR-637 | C/T | C | 147 | C/T | −13.14% |
| rs876755 | ACAD9 | CDS | hsa-miR-638 | C/T | T | 102 | C/T | 8.56% |
| rs876755 | ACAD9 | CDS | hsa-miR-639 | C/T | C | 99 | C/T | −10.45% |
| rs876755 | ACAD9 | CDS | hsa-miR-650 | C/T | T | 80 | C/T | 9.73% |
| rs876755 | ACAD9 | CDS | hsa-miR-652 | C/T | T | 128 | C/T | 21.18% |
| rs876755 | ACAD9 | CDS | hsa-miR-657 | C/T | T | 84 | C/T | 26.16% |
| rs876755 | ACAD9 | CDS | hsa-miR-661 | C/T | C | 134 | C/T | −58.84% |
| rs876755 | ACAD9 | CDS | hsa-miR-663 | C/T | C | 129 | C/T | −12.96% |
| rs876755 | ACAD9 | CDS | hsa-miR- | C/T | C | 131 | C/T | −12.74% |
| | | | 663b | | | | | |
| rs876755 | ACAD9 | CDS | hsa-miR-744 | C/T | T | 133 | C/T | 19.20% |
| rs876755 | ACAD9 | CDS | hsa-miR-760 | C/T | C | 140 | C/T | −14.76% |
| rs876755 | ACAD9 | CDS | hsa-miR-769- | C/T | C | 110 | C/T | −17.93% |
| | | | 3p | | | | | |
| rs876755 | ACAD9 | CDS | hsa-miR-885- | C/T | C | 96 | C/T | −12.26% |
| | | | 3p | | | | | |
| rs876755 | ACAD9 | CDS | hsa-miR- | C/T | C | 103 | C/T | −26.55% |
| | | | 888* | | | | | |
| rs876755 | ACAD9 | CDS | hsa-miR- | C/T | C | 112 | C/T | −9.08% |
| | | | 892b | | | | | |
| rs876755 | ACAD9 | CDS | hsa-miR-921 | C/T | T | 107 | C/T | 27.11% |
| rs876755 | ACAD9 | CDS | hsa-miR-92a | C/T | C | 119 | C/T | −11.89% |
| rs876755 | ACAD9 | CDS | hsa-miR-93* | C/T | T | 88 | C/T | 76.17% |
| rs876755 | ACAD9 | CDS | hsa-miR-939 | C/T | C | 158 | C/T | −12.49% |
| rs876755 | ACAD9 | CDS | hsa-miR-940 | C/T | T | 123 | C/T | 14.36% |
| rs876755 | ACAD9 | CDS | hsa-miR-943 | C/T | C | 112 | C/T | −106.5% |
| rs2241766 | ADIPOQ | CDS | hsa-let-7a | G/T | T | 87 | G/T | 12.51% |
| rs2241766 | ADIPOQ | CDS | hsa-miR-103- | G/T | G | 119 | G/T | −42.07% |
| | | | as | | | | | |
| rs2241766 | ADIPOQ | CDS | hsa-miR- | G/T | G | 114 | G/T | −14.85% |
| | | | 1180 | | | | | |
| rs2241766 | ADIPOQ | CDS | hsa-miR- | G/T | T | 91 | G/T | 11.66% |
| | | | 1183 | | | | | |
| rs2241766 | ADIPOQ | CDS | hsa-miR- | G/T | T | 90 | G/T | 8.38% |
| | | | 1202 | | | | | |
| rs2241766 | ADIPOQ | CDS | hsa-miR- | G/T | G | 129 | G/T | −36.91% |
| | | | 1203 | | | | | |
| rs2241766 | ADIPOQ | CDS | hsa-miR- | G/T | T | 103 | G/T | 20.72% |
| | | | 1208 | | | | | |
| rs2241766 | ADIPOQ | CDS | hsa-miR- | G/T | T | 100 | G/T | 21.13% |
| | | | 1224-5p | | | | | |
| rs2241766 | ADIPOQ | CDS | hsa-miR- | G/T | T | 102 | G/T | 8.73% |
| | | | 1225-5p | | | | | |
| rs2241766 | ADIPOQ | CDS | hsa-miR- | G/T | G | 90 | G/T | −35.06% |
| | | | 1226* | | | | | |
| rs2241766 | ADIPOQ | CDS | hsa-miR- | G/T | G | 140 | G/T | −18.86% |
| | | | 1231 | | | | | |
| rs2241766 | ADIPOQ | CDS | hsa-miR- | G/T | G | 108 | G/T | −27.50% |
| | | | 1233 | | | | | |
| rs2241766 | ADIPOQ | CDS | hsa-miR- | G/T | G | 132 | G/T | −31.32% |
| | | | 1238 | | | | | |
| rs2241766 | ADIPOQ | CDS | hsa-miR- | G/T | G | 105 | G/T | −26.23% |
| | | | 1247 | | | | | |
| rs2241766 | ADIPOQ | CDS | hsa-miR- | G/T | T | 83 | G/T | 15.62% |
| | | | 1250 | | | | | |
| rs2241766 | ADIPOQ | CDS | hsa-miR- | G/T | G | 139 | G/T | −23.71% |
| | | | 1254 | | | | | |
| rs2241766 | ADIPOQ | CDS | hsa-miR- | G/T | T | 100 | G/T | 11.83% |
| | | | 125a-3p | | | | | |
| rs2241766 | ADIPOQ | CDS | hsa-miR- | G/T | G | 125 | G/T | −15.86% |
| | | | 125b-1* | | | | | |
| rs2241766 | ADIPOQ | CDS | hsa-miR-126 | G/T | T | 116 | G/T | 20.92% |
| rs2241766 | ADIPOQ | CDS | hsa-miR- | G/T | T | 119 | G/T | 9.80% |
| | | | 1263 | | | | | |
| rs2241766 | ADIPOQ | CDS | hsa-miR- | G/T | G | 112 | G/T | −13.06% |
| | | | 1269 | | | | | |
| rs2241766 | ADIPOQ | CDS | hsa-miR- | G/T | G | 114 | G/T | −46.30% |
| | | | 1288 | | | | | |
| rs2241766 | ADIPOQ | CDS | hsa-miR-129- | G/T | G | 92 | G/T | −26.72% |
| | | | 5p | | | | | |
| rs2241766 | ADIPOQ | CDS | hsa-miR- | G/T | T | 128 | G/T | 27.91% |
| | | | 1291 | | | | | |
| rs2241766 | ADIPOQ | CDS | hsa-miR- | G/T | G | 89 | G/T | −33.82% |
| | | | 1292 | | | | | |
| rs2241766 | ADIPOQ | CDS | hsa-miR- | G/T | G | 92 | G/T | −9.67% |
| | | | 1299 | | | | | |
| rs2241766 | ADIPOQ | CDS | hsa-miR- | G/T | T | 88 | G/T | 11.68% |
| | | | 1308 | | | | | |
| rs2241766 | ADIPOQ | CDS | hsa-miR-134 | G/T | T | 122 | G/T | 36.69% |
| rs2241766 | ADIPOQ | CDS | hsa-miR- | G/T | T | 136 | G/T | 10.69% |
| | | | 145* | | | | | |
| rs2241766 | ADIPOQ | CDS | hsa-miR- | G/T | G | 114 | G/T | −17.59% |
| | | | 1469 | | | | | |
| rs2241766 | ADIPOQ | CDS | hsa-miR- | G/T | G | 112 | G/T | −41.55% |
| | | | 146b-3p | | | | | |
| rs2241766 | ADIPOQ | CDS | hsa-miR- | G/T | G | 127 | G/T | −46.00% |
| | | | 1471 | | | | | |
| rs2241766 | ADIPOQ | CDS | hsa-miR- | G/T | G | 131 | G/T | −14.02% |
| | | | 150* | | | | | |
| rs2241766 | ADIPOQ | CDS | hsa-miR- | G/T | G | 165 | G/T | −35.37% |
| | | | 1538 | | | | | |
| rs2241766 | ADIPOQ | CDS | hsa-miR-17* | G/T | T | 82 | G/T | 9.51% |
| rs2241766 | ADIPOQ | CDS | hsa-miR- | G/T | T | 130 | G/T | 15.97% |
| | | | 1912 | | | | | |
| rs2241766 | ADIPOQ | CDS | hsa-miR- | G/T | G | 123 | G/T | −47.17% |
| | | | 1914 | | | | | |
| rs2241766 | ADIPOQ | CDS | hsa-miR- | G/T | G | 128 | G/T | −20.78% |
| | | | 1915 | | | | | |
| rs2241766 | ADIPOQ | CDS | hsa-miR- | G/T | G | 125 | G/T | −34.52% |
| | | | 1915* | | | | | |
| rs2241766 | ADIPOQ | CDS | hsa-miR- | G/T | G | 118 | G/T | −13.18% |
| | | | 1972 | | | | | |
| rs2241766 | ADIPOQ | CDS | hsa-miR- | G/T | G | 105 | G/T | −22.52% |
| | | | 1974 | | | | | |
| rs2241766 | ADIPOQ | CDS | hsa-miR- | G/T | T | 93 | G/T | 25.79% |
| | | | 1977 | | | | | |
| rs2241766 | ADIPOQ | CDS | hsa-miR-198 | G/T | T | 106 | G/T | 32.19% |
| rs2241766 | ADIPOQ | CDS | hsa-miR-205 | G/T | G | 119 | G/T | −11.63% |
| rs2241766 | ADIPOQ | CDS | hsa-miR-210 | G/T | T | 100 | G/T | 11.66% |
| rs2241766 | ADIPOQ | CDS | hsa-miR-218- | G/T | G | 137 | G/T | −14.98% |
| | | | 1* | | | | | |
| rs2241766 | ADIPOQ | CDS | hsa-miR-218- | G/T | G | 120 | G/T | −8.51% |
| | | | 2* | | | | | |
| rs2241766 | ADIPOQ | CDS | hsa-miR-219- | G/T | G | 129 | G/T | −23.84% |
| | | | 1-3p | | | | | |
| rs2241766 | ADIPOQ | CDS | hsa-miR- | G/T | T | 91 | G/T | 11.11% |
| | | | 223* | | | | | |
| rs2241766 | ADIPOQ | CDS | hsa-miR-296- | G/T | G | 101 | G/T | −22.36% |
| | | | 3p | | | | | |
| rs2241766 | ADIPOQ | CDS | hsa-miR- | G/T | T | 93 | G/T | 88.66% |
| | | | 320a | | | | | |
| rs2241766 | ADIPOQ | CDS | hsa-miR- | G/T | T | 95 | G/T | 91.84% |
| | | | 320b | | | | | |
| rs2241766 | ADIPOQ | CDS | hsa-miR-323- | G/T | G | 116 | G/T | −30.60% |
| | | | 5p | | | | | |
| rs2241766 | ADIPOQ | CDS | hsa-miR-324- | G/T | G | 119 | G/T | −34.02% |
| | | | 3p | | | | | |
| rs2241766 | ADIPOQ | CDS | hsa-miR-324- | G/T | G | 117 | G/T | −31.37% |
| | | | 5p | | | | | |
| rs2241766 | ADIPOQ | CDS | hsa-miR- | G/T | G | 124 | G/T | −37.40% |
| | | | 33b* | | | | | |
| rs2241766 | ADIPOQ | CDS | hsa-miR-346 | G/T | G | 136 | G/T | −62.52% |
| rs2241766 | ADIPOQ | CDS | hsa-miR-362- | G/T | G | 109 | G/T | −15.05% |
| | | | 5p | | | | | |
| rs2241766 | ADIPOQ | CDS | hsa-miR-370 | G/T | G | 99 | G/T | −9.22% |
| rs2241766 | ADIPOQ | CDS | hsa-miR-382 | G/T | T | 110 | G/T | 17.67% |
| rs2241766 | ADIPOQ | CDS | hsa-miR-423- | G/T | G | 130 | G/T | −17.47% |
| | | | 3p | | | | | |
| rs2241766 | ADIPOQ | CDS | hsa-miR-453 | G/T | G | 118 | G/T | −27.81% |
| rs2241766 | ADIPOQ | CDS | hsa-miR-496 | G/T | T | 85 | G/T | 17.03% |
| rs2241766 | ADIPOQ | CDS | hsa-miR-500 | G/T | G | 103 | G/T | −17.97% |
| rs2241766 | ADIPOQ | CDS | hsa-miR- | G/T | G | 90 | G/T | −47.64% |
| | | | 500* | | | | | |
| rs2241766 | ADIPOQ | CDS | hsa-miR-501- | G/T | G | 120 | G/T | −16.94% |
| | | | 3p | | | | | |
| rs2241766 | ADIPOQ | CDS | hsa-miR-501- | G/T | G | 95 | G/T | −16.40% |
| | | | 5p | | | | | |
| rs2241766 | ADIPOQ | CDS | hsa-miR-502- | G/T | G | 120 | G/T | −8.68% |
| | | | 3p | | | | | |
| rs2241766 | ADIPOQ | CDS | hsa-miR-504 | G/T | G | 115 | G/T | −13.77% |
| rs2241766 | ADIPOQ | CDS | hsa-miR-509- | G/T | G | 104 | G/T | −9.11% |
| | | | 3p | | | | | |
| rs2241766 | ADIPOQ | CDS | hsa-miR-532- | G/T | G | 114 | G/T | −37.98% |
| | | | 5p | | | | | |
| rs2241766 | ADIPOQ | CDS | hsa-miR-542- | G/T | G | 112 | G/T | −55.19% |
| | | | 5p | | | | | |
| rs2241766 | ADIPOQ | CDS | hsa-miR-550 | G/T | G | 140 | G/T | −8.56% |
| rs2241766 | ADIPOQ | CDS | hsa-miR- | G/T | G | 80 | G/T | −39.71% |
| | | | 551b* | | | | | |
| rs2241766 | ADIPOQ | CDS | hsa-miR-552 | G/T | T | 85 | G/T | 60.48% |
| rs2241766 | ADIPOQ | CDS | hsa-miR-557 | G/T | G | 108 | G/T | −20.52% |
| rs2241766 | ADIPOQ | CDS | hsa-miR-572 | G/T | G | 97 | G/T | −31.18% |
| rs2241766 | ADIPOQ | CDS | hsa-miR-595 | G/T | G | 106 | G/T | −23.84% |
| rs2241766 | ADIPOQ | CDS | hsa-miR-596 | G/T | G | 102 | G/T | −16.52% |
| rs2241766 | ADIPOQ | CDS | hsa-miR-601 | G/T | T | 109 | G/T | 11.45% |
| rs2241766 | ADIPOQ | CDS | hsa-miR-602 | G/T | T | 133 | G/T | 11.97% |
| rs2241766 | ADIPOQ | CDS | hsa-miR-608 | G/T | G | 123 | G/T | −8.03% |
| rs2241766 | ADIPOQ | CDS | hsa-miR-611 | G/T | T | 95 | G/T | 15.06% |
| rs2241766 | ADIPOQ | CDS | hsa-miR-614 | G/T | G | 105 | G/T | −11.95% |
| rs2241766 | ADIPOQ | CDS | hsa-miR-615- | G/T | G | 112 | G/T | −23.77% |
| | | | 5p | | | | | |
| rs2241766 | ADIPOQ | CDS | hsa-miR-616 | G/T | T | 110 | G/T | 9.85% |
| rs2241766 | ADIPOQ | CDS | hsa-miR-619 | G/T | G | 139 | G/T | −13.74% |
| rs2241766 | ADIPOQ | CDS | hsa-miR-622 | G/T | T | 95 | G/T | 13.88% |
| rs2241766 | ADIPOQ | CDS | hsa-miR-623 | G/T | G | 103 | G/T | −23.28% |
| rs2241766 | ADIPOQ | CDS | hsa-miR-636 | G/T | G | 111 | G/T | −41.31% |
| rs2241766 | ADIPOQ | CDS | hsa-miR-637 | G/T | T | 100 | G/T | 10.22% |
| rs2241766 | ADIPOQ | CDS | hsa-miR-638 | G/T | T | 113 | G/T | 16.08% |
| rs2241766 | ADIPOQ | CDS | hsa-miR-659 | G/T | G | 104 | G/T | −35.81% |
| rs2241766 | ADIPOQ | CDS | hsa-miR-662 | G/T | T | 102 | G/T | 12.04% |
| rs2241766 | ADIPOQ | CDS | hsa-miR-663 | G/T | T | 117 | G/T | 14.47% |
| rs2241766 | ADIPOQ | CDS | hsa-miR- | G/T | G | 123 | G/T | −29.09% |
| | | | 663b | | | | | |
| rs2241766 | ADIPOQ | CDS | hsa-miR- | G/T | T | 103 | G/T | 21.32% |
| | | | 664* | | | | | |
| rs2241766 | ADIPOQ | CDS | hsa-miR-671- | G/T | G | 107 | G/T | −9.96% |
| | | | 3p | | | | | |
| rs2241766 | ADIPOQ | CDS | hsa-miR-671- | G/T | G | 120 | G/T | −21.08% |
| | | | 5p | | | | | |
| rs2241766 | ADIPOQ | CDS | hsa-miR-758 | G/T | G | 99 | G/T | −28.57% |
| rs2241766 | ADIPOQ | CDS | hsa-miR-767- | G/T | G | 132 | G/T | −15.17% |
| | | | 3p | | | | | |
| rs2241766 | ADIPOQ | CDS | hsa-miR-767- | G/T | T | 82 | G/T | 8.30% |
| | | | 5p | | | | | |
| rs2241766 | ADIPOQ | CDS | hsa-miR-770- | G/T | G | 91 | G/T | −15.49% |
| | | | 5p | | | | | |
| rs2241766 | ADIPOQ | CDS | hsa-miR-874 | G/T | T | 110 | G/T | 12.73% |
| rs2241766 | ADIPOQ | CDS | hsa-miR-92a- | G/T | T | 114 | G/T | 26.31% |
| | | | 1* | | | | | |
| rs2241766 | ADIPOQ | CDS | hsa-miR- | G/T | T | 106 | G/T | 10.93% |
| | | | 92b* | | | | | |
| rs7539542 | ADIPOR1 | 3′ UTR | hsa-miR- | C/G | G | 150 | C/G | 25.55% |
| | | | 1909* | | | | | |
| rs7539542 | ADIPOR1 | 3′ UTR | hsa-miR- | C/G | G | 133 | C/G | 21.25% |
| | | | 199b-5p | | | | | |
| rs7539542 | ADIPOR1 | 3′ UTR | hsa-miR- | C/G | C | 120 | C/G | −12.48% |
| | | | 214* | | | | | |
| rs7539542 | ADIPOR1 | 3′ UTR | hsa-miR-25 | C/G | C | 89 | C/G | −213.35% |
| rs7539542 | ADIPOR1 | 3′ UTR | hsa-miR-34a | C/G | G | 112 | C/G | 15.20% |
| rs7539542 | ADIPOR1 | 3′ UTR | hsa-miR-361- | C/G | G | 101 | C/G | 29.55% |
| | | | 3p | | | | | |
| rs7539542 | ADIPOR1 | 3′ UTR | hsa-miR-511 | C/G | G | 132 | C/G | 39.95% |
| rs7539542 | ADIPOR1 | 3′ UTR | hsa-miR-515- | C/G | C | 116 | C/G | −67.22% |
| | | | 3p | | | | | |
| rs7539542 | ADIPOR1 | 3′ UTR | hsa-miR- | C/G | C | 94 | C/G | −27.97% |
| | | | 548d-3p | | | | | |
| rs7539542 | ADIPOR1 | 3′ UTR | hsa-miR-589 | C/G | C | 103 | C/G | −29.65% |
| rs7539542 | ADIPOR1 | 3′ UTR | hsa-miR-593 | C/G | C | 140 | C/G | −25.92% |
| rs7539542 | ADIPOR1 | 3′ UTR | hsa-miR-614 | C/G | G | 98 | C/G | 26.84% |
| rs7539542 | ADIPOR1 | 3′ UTR | hsa-miR-661 | C/G | C | 99 | C/G | −26.05% |
| rs7539542 | ADIPOR1 | 3′ UTR | hsa-miR-939 | C/G | C | 87 | C/G | −15.15% |
| rs7539542 | ADIPOR1 | 3′ UTR | hsa-miR- | C/G | C | 100 | C/G | −30.45% |
| | | | 99b* | | | | | |
| rs1130409 | APEX1 | CDS | hsa-let-7a-2* | G/T | G | 89 | G/T | −27.66% |
| rs1130409 | APEX1 | CDS | hsa-let-7g* | G/T | G | 86 | G/T | −21.77% |
| rs1130409 | APEX1 | CDS | hsa-let-7i* | G/T | G | 93 | G/T | −16.02% |
| rs1130409 | APEX1 | CDS | hsa-miR-103- | G/T | G | 120 | G/T | −11.93% |
| | | | 2* | | | | | |
| rs1130409 | APEX1 | CDS | hsa-miR-105 | G/T | G | 107 | G/T | −10.43% |
| rs1130409 | APEX1 | CDS | hsa-miR- | G/T | G | 117 | G/T | −31.91% |
| | | | 1178 | | | | | |
| rs1130409 | APEX1 | CDS | hsa-miR- | G/T | G | 134 | G/T | −15.57% |
| | | | 1200 | | | | | |
| rs1130409 | APEX1 | CDS | hsa-miR- | G/T | T | 120 | G/T | 45.37% |
| | | | 1224-3p | | | | | |
| rs1130409 | APEX1 | CDS | hsa-miR- | G/T | G | 112 | G/T | −52.63% |
| | | | 1228 | | | | | |
| rs1130409 | APEX1 | CDS | hsa-miR- | G/T | G | 99 | G/T | −42.68% |
| | | | 1233 | | | | | |
| rs1130409 | APEX1 | CDS | hsa-miR- | G/T | G | 139 | G/T | −14.01% |
| | | | 1234 | | | | | |
| rs1130409 | APEX1 | CDS | hsa-miR- | G/T | G | 145 | G/T | −38.62% |
| | | | 1236 | | | | | |
| rs1130409 | APEX1 | CDS | hsa-miR- | G/T | G | 131 | G/T | −25.09% |
| | | | 1237 | | | | | |
| rs1130409 | APEX1 | CDS | hsa-miR- | G/T | G | 156 | G/T | −23.55% |
| | | | 1238 | | | | | |
| rs1130409 | APEX1 | CDS | hsa-miR- | G/T | G | 144 | G/T | −39.47% |
| | | | 1247 | | | | | |
| rs1130409 | APEX1 | CDS | hsa-miR- | G/T | T | 136 | G/T | 8.56% |
| | | | 1248 | | | | | |
| rs1130409 | APEX1 | CDS | hsa-miR- | G/T | G | 137 | G/T | −11.06% |
| | | | 1249 | | | | | |
| rs1130409 | APEX1 | CDS | hsa-miR- | G/T | G | 86 | G/T | −59.71% |
| | | | 1250 | | | | | |
| rs1130409 | APEX1 | CDS | hsa-miR- | G/T | T | 94 | G/T | 18.83% |
| | | | 125b-1* | | | | | |
| rs1130409 | APEX1 | CDS | hsa-miR- | G/T | T | 98 | G/T | 8.34% |
| | | | 1266 | | | | | |
| rs1130409 | APEX1 | CDS | hsa-miR- | G/T | G | 86 | G/T | −23.29% |
| | | | 1269 | | | | | |
| rs1130409 | APEX1 | CDS | hsa-miR- | G/T | G | 144 | G/T | −45.71% |
| | | | 1281 | | | | | |
| rs1130409 | APEX1 | CDS | hsa-miR- | G/T | G | 125 | G/T | −13.46% |
| | | | 1296 | | | | | |
| rs1130409 | APEX1 | CDS | hsa-miR- | G/T | T | 98 | G/T | 33.03% |
| | | | 135a | | | | | |
| rs1130409 | APEX1 | CDS | hsa-miR- | G/T | T | 104 | G/T | 30.41% |
| | | | 135b | | | | | |
| rs1130409 | APEX1 | CDS | hsa-miR- | G/T | T | 134 | G/T | 42.76% |
| | | | 136* | | | | | |
| rs1130409 | APEX1 | CDS | hsa-miR- | G/T | G | 106 | G/T | −48.22% |
| | | | 1469 | | | | | |
| rs1130409 | APEX1 | CDS | hsa-miR- | G/T | G | 127 | G/T | −14.40% |
| | | | 1470 | | | | | |
| rs1130409 | APEX1 | CDS | hsa-miR- | G/T | T | 87 | G/T | 51.76% |
| | | | 181a | | | | | |
| rs1130409 | APEX1 | CDS | hsa-miR- | G/T | T | 84 | G/T | 102.85 |
| | | | 181b | | | | | |
| rs1130409 | APEX1 | CDS | hsa-miR- | G/T | G | 82 | G/T | −39.62% |
| | | | 182* | | | | | |
| rs1130409 | APEX1 | CDS | hsa-miR- | G/T | G | 111 | G/T | −20.60% |
| | | | 1825 | | | | | |
| rs1130409 | APEX1 | CDS | hsa-miR- | G/T | G | 126 | G/T | −27.30% |
| | | | 18a* | | | | | |
| rs1130409 | APEX1 | CDS | hsa-miR- | G/T | G | 110 | G/T | −53.18% |
| | | | 18b* | | | | | |
| rs1130409 | APEX1 | CDS | hsa-miR- | G/T | G | 82 | G/T | −12.99% |
| | | | 191* | | | | | |
| rs1130409 | APEX1 | CDS | hsa-miR- | G/T | G | 129 | G/T | −17.40% |
| | | | 1913 | | | | | |
| rs1130409 | APEX1 | CDS | hsa-miR- | G/T | G | 150 | G/T | −28.25% |
| | | | 1976 | | | | | |
| rs1130409 | APEX1 | CDS | hsa-miR-204 | G/T | G | 106 | G/T | −38.48% |
| rs1130409 | APEX1 | CDS | hsa-miR-205 | G/T | G | 117 | G/T | −11.05% |
| rs1130409 | APEX1 | CDS | hsa-miR-211 | G/T | T | 112 | G/T | 19.35% |
| rs1130409 | APEX1 | CDS | hsa-miR-218- | G/T | G | 100 | G/T | −34.30% |
| | | | 2* | | | | | |
| rs1130409 | APEX1 | CDS | hsa-miR-221 | G/T | T | 92 | G/T | 19.60% |
| rs1130409 | APEX1 | CDS | hsa-miR-25 | G/T | G | 96 | G/T | −29.82% |
| rs1130409 | APEX1 | CDS | hsa-miR- | G/T | G | 100 | G/T | −13.76% |
| | | | 26b* | | | | | |
| rs1130409 | APEX1 | CDS | hsa-miR- | G/T | G | 90 | G/T | −14.39% |
| | | | 29c* | | | | | |
| rs1130409 | APEX1 | CDS | hsa-miR-330- | G/T | G | 107 | G/T | −10.39% |
| | | | 5p | | | | | |
| rs1130409 | APEX1 | CDS | hsa-miR-331- | G/T | G | 114 | G/T | −8.04% |
| | | | 3p | | | | | |
| rs1130409 | APEX1 | CDS | hsa-miR-337- | G/T | G | 98 | G/T | −23.96% |
| | | | 3p | | | | | |
| rs1130409 | APEX1 | CDS | hsa-miR-339- | G/T | T | 124 | G/T | 12.82% |
| | | | 3p | | | | | |
| rs1130409 | APEX1 | CDS | hsa-miR-346 | G/T | G | 136 | G/T | −12.48% |
| rs1130409 | APEX1 | CDS | hsa-miR-362- | G/T | G | 127 | G/T | −8.49% |
| | | | 5p | | | | | |
| rs1130409 | APEX1 | CDS | hsa-miR- | G/T | G | 89 | G/T | −37.08% |
| | | | 376a* | | | | | |
| rs1130409 | APEX1 | CDS | hsa-miR- | G/T | G | 87 | G/T | −14.68% |
| | | | 432* | | | | | |
| rs1130409 | APEX1 | CDS | hsa-miR- | G/T | T | 114 | G/T | 37.57% |
| | | | 452* | | | | | |
| rs1130409 | APEX1 | CDS | hsa-miR-483- | G/T | G | 132 | G/T | −15.54% |
| | | | 3p | | | | | |
| rs1130409 | APEX1 | CDS | hsa-miR-484 | G/T | T | 135 | G/T | 20.04% |
| rs1130409 | APEX1 | CDS | hsa-miR-485- | G/T | G | 99 | G/T | −44.07% |
| | | | 3p | | | | | |
| rs1130409 | APEX1 | CDS | hsa-miR-486- | G/T | G | 112 | G/T | −26.27% |
| | | | 5p | | | | | |
| rs1130409 | APEX1 | CDS | hsa-miR-501- | G/T | G | 102 | G/T | −13.35% |
| | | | 5p | | | | | |
| rs1130409 | APEX1 | CDS | hsa-miR- | G/T | T | 84 | G/T | 81.02% |
| | | | 513c | | | | | |
| rs1130409 | APEX1 | CDS | hsa-miR- | G/T | G | 110 | G/T | −28.98% |
| | | | 517a | | | | | |
| rs1130409 | APEX1 | CDS | hsa-miR- | G/T | G | 94 | G/T | −25.13% |
| | | | 517b | | | | | |
| rs1130409 | APEX1 | CDS | hsa-miR- | G/T | G | 113 | G/T | −11.71% |
| | | | 517c | | | | | |
| rs1130409 | APEX1 | CDS | hsa-miR- | G/T | G | 96 | G/T | −16.76% |
| | | | 518a-3p | | | | | |
| rs1130409 | APEX1 | CDS | hsa-miR- | G/T | G | 102 | G/T | −35.61% |
| | | | 520a-3p | | | | | |
| rs1130409 | APEX1 | CDS | hsa-miR- | G/T | G | 106 | G/T | −29.15% |
| | | | 520d-3p | | | | | |
| rs1130409 | APEX1 | CDS | hsa-miR- | G/T | T | 91 | G/T | 8.05% |
| | | | 548b-3p | | | | | |
| rs1130409 | APEX1 | CDS | hsa-miR-554 | G/T | T | 88 | G/T | 21.11% |
| rs1130409 | APEX1 | CDS | hsa-miR-572 | G/T | G | 104 | G/T | −23.95% |
| rs1130409 | APEX1 | CDS | hsa-miR-576- | G/T | G | 106 | G/T | −38.49% |
| | | | 5p | | | | | |
| rs1130409 | APEX1 | CDS | hsa-miR-591 | G/T | T | 131 | G/T | 41.26% |
| rs1130409 | APEX1 | CDS | hsa-miR-596 | G/T | T | 131 | G/T | 8.75% |
| rs1130409 | APEX1 | CDS | hsa-miR-598 | G/T | T | 103 | G/T | 37.05% |
| rs1130409 | APEX1 | CDS | hsa-miR-613 | G/T | G | 96 | G/T | −23.73% |
| rs1130409 | APEX1 | CDS | hsa-miR-615- | G/T | G | 129 | G/T | −11.52% |
| | | | 3p | | | | | |
| rs1130409 | APEX1 | CDS | hsa-miR-623 | G/T | G | 110 | G/T | −32.13% |
| rs1130409 | APEX1 | CDS | hsa-miR- | G/T | G | 115 | G/T | −14.90% |
| | | | 629* | | | | | |
| rs1130409 | APEX1 | CDS | hsa-miR-631 | G/T | T | 118 | G/T | 36.56% |
| rs1130409 | APEX1 | CDS | hsa-miR-636 | G/T | G | 101 | G/T | −28.52% |
| rs1130409 | APEX1 | CDS | hsa-miR-659 | G/T | G | 84 | G/T | −21.70% |
| rs1130409 | APEX1 | CDS | hsa-miR-664 | G/T | G | 87 | G/T | −23.49% |
| rs1130409 | APEX1 | CDS | hsa-miR-720 | G/T | G | 92 | G/T | −23.30% |
| rs1130409 | APEX1 | CDS | hsa-miR-766 | G/T | T | 105 | G/T | 19.73% |
| rs1130409 | APEX1 | CDS | hsa-miR-767- | G/T | T | 133 | G/T | 16.16% |
| | | | 3p | | | | | |
| rs1130409 | APEX1 | CDS | hsa-miR-767- | G/T | T | 98 | G/T | 15.31% |
| | | | 5p | | | | | |
| rs1130409 | APEX1 | CDS | hsa-miR-769- | G/T | G | 85 | G/T | −15.90% |
| | | | 3p | | | | | |
| rs1130409 | APEX1 | CDS | hsa-miR-874 | G/T | G | 99 | G/T | −23.41% |
| rs1130409 | APEX1 | CDS | hsa-miR- | G/T | G | 141 | G/T | −28.14% |
| | | | 877* | | | | | |
| rs1130409 | APEX1 | CDS | hsa-miR- | G/T | G | 106 | G/T | −35.35% |
| | | | 888* | | | | | |
| rs1130409 | APEX1 | CDS | hsa-miR-9 | G/T | T | 89 | G/T | 25.21% |
| rs1130409 | APEX1 | CDS | hsa-miR-93* | G/T | G | 116 | G/T | −44.64% |
| rs1130409 | APEX1 | CDS | hsa-miR-935 | G/T | G | 81 | G/T | −22.21% |
| rs1130409 | APEX1 | CDS | hsa-miR-937 | G/T | T | 106 | G/T | 9.88% |
| rs1130409 | APEX1 | CDS | hsa-miR-940 | G/T | G | 81 | G/T | −10.84% |
| rs1130409 | APEX1 | CDS | hsa-miR-942 | G/T | G | 120 | G/T | −13.09% |
| rs1130409 | APEX1 | CDS | hsa-miR-943 | G/T | G | 92 | G/T | −20.64% |
| rs1801516 | ATM | CDS | hsa-miR-330- | A/G | G | 97 | A/G | 51.10% |
| | | | 5p | | | | | |
| rs1801516 | ATM | CDS | hsa-miR-432 | A/G | G | 164 | A/G | 35.40% |
| rs1801516 | ATM | CDS | hsa-miR-623 | A/G | G | 91 | A/G | 20.49% |
| rs28997576 | BARD1 | CDS | hsa-let-7b | C/G | C | 123 | C/G | −15.91% |
| rs28997576 | BARD1 | CDS | hsa-let-7c | C/G | C | 120 | C/G | −88.31% |
| rs28997576 | BARD1 | CDS | hsa-let-7i | C/G | C | 120 | C/G | −17.37% |
| rs28997576 | BARD1 | CDS | hsa-miR-103 | C/G | G | 94 | C/G | 93.76% |
| rs28997576 | BARD1 | CDS | hsa-miR- | C/G | G | 102 | C/G | 43.37% |
| | | | 105* | | | | | |
| rs28997576 | BARD1 | CDS | hsa-miR- | C/G | C | 109 | C/G | −22.34% |
| | | | 106b* | | | | | |
| rs28997576 | BARD1 | CDS | hsa-miR-107 | C/G | G | 93 | C/G | 20.52% |
| rs28997576 | BARD1 | CDS | hsa-miR- | C/G | G | 114 | C/G | 65.39% |
| | | | 1181 | | | | | |
| rs28997576 | BARD1 | CDS | hsa-miR- | C/G | G | 126 | C/G | 46.62% |
| | | | 1207-5p | | | | | |
| rs28997576 | BARD1 | CDS | hsa-miR- | C/G | G | 139 | C/G | 17.67% |
| | | | 1228* | | | | | |
| rs28997576 | BARD1 | CDS | hsa-miR- | C/G | G | 115 | C/G | 38.68% |
| | | | 1244 | | | | | |
| rs28997576 | BARD1 | CDS | hsa-miR- | C/G | G | 125 | C/G | 16.86% |
| | | | 1266 | | | | | |
| rs28997576 | BARD1 | CDS | hsa-miR- | C/G | G | 123 | C/G | 17.48% |
| | | | 1269 | | | | | |
| rs28997576 | BARD1 | CDS | hsa-miR-127- | C/G | G | 85 | C/G | 27.62% |
| | | | 3p | | | | | |
| rs28997576 | BARD1 | CDS | hsa-miR- | C/G | C | 95 | C/G | −31.34% |
| | | | 1270 | | | | | |
| rs28997576 | BARD1 | CDS | hsa-miR- | C/G | C | 116 | C/G | −36.87% |
| | | | 1275 | | | | | |
| rs28997576 | BARD1 | CDS | hsa-miR- | C/G | G | 123 | C/G | 11.28% |
| | | | 1291 | | | | | |
| rs28997576 | BARD1 | CDS | hsa-miR- | C/G | C | 130 | C/G | −10.92% |
| | | | 1293 | | | | | |
| rs28997576 | BARD1 | CDS | hsa-miR- | C/G | C | 115 | C/G | −41.04% |
| | | | 1294 | | | | | |
| rs28997576 | BARD1 | CDS | hsa-miR- | C/G | C | 105 | C/G | −15.42% |
| | | | 1301 | | | | | |
| rs28997576 | BARD1 | CDS | hsa-miR- | C/G | C | 104 | C/G | −22.39%. |
| | | | 1323 | | | | | |
| rs28997576 | BARD1 | CDS | hsa-miR- | C/G | G | 82 | C/G | 62.14% |
| | | | 133a | | | | | |
| rs28997576 | BARD1 | CDS | hsa-miR-138 | C/G | G | 110 | C/G | 29.06% |
| rs28997576 | BARD1 | CDS | hsa-miR-139- | C/G | C | 119 | C/G | −38.00% |
| | | | 3p | | | | | |
| rs28997576 | BARD1 | CDS | hsa-miR-140- | C/G | G | 128 | C/G | 22.72% |
| | | | 5p | | | | | |
| rs28997576 | BARD1 | CDS | hsa-miR- | C/G | C | 115 | C/G | −9.10% |
| | | | 143* | | | | | |
| rs28997576 | BARD1 | CDS | hsa-miR- | C/G | C | 118 | C/G | −32.41% |
| | | | 149* | | | | | |
| rs28997576 | BARD1 | CDS | hsa-miR- | C/G | G | 131 | C/G | 13.86% |
| | | | 1538 | | | | | |
| rs28997576 | BARD1 | CDS | hsa-miR- | C/G | C | 103 | C/G | −128.51 |
| | | | 1539 | | | | | |
| rs28997576 | BARD1 | CDS | hsa-miR-16- | C/G | C | 126 | C/G | −21.74% |
| | | | 1* | | | | | |
| rs28997576 | BARD1 | CDS | hsa-miR- | C/G | G | 114 | C/G | 16.53% |
| | | | 181b | | | | | |
| rs28997576 | BARD1 | CDS | hsa-miR- | C/G | G | 108 | C/G | 19.07% |
| | | | 181d | | | | | |
| rs28997576 | BARD1 | CDS | hsa-miR-188- | C/G | G | 89 | C/G | 81.11% |
| | | | 3p | | | | | |
| rs28997576 | BARD1 | CDS | hsa-miR- | C/G | G | 124 | C/G | 75.63% |
| | | | 1912 | | | | | |
| rs28997576 | BARD1 | CDS | hsa-miR- | C/G | C | 146 | C/G | −31.87% |
| | | | 1914* | | | | | |
| rs28997576 | BARD1 | CDS | hsa-miR- | C/G | G | 135 | C/G | 20.49% |
| | | | 1915 | | | | | |
| rs28997576 | BARD1 | CDS | hsa-miR-192 | C/G | G | 88 | C/G | 83.28% |
| rs28997576 | BARD1 | CDS | hsa-miR- | C/G | G | 102 | C/G | 123.62 |
| | | | 193a-3p | | | | | |
| rs28997576 | BARD1 | CDS | hsa-miR- | C/G | G | 94 | C/G | 18.49% |
| | | | 194* | | | | | |
| rs28997576 | BARD1 | CDS | hsa-miR- | C/G | G | 112 | C/G | 24.64% |
| | | | 1972 | | | | | |
| rs28997576 | BARD1 | CDS | hsa-miR- | C/G | G | 102 | C/G | 24.04% |
| | | | 1975 | | | | | |
| rs28997576 | BARD1 | CDS | hsa-miR- | C/G | C | 97 | C/G | −21.22% |
| | | | 1977 | | | | | |
| rs28997576 | BARD1 | CDS | hsa-miR- | C/G | G | 114 | C/G | 23.65% |
| | | | 200a* | | | | | |
| rs28997576 | BARD1 | CDS | hsa-miR- | C/G | G | 116 | C/G | 40.82% |
| | | | 200b* | | | | | |
| rs28997576 | BARD1 | CDS | hsa-miR- | C/G | G | 117 | C/G | 47.94% |
| | | | 200c* | | | | | |
| rs28997576 | BARD1 | CDS | hsa-miR-21* | C/G | G | 83 | C/G | 82.70% |
| rs28997576 | BARD1 | CDS | hsa-miR-210 | C/G | G | 116 | C/G | 41.09% |
| rs28997576 | BARD1 | CDS | hsa-miR- | C/G | C | 120 | C/G | −29.58% |
| | | | 2110 | | | | | |
| rs28997576 | BARD1 | CDS | hsa-miR- | C/G | G | 111 | C/G | 9.42% |
| | | | 214* | | | | | |
| rs28997576 | BARD1 | CDS | hsa-miR-22* | C/G | G | 105 | C/G | 21.89% |
| rs28997576 | BARD1 | CDS | hsa-miR- | C/G | G | 83 | C/G | 63.50% |
| | | | 220c | | | | | |
| rs28997576 | BARD1 | CDS | hsa-miR-221 | C/G | G | 82 | C/G | 55.88% |
| rs28997576 | BARD1 | CDS | hsa-miR- | C/G | C | 100 | C/G | −54.66% |
| | | | 23a* | | | | | |
| rs28997576 | BARD1 | CDS | hsa-miR-24 | C/G | G | 130 | C/G | 18.96% |
| rs28997576 | BARD1 | CDS | hsa-miR-24- | C/G | G | 111 | C/G | 10.56% |
| | | | 1* | | | | | |
| rs28997576 | BARD1 | CDS | hsa-miR-27a | C/G | G | 108 | C/G | 92.00% |
| rs28997576 | BARD1 | CDS | hsa-miR-27b | C/G | G | 110 | C/G | 51.24% |
| rs28997576 | BARD1 | CDS | hsa-miR-296- | C/G | C | 118 | C/G | −30.21% |
| | | | 3p | | | | | |
| rs28997576 | BARD1 | CDS | hsa-miR-298 | C/G | G | 114 | C/G | 18.34% |
| rs28997576 | BARD1 | CDS | hsa-miR-29a | C/G | G | 81 | C/G | 44.81% |
| rs28997576 | BARD1 | CDS | hsa-miR-29b | C/G | G | 86 | C/G | 42.11% |
| rs28997576 | BARD1 | CDS | hsa-miR-30c- | C/G | C | 111 | C/G | −36.23% |
| | | | 1* | | | | | |
| rs28997576 | BARD1 | CDS | hsa-miR-30c- | C/G | C | 107 | C/G | −34.08% |
| | | | 2* | | | | | |
| rs28997576 | BARD1 | CDS | hsa-miR-32 | C/G | G | 92 | C/G | 38.09% |
| rs28997576 | BARD1 | CDS | hsa-miR- | C/G | G | 99 | C/G | 15.70% |
| | | | 320a | | | | | |
| rs28997576 | BARD1 | CDS | hsa-miR- | C/G | G | 99 | C/G | 15.37% |
| | | | 320b | | | | | |
| rs28997576 | BARD1 | CDS | hsa-miR-337- | C/G | G | 85 | C/G | 18.77% |
| | | | 5p | | | | | |
| rs28997576 | BARD1 | CDS | hsa-miR-339- | C/G | G | 105 | C/G | 44.32% |
| | | | 3p | | | | | |
| rs28997576 | BARD1 | CDS | hsa-miR- | C/G | G | 94 | C/G | 47.33% |
| | | | 33b* | | | | | |
| rs28997576 | BARD1 | CDS | hsa-miR-342- | C/G | C | 140 | C/G | −32.95% |
| | | | 5p | | | | | |
| rs28997576 | BARD1 | CDS | hsa-miR-34a | C/G | G | 156 | C/G | 23.04% |
| rs28997576 | BARD1 | CDS | hsa-miR- | C/G | G | 137 | C/G | 13.07% |
| | | | 34b* | | | | | |
| rs28997576 | BARD1 | CDS | hsa-miR-34c- | C/G | G | 158 | C/G | 8.33% |
| | | | 5p | | | | | |
| rs28997576 | BARD1 | CDS | hsa-miR-371- | C/G | G | 95 | C/G | 56.56% |
| | | | 3p | | | | | |
| rs28997576 | BARD1 | CDS | hsa-miR-373 | C/G | C | 109 | C/G | −20.04% |
| rs28997576 | BARD1 | CDS | hsa-miR-375 | C/G | G | 117 | C/G | 8.42% |
| rs28997576 | BARD1 | CDS | hsa-miR-383 | C/G | C | 82 | C/G | −55.42% |
| rs28997576 | BARD1 | CDS | hsa-miR-412 | C/G | G | 93 | C/G | 156.17 |
| rs28997576 | BARD1 | CDS | hsa-miR-432 | C/G | C | 106 | C/G | −58.23% |
| rs28997576 | BARD1 | CDS | hsa-miR- | C/G | G | 150 | C/G | 65.66% |
| | | | 449a | | | | | |
| rs28997576 | BARD1 | CDS | hsa-miR- | C/G | G | 150 | C/G | 43.22% |
| | | | 449b | | | | | |
| rs28997576 | BARD1 | CDS | hsa-miR-452 | C/G | G | 95 | C/G | 31.16% |
| rs28997576 | BARD1 | CDS | hsa-miR-453 | C/G | G | 112 | C/G | 9.12% |
| rs28997576 | BARD1 | CDS | hsa-miR-454 | C/G | G | 91 | C/G | 10.90% |
| rs28997576 | BARD1 | CDS | hsa-miR-486- | C/G | G | 96 | C/G | 21.68% |
| | | | 5p | | | | | |
| rs28997576 | BARD1 | CDS | hsa-miR-506 | C/G | G | 81 | C/G | 38.18% |
| rs28997576 | BARD1 | CDS | hsa-miR-509- | C/G | G | 126 | C/G | 23.28% |
| | | | 5p | | | | | |
| rs28997576 | BARD1 | CDS | hsa-miR-510 | C/G | C | 138 | C/G | −8.57% |
| rs28997576 | BARD1 | CDS | hsa-miR-511 | C/G | G | 96 | C/G | 59.43% |
| rs28997576 | BARD1 | CDS | hsa-miR-515- | C/G | G | 92 | C/G | 41.70% |
| | | | 3p | | | | | |
| rs28997576 | BARD1 | CDS | hsa-miR- | C/G | G | 106 | C/G | 35.00% |
| | | | 516a-3p | | | | | |
| rs28997576 | BARD1 | CDS | hsa-miR- | C/G | G | 106 | C/G | 35.00% |
| | | | 516b* | | | | | |
| rs28997576 | BARD1 | CDS | hsa-miR- | C/G | C | 111 | C/G | −15.91% |
| | | | 518e | | | | | |
| rs28997576 | BARD1 | CDS | hsa-miR- | C/G | G | 86 | C/G | 28.34% |
| | | | 519e | | | | | |
| rs28997576 | BARD1 | CDS | hsa-miR-523 | C/G | G | 117 | C/G | 30.13% |
| rs28997576 | BARD1 | CDS | hsa-miR-525- | C/G | G | 95 | C/G | 57.29% |
| | | | 3p | | | | | |
| rs28997576 | BARD1 | CDS | hsa-miR-550 | C/G | G | 89 | C/G | 28.26% |
| rs28997576 | BARD1 | CDS | hsa-miR-554 | C/G | G | 101 | C/G | 41.78% |
| rs28997576 | BARD1 | CDS | hsa-miR-557 | C/G | G | 91 | C/G | 74.16% |
| rs28997576 | BARD1 | CDS | hsa-miR-564 | C/G | G | 104 | C/G | 43.01% |
| rs28997576 | BARD1 | CDS | hsa-miR-571 | C/G | G | 126 | C/G | 10.45% |
| rs28997576 | BARD1 | CDS | hsa-miR-574- | C/G | G | 119 | C/G | 17.95% |
| | | | 5p | | | | | |
| rs28997576 | BARD1 | CDS | hsa-miR-581 | C/G | G | 121 | C/G | 26.61% |
| rs28997576 | BARD1 | CDS | hsa-miR-601 | C/G | C | 117 | C/G | −24.83% |
| rs28997576 | BARD1 | CDS | hsa-miR-608 | C/G | C | 157 | C/G | −27.01% |
| rs28997576 | BARD1 | CDS | hsa-miR-610 | C/G | G | 95 | C/G | 29.69% |
| rs28997576 | BARD1 | CDS | hsa-miR-612 | C/G | G | 163 | C/G | 25.10% |
| rs28997576 | BARD1 | CDS | hsa-miR-614 | C/G | G | 97 | C/G | 31.48% |
| rs28997576 | BARD1 | CDS | hsa-miR-617 | C/G | G | 95 | C/G | 27.59% |
| rs28997576 | BARD1 | CDS | hsa-miR-619 | C/G | G | 82 | C/G | 26.64% |
| rs28997576 | BARD1 | CDS | hsa-miR-622 | C/G | G | 111 | C/G | 9.96% |
| rs28997576 | BARD1 | CDS | hsa-miR-623 | C/G | G | 87 | C/G | 17.48% |
| rs28997576 | BARD1 | CDS | hsa-miR-628- | C/G | C | 122 | C/G | −9.65% |
| | | | 5p | | | | | |
| rs28997576 | BARD1 | CDS | hsa-miR-637 | C/G | C | 134 | C/G | −34.04% |
| rs28997576 | BARD1 | CDS | hsa-miR-639 | C/G | G | 149 | C/G | 45.49% |
| rs28997576 | BARD1 | CDS | hsa-miR-650 | C/G | C | 103 | C/G | −12.97% |
| rs28997576 | BARD1 | CDS | hsa-miR-661 | C/G | G | 113 | C/G | 24.44% |
| rs28997576 | BARD1 | CDS | hsa-miR-663 | C/G | G | 108 | C/G | 13.53% |
| rs28997576 | BARD1 | CDS | hsa-miR-665 | C/G | C | 124 | C/G | −15.91% |
| rs28997576 | BARD1 | CDS | hsa-miR-671- | C/G | G | 86 | C/G | 17.98% |
| | | | 5p | | | | | |
| rs28997576 | BARD1 | CDS | hsa-miR-744 | C/G | G | 127 | C/G | 33.63% |
| rs28997576 | BARD1 | CDS | hsa-miR-760 | C/G | G | 95 | C/G | 16.00% |
| rs28997576 | BARD1 | CDS | hsa-miR-765 | C/G | C | 146 | C/G | −37.82% |
| rs28997576 | BARD1 | CDS | hsa-miR-767- | C/G | G | 107 | C/G | 24.64% |
| | | | 5p | | | | | |
| rs28997576 | BARD1 | CDS | hsa-miR-874 | C/G | C | 135 | C/G | −14.17% |
| rs28997576 | BARD1 | CDS | hsa-miR-877 | C/G | C | 135 | C/G | −39.10% |
| rs28997576 | BARD1 | CDS | hsa-miR- | C/G | G | 92 | C/G | 43.71% |
| | | | 892b | | | | | |
| rs28997576 | BARD1 | CDS | hsa-miR-93 | C/G | C | 80 | C/G | −18.83% |
| rs28997576 | BARD1 | CDS | hsa-miR- | C/G | G | 80 | C/G | 27.21% |
| | | | 99a* | | | | | |
| rs28997576 | BARD1 | CDS | hsa-miR- | C/G | G | 81 | C/G | 38.87% |
| | | | 99b* | | | | | |
| rs1045485 | CASP8 | CDS | hsa-let-7b | C/G | G | 90 | C/G | 27.32% |
| rs1045485 | CASP8 | CDS | hsa-miR-103 | C/G | G | 143 | C/G | 25.89% |
| rs1045485 | CASP8 | CDS | hsa-miR-107 | C/G | G | 141 | C/G | 25.49% |
| rs1045485 | CASP8 | CDS | hsa-miR- | C/G | G | 82 | C/G | 16.92% |
| | | | 1180 | | | | | |
| rs1045485 | CASP8 | CDS | hsa-miR- | C/G | C | 127 | C/G | −25.31% |
| | | | 1183 | | | | | |
| rs1045485 | CASP8 | CDS | hsa-miR- | C/G | G | 96 | C/G | 15.54% |
| | | | 1202 | | | | | |
| rs1045485 | CASP8 | CDS | hsa-miR- | C/G | G | 119 | C/G | 32.75% |
| | | | 1204 | | | | | |
| rs1045485 | CASP8 | CDS | hsa-miR- | C/G | C | 120 | C/G | −17.42% |
| | | | 1208 | | | | | |
| rs1045485 | CASP8 | CDS | hsa-miR- | C/G | C | 86 | C/G | −126.81 |
| | | | 1268 | | | | | |
| rs1045485 | CASP8 | CDS | hsa-miR- | C/G | G | 129 | C/G | 18.76% |
| | | | 1278 | | | | | |
| rs1045485 | CASP8 | CDS | hsa-miR- | C/G | G | 106 | C/G | 9.99% |
| | | | 1307 | | | | | |
| rs1045485 | CASP8 | CDS | hsa-miR- | C/G | C | 123 | C/G | −21.20% |
| | | | 130b | | | | | |
| rs1045485 | CASP8 | CDS | hsa-miR- | C/G | G | 135 | C/G | 66.01% |
| | | | 136* | | | | | |
| rs1045485 | CASP8 | CDS | hsa-miR- | C/G | G | 107 | C/G | 27.45% |
| | | | 1469 | | | | | |
| rs1045485 | CASP8 | CDS | hsa-miR- | C/G | C | 102 | C/G | −8.13% |
| | | | 181b | | | | | |
| rs1045485 | CASP8 | CDS | hsa-miR- | C/G | G | 118 | C/G | 24.15% |
| | | | 1909 | | | | | |
| rs1045485 | CASP8 | CDS | hsa-miR- | C/G | C | 117 | C/G | −25.18% |
| | | | 1914* | | | | | |
| rs1045485 | CASP8 | CDS | hsa-miR-221 | C/G | G | 85 | C/G | 12.34% |
| rs1045485 | CASP8 | CDS | hsa-miR-24- | C/G | G | 147 | C/G | 21.59% |
| | | | 1* | | | | | |
| rs1045485 | CASP8 | CDS | hsa-miR-296- | C/G | C | 103 | C/G | −39.84% |
| | | | 3p | | | | | |
| rs1045485 | CASP8 | CDS | hsa-miR- | C/G | C | 83 | C/G | −67.97% |
| | | | 302d* | | | | | |
| rs1045485 | CASP8 | CDS | hsa-miR-323- | C/G | G | 121 | C/G | 24.94% |
| | | | 5p | | | | | |
| rs1045485 | CASP8 | CDS | hsa-miR-330- | C/G | G | 115 | C/G | 10.08% |
| | | | 5p | | | | | |
| rs1045485 | CASP8 | CDS | hsa-miR-345 | C/G | G | 89 | C/G | 75.82% |
| rs1045485 | CASP8 | CDS | hsa-miR-370 | C/G | C | 121 | C/G | −23.02% |
| rs1045485 | CASP8 | CDS | hsa-miR-375 | C/G | G | 106 | C/G | 26.60% |
| rs1045485 | CASP8 | CDS | hsa-miR-423- | C/G | G | 136 | C/G | 14.58% |
| | | | 5p | | | | | |
| rs1045485 | CASP8 | CDS | hsa-miR- | C/G | G | 115 | C/G | 20.94% |
| | | | 431* | | | | | |
| rs1045485 | CASP8 | CDS | hsa-miR- | C/G | C | 102 | C/G | −31.08% |
| | | | 520d-3p | | | | | |
| rs1045485 | CASP8 | CDS | hsa-miR-541 | C/G | C | 140 | C/G | −40.82% |
| rs1045485 | CASP8 | CDS | hsa-miR-557 | C/G | C | 133 | C/G | −10.52% |
| rs1045485 | CASP8 | CDS | hsa-miR-595 | C/G | C | 120 | C/G | −26.20% |
| rs1045485 | CASP8 | CDS | hsa-miR-616 | C/G | G | 157 | C/G | 18.89% |
| rs1045485 | CASP8 | CDS | hsa-miR-632 | C/G | G | 105 | C/G | 9.53% |
| rs1045485 | CASP8 | CDS | hsa-miR-638 | C/G | G | 101 | C/G | 59.05% |
| rs1045485 | CASP8 | CDS | hsa-miR-654- | C/G | C | 140 | C/G | −38.70% |
| | | | 5p | | | | | |
| rs1045485 | CASP8 | CDS | hsa-miR-663 | C/G | C | 138 | C/G | −23.66% |
| rs1045485 | CASP8 | CDS | hsa-miR-671- | C/G | C | 115 | C/G | −15.81% |
| | | | 5p | | | | | |
| rs1045485 | CASP8 | CDS | hsa-miR-675 | C/G | C | 146 | C/G | −11.09% |
| rs1045485 | CASP8 | CDS | hsa-miR-744 | C/G | G | 148 | C/G | 18.08% |
| rs1045485 | CASP8 | CDS | hsa-miR-767- | C/G | C | 106 | C/G | −33.85% |
| | | | 5p | | | | | |
| rs1045485 | CASP8 | CDS | hsa-miR-769- | C/G | G | 93 | C/G | 20.23% |
| | | | 3p | | | | | |
| rs1045485 | CASP8 | CDS | hsa-miR-770- | C/G | G | 100 | C/G | 20.84% |
| | | | 5p | | | | | |
| rs1045485 | CASP8 | CDS | hsa-miR-92a- | C/G | C | 173 | C/G | −31.78% |
| | | | 2* | | | | | |
| rs34330 | CDKN1B | 5′ UTR | hsa-let-7a-2* | C/T | T | 96 | C/T | 17.06% |
| rs34330 | CDKN1B | 5′ UTR | hsa-let-7e* | C/T | C | 120 | C/T | −15.29% |
| rs34330 | CDKN1B | 5′ UTR | hsa-let-7f-2* | C/T | T | 108 | C/T | 43.63% |
| rs34330 | CDKN1B | 5′ UTR | hsa-let-7g* | C/T | T | 94 | C/T | 42.68% |
| rs34330 | CDKN1B | 5′ UTR | hsa-let-7i* | C/T | T | 112 | C/T | 30.83% |
| rs34330 | CDKN1B | 5′ UTR | hsa-miR-103- | C/T | T | 97 | C/T | 23.70% |
| | | | 2* | | | | | |
| rs34330 | CDKN1B | 5′ UTR | hsa-miR-105 | C/T | C | 105 | C/T | −15.01% |
| rs34330 | CDKN1B | 5′ UTR | hsa-miR- | C/T | T | 134 | C/T | 36.27% |
| | | | 106b | | | | | |
| rs34330 | CDKN1B | 5′ UTR | hsa-miR- | C/T | C | 121 | C/T | −21.31% |
| | | | 1203 | | | | | |
| rs34330 | CDKN1B | 5′ UTR | hsa-miR- | C/T | C | 166 | C/T | −19.90% |
| | | | 1204 | | | | | |
| rs34330 | CDKN1B | 5′ UTR | hsa-miR- | C/T | C | 87 | C/T | −117.66% |
| | | | 1207-5p | | | | | |
| rs34330 | CDKN1B | 5′ UTR | hsa-miR- | C/T | C | 87 | C/T | −71.75% |
| | | | 1225-5p | | | | | |
| rs34330 | CDKN1B | 5′ UTR | hsa-miR- | C/T | T | 120 | C/T | 30.36% |
| | | | 1226 | | | | | |
| rs34330 | CDKN1B | 5′ UTR | hsa-miR- | C/T | C | 104 | C/T | −10.19% |
| | | | 1226* | | | | | |
| rs34330 | CDKN1B | 5′ UTR | hsa-miR- | C/T | C | 167 | C/T | −10.24% |
| | | | 1234 | | | | | |
| rs34330 | CDKN1B | 5′ UTR | hsa-miR-124 | C/T | C | 111 | C/T | −14.28% |
| rs34330 | CDKN1B | 5′ UTR | hsa-miR- | C/T | C | 86 | C/T | −14.42% |
| | | | 124* | | | | | |
| rs34330 | CDKN1B | 5′ UTR | hsa-miR- | C/T | C | 92 | C/T | −15.54% |
| | | | 1245 | | | | | |
| rs34330 | CDKN1B | 5′ UTR | hsa-miR- | C/T | T | 131 | C/T | 17.82% |
| | | | 1247 | | | | | |
| rs34330 | CDKN1B | 5′ UTR | hsa-miR- | C/T | C | 92 | C/T | −68.59% |
| | | | 1250 | | | | | |
| rs34330 | CDKN1B | 5′ UTR | hsa-miR- | C/T | T | 134 | C/T | 8.40% |
| | | | 1254 | | | | | |
| rs34330 | CDKN1B | 5′ UTR | hsa-miR- | C/T | C | 89 | C/T | −16.12% |
| | | | 1256 | | | | | |
| rs34330 | CDKN1B | 5′ UTR | hsa-miR- | C/T | T | 95 | C/T | 10.09% |
| | | | 125a-3p | | | | | |
| rs34330 | CDKN1B | 5′ UTR | hsa-miR- | C/T | C | 106 | C/T | −17.31% |
| | | | 1269 | | | | | |
| rs34330 | CDKN1B | 5′ UTR | hsa-miR- | C/T | C | 95 | C/T | −51.05% |
| | | | 1271 | | | | | |
| rs34330 | CDKN1B | 5′ UTR | hsa-miR- | C/T | C | 111 | C/T | −9.88% |
| | | | 1274a | | | | | |
| rs34330 | CDKN1B | 5′ UTR | hsa-miR- | C/T | T | 113 | C/T | 71.93% |
| | | | 1284 | | | | | |
| rs34330 | CDKN1B | 5′ UTR | hsa-miR- | C/T | C | 90 | C/T | −34.39% |
| | | | 1285 | | | | | |
| rs34330 | CDKN1B | 5′ UTR | hsa-miR- | C/T | C | 97 | C/T | −17.45% |
| | | | 1288 | | | | | |
| rs34330 | CDKN1B | 5′ UTR | hsa-miR- | C/T | T | 131 | C/T | 12.57% |
| | | | 129* | | | | | |
| rs34330 | CDKN1B | 5′ UTR | hsa-miR-129- | C/T | T | 131 | C/T | 12.57% |
| | | | 3p | | | | | |
| rs34330 | CDKN1B | 5′ UTR | hsa-miR-129- | C/T | C | 88 | C/T | −11.75% |
| | | | 5p | | | | | |
| rs34330 | CDKN1B | 5′ UTR | hsa-miR- | C/T | C | 137 | C/T | −20.97% |
| | | | 1291 | | | | | |
| rs34330 | CDKN1B | 5′ UTR | hsa-miR- | C/T | C | 93 | C/T | −36.45% |
| | | | 1292 | | | | | |
| rs34330 | CDKN1B | 5′ UTR | hsa-miR- | C/T | C | 88 | C/T | −67.71% |
| | | | 1293 | | | | | |
| rs34330 | CDKN1B | 5′ UTR | hsa-miR- | C/T | C | 130 | C/T | −17.19% |
| | | | 1296 | | | | | |
| rs34330 | CDKN1B | 5′ UTR | hsa-miR- | C/T | T | 122 | C/T | 20.13% |
| | | | 1301 | | | | | |
| rs34330 | CDKN1B | 5′ UTR | hsa-miR- | C/T | C | 117 | C/T | −34.19% |
| | | | 1307 | | | | | |
| rs34330 | CDKN1B | 5′ UTR | hsa-miR- | C/T | C | 87 | C/T | −58.51% |
| | | | 1324 | | | | | |
| rs34330 | CDKN1B | 5′ UTR | hsa-miR-139- | C/T | C | 119 | C/T | −12.01% |
| | | | 3p | | | | | |
| rs34330 | CDKN1B | 5′ UTR | hsa-miR-142- | C/T | C | 82 | C/T | −33.09% |
| | | | 3p | | | | | |
| rs34330 | CDKN1B | 5′ UTR | hsa-miR- | C/T | T | 109 | C/T | 39.87% |
| | | | 143* | | | | | |
| rs34330 | CDKN1B | 5′ UTR | hsa-miR- | C/T | C | 126 | C/T | −10.01% |
| | | | 1469 | | | | | |
| rs34330 | CDKN1B | 5′ UTR | hsa-miR- | C/T | C | 122 | C/T | −17.88% |
| | | | 147b | | | | | |
| rs34330 | CDKN1B | 5′ UTR | hsa-miR-149 | C/T | C | 108 | C/T | −10.58% |
| rs34330 | CDKN1B | 5′ UTR | hsa-miR-151- | C/T | T | 104 | C/T | 12.41% |
| | | | 3p | | | | | |
| rs34330 | CDKN1B | 5′ UTR | hsa-miR-151- | C/T | T | 94 | C/T | 14.75% |
| | | | 5p | | | | | |
| rs34330 | CDKN1B | 5′ UTR | hsa-miR- | C/T | C | 165 | C/T | −10.86% |
| | | | 1538 | | | | | |
| rs34330 | CDKN1B | 5′ UTR | hsa-miR- | C/T | T | 113 | C/T | 30.91% |
| | | | 155* | | | | | |
| rs34330 | CDKN1B | 5′ UTR | hsa-miR- | C/T | T | 90 | C/T | 10.65% |
| | | | 15a* | | | | | |
| rs34330 | CDKN1B | 5′ UTR | hsa-miR- | C/T | C | 103 | C/T | −18.66% |
| | | | 185* | | | | | |
| rs34330 | CDKN1B | 5′ UTR | hsa-miR-187 | C/T | T | 97 | C/T | 20.44% |
| rs34330 | CDKN1B | 5′ UTR | hsa-miR-18a | C/T | T | 129 | C/T | 30.86% |
| rs34330 | CDKN1B | 5′ UTR | hsa-miR-18b | C/T | T | 132 | C/T | 29.32% |
| rs34330 | CDKN1B | 5′ UTR | hsa-miR- | C/T | C | 98 | C/T | −45.73% |
| | | | 1908 | | | | | |
| rs34330 | CDKN1B | 5′ UTR | hsa-miR- | C/T | T | 95 | C/T | 32.62% |
| | | | 1909 | | | | | |
| rs34330 | CDKN1B | 5′ UTR | hsa-miR-191 | C/T | T | 111 | C/T | 34.03% |
| rs34330 | CDKN1B | 5′ UTR | hsa-miR- | C/T | C | 123 | C/T | −10.13% |
| | | | 191* | | | | | |
| rs34330 | CDKN1B | 5′ UTR | hsa-miR- | C/T | C | 92 | C/T | −14.28% |
| | | | 1910 | | | | | |
| rs34330 | CDKN1B | 5′ UTR | hsa-miR- | C/T | C | 93 | C/T | −21.38% |
| | | | 1914* | | | | | |
| rs34330 | CDKN1B | 5′ UTR | hsa-miR- | C/T | C | 102 | C/T | −14.11% |
| | | | 1915 | | | | | |
| rs34330 | CDKN1B | 5′ UTR | hsa-miR-192 | C/T | T | 96 | C/T | 36.39% |
| rs34330 | CDKN1B | 5′ UTR | hsa-miR- | C/T | C | 115 | C/T | −9.42% |
| | | | 193a-3p | | | | | |
| rs34330 | CDKN1B | 5′ UTR | hsa-miR- | C/T | C | 90 | C/T | −17.95% |
| | | | 193a-5p | | | | | |
| rs34330 | CDKN1B | 5′ UTR | hsa-miR- | C/T | C | 124 | C/T | −33.13% |
| | | | 193b | | | | | |
| rs34330 | CDKN1B | 5′ UTR | hsa-miR-194 | C/T | C | 94 | C/T | −16.94% |
| rs34330 | CDKN1B | 5′ UTR | hsa-miR- | C/T | C | 80 | C/T | −43.59% |
| | | | 196a* | | | | | |
| rs34330 | CDKN1B | 5′ UTR | hsa-miR- | C/T | T | 131 | C/T | 34.44% |
| | | | 196b* | | | | | |
| rs34330 | CDKN1B | 5′ UTR | hsa-miR- | C/T | C | 87 | C/T | −79.79% |
| | | | 1978 | | | | | |
| rs34330 | CDKN1B | 5′ UTR | hsa-miR- | C/T | C | 93 | C/T | −16.38% |
| | | | 200b* | | | | | |
| rs34330 | CDKN1B | 5′ UTR | hsa-miR- | C/T | T | 87 | C/T | 64.85% |
| | | | 20b* | | | | | |
| rs34330 | CDKN1B | 5′ UTR | hsa-miR-212 | C/T | C | 94 | C/T | −12.68% |
| rs34330 | CDKN1B | 5′ UTR | hsa-miR-217 | C/T | T | 88 | C/T | 11.28% |
| rs34330 | CDKN1B | 5′ UTR | hsa-miR-218- | C/T | C | 81 | C/T | −35.82% |
| | | | 1* | | | | | |
| rs34330 | CDKN1B | 5′ UTR | hsa-miR-219- | C/T | T | 81 | C/T | 25.88% |
| | | | 5p | | | | | |
| rs34330 | CDKN1B | 5′ UTR | hsa-miR-223 | C/T | T | 80 | C/T | 58.78% |
| rs34330 | CDKN1B | 5′ UTR | hsa-miR-224 | C/T | C | 88 | C/T | −37.08% |
| rs34330 | CDKN1B | 5′ UTR | hsa-miR- | C/T | C | 97 | C/T | −15.70% |
| | | | 23a* | | | | | |
| rs34330 | CDKN1B | 5′ UTR | hsa-miR- | C/T | T | 106 | C/T | 14.88% |
| | | | 23b* | | | | | |
| rs34330 | CDKN1B | 5′ UTR | hsa-miR-25 | C/T | C | 97 | C/T | −18.24% |
| rs34330 | CDKN1B | 5′ UTR | hsa-miR- | C/T | C | 86 | C/T | −39.06% |
| | | | 27a* | | | | | |
| rs34330 | CDKN1B | 5′ UTR | hsa-miR-296- | C/T | C | 147 | C/T | −22.44% |
| | | | 5p | | | | | |
| rs34330 | CDKN1B | 5′ UTR | hsa-miR-298 | C/T | C | 79 | C/T | −12.57% |
| rs34330 | CDKN1B | 5′ UTR | hsa-miR-299- | C/T | C | 98 | C/T | −32.04% |
| | | | 3p | | | | | |
| rs34330 | CDKN1B | 5′ UTR | hsa-miR-29a | C/T | C | 100 | C/T | −17.91% |
| rs34330 | CDKN1B | 5′ UTR | hsa-miR-300 | C/T | T | 95 | C/T | 10.67% |
| rs34330 | CDKN1B | 5′ UTR | hsa-miR-323- | C/T | C | 105 | C/T | −13.75% |
| | | | 3p | | | | | |
| rs34330 | CDKN1B | 5′ UTR | hsa-miR-323- | C/T | C | 124 | C/T | −24.08% |
| | | | 5p | | | | | |
| rs34330 | CDKN1B | 5′ UTR | hsa-miR-325 | C/T | T | 97 | C/T | 30.49% |
| rs34330 | CDKN1B | 5′ UTR | hsa-miR-328 | C/T | C | 129 | C/T | −21.25% |
| rs34330 | CDKN1B | 5′ UTR | hsa-miR-330- | C/T | C | 131 | C/T | −10.82% |
| | | | 3p | | | | | |
| rs34330 | CDKN1B | 5′ UTR | hsa-miR-331- | C/T | C | 129 | C/T | −27.19% |
| | | | 3p | | | | | |
| rs34330 | CDKN1B | 5′ UTR | hsa-miR- | C/T | T | 149 | C/T | 23.76% |
| | | | 33b* | | | | | |
| rs34330 | CDKN1B | 5′ UTR | hsa-miR-342- | C/T | T | 97 | C/T | 74.19% |
| | | | 3p | | | | | |
| rs34330 | CDKN1B | 5′ UTR | hsa-miR-361- | C/T | C | 104 | C/T | −13.37% |
| | | | 3p | | | | | |
| rs34330 | CDKN1B | 5′ UTR | hsa-miR-370 | C/T | C | 104 | C/T | −16.87% |
| rs34330 | CDKN1B | 5′ UTR | hsa-miR-373 | C/T | C | 114 | C/T | −16.37% |
| rs34330 | CDKN1B | 5′ UTR | hsa-miR-383 | C/T | C | 95 | C/T | −41.45% |
| rs34330 | CDKN1B | 5′ UTR | hsa-miR-409- | C/T | C | 94 | C/T | −8.90% |
| | | | 3p | | | | | |
| rs34330 | CDKN1B | 5′ UTR | hsa-miR-423- | C/T | C | 126 | C/T | −8.14% |
| | | | 3p | | | | | |
| rs34330 | CDKN1B | 5′ UTR | hsa-miR- | C/T | T | 108 | C/T | 49.32% |
| | | | 449b* | | | | | |
| rs34330 | CDKN1B | 5′ UTR | hsa-miR-484 | C/T | C | 133 | C/T | −9.59% |
| rs34330 | CDKN1B | 5′ UTR | hsa-miR-485- | C/T | C | 105 | C/T | −11.55% |
| | | | 3p | | | | | |
| rs34330 | CDKN1B | 5′ UTR | hsa-miR-486- | C/T | C | 98 | C/T | −22.04% |
| | | | 3p | | | | | |
| rs34330 | CDKN1B | 5′ UTR | hsa-miR-490- | C/T | C | 94 | C/T | −11.78% |
| | | | 3p | | | | | |
| rs34330 | CDKN1B | 5′ UTR | hsa-miR-491- | C/T | T | 118 | C/T | 9.77% |
| | | | 3p | | | | | |
| rs34330 | CDKN1B | 5′ UTR | hsa-miR-491- | C/T | C | 99 | C/T | −101.16% |
| | | | 5p | | | | | |
| rs34330 | CDKN1B | 5′ UTR | hsa-miR-492 | C/T | C | 91 | C/T | −39.22% |
| rs34330 | CDKN1B | 5′ UTR | hsa-miR-497 | C/T | C | 90 | C/T | −54.83% |
| rs34330 | CDKN1B | 5′ UTR | hsa-miR-498 | C/T | T | 82 | C/T | 16.52% |
| rs34330 | CDKN1B | 5′ UTR | hsa-miR-502- | C/T | C | 104 | C/T | −14.15% |
| | | | 3p | | | | | |
| rs34330 | CDKN1B | 5′ UTR | hsa-miR-503 | C/T | T | 128 | C/T | 10.43% |
| rs34330 | CDKN1B | 5′ UTR | hsa-miR-504 | C/T | C | 120 | C/T | −11.10% |
| rs34330 | CDKN1B | 5′ UTR | hsa-miR-506 | C/T | T | 117 | C/T | 13.63% |
| rs34330 | CDKN1B | 5′ UTR | hsa-miR-511 | C/T | T | 84 | C/T | 9.57% |
| rs34330 | CDKN1B | 5′ UTR | hsa-miR-512- | C/T | T | 94 | C/T | 35.96% |
| | | | 3p | | | | | |
| rs34330 | CDKN1B | 5′ UTR | hsa-miR-512- | C/T | T | 136 | C/T | 42.80% |
| | | | 5p | | | | | |
| rs34330 | CDKN1B | 5′ UTR | hsa-miR- | C/T | C | 169 | C/T | −13.40% |
| | | | 518a-3p | | | | | |
| rs34330 | CDKN1B | 5′ UTR | hsa-miR- | C/T | T | 164 | C/T | 17.92% |
| | | | 518c | | | | | |
| rs34330 | CDKN1B | 5′ UTR | hsa-miR- | C/T | T | 178 | C/T | 8.49% |
| | | | 518d-3p | | | | | |
| rs34330 | CDKN1B | 5′ UTR | hsa-miR- | C/T | T | 145 | C/T | 14.69% |
| | | | 518e | | | | | |
| rs34330 | CDKN1B | 5′ UTR | hsa-miR- | C/T | C | 97 | C/T | −10.16% |
| | | | 518e* | | | | | |
| rs34330 | CDKN1B | 5′ UTR | hsa-miR- | C/T | C | 94 | C/T | −14.94% |
| | | | 518f* | | | | | |
| rs34330 | CDKN1B | 5′ UTR | hsa-miR- | C/T | C | 97 | C/T | −10.16% |
| | | | 519a* | | | | | |
| rs34330 | CDKN1B | 5′ UTR | hsa-miR- | C/T | C | 97 | C/T | −10.16% |
| | | | 519b-5p | | | | | |
| rs34330 | CDKN1B | 5′ UTR | hsa-miR- | C/T | C | 97 | C/T | −10.16% |
| | | | 519c-5p | | | | | |
| rs34330 | CDKN1B | 5′ UTR | hsa-miR- | C/T | T | 139 | C/T | 10.68% |
| | | | 519d | | | | | |
| rs34330 | CDKN1B | 5′ UTR | hsa-miR- | C/T | T | 123 | C/T | 19.95% |
| | | | 519e | | | | | |
| rs34330 | CDKN1B | 5′ UTR | hsa-miR- | C/T | C | 87 | C/T | −11.02% |
| | | | 519e* | | | | | |
| rs34330 | CDKN1B | 5′ UTR | hsa-miR- | C/T | C | 126 | C/T | −16.67% |
| | | | 520a-3p | | | | | |
| rs34330 | CDKN1B | 5′ UTR | hsa-miR- | C/T | C | 124 | C/T | −27.32% |
| | | | 520d-3p | | | | | |
| rs34330 | CDKN1B | 5′ UTR | hsa-miR- | C/T | C | 118 | C/T | −12.09% |
| | | | 520e | | | | | |
| rs34330 | CDKN1B | 5′ UTR | hsa-miR- | C/T | T | 143 | C/T | 10.70% |
| | | | 520h | | | | | |
| rs34330 | CDKN1B | 5′ UTR | hsa-miR-521 | C/T | T | 111 | C/T | 29.31% |
| rs34330 | CDKN1B | 5′ UTR | hsa-miR- | C/T | C | 97 | C/T | −10.16% |
| | | | 522* | | | | | |
| rs34330 | CDKN1B | 5′ UTR | hsa-miR-523 | C/T | C | 139 | C/T | −9.20% |
| rs34330 | CDKN1B | 5′ UTR | hsa-miR- | C/T | C | 97 | C/T | −10.16% |
| | | | 523* | | | | | |
| rs34330 | CDKN1B | 5′ UTR | hsa-miR-532- | C/T | T | 88 | C/T | 9.54% |
| | | | 5p | | | | | |
| rs34330 | CDKN1B | 5′ UTR | hsa-miR-550 | C/T | T | 116 | C/T | 14.42% |
| rs34330 | CDKN1B | 5′ UTR | hsa-miR-554 | C/T | T | 107 | C/T | 43.14% |
| rs34330 | CDKN1B | 5′ UTR | hsa-miR-574- | C/T | C | 103 | C/T | −10.11% |
| | | | 3p | | | | | |
| rs34330 | CDKN1B | 5′ UTR | hsa-miR-592 | C/T | C | 83 | C/T | −9.47% |
| rs34330 | CDKN1B | 5′ UTR | hsa-miR-593 | C/T | C | 101 | C/T | −15.48% |
| rs34330 | CDKN1B | 5′ UTR | hsa-miR- | C/T | C | 112 | C/T | −43.09% |
| | | | 593* | | | | | |
| rs34330 | CDKN1B | 5′ UTR | hsa-miR-595 | C/T | C | 89 | C/T | −18.08% |
| rs34330 | CDKN1B | 5′ UTR | hsa-miR-602 | C/T | T | 97 | C/T | 15.08% |
| rs34330 | CDKN1B | 5′ UTR | hsa-miR-608 | C/T | C | 89 | C/T | −16.03% |
| rs34330 | CDKN1B | 5′ UTR | hsa-miR-614 | C/T | T | 98 | C/T | 9.16% |
| rs34330 | CDKN1B | 5′ UTR | hsa-miR-615- | C/T | T | 120 | C/T | 17.98% |
| | | | 5p | | | | | |
| rs34330 | CDKN1B | 5′ UTR | hsa-miR-623 | C/T | C | 109 | C/T | −14.45% |
| rs34330 | CDKN1B | 5′ UTR | hsa-miR- | C/T | C | 109 | C/T | −14.46% |
| | | | 629* | | | | | |
| rs34330 | CDKN1B | 5′ UTR | hsa-miR-631 | C/T | C | 113 | C/T | −27.33% |
| rs34330 | CDKN1B | 5′ UTR | hsa-miR-634 | C/T | T | 109 | C/T | 13.31% |
| rs34330 | CDKN1B | 5′ UTR | hsa-miR-636 | C/T | C | 97 | C/T | −20.57% |
| rs34330 | CDKN1B | 5′ UTR | hsa-miR-637 | C/T | C | 91 | C/T | −32.12% |
| rs34330 | CDKN1B | 5′ UTR | hsa-miR-638 | C/T | C | 95 | C/T | −31.85% |
| rs34330 | CDKN1B | 5′ UTR | hsa-miR-639 | C/T | C | 116 | C/T | −16.65% |
| rs34330 | CDKN1B | 5′ UTR | hsa-miR-640 | C/T | T | 80 | C/T | 8.59% |
| rs34330 | CDKN1B | 5′ UTR | hsa-miR-644 | C/T | C | 101 | C/T | −29.18% |
| rs34330 | CDKN1B | 5′ UTR | hsa-miR-648 | C/T | C | 86 | C/T | −23.07% |
| rs34330 | CDKN1B | 5′ UTR | hsa-miR-652 | C/T | C | 120 | C/T | −14.67% |
| rs34330 | CDKN1B | 5′ UTR | hsa-miR-657 | C/T | C | 107 | C/T | −10.04% |
| rs34330 | CDKN1B | 5′ UTR | hsa-miR-662 | C/T | C | 124 | C/T | −22.07% |
| rs34330 | CDKN1B | 5′ UTR | hsa-miR-663 | C/T | T | 89 | C/T | 52.68% |
| rs34330 | CDKN1B | 5′ UTR | hsa-miR- | C/T | C | 166 | C/T | −21.80% |
| | | | 663b | | | | | |
| rs34330 | CDKN1B | 5′ UTR | hsa-miR-665 | C/T | C | 90 | C/T | −11.68% |
| rs34330 | CDKN1B | 5′ UTR | hsa-miR-668 | C/T | C | 104 | C/T | −11.30% |
| rs34330 | CDKN1B | 5′ UTR | hsa-miR-671- | C/T | T | 97 | C/T | 14.52% |
| | | | 3p | | | | | |
| rs34330 | CDKN1B | 5′ UTR | hsa-miR-675 | C/T | C | 111 | C/T | −41.15% |
| rs34330 | CDKN1B | 5′ UTR | hsa-miR-708 | C/T | C | 89 | C/T | −39.87% |
| rs34330 | CDKN1B | 5′ UTR | hsa-miR-744 | C/T | C | 121 | C/T | −16.58% |
| rs34330 | CDKN1B | 5′ UTR | hsa-miR-758 | C/T | C | 88 | C/T | −17.97% |
| rs34330 | CDKN1B | 5′ UTR | hsa-miR-760 | C/T | C | 108 | C/T | −45.19% |
| rs34330 | CDKN1B | 5′ UTR | hsa-miR-767- | C/T | C | 97 | C/T | −13.32% |
| | | | 3p | | | | | |
| rs34330 | CDKN1B | 5′ UTR | hsa-miR-873 | C/T | T | 109 | C/T | 28.76% |
| rs34330 | CDKN1B | 5′ UTR | hsa-miR-874 | C/T | C | 112 | C/T | −12.55% |
| rs34330 | CDKN1B | 5′ UTR | hsa-miR-886- | C/T | C | 102 | C/T | −76.53% |
| | | | 3p | | | | | |
| rs34330 | CDKN1B | 5′ UTR | hsa-miR-886- | C/T | C | 111 | C/T | −75.20% |
| | | | 5p | | | | | |
| rs34330 | CDKN1B | 5′ UTR | hsa-miR-887 | C/T | T | 101 | C/T | 23.16% |
| rs34330 | CDKN1B | 5′ UTR | hsa-miR- | C/T | T | 101 | C/T | 35.78% |
| | | | 891a | | | | | |
| rs34330 | CDKN1B | 5′ UTR | hsa-miR-922 | C/T | C | 91 | C/T | −11.92% |
| rs34330 | CDKN1B | 5′ UTR | hsa-miR-92a | C/T | C | 99 | C/T | −16.68% |
| rs34330 | CDKN1B | 5′ UTR | hsa-miR-933 | C/T | T | 134 | C/T | 9.11% |
| rs34330 | CDKN1B | 5′ UTR | hsa-miR-935 | C/T | C | 76 | C/T | −13.84% |
| rs34330 | CDKN1B | 5′ UTR | hsa-miR-940 | C/T | C | 108 | C/T | −18.30% |
| rs34330 | CDKN1B | 5′ UTR | hsa-miR- | C/T | C | 89 | C/T | −42.42% |
| | | | 99a* | | | | | |
| rs34330 | CDKN1B | 5′ UTR | hsa-miR-99b | C/T | T | 116 | C/T | 35.26% |
| rs34330 | CDKN1B | 5′ UTR | hsa-miR- | C/T | C | 111 | C/T | −15.67% |
| | | | 99b* | | | | | |
| rs4148946 | CHST3 | 3′ UTR | hsa-let-7d | C/T | C | 118 | C/T | −13.60% |
| rs4148946 | CHST3 | 3′ UTR | hsa-miR- | C/T | C | 113 | C/T | −8.51% |
| | | | 1202 | | | | | |
| rs4148946 | CHST3 | 3′ UTR | hsa-miR- | C/T | C | 89 | C/T | −31.10% |
| | | | 1207-5p | | | | | |
| rs4148946 | CHST3 | 3′ UTR | hsa-miR- | C/T | T | 124 | C/T | 10.47% |
| | | | 1231 | | | | | |
| rs4148946 | CHST3 | 3′ UTR | hsa-miR- | C/T | C | 132 | C/T | −12.77% |
| | | | 1254 | | | | | |
| rs4148946 | CHST3 | 3′ UTR | hsa-miR- | C/T | C | 108 | C/T | −11.89% |
| | | | 125a-3p | | | | | |
| rs4148946 | CHST3 | 3′ UTR | hsa-miR- | C/T | T | 86 | C/T | 23.36% |
| | | | 125a-5p | | | | | |
| rs4148946 | CHST3 | 3′ UTR | hsa-miR- | C/T | C | 123 | C/T | −9.33% |
| | | | 1292 | | | | | |
| rs4148946 | CHST3 | 3′ UTR | hsa-miR-132 | C/T | T | 102 | C/T | 55.82% |
| rs4148946 | CHST3 | 3′ UTR | hsa-miR- | C/T | T | 104 | C/T | 46.34% |
| | | | 1323 | | | | | |
| rs4148946 | CHST3 | 3′ UTR | hsa-miR-134 | C/T | C | 88 | C/T | −11.96% |
| rs4148946 | CHST3 | 3′ UTR | hsa-miR- | C/T | T | 106 | C/T | 64.75% |
| | | | 135a* | | | | | |
| rs4148946 | CHST3 | 3′ UTR | hsa-miR- | C/T | C | 113 | C/T | −39.95% |
| | | | 135b* | | | | | |
| rs4148946 | CHST3 | 3′ UTR | hsa-miR-139- | C/T | T | 98 | C/T | 72.63% |
| | | | 3p | | | | | |
| rs4148946 | CHST3 | 3′ UTR | hsa-miR-140- | C/T | C | 110 | C/T | −10.04% |
| | | | 3p | | | | | |
| rs4148946 | CHST3 | 3′ UTR | hsa-miR- | C/T | T | 94 | C/T | 9.92% |
| | | | 141* | | | | | |
| rs4148946 | CHST3 | 3′ UTR | hsa-miR- | C/T | C | 115 | C/T | −30.46% |
| | | | 143* | | | | | |
| rs4148946 | CHST3 | 3′ UTR | hsa-miR-145 | C/T | T | 110 | C/T | 13.74% |
| rs4148946 | CHST3 | 3′ UTR | hsa-miR- | C/T | C | 96 | C/T | −14.16% |
| | | | 150* | | | | | |
| rs4148946 | CHST3 | 3′ UTR | hsa-miR-16 | C/T | C | 113 | C/T | −32.15% |
| rs4148946 | CHST3 | 3′ UTR | hsa-miR-17* | C/T | C | 94 | C/T | −31.12% |
| rs4148946 | CHST3 | 3′ UTR | hsa-miR- | C/T | T | 81 | C/T | 17.95% |
| | | | 186* | | | | | |
| rs4148946 | CHST3 | 3′ UTR | hsa-miR- | C/T | C | 128 | C/T | −45.10% |
| | | | 1909 | | | | | |
| rs4148946 | CHST3 | 3′ UTR | hsa-miR-191 | C/T | C | 118 | C/T | −13.04% |
| rs4148946 | CHST3 | 3′ UTR | hsa-miR- | C/T | T | 117 | C/T | 27.73% |
| | | | 1912 | | | | | |
| rs4148946 | CHST3 | 3′ UTR | hsa-miR- | C/T | C | 96 | C/T | −12.99% |
| | | | 1914* | | | | | |
| rs4148946 | CHST3 | 3′ UTR | hsa-miR- | C/T | T | 135 | C/T | 12.16% |
| | | | 1972 | | | | | |
| rs4148946 | CHST3 | 3′ UTR | hsa-miR-198 | C/T | T | 130 | C/T | 8.52% |
| rs4148946 | CHST3 | 3′ UTR | hsa-miR- | C/T | C | 92 | C/T | −9.23% |
| | | | 20b* | | | | | |
| rs4148946 | CHST3 | 3′ UTR | hsa-miR- | C/T | C | 110 | C/T | −10.90% |
| | | | 2110 | | | | | |
| rs4148946 | CHST3 | 3′ UTR | hsa-miR-212 | C/T | T | 102 | C/T | 57.75% |
| rs4148946 | CHST3 | 3′ UTR | hsa-miR-24 | C/T | T | 108 | C/T | 30.85% |
| rs4148946 | CHST3 | 3′ UTR | hsa-miR-25* | C/T | C | 138 | C/T | −8.98% |
| rs4148946 | CHST3 | 3′ UTR | hsa-miR-298 | C/T | C | 152 | C/T | −8.12% |
| rs4148946 | CHST3 | 3′ UTR | hsa-miR-31 | C/T | C | 93 | C/T | −115.58 |
| rs4148946 | CHST3 | 3′ UTR | hsa-miR-325 | C/T | C | 96 | C/T | −29.90% |
| rs4148946 | CHST3 | 3′ UTR | hsa-miR-339- | C/T | C | 105 | C/T | −26.08% |
| | | | 5p | | | | | |
| rs4148946 | CHST3 | 3′ UTR | hsa-miR- | C/T | C | 105 | C/T | −15.60% |
| | | | 34a* | | | | | |
| rs4148946 | CHST3 | 3′ UTR | hsa-miR-381 | C/T | T | 101 | C/T | 71.87% |
| rs4148946 | CHST3 | 3′ UTR | hsa-miR-409- | C/T | C | 115 | C/T | −15.16% |
| | | | 5p | | | | | |
| rs4148946 | CHST3 | 3′ UTR | hsa-miR- | C/T | C | 108 | C/T | −79.22% |
| | | | 425* | | | | | |
| rs4148946 | CHST3 | 3′ UTR | hsa-miR-432 | C/T | C | 107 | C/T | −9.40% |
| rs4148946 | CHST3 | 3′ UTR | hsa-miR- | C/T | C | 122 | C/T | −38.62% |
| | | | 449b | | | | | |
| rs4148946 | CHST3 | 3′ UTR | hsa-miR-483- | C/T | C | 102 | C/T | −14.25% |
| | | | 5p | | | | | |
| rs4148946 | CHST3 | 3′ UTR | hsa-miR-486- | C/T | T | 91 | C/T | 8.59% |
| | | | 5p | | | | | |
| rs4148946 | CHST3 | 3′ UTR | hsa-miR- | C/T | T | 122 | C/T | 82.63% |
| | | | 488* | | | | | |
| rs4148946 | CHST3 | 3′ UTR | hsa-miR-494 | C/T | T | 125 | C/T | 35.12% |
| rs4148946 | CHST3 | 3′ UTR | hsa-miR-498 | C/T | C | 101 | C/T | −15.21% |
| rs4148946 | CHST3 | 3′ UTR | hsa-miR-505 | C/T | T | 89 | C/T | 213.13% |
| rs4148946 | CHST3 | 3′ UTR | hsa-miR- | C/T | C | 112 | C/T | −15.12% |
| | | | 516a-5p | | | | | |
| rs4148946 | CHST3 | 3′ UTR | hsa-miR- | C/T | C | 107 | C/T | −13.36% |
| | | | 516b | | | | | |
| rs4148946 | CHST3 | 3′ UTR | hsa-miR- | C/T | C | 102 | C/T | −19.51% |
| | | | 518e* | | | | | |
| rs4148946 | CHST3 | 3′ UTR | hsa-miR- | C/T | C | 102 | C/T | −19.51% |
| | | | 519a* | | | | | |
| rs4148946 | CHST3 | 3′ UTR | hsa-miR- | C/T | C | 102 | C/T | −19.51% |
| | | | 519b-5p | | | | | |
| rs4148946 | CHST3 | 3′ UTR | hsa-miR- | C/T | C | 102 | C/T | −19.51% |
| | | | 519c-5p | | | | | |
| rs4148946 | CHST3 | 3′ UTR | hsa-miR- | C/T | C | 102 | C/T | −19.51% |
| | | | 522* | | | | | |
| rs4148946 | CHST3 | 3′ UTR | hsa-miR- | C/T | C | 102 | C/T | −19.51% |
| | | | 523* | | | | | |
| rs4148946 | CHST3 | 3′ UTR | hsa-miR-542- | C/T | T | 96 | C/T | 11.95% |
| | | | 5p | | | | | |
| rs4148946 | CHST3 | 3′ UTR | hsa-miR-557 | C/T | C | 88 | C/T | −10.62% |
| rs4148946 | CHST3 | 3′ UTR | hsa-miR-582- | C/T | T | 84 | C/T | 12.71% |
| | | | 5p | | | | | |
| rs4148946 | CHST3 | 3′ UTR | hsa-miR-601 | C/T | C | 125 | C/T | −11.94% |
| rs4148946 | CHST3 | 3′ UTR | hsa-miR-608 | C/T | C | 125 | C/T | −41.34% |
| rs4148946 | CHST3 | 3′ UTR | hsa-miR-616 | C/T | C | 90 | C/T | −14.58% |
| rs4148946 | CHST3 | 3′ UTR | hsa-miR-622 | C/T | T | 94 | C/T | 9.63% |
| rs4148946 | CHST3 | 3′ UTR | hsa-miR-628- | C/T | T | 114 | C/T | 71.03% |
| | | | 3p | | | | | |
| rs4148946 | CHST3 | 3′ UTR | hsa-miR-657 | C/T | C | 112 | C/T | −11.54% |
| rs4148946 | CHST3 | 3′ UTR | hsa-miR-658 | C/T | C | 123 | C/T | −9.03% |
| rs4148946 | CHST3 | 3′ UTR | hsa-miR-665 | C/T | C | 162 | C/T | −29.08% |
| rs4148946 | CHST3 | 3′ UTR | hsa-miR-675 | C/T | C | 113 | C/T | −49.90% |
| rs4148946 | CHST3 | 3′ UTR | hsa-miR-744 | C/T | C | 148 | C/T | −78.28% |
| rs4148946 | CHST3 | 3′ UTR | hsa-miR-802 | C/T | T | 125 | C/T | 21.78% |
| rs4148946 | CHST3 | 3′ UTR | hsa-miR-875- | C/T | C | 137 | C/T | −9.59% |
| | | | 3p | | | | | |
| rs4148946 | CHST3 | 3′ UTR | hsa-miR-877 | C/T | C | 101 | C/T | −11.22% |
| rs4148946 | CHST3 | 3′ UTR | hsa-miR-885- | C/T | C | 87 | C/T | −41.02% |
| | | | 3p | | | | | |
| rs4148946 | CHST3 | 3′ UTR | hsa-miR-920 | C/T | C | 127 | C/T | −8.80% |
| rs4148946 | CHST3 | 3′ UTR | hsa-miR-922 | C/T | C | 132 | C/T | −13.95% |
| rs4148946 | CHST3 | 3′ UTR | hsa-miR- | C/T | T | 116 | C/T | 16.93% |
| | | | 92b* | | | | | |
| rs4148946 | CHST3 | 3′ UTR | hsa-miR-93* | C/T | C | 109 | C/T | −16.62% |
| rs4148946 | CHST3 | 3′ UTR | hsa-miR-934 | C/T | T | 94 | C/T | 19.97% |
| rs4148946 | CHST3 | 3′ UTR | hsa-miR-936 | C/T | C | 113 | C/T | −20.46% |
| rs4148946 | CHST3 | 3′ UTR | hsa-miR-939 | C/T | C | 134 | C/T | −8.75% |
| rs4148946 | CHST3 | 3′ UTR | hsa-miR-940 | C/T | C | 124 | C/T | −35.54% |
| rs4148949 | CHST3 | 3′ UTR | hsa-let-7f-2* | C/T | T | 120 | C/T | 30.40% |
| rs4148949 | CHST3 | 3′ UTR | hsa-miR- | C/T | C | 123 | C/T | −72.28% |
| | | | 106a | | | | | |
| rs4148949 | CHST3 | 3′ UTR | hsa-miR- | C/T | C | 127 | C/T | −45.91% |
| | | | 106b | | | | | |
| rs4148949 | CHST3 | 3′ UTR | hsa-miR- | C/T | C | 86 | C/T | −63.96% |
| | | | 106b* | | | | | |
| rs4148949 | CHST3 | 3′ UTR | hsa-miR- | C/T | T | 119 | C/T | 15.53% |
| | | | 1208 | | | | | |
| rs4148949 | CHST3 | 3′ UTR | hsa-miR- | C/T | C | 116 | C/T | −37.29% |
| | | | 1226* | | | | | |
| rs4148949 | CHST3 | 3′ UTR | hsa-miR- | C/T | C | 107 | C/T | −15.98% |
| | | | 1228* | | | | | |
| rs4148949 | CHST3 | 3′ UTR | hsa-miR- | C/T | T | 119 | C/T | 14.12% |
| | | | 1247 | | | | | |
| rs4148949 | CHST3 | 3′ UTR | hsa-miR- | C/T | C | 95 | C/T | −17.60% |
| | | | 1294 | | | | | |
| rs4148949 | CHST3 | 3′ UTR | hsa-miR- | C/T | C | 144 | C/T | −8.51% |
| | | | 1300 | | | | | |
| rs4148949 | CHST3 | 3′ UTR | hsa-miR-138- | C/T | T | 145 | C/T | 11.20% |
| | | | 1* | | | | | |
| rs4148949 | CHST3 | 3′ UTR | hsa-miR-139- | C/T | T | 134 | C/T | 9.71% |
| | | | 5p | | | | | |
| rs4148949 | CHST3 | 3′ UTR | hsa-miR- | C/T | C | 70 | C/T | −38.08% |
| | | | 1469 | | | | | |
| rs4148949 | CHST3 | 3′ UTR | hsa-miR- | C/T | C | 92 | C/T | −12.70% |
| | | | 1470 | | | | | |
| rs4148949 | CHST3 | 3′ UTR | hsa-miR- | C/T | C | 116 | C/T | −8.99% |
| | | | 148b* | | | | | |
| rs4148949 | CHST3 | 3′ UTR | hsa-miR- | C/T | C | 83 | C/T | −11.31% |
| | | | 1538 | | | | | |
| rs4148949 | CHST3 | 3′ UTR | hsa-miR-17 | C/T | C | 123 | C/T | −74.14% |
| rs4148949 | CHST3 | 3′ UTR | hsa-miR- | C/T | T | 107 | C/T | 88.09% |
| | | | 18a* | | | | | |
| rs4148949 | CHST3 | 3′ UTR | hsa-miR- | C/T | C | 93 | C/T | −75.91% |
| | | | 191* | | | | | |
| rs4148949 | CHST3 | 3′ UTR | hsa-miR- | C/T | C | 91 | C/T | −11.28% |
| | | | 1913 | | | | | |
| rs4148949 | CHST3 | 3′ UTR | hsa-miR-20a | C/T | C | 121 | C/T | −61.27% |
| rs4148949 | CHST3 | 3′ UTR | hsa-miR- | C/T | C | 106 | C/T | −8.97% |
| | | | 20b* | | | | | |
| rs4148949 | CHST3 | 3′ UTR | hsa-miR- | C/T | C | 140 | C/T | −12.14% |
| | | | 214* | | | | | |
| rs4148949 | CHST3 | 3′ UTR | hsa-miR- | C/T | C | 106 | C/T | −12.45% |
| | | | 27a* | | | | | |
| rs4148949 | CHST3 | 3′ UTR | hsa-miR-28- | C/T | T | 112 | C/T | 19.93% |
| | | | 5p | | | | | |
| rs4148949 | CHST3 | 3′ UTR | hsa-miR-296- | C/T | C | 116 | C/T | −11.35% |
| | | | 3p | | | | | |
| rs4148949 | CHST3 | 3′ UTR | hsa-miR-299- | C/T | T | 125 | C/T | 35.24% |
| | | | 5p | | | | | |
| rs4148949 | CHST3 | 3′ UTR | hsa-miR- | C/T | C | 126 | C/T | −11.21% |
| | | | 302b | | | | | |
| rs4148949 | CHST3 | 3′ UTR | hsa-miR- | C/T | C | 125 | C/T | −11.24% |
| | | | 302c | | | | | |
| rs4148949 | CHST3 | 3′ UTR | hsa-miR- | C/T | T | 134 | C/T | 33.02% |
| | | | 30b* | | | | | |
| rs4148949 | CHST3 | 3′ UTR | hsa-miR-30c- | C/T | C | 112 | C/T | −9.76% |
| | | | 1* | | | | | |
| rs4148949 | CHST3 | 3′ UTR | hsa-miR- | C/T | C | 118 | C/T | −11.34% |
| | | | 320b | | | | | |
| rs4148949 | CHST3 | 3′ UTR | hsa-miR-345 | C/T | C | 110 | C/T | −10.79% |
| rs4148949 | CHST3 | 3′ UTR | hsa-miR-346 | C/T | C | 140 | C/T | −8.20% |
| rs4148949 | CHST3 | 3′ UTR | hsa-miR-370 | C/T | C | 132 | C/T | −10.58% |
| rs4148949 | CHST3 | 3′ UTR | hsa-miR-372 | C/T | C | 131 | C/T | −11.98% |
| rs4148949 | CHST3 | 3′ UTR | hsa-miR-409- | C/T | C | 95 | C/T | −17.44% |
| | | | 3p | | | | | |
| rs4148949 | CHST3 | 3′ UTR | hsa-miR-423- | C/T | T | 85 | C/T | 14.18% |
| | | | 3p | | | | | |
| rs4148949 | CHST3 | 3′ UTR | hsa-miR-423- | C/T | T | 114 | C/T | 8.50% |
| | | | 5p | | | | | |
| rs4148949 | CHST3 | 3′ UTR | hsa-miR- | C/T | C | 118 | C/T | −12.88% |
| | | | 450b-3p | | | | | |
| rs4148949 | CHST3 | 3′ UTR | hsa-miR-453 | C/T | C | 117 | C/T | −20.23% |
| rs4148949 | CHST3 | 3′ UTR | hsa-miR-493 | C/T | T | 133 | C/T | 25.69% |
| rs4148949 | CHST3 | 3′ UTR | hsa-miR-512- | C/T | C | 135 | C/T | −9.03% |
| | | | 3p | | | | | |
| rs4148949 | CHST3 | 3′ UTR | hsa-miR- | C/T | C | 97 | C/T | −10.28% |
| | | | 518c* | | | | | |
| rs4148949 | CHST3 | 3′ UTR | hsa-miR- | C/T | C | 129 | C/T | −10.17% |
| | | | 520b | | | | | |
| rs4148949 | CHST3 | 3′ UTR | hsa-miR- | C/T | C | 129 | C/T | −9.16% |
| | | | 520c-3p | | | | | |
| rs4148949 | CHST3 | 3′ UTR | hsa-miR-520f | C/T | C | 101 | C/T | −15.19% |
| rs4148949 | CHST3 | 3′ UTR | hsa-miR-523 | C/T | C | 85 | C/T | −58.01% |
| rs4148949 | CHST3 | 3′ UTR | hsa-miR- | C/T | C | 123 | C/T | −11.59% |
| | | | 526b* | | | | | |
| rs4148949 | CHST3 | 3′ UTR | hsa-miR-558 | C/T | C | 120 | C/T | −12.82% |
| rs4148949 | CHST3 | 3′ UTR | hsa-miR-562 | C/T | C | 116 | C/T | −146.01% |
| rs4148949 | CHST3 | 3′ UTR | hsa-miR-596 | C/T | C | 131 | C/T | −11.56% |
| rs4148949 | CHST3 | 3′ UTR | hsa-miR-608 | C/T | T | 107 | C/T | 8.64% |
| rs4148949 | CHST3 | 3′ UTR | hsa-miR-618 | C/T | T | 125 | C/T | 37.01% |
| rs4148949 | CHST3 | 3′ UTR | hsa-miR-622 | C/T | C | 141 | C/T | −11.97% |
| rs4148949 | CHST3 | 3′ UTR | hsa-miR-625 | C/T | T | 135 | C/T | 20.34% |
| rs4148949 | CHST3 | 3′ UTR | hsa-miR-654- | C/T | C | 139 | C/T | −11.63% |
| | | | 3p | | | | | |
| rs4148949 | CHST3 | 3′ UTR | hsa-miR-675 | C/T | C | 100 | C/T | −24.05% |
| rs4148949 | CHST3 | 3′ UTR | hsa-miR-708 | C/T | C | 110 | C/T | −9.73% |
| rs4148949 | CHST3 | 3′ UTR | hsa-miR-720 | C/T | C | 107 | C/T | −36.95% |
| rs4148949 | CHST3 | 3′ UTR | hsa-miR- | C/T | C | 113 | C/T | −16.66% |
| | | | 744* | | | | | |
| rs4148949 | CHST3 | 3′ UTR | hsa-miR-760 | C/T | C | 90 | C/T | −10.79% |
| rs4148949 | CHST3 | 3′ UTR | hsa-miR-765 | C/T | C | 141 | C/T | −8.17% |
| rs4148949 | CHST3 | 3′ UTR | hsa-miR-767- | C/T | C | 116 | C/T | −10.80% |
| | | | 3p | | | | | |
| rs4148949 | CHST3 | 3′ UTR | hsa-miR-874 | C/T | C | 80 | C/T | −8.33% |
| rs4148949 | CHST3 | 3′ UTR | hsa-miR-92a- | C/T | C | 124 | C/T | −13.66% |
| | | | 2* | | | | | |
| rs4148949 | CHST3 | 3′ UTR | hsa-miR-92b | C/T | T | 104 | C/T | 17.39% |
| rs4148949 | CHST3 | 3′ UTR | hsa-miR-93 | C/T | C | 130 | C/T | −8.53% |
| rs4148949 | CHST3 | 3′ UTR | hsa-miR-93* | C/T | C | 138 | C/T | −10.92% |
| rs10046 | CYP19A1 | 3′ UTR | hsa-miR- | C/T | T | 132 | C/T | 12.89% |
| | | | 106a | | | | | |
| rs10046 | CYP19A1 | 3′ UTR | hsa-miR- | C/T | C | 82 | C/T | −17.29% |
| | | | 1180 | | | | | |
| rs10046 | CYP19A1 | 3′ UTR | hsa-miR- | C/T | C | 122 | C/T | −11.68% |
| | | | 1183 | | | | | |
| rs10046 | CYP19A1 | 3′ UTR | hsa-miR- | C/T | T | 93 | C/T | 32.04% |
| | | | 1197 | | | | | |
| rs10046 | CYP19A1 | 3′ UTR | hsa-miR- | C/T | C | 86 | C/T | −9.25% |
| | | | 1202 | | | | | |
| rs10046 | CYP19A1 | 3′ UTR | hsa-miR- | C/T | T | 118 | C/T | 50.24% |
| | | | 1203 | | | | | |
| rs10046 | CYP19A1 | 3′ UTR | hsa-miR- | C/T | T | 102 | C/T | 13.53% |
| | | | 1207-5p | | | | | |
| rs10046 | CYP19A1 | 3′ UTR | hsa-miR- | C/T | C | 103 | C/T | −8.27% |
| | | | 1224-5p | | | | | |
| rs10046 | CYP19A1 | 3′ UTR | hsa-miR- | C/T | C | 172 | C/T | −12.75% |
| | | | 1225-5p | | | | | |
| rs10046 | CYP19A1 | 3′ UTR | hsa-miR- | C/T | C | 97 | C/T | −12.19% |
| | | | 1226* | | | | | |
| rs10046 | CYP19A1 | 3′ UTR | hsa-miR- | C/T | T | 115 | C/T | 44.26% |
| | | | 1253 | | | | | |
| rs10046 | CYP19A1 | 3′ UTR | hsa-miR- | C/T | T | 105 | C/T | 25.03% |
| | | | 1254 | | | | | |
| rs10046 | CYP19A1 | 3′ UTR | hsa-miR- | C/T | T | 121 | C/T | 10.47% |
| | | | 1256 | | | | | |
| rs10046 | CYP19A1 | 3′ UTR | hsa-miR- | C/T | C | 109 | C/T | −14.69% |
| | | | 1257 | | | | | |
| rs10046 | CYP19A1 | 3′ UTR | hsa-miR-127- | C/T | C | 115 | C/T | −9.56% |
| | | | 5p | | | | | |
| rs10046 | CYP19A1 | 3′ UTR | hsa-miR-128 | C/T | C | 126 | C/T | −8.55% |
| rs10046 | CYP19A1 | 3′ UTR | hsa-miR- | C/T | C | 116 | C/T | −16.52% |
| | | | 1292 | | | | | |
| rs10046 | CYP19A1 | 3′ UTR | hsa-miR- | C/T | C | 90 | C/T | −19.18% |
| | | | 1307 | | | | | |
| rs10046 | CYP19A1 | 3′ UTR | hsa-miR- | C/T | T | 109 | C/T | 21.14% |
| | | | 1321 | | | | | |
| rs10046 | CYP19A1 | 3′ UTR | hsa-miR-134 | C/T | C | 95 | C/T | −16.23% |
| rs10046 | CYP19A1 | 3′ UTR | hsa-miR-140- | C/T | T | 110 | C/T | 15.27% |
| | | | 3p | | | | | |
| rs10046 | CYP19A1 | 3′ UTR | hsa-miR- | C/T | C | 134 | C/T | −13.20% |
| | | | 146a | | | | | |
| rs10046 | CYP19A1 | 3′ UTR | hsa-miR- | C/T | T | 128 | C/T | 15.78% |
| | | | 146b-5p | | | | | |
| rs10046 | CYP19A1 | 3′ UTR | hsa-miR-154 | C/T | T | 96 | C/T | 27.22% |
| rs10046 | CYP19A1 | 3′ UTR | hsa-miR-17 | C/T | T | 132 | C/T | 12.89% |
| rs10046 | CYP19A1 | 3′ UTR | hsa-miR-18a | C/T | T | 108 | C/T | 62.25% |
| rs10046 | CYP19A1 | 3′ UTR | hsa-miR-18b | C/T | T | 108 | C/T | 62.25% |
| rs10046 | CYP19A1 | 3′ UTR | hsa-miR- | C/T | C | 114 | C/T | −11.41% |
| | | | 1909 | | | | | |
| rs10046 | CYP19A1 | 3′ UTR | hsa-miR- | C/T | C | 134 | C/T | −20.49% |
| | | | 1914* | | | | | |
| rs10046 | CYP19A1 | 3′ UTR | hsa-miR- | C/T | C | 146 | C/T | −16.74% |
| | | | 194* | | | | | |
| rs10046 | CYP19A1 | 3′ UTR | hsa-miR- | C/T | C | 101 | C/T | −8.65% |
| | | | 1978 | | | | | |
| rs10046 | CYP19A1 | 3′ UTR | hsa-miR-198 | C/T | C | 139 | C/T | −15.24% |
| rs10046 | CYP19A1 | 3′ UTR | hsa-miR-202 | C/T | C | 97 | C/T | −21.93% |
| rs10046 | CYP19A1 | 3′ UTR | hsa-miR-203 | C/T | T | 129 | C/T | 54.80% |
| rs10046 | CYP19A1 | 3′ UTR | hsa-miR- | C/T | C | 98 | C/T | −16.94% |
| | | | 20b* | | | | | |
| rs10046 | CYP19A1 | 3′ UTR | hsa-miR- | C/T | C | 109 | C/T | −16.19% |
| | | | 23a* | | | | | |
| rs10046 | CYP19A1 | 3′ UTR | hsa-miR- | C/T | C | 104 | C/T | −25.14% |
| | | | 23b* | | | | | |
| rs10046 | CYP19A1 | 3′ UTR | hsa-miR-296- | C/T | C | 110 | C/T | −15.57% |
| | | | 3p | | | | | |
| rs10046 | CYP19A1 | 3′ UTR | hsa-miR-298 | C/T | T | 102 | C/T | 17.24% |
| rs10046 | CYP19A1 | 3′ UTR | hsa-miR-324- | C/T | C | 127 | C/T | −9.11% |
| | | | 3p | | | | | |
| rs10046 | CYP19A1 | 3′ UTR | hsa-miR-325 | C/T | T | 122 | C/T | 11.81% |
| rs10046 | CYP19A1 | 3′ UTR | hsa-miR- | C/T | T | 94 | C/T | 36.07% |
| | | | 33b* | | | | | |
| rs10046 | CYP19A1 | 3′ UTR | hsa-miR-34c- | C/T | T | 111 | C/T | 42.68% |
| | | | 5p | | | | | |
| rs10046 | CYP19A1 | 3′ UTR | hsa-miR-361- | C/T | C | 120 | C/T | −9.10% |
| | | | 5p | | | | | |
| rs10046 | CYP19A1 | 3′ UTR | hsa-miR-373 | C/T | C | 137 | C/T | −12.58% |
| rs10046 | CYP19A1 | 3′ UTR | hsa-miR-423- | C/T | T | 103 | C/T | 10.75% |
| | | | 3p | | | | | |
| rs10046 | CYP19A1 | 3′ UTR | hsa-miR-432 | C/T | T | 105 | C/T | 32.76% |
| rs10046 | CYP19A1 | 3′ UTR | hsa-miR-433 | C/T | C | 100 | C/T | −11.67% |
| rs10046 | CYP19A1 | 3′ UTR | hsa-miR-490- | C/T | C | 96 | C/T | −13.00% |
| | | | 5p | | | | | |
| rs10046 | CYP19A1 | 3′ UTR | hsa-miR-491- | C/T | C | 133 | C/T | −16.65% |
| | | | 5p | | | | | |
| rs10046 | CYP19A1 | 3′ UTR | hsa-miR-496 | C/T | T | 103 | C/T | 38.27% |
| rs10046 | CYP19A1 | 3′ UTR | hsa-miR- | C/T | T | 118 | C/T | 12.73% |
| | | | 500* | | | | | |
| rs10046 | CYP19A1 | 3′ UTR | hsa-miR-508- | C/T | C | 155 | C/T | −16.64% |
| | | | 5p | | | | | |
| rs10046 | CYP19A1 | 3′ UTR | hsa-miR- | C/T | T | 90 | C/T | 21.51% |
| | | | 513a-5p | | | | | |
| rs10046 | CYP19A1 | 3′ UTR | hsa-miR- | C/T | C | 144 | C/T | −19.50% |
| | | | 517* | | | | | |
| rs10046 | CYP19A1 | 3′ UTR | hsa-miR- | C/T | C | 108 | C/T | −13.03% |
| | | | 519a | | | | | |
| rs10046 | CYP19A1 | 3′ UTR | hsa-miR- | C/T | T | 117 | C/T | 33.42% |
| | | | 519e* | | | | | |
| rs10046 | CYP19A1 | 3′ UTR | hsa-miR- | C/T | C | 129 | C/T | −12.34% |
| | | | 520c-3p | | | | | |
| rs10046 | CYP19A1 | 3′ UTR | hsa-miR- | C/T | C | 132 | C/T | −15.23% |
| | | | 520d-3p | | | | | |
| rs10046 | CYP19A1 | 3′ UTR | hsa-miR-520f | C/T | C | 117 | C/T | −11.57% |
| rs10046 | CYP19A1 | 3′ UTR | hsa-miR- | C/T | C | 124 | C/T | −9.04% |
| | | | 520g | | | | | |
| rs10046 | CYP19A1 | 3′ UTR | hsa-miR-532- | C/T | C | 95 | C/T | −22.79% |
| | | | 5p | | | | | |
| rs10046 | CYP19A1 | 3′ UTR | hsa-miR-550 | C/T | C | 101 | C/T | −21.07% |
| rs10046 | CYP19A1 | 3′ UTR | hsa-miR- | C/T | C | 96 | C/T | −13.44% |
| | | | 551b* | | | | | |
| rs10046 | CYP19A1 | 3′ UTR | hsa-miR-552 | C/T | C | 117 | C/T | −9.05% |
| rs10046 | CYP19A1 | 3′ UTR | hsa-miR-554 | C/T | T | 80 | C/T | 16.67% |
| rs10046 | CYP19A1 | 3′ UTR | hsa-miR-557 | C/T | C | 107 | C/T | −16.73% |
| rs10046 | CYP19A1 | 3′ UTR | hsa-miR-584 | C/T | C | 90 | C/T | −19.72% |
| rs10046 | CYP19A1 | 3′ UTR | hsa-miR- | C/T | T | 138 | C/T | 10.58% |
| | | | 593* | | | | | |
| rs10046 | CYP19A1 | 3′ UTR | hsa-miR-595 | C/T | T | 94 | C/T | 60.15% |
| rs10046 | CYP19A1 | 3′ UTR | hsa-miR-605 | C/T | T | 96 | C/T | 46.68% |
| rs10046 | CYP19A1 | 3′ UTR | hsa-miR-611 | C/T | C | 102 | C/T | −11.37% |
| rs10046 | CYP19A1 | 3′ UTR | hsa-miR-632 | C/T | C | 88 | C/T | −19.61% |
| rs10046 | CYP19A1 | 3′ UTR | hsa-miR-637 | C/T | C | 100 | C/T | −32.52% |
| rs10046 | CYP19A1 | 3′ UTR | hsa-miR-638 | C/T | C | 89 | C/T | −13.72% |
| rs10046 | CYP19A1 | 3′ UTR | hsa-miR-657 | C/T | T | 123 | C/T | 11.56% |
| rs10046 | CYP19A1 | 3′ UTR | hsa-miR-659 | C/T | C | 110 | C/T | −12.44% |
| rs10046 | CYP19A1 | 3′ UTR | hsa-miR-663 | C/T | T | 82 | C/T | 20.31% |
| rs10046 | CYP19A1 | 3′ UTR | hsa-miR-665 | C/T | T | 112 | C/T | 8.07% |
| rs10046 | CYP19A1 | 3′ UTR | hsa-miR-671- | C/T | C | 102 | C/T | −14.63% |
| | | | 5p | | | | | |
| rs10046 | CYP19A1 | 3′ UTR | hsa-miR-744 | C/T | T | 85 | C/T | 62.49% |
| rs10046 | CYP19A1 | 3′ UTR | hsa-miR-760 | C/T | C | 111 | C/T | −15.14% |
| rs10046 | CYP19A1 | 3′ UTR | hsa-miR-765 | C/T | T | 107 | C/T | 20.32% |
| rs10046 | CYP19A1 | 3′ UTR | hsa-miR-769- | C/T | C | 100 | C/T | −12.02% |
| | | | 5p | | | | | |
| rs10046 | CYP19A1 | 3′ UTR | hsa-miR-886- | C/T | C | 130 | C/T | −19.42% |
| | | | 3p | | | | | |
| rs10046 | CYP19A1 | 3′ UTR | hsa-miR-921 | C/T | T | 108 | C/T | 14.60% |
| rs10046 | CYP19A1 | 3′ UTR | hsa-miR-922 | C/T | T | 112 | C/T | 10.92% |
| rs10046 | CYP19A1 | 3′ UTR | hsa-miR-92a- | C/T | C | 120 | C/T | −18.79% |
| | | | 2* | | | | | |
| rs10046 | CYP19A1 | 3′ UTR | hsa-miR-938 | C/T | T | 79 | C/T | 11.91% |
| rs10046 | CYP19A1 | 3′ UTR | hsa-miR- | C/T | C | 98 | C/T | −9.34% |
| | | | 99a* | | | | | |
| rs10046 | CYP19A1 | 3′ UTR | hsa-miR- | C/T | C | 105 | C/T | −20.28% |
| | | | 99b* | | | | | |
| rs13181 | ERCC2 | CDS | hsa-let-7i* | G/T | G | 137 | G/T | −17.38% |
| rs13181 | ERCC2 | CDS | hsa-miR-103- | G/T | T | 120 | G/T | 41.42% |
| | | | 2* | | | | | |
| rs13181 | ERCC2 | CDS | hsa-miR-105 | G/T | G | 105 | G/T | −13.72% |
| rs13181 | ERCC2 | CDS | hsa-miR-10a | G/T | G | 94 | G/T | −57.55% |
| rs13181 | ERCC2 | CDS | hsa-miR- | G/T | G | 176 | G/T | −34.49% |
| | | | 1184 | | | | | |
| rs13181 | ERCC2 | CDS | hsa-miR- | G/T | G | 155 | G/T | −28.46% |
| | | | 1205 | | | | | |
| rs13181 | ERCC2 | CDS | hsa-miR- | G/T | T | 102 | G/T | 27.36% |
| | | | 1224-3p | | | | | |
| rs13181 | ERCC2 | CDS | hsa-miR- | G/T | G | 118 | G/T | −30.17% |
| | | | 1229 | | | | | |
| rs13181 | ERCC2 | CDS | hsa-miR- | G/T | T | 142 | G/T | 11.64% |
| | | | 1236 | | | | | |
| rs13181 | ERCC2 | CDS | hsa-miR- | G/T | G | 136 | G/T | −20.31% |
| | | | 1237 | | | | | |
| rs13181 | ERCC2 | CDS | hsa-miR- | G/T | G | 86 | G/T | −41.92% |
| | | | 124* | | | | | |
| rs13181 | ERCC2 | CDS | hsa-miR- | G/T | G | 119 | G/T | −19.03% |
| | | | 1251 | | | | | |
| rs13181 | ERCC2 | CDS | hsa-miR- | G/T | G | 113 | G/T | −17.72% |
| | | | 1256 | | | | | |
| rs13181 | ERCC2 | CDS | hsa-miR- | G/T | G | 129 | G/T | −28.92% |
| | | | 125a-5p | | | | | |
| rs13181 | ERCC2 | CDS | hsa-miR-127- | G/T | G | 108 | G/T | −51.59% |
| | | | 5p | | | | | |
| rs13181 | ERCC2 | CDS | hsa-miR- | G/T | G | 109 | G/T | −63.62% |
| | | | 1288 | | | | | |
| rs13181 | ERCC2 | CDS | hsa-miR- | G/T | T | 126 | G/T | 10.47% |
| | | | 1307 | | | | | |
| rs13181 | ERCC2 | CDS | hsa-miR- | G/T | G | 133 | G/T | −25.77% |
| | | | 130b* | | | | | |
| rs13181 | ERCC2 | CDS | hsa-miR- | G/T | T | 128 | G/T | 13.88% |
| | | | 136* | | | | | |
| rs13181 | ERCC2 | CDS | hsa-miR-139- | G/T | G | 111 | G/T | −15.86% |
| | | | 5p | | | | | |
| rs13181 | ERCC2 | CDS | hsa-miR-142- | G/T | G | 135 | G/T | −23.83% |
| | | | 3p | | | | | |
| rs13181 | ERCC2 | CDS | hsa-miR- | G/T | G | 131 | G/T | −19.90% |
| | | | 143* | | | | | |
| rs13181 | ERCC2 | CDS | hsa-miR- | G/T | G | 106 | G/T | −21.13% |
| | | | 1469 | | | | | |
| rs13181 | ERCC2 | CDS | hsa-miR- | G/T | G | 108 | G/T | −35.40% |
| | | | 147b | | | | | |
| rs13181 | ERCC2 | CDS | hsa-miR- | G/T | G | 128 | G/T | −39.96% |
| | | | 1539 | | | | | |
| rs13181 | ERCC2 | CDS | hsa-miR- | G/T | G | 98 | G/T | −44.97% |
| | | | 185* | | | | | |
| rs13181 | ERCC2 | CDS | hsa-miR- | G/T | G | 129 | G/T | −20.09% |
| | | | 191* | | | | | |
| rs13181 | ERCC2 | CDS | hsa-miR- | G/T | G | 133 | G/T | −30.87% |
| | | | 1913 | | | | | |
| rs13181 | ERCC2 | CDS | hsa-miR- | G/T | G | 136 | G/T | −32.67% |
| | | | 1914 | | | | | |
| rs13181 | ERCC2 | CDS | hsa-miR- | G/T | G | 80 | G/T | −26.87% |
| | | | 1915 | | | | | |
| rs13181 | ERCC2 | CDS | hsa-miR- | G/T | T | 89 | G/T | 8.74% |
| | | | 194* | | | | | |
| rs13181 | ERCC2 | CDS | hsa-miR- | G/T | G | 120 | G/T | −35.15% |
| | | | 1976 | | | | | |
| rs13181 | ERCC2 | CDS | hsa-miR- | G/T | G | 93 | G/T | −16.10% |
| | | | 200c* | | | | | |
| rs13181 | ERCC2 | CDS | hsa-miR- | G/T | G | 136 | G/T | −118.79 |
| | | | 202* | | | | | |
| rs13181 | ERCC2 | CDS | hsa-miR-204 | G/T | T | 111 | G/T | 47.17% |
| rs13181 | ERCC2 | CDS | hsa-miR-20b | G/T | G | 100 | G/T | −26.81% |
| rs13181 | ERCC2 | CDS | hsa-miR-210 | G/T | G | 110 | G/T | −27.77% |
| rs13181 | ERCC2 | CDS | hsa-miR- | G/T | G | 105 | G/T | −52.94% |
| | | | 2110 | | | | | |
| rs13181 | ERCC2 | CDS | hsa-miR- | G/T | G | 116 | G/T | −19.07% |
| | | | 214* | | | | | |
| rs13181 | ERCC2 | CDS | hsa-miR- | G/T | G | 108 | G/T | −49.07% |
| | | | 216b | | | | | |
| rs13181 | ERCC2 | CDS | hsa-miR-218- | G/T | G | 87 | G/T | −19.55% |
| | | | 2* | | | | | |
| rs13181 | ERCC2 | CDS | hsa-miR-22* | G/T | T | 115 | G/T | 56.07% |
| rs13181 | ERCC2 | CDS | hsa-miR-221 | G/T | G | 115 | G/T | −41.96% |
| rs13181 | ERCC2 | CDS | hsa-miR-25 | G/T | G | 101 | G/T | −54.04% |
| rs13181 | ERCC2 | CDS | hsa-miR- | G/T | G | 87 | G/T | −35.40% |
| | | | 27a* | | | | | |
| rs13181 | ERCC2 | CDS | hsa-miR- | G/T | G | 106 | G/T | −27.33% |
| | | | 29c* | | | | | |
| rs13181 | ERCC2 | CDS | hsa-miR- | G/T | T | 102 | G/T | 33.71% |
| | | | 302c | | | | | |
| rs13181 | ERCC2 | CDS | hsa-miR-326 | G/T | G | 128 | G/T | −59.90% |
| rs13181 | ERCC2 | CDS | hsa-miR-328 | G/T | G | 101 | G/T | −11.65% |
| rs13181 | ERCC2 | CDS | hsa-miR-330- | G/T | G | 108 | G/T | −20.86% |
| | | | 3p | | | | | |
| rs13181 | ERCC2 | CDS | hsa-miR-330- | G/T | G | 113 | G/T | −11.75% |
| | | | 5p | | | | | |
| rs13181 | ERCC2 | CDS | hsa-miR-346 | G/T | T | 112 | G/T | 22.96% |
| rs13181 | ERCC2 | CDS | hsa-miR-34a | G/T | G | 145 | G/T | −12.36% |
| rs13181 | ERCC2 | CDS | hsa-miR- | G/T | G | 122 | G/T | −17.34% |
| | | | 34b* | | | | | |
| rs13181 | ERCC2 | CDS | hsa-miR-34c- | G/T | G | 131 | G/T | −17.22% |
| | | | 5p | | | | | |
| rs13181 | ERCC2 | CDS | hsa-miR-370 | G/T | G | 104 | G/T | −33.41% |
| rs13181 | ERCC2 | CDS | hsa-miR-423- | G/T | G | 118 | G/T | −11.14% |
| | | | 3p | | | | | |
| rs13181 | ERCC2 | CDS | hsa-miR- | G/T | G | 131 | G/T | −13.32% |
| | | | 449a | | | | | |
| rs13181 | ERCC2 | CDS | hsa-miR- | G/T | G | 131 | G/T | −17.42% |
| | | | 449b | | | | | |
| rs13181 | ERCC2 | CDS | hsa-miR- | G/T | G | 92 | G/T | −40.40% |
| | | | 450a | | | | | |
| rs13181 | ERCC2 | CDS | hsa-miR-489 | G/T | G | 89 | G/T | −10.97% |
| rs13181 | ERCC2 | CDS | hsa-miR-501- | G/T | G | 123 | G/T | −13.12% |
| | | | 5p | | | | | |
| rs13181 | ERCC2 | CDS | hsa-miR-503 | G/T | G | 95 | G/T | −29.35% |
| rs13181 | ERCC2 | CDS | hsa-miR-504 | G/T | T | 95 | G/T | 25.96% |
| rs13181 | ERCC2 | CDS | hsa-miR-507 | G/T | G | 113 | G/T | −21.67% |
| rs13181 | ERCC2 | CDS | hsa-miR-511 | G/T | G | 102 | G/T | −11.12% |
| rs13181 | ERCC2 | CDS | hsa-miR- | G/T | T | 92 | G/T | 49.56% |
| | | | 517a | | | | | |
| rs13181 | ERCC2 | CDS | hsa-miR- | G/T | T | 84 | G/T | 36.60% |
| | | | 517b | | | | | |
| rs13181 | ERCC2 | CDS | hsa-miR- | G/T | T | 90 | G/T | 99.07% |
| | | | 517c | | | | | |
| rs13181 | ERCC2 | CDS | hsa-miR- | G/T | G | 125 | G/T | −29.99% |
| | | | 518c* | | | | | |
| rs13181 | ERCC2 | CDS | hsa-miR- | G/T | T | 113 | G/T | 13.21% |
| | | | 518e | | | | | |
| rs13181 | ERCC2 | CDS | hsa-miR- | G/T | T | 88 | G/T | 66.80% |
| | | | 519a | | | | | |
| rs13181 | ERCC2 | CDS | hsa-miR- | G/T | T | 84 | G/T | 50.27% |
| | | | 519b-3p | | | | | |
| rs13181 | ERCC2 | CDS | hsa-miR- | G/T | T | 105 | G/T | 10.20% |
| | | | 519d | | | | | |
| rs13181 | ERCC2 | CDS | hsa-miR- | G/T | T | 102 | G/T | 18.43% |
| | | | 520d-3p | | | | | |
| rs13181 | ERCC2 | CDS | hsa-miR- | G/T | T | 109 | G/T | 23.43% |
| | | | 520h | | | | | |
| rs13181 | ERCC2 | CDS | hsa-miR-524- | G/T | G | 91 | G/T | −30.30% |
| | | | 3p | | | | | |
| rs13181 | ERCC2 | CDS | hsa-miR-555 | G/T | G | 103 | G/T | −24.43% |
| rs13181 | ERCC2 | CDS | hsa-miR-589 | G/T | G | 103 | G/T | −9.39% |
| rs13181 | ERCC2 | CDS | hsa-miR-593 | G/T | G | 127 | G/T | −18.65% |
| rs13181 | ERCC2 | CDS | hsa-miR-612 | G/T | G | 155 | G/T | −24.26% |
| rs13181 | ERCC2 | CDS | hsa-miR- | G/T | T | 90 | G/T | 30.63% |
| | | | 616* | | | | | |
| rs13181 | ERCC2 | CDS | hsa-miR-618 | G/T | G | 117 | G/T | −15.54% |
| rs13181 | ERCC2 | CDS | hsa-miR-622 | G/T | G | 132 | G/T | −34.24% |
| rs13181 | ERCC2 | CDS | hsa-miR-623 | G/T | G | 123 | G/T | −26.83% |
| rs13181 | ERCC2 | CDS | hsa-miR-632 | G/T | G | 123 | G/T | −15.04% |
| rs13181 | ERCC2 | CDS | hsa-miR-636 | G/T | G | 88 | G/T | −18.86% |
| rs13181 | ERCC2 | CDS | hsa-miR-637 | G/T | G | 96 | G/T | −36.82% |
| rs13181 | ERCC2 | CDS | hsa-miR-646 | G/T | G | 123 | G/T | −10.63% |
| rs13181 | ERCC2 | CDS | hsa-miR-650 | G/T | G | 95 | G/T | −19.23% |
| rs13181 | ERCC2 | CDS | hsa-miR-661 | G/T | G | 119 | G/T | −14.08% |
| rs13181 | ERCC2 | CDS | hsa-miR-720 | G/T | G | 106 | G/T | −86.00% |
| rs13181 | ERCC2 | CDS | hsa-miR-760 | G/T | G | 119 | G/T | −29.28% |
| rs13181 | ERCC2 | CDS | hsa-miR- | G/T | T | 170 | G/T | 22.05% |
| | | | 877* | | | | | |
| rs13181 | ERCC2 | CDS | hsa-miR- | G/T | T | 98 | G/T | 24.42% |
| | | | 888* | | | | | |
| rs13181 | ERCC2 | CDS | hsa-miR- | G/T | T | 101 | G/T | 21.79% |
| | | | 892a | | | | | |
| rs13181 | ERCC2 | CDS | hsa-miR- | G/T | G | 96 | G/T | −32.80% |
| | | | 892b | | | | | |
| rs13181 | ERCC2 | CDS | hsa-miR-93 | G/T | G | 100 | G/T | −29.30% |
| rs13181 | ERCC2 | CDS | hsa-miR-937 | G/T | T | 127 | G/T | 10.30% |
| rs13181 | ERCC2 | CDS | hsa-miR-939 | G/T | G | 103 | G/T | −30.98% |
| rs13181 | ERCC2 | CDS | hsa-miR- | G/T | G | 97 | G/T | −61.01% |
| | | | 99b* | | | | | |
| rs17655 | ERCC5 | CDS | hsa-miR- | C/G | G | 135 | C/G | 28.89% |
| | | | 124* | | | | | |
| rs17655 | ERCC5 | CDS | hsa-miR- | C/G | C | 78 | C/G | −8.81% |
| | | | 1244 | | | | | |
| rs17655 | ERCC5 | CDS | hsa-miR- | C/G | C | 128 | C/G | −11.75% |
| | | | 1272 | | | | | |
| rs17655 | ERCC5 | CDS | hsa-miR-129- | C/G | C | 110 | C/G | −37.46% |
| | | | 5p | | | | | |
| rs17655 | ERCC5 | CDS | hsa-miR- | C/G | C | 118 | C/G | −66.53% |
| | | | 1305 | | | | | |
| rs17655 | ERCC5 | CDS | hsa-miR- | C/G | C | 88 | C/G | −21.44% |
| | | | 1469 | | | | | |
| rs17655 | ERCC5 | CDS | hsa-miR- | C/G | C | 85 | C/G | −39.16% |
| | | | 1471 | | | | | |
| rs17655 | ERCC5 | CDS | hsa-miR- | C/G | C | 80 | C/G | −221.65 |
| | | | 148b | | | | | |
| rs17655 | ERCC5 | CDS | hsa-miR-17* | C/G | C | 93 | C/G | −20.51% |
| rs17655 | ERCC5 | CDS | hsa-miR-185 | C/G | G | 87 | C/G | 22.39% |
| rs17655 | ERCC5 | CDS | hsa-miR- | C/G | C | 115 | C/G | −8.14% |
| | | | 191* | | | | | |
| rs17655 | ERCC5 | CDS | hsa-miR-195 | C/G | C | 106 | C/G | −18.06% |
| rs17655 | ERCC5 | CDS | hsa-miR- | C/G | G | 92 | C/G | 36.12% |
| | | | 20b* | | | | | |
| rs17655 | ERCC5 | CDS | hsa-miR-29a | C/G | C | 107 | C/G | −79.07% |
| rs17655 | ERCC5 | CDS | hsa-miR- | C/G | G | 100 | C/G | 32.85% |
| | | | 302d* | | | | | |
| rs17655 | ERCC5 | CDS | hsa-miR- | C/G | G | 138 | C/G | 29.46% |
| | | | 30d* | | | | | |
| rs17655 | ERCC5 | CDS | hsa-miR- | C/G | G | 138 | C/G | 29.61% |
| | | | 30e* | | | | | |
| rs17655 | ERCC5 | CDS | hsa-miR-323- | C/G | G | 105 | C/G | 22.36% |
| | | | 5p | | | | | |
| rs17655 | ERCC5 | CDS | hsa-miR- | C/G | C | 82 | C/G | −32.03% |
| | | | 33b* | | | | | |
| rs17655 | ERCC5 | CDS | hsa-miR- | C/G | G | 118 | C/G | 9.72% |
| | | | 365* | | | | | |
| rs17655 | ERCC5 | CDS | hsa-miR-372 | C/G | C | 80 | C/G | −33.55% |
| rs17655 | ERCC5 | CDS | hsa-miR-412 | C/G | C | 133 | C/G | −28.72% |
| rs17655 | ERCC5 | CDS | hsa-miR-423- | C/G | C | 80 | C/G | −21.74% |
| | | | 3p | | | | | |
| rs17655 | ERCC5 | CDS | hsa-miR-505 | C/G | C | 95 | C/G | −26.30% |
| rs17655 | ERCC5 | CDS | hsa-miR-541 | C/G | C | 114 | C/G | −57.84% |
| rs17655 | ERCC5 | CDS | hsa-miR-567 | C/G | G | 130 | C/G | 20.04% |
| rs17655 | ERCC5 | CDS | hsa-miR-596 | C/G | C | 92 | C/G | −84.76% |
| rs17655 | ERCC5 | CDS | hsa-miR-598 | C/G | C | 86 | C/G | −86.88% |
| rs17655 | ERCC5 | CDS | hsa-miR-614 | C/G | C | 74 | C/G | −40.77% |
| rs17655 | ERCC5 | CDS | hsa-miR-654- | C/G | G | 107 | C/G | 35.34% |
| | | | 5p | | | | | |
| rs17655 | ERCC5 | CDS | hsa-miR-659 | C/G | G | 102 | C/G | 28.17% |
| rs17655 | ERCC5 | CDS | hsa-miR-766 | C/G | C | 103 | C/G | −35.19% |
| rs1061646 | ZNF276 | 3′ UTR | hsa-let-7g* | C/T | C | 123 | C/T | −34.12% |
| rs1061646 | ZNF276 | 3′ UTR | hsa-miR- | C/T | C | 84 | C/T | −14.42% |
| | | | 1183 | | | | | |
| rs1061646 | ZNF276 | 3′ UTR | hsa-miR- | C/T | C | 127 | C/T | −43.04% |
| | | | 1184 | | | | | |
| rs1061646 | ZNF276 | 3′ UTR | hsa-miR- | C/T | C | 151 | C/T | −55.63% |
| | | | 1225-3p | | | | | |
| rs1061646 | ZNF276 | 3′ UTR | hsa-miR- | C/T | C | 130 | C/T | −22.21% |
| | | | 1234 | | | | | |
| rs1061646 | ZNF276 | 3′ UTR | hsa-miR- | C/T | C | 145 | C/T | −10.71% |
| | | | 125a-5p | | | | | |
| rs1061646 | ZNF276 | 3′ UTR | hsa-miR- | C/T | C | 149 | C/T | −8.76% |
| | | | 125b | | | | | |
| rs1061646 | ZNF276 | 3′ UTR | hsa-miR- | C/T | C | 104 | C/T | −10.32% |
| | | | 1276 | | | | | |
| rs1061646 | ZNF276 | 3′ UTR | hsa-miR- | C/T | C | 121 | C/T | −12.90% |
| | | | 1285 | | | | | |
| rs1061646 | ZNF276 | 3′ UTR | hsa-miR- | C/T | C | 102 | C/T | −14.64% |
| | | | 1295 | | | | | |
| rs1061646 | ZNF276 | 3′ UTR | hsa-miR- | C/T | C | 100 | C/T | −21.79% |
| | | | 1296 | | | | | |
| rs1061646 | ZNF276 | 3′ UTR | hsa-miR- | C/T | C | 118 | C/T | −50.86% |
| | | | 1301 | | | | | |
| rs1061646 | ZNF276 | 3′ UTR | hsa-miR-138- | C/T | C | 129 | C/T | −47.23% |
| | | | 1* | | | | | |
| rs1061646 | ZNF276 | 3′ UTR | hsa-miR-140- | C/T | C | 111 | C/T | −24.08% |
| | | | 3p | | | | | |
| rs1061646 | ZNF276 | 3′ UTR | hsa-miR-142- | C/T | C | 104 | C/T | −22.70% |
| | | | 3p | | | | | |
| rs1061646 | ZNF276 | 3′ UTR | hsa-miR- | C/T | C | 81 | C/T | −15.90% |
| | | | 1469 | | | | | |
| rs1061646 | ZNF276 | 3′ UTR | hsa-miR- | C/T | C | 100 | C/T | −14.62% |
| | | | 146b-3p | | | | | |
| rs1061646 | ZNF276 | 3′ UTR | hsa-miR- | C/T | C | 91 | C/T | −92.48% |
| | | | 146b-5p | | | | | |
| rs1061646 | ZNF276 | 3′ UTR | hsa-miR- | C/T | T | 100 | C/T | 42.13% |
| | | | 148b* | | | | | |
| rs1061646 | ZNF276 | 3′ UTR | hsa-miR- | C/T | C | 116 | C/T | −13.05% |
| | | | 1538 | | | | | |
| rs1061646 | ZNF276 | 3′ UTR | hsa-miR-16- | C/T | C | 107 | C/T | −25.60% |
| | | | 1* | | | | | |
| rs1061646 | ZNF276 | 3′ UTR | hsa-miR- | C/T | C | 101 | C/T | −12.97% |
| | | | 18b* | | | | | |
| rs1061646 | ZNF276 | 3′ UTR | hsa-miR- | C/T | C | 99 | C/T | −16.64% |
| | | | 1909 | | | | | |
| rs1061646 | ZNF276 | 3′ UTR | hsa-miR- | C/T | C | 140 | C/T | −20.78% |
| | | | 1914 | | | | | |
| rs1061646 | ZNF276 | 3′ UTR | hsa-miR- | C/T | C | 83 | C/T | −17.97% |
| | | | 195* | | | | | |
| rs1061646 | ZNF276 | 3′ UTR | hsa-miR- | C/T | C | 108 | C/T | −29.38% |
| | | | 1976 | | | | | |
| rs1061646 | ZNF276 | 3′ UTR | hsa-miR- | C/T | C | 107 | C/T | −34.38% |
| | | | 199a-3p | | | | | |
| rs1061646 | ZNF276 | 3′ UTR | hsa-miR- | C/T | C | 107 | C/T | −34.38% |
| | | | 199b-3p | | | | | |
| rs1061646 | ZNF276 | 3′ UTR | hsa-miR- | C/T | C | 130 | C/T | −15.47% |
| | | | 214* | | | | | |
| rs1061646 | ZNF276 | 3′ UTR | hsa-miR-221 | C/T | C | 110 | C/T | −70.67% |
| rs1061646 | ZNF276 | 3′ UTR | hsa-miR- | C/T | C | 98 | C/T | −8.55% |
| | | | 27b* | | | | | |
| rs1061646 | ZNF276 | 3′ UTR | hsa-miR-29b- | C/T | T | 88 | C/T | 53.19% |
| | | | 1* | | | | | |
| rs1061646 | ZNF276 | 3′ UTR | hsa-miR- | C/T | C | 92 | C/T | −31.44% |
| | | | 30b* | | | | | |
| rs1061646 | ZNF276 | 3′ UTR | hsa-miR-326 | C/T | C | 113 | C/T | −40.51% |
| rs1061646 | ZNF276 | 3′ UTR | hsa-miR-339- | C/T | C | 101 | C/T | −24.14% |
| | | | 5p | | | | | |
| rs1061646 | ZNF276 | 3′ UTR | hsa-miR- | C/T | C | 126 | C/T | −10.41% |
| | | | 33b* | | | | | |
| rs1061646 | ZNF276 | 3′ UTR | hsa-miR- | C/T | C | 89 | C/T | −16.24% |
| | | | 431* | | | | | |
| rs1061646 | ZNF276 | 3′ UTR | hsa-miR-500 | C/T | C | 85 | C/T | −19.12% |
| rs1061646 | ZNF276 | 3′ UTR | hsa-miR-508- | C/T | C | 132 | C/T | −19.45% |
| | | | 5p | | | | | |
| rs1061646 | ZNF276 | 3′ UTR | hsa-miR-511 | C/T | C | 80 | C/T | −64.86% |
| rs1061646 | ZNF276 | 3′ UTR | hsa-miR-512- | C/T | C | 102 | C/T | −24.55% |
| | | | 5p | | | | | |
| rs1061646 | ZNF276 | 3′ UTR | hsa-miR-514 | C/T | C | 113 | C/T | −176.57% |
| rs1061646 | ZNF276 | 3′ UTR | hsa-miR- | C/T | C | 90 | C/T | −15.71% |
| | | | 519e* | | | | | |
| rs1061646 | ZNF276 | 3′ UTR | hsa-miR- | C/T | C | 152 | C/T | −15.91% |
| | | | 520a-5p | | | | | |
| rs1061646 | ZNF276 | 3′ UTR | hsa-miR-550 | C/T | C | 143 | C/T | −9.39% |
| rs1061646 | ZNF276 | 3′ UTR | hsa-miR- | C/T | C | 131 | C/T | −32.75% |
| | | | 551a | | | | | |
| rs1061646 | ZNF276 | 3′ UTR | hsa-miR-564 | C/T | C | 98 | C/T | −29.07% |
| rs1061646 | ZNF276 | 3′ UTR | hsa-miR-587 | C/T | C | 122 | C/T | −9.91% |
| rs1061646 | ZNF276 | 3′ UTR | hsa-miR-588 | C/T | C | 133 | C/T | −23.05% |
| rs1061646 | ZNF276 | 3′ UTR | hsa-miR-640 | C/T | C | 113 | C/T | −8.20% |
| rs1061646 | ZNF276 | 3′ UTR | hsa-miR-652 | C/T | C | 89 | C/T | −23.28% |
| rs1061646 | ZNF276 | 3′ UTR | hsa-miR-661 | C/T | T | 154 | C/T | 12.98% |
| rs1061646 | ZNF276 | 3′ UTR | hsa-miR- | C/T | C | 110 | C/T | −10.83% |
| | | | 663b | | | | | |
| rs1061646 | ZNF276 | 3′ UTR | hsa-miR- | C/T | C | 113 | C/T | −23.47% |
| | | | 888* | | | | | |
| rs1061646 | ZNF276 | 3′ UTR | hsa-miR- | C/T | C | 83 | C/T | −27.52% |
| | | | 891a | | | | | |
| rs1061646 | ZNF276 | 3′ UTR | hsa-miR- | C/T | T | 91 | C/T | 154.07% |
| | | | 891b | | | | | |
| rs1061646 | ZNF276 | 3′ UTR | hsa-miR-921 | C/T | C | 89 | C/T | −10.17% |
| rs1061646 | ZNF276 | 3′ UTR | hsa-miR-93* | C/T | C | 118 | C/T | −24.06% |
| rs1061646 | ZNF276 | 3′ UTR | hsa-miR-938 | C/T | C | 106 | C/T | −173.21% |
| rs1061646 | ZNF276 | 3′ UTR | hsa-miR-939 | C/T | C | 107 | C/T | −9.72% |
| rs1061646 | ZNF276 | 3′ UTR | hsa-miR-941 | C/T | C | 84 | C/T | −79.26% |
| rs1061646 | ZNF276 | 3′ UTR | hsa-miR- | C/T | C | 92 | C/T | −41.28% |
| | | | 99a* | | | | | |
| rs572169 | GHSR | CDS | hsa-miR-105 | A/G | G | 81 | A/G | 24.00% |
| rs572169 | GHSR | CDS | hsa-miR- | A/G | G | 103 | A/G | 30.90% |
| | | | 106b* | | | | | |
| rs572169 | GHSR | CDS | hsa-miR- | A/G | A | 118 | A/G | −38.75% |
| | | | 1181 | | | | | |
| rs572169 | GHSR | CDS | hsa-miR- | A/G | G | 107 | A/G | 17.83% |
| | | | 1184 | | | | | |
| rs572169 | GHSR | CDS | hsa-miR- | A/G | G | 154 | A/G | 35.63% |
| | | | 1226 | | | | | |
| rs572169 | GHSR | CDS | hsa-miR- | A/G | G | 121 | A/G | 11.89% |
| | | | 1229 | | | | | |
| rs572169 | GHSR | CDS | hsa-miR- | A/G | G | 105 | A/G | 23.82% |
| | | | 1233 | | | | | |
| rs572169 | GHSR | CDS | hsa-miR- | A/G | G | 167 | A/G | 41.58% |
| | | | 1247 | | | | | |
| rs572169 | GHSR | CDS | hsa-miR- | A/G | G | 134 | A/G | 36.60% |
| | | | 1260 | | | | | |
| rs572169 | GHSR | CDS | hsa-miR-127- | A/G | G | 90 | A/G | 14.64% |
| | | | 3p | | | | | |
| rs572169 | GHSR | CDS | hsa-miR- | A/G | G | 105 | A/G | 18.37% |
| | | | 1280 | | | | | |
| rs572169 | GHSR | CDS | hsa-miR- | A/G | G | 100 | A/G | 13.55% |
| | | | 1288 | | | | | |
| rs572169 | GHSR | CDS | hsa-miR- | A/G | G | 90 | A/G | 63.76% |
| | | | 1289 | | | | | |
| rs572169 | GHSR | CDS | hsa-miR- | A/G | G | 120 | A/G | 10.23% |
| | | | 129* | | | | | |
| rs572169 | GHSR | CDS | hsa-miR-129- | A/G | G | 120 | A/G | 10.23% |
| | | | 3p | | | | | |
| rs572169 | GHSR | CDS | hsa-miR- | A/G | G | 98 | A/G | 16.51% |
| | | | 1295 | | | | | |
| rs572169 | GHSR | CDS | hsa-miR- | A/G | A | 90 | A/G | −13.12% |
| | | | 1303 | | | | | |
| rs572169 | GHSR | CDS | hsa-miR- | A/G | A | 107 | A/G | −10.90% |
| | | | 1307 | | | | | |
| rs572169 | GHSR | CDS | hsa-miR-134 | A/G | A | 101 | A/G | −23.90% |
| rs572169 | GHSR | CDS | hsa-miR-139- | A/G | G | 84 | A/G | 9.35% |
| | | | 5p | | | | | |
| rs572169 | GHSR | CDS | hsa-miR-140- | A/G | G | 63 | A/G | 8.19% |
| | | | 5p | | | | | |
| rs572169 | GHSR | CDS | hsa-miR- | A/G | G | 90 | A/G | 12.71% |
| | | | 143* | | | | | |
| rs572169 | GHSR | CDS | hsa-miR-145 | A/G | G | 130 | A/G | 37.76% |
| rs572169 | GHSR | CDS | hsa-miR- | A/G | G | 124 | A/G | 11.72% |
| | | | 1470 | | | | | |
| rs572169 | GHSR | CDS | hsa-miR- | A/G | G | 124 | A/G | 30.37% |
| | | | 1471 | | | | | |
| rs572169 | GHSR | CDS | hsa-miR-150 | A/G | G | 104 | A/G | 36.10% |
| rs572169 | GHSR | CDS | hsa-miR-17* | A/G | A | 85 | A/G | −16.03% |
| rs572169 | GHSR | CDS | hsa-miR- | A/G | A | 92 | A/G | −32.93% |
| | | | 181b | | | | | |
| rs572169 | GHSR | CDS | hsa-miR-185 | A/G | G | 103 | A/G | 37.73% |
| rs572169 | GHSR | CDS | hsa-miR- | A/G | A | 97 | A/G | −14.34% |
| | | | 185* | | | | | |
| rs572169 | GHSR | CDS | hsa-miR- | A/G | G | 95 | A/G | 31.73% |
| | | | 1909* | | | | | |
| rs572169 | GHSR | CDS | hsa-miR- | A/G | G | 125 | A/G | 14.15% |
| | | | 1910 | | | | | |
| rs572169 | GHSR | CDS | hsa-miR- | A/G | G | 103 | A/G | 15.85% |
| | | | 1911 | | | | | |
| rs572169 | GHSR | CDS | hsa-miR- | A/G | G | 116 | A/G | 22.03% |
| | | | 1911* | | | | | |
| rs572169 | GHSR | CDS | hsa-miR- | A/G | G | 162 | A/G | 23.64% |
| | | | 1913 | | | | | |
| rs572169 | GHSR | CDS | hsa-miR-194 | A/G | A | 87 | A/G | −50.40% |
| rs572169 | GHSR | CDS | hsa-miR-197 | A/G | G | 111 | A/G | 16.84% |
| rs572169 | GHSR | CDS | hsa-miR- | A/G | G | 137 | A/G | 24.20% |
| | | | 1976 | | | | | |
| rs572169 | GHSR | CDS | hsa-miR- | A/G | A | 100 | A/G | −23.47% |
| | | | 1979 | | | | | |
| rs572169 | GHSR | CDS | hsa-miR- | A/G | G | 103 | A/G | 34.44% |
| | | | 199a-5p | | | | | |
| rs572169 | GHSR | CDS | hsa-miR- | A/G | G | 117 | A/G | 77.05% |
| | | | 220a | | | | | |
| rs572169 | GHSR | CDS | hsa-miR-222 | A/G | G | 126 | A/G | 31.48% |
| rs572169 | GHSR | CDS | hsa-miR-26a- | A/G | G | 83 | A/G | 15.25% |
| | | | 1* | | | | | |
| rs572169 | GHSR | CDS | hsa-miR- | A/G | G | 96 | A/G | 46.74% |
| | | | 26b* | | | | | |
| rs572169 | GHSR | CDS | hsa-miR-296- | A/G | G | 128 | A/G | 32.02% |
| | | | 5p | | | | | |
| rs572169 | GHSR | CDS | hsa-miR-299- | A/G | G | 106 | A/G | 9.65% |
| | | | 5p | | | | | |
| rs572169 | GHSR | CDS | hsa-miR-29b- | A/G | G | 105 | A/G | 42.51% |
| | | | 2* | | | | | |
| rs572169 | GHSR | CDS | hsa-miR-324- | A/G | G | 121 | A/G | 9.99% |
| | | | 5p | | | | | |
| rs572169 | GHSR | CDS | hsa-miR-329 | A/G | G | 114 | A/G | 36.59% |
| rs572169 | GHSR | CDS | hsa-miR-339- | A/G | G | 122 | A/G | 28.61% |
| | | | 5p | | | | | |
| rs572169 | GHSR | CDS | hsa-miR- | A/G | G | 124 | A/G | 26.28% |
| | | | 340* | | | | | |
| rs572169 | GHSR | CDS | hsa-miR-342- | A/G | A | 97 | A/G | −29.92% |
| | | | 3p | | | | | |
| rs572169 | GHSR | CDS | hsa-miR-346 | A/G | G | 113 | A/G | 22.65% |
| rs572169 | GHSR | CDS | hsa-miR- | A/G | G | 119 | A/G | 16.72% |
| | | | 34a* | | | | | |
| rs572169 | GHSR | CDS | hsa-miR-34c- | A/G | A | 106 | A/G | −30.16% |
| | | | 3p | | | | | |
| rs572169 | GHSR | CDS | hsa-miR-373 | A/G | A | 82 | A/G | −52.28% |
| rs572169 | GHSR | CDS | hsa-miR- | A/G | G | 102 | A/G | 29.13% |
| | | | 378* | | | | | |
| rs572169 | GHSR | CDS | hsa-miR-409- | A/G | A | 80 | A/G | −39.32% |
| | | | 3p | | | | | |
| rs572169 | GHSR | CDS | hsa-miR-412 | A/G | G | 165 | A/G | 23.34% |
| rs572169 | GHSR | CDS | hsa-miR- | A/G | G | 86 | A/G | 27.78% |
| | | | 425* | | | | | |
| rs572169 | GHSR | CDS | hsa-miR-433 | A/G | G | 96 | A/G | 28.37% |
| rs572169 | GHSR | CDS | hsa-miR-451 | A/G | A | 84 | A/G | −69.58% |
| rs572169 | GHSR | CDS | hsa-miR-453 | A/G | A | 91 | A/G | −41.75% |
| rs572169 | GHSR | CDS | hsa-miR-455- | A/G | G | 100 | A/G | 9.88% |
| | | | 5p | | | | | |
| rs572169 | GHSR | CDS | hsa-miR-483- | A/G | A | 117 | A/G | −26.81% |
| | | | 3p | | | | | |
| rs572169 | GHSR | CDS | hsa-miR-484 | A/G | G | 107 | A/G | 11.23% |
| rs572169 | GHSR | CDS | hsa-miR-486- | A/G | G | 100 | A/G | 17.12% |
| | | | 5p | | | | | |
| rs572169 | GHSR | CDS | hsa-miR-492 | A/G | G | 118 | A/G | 13.89% |
| rs572169 | GHSR | CDS | hsa-miR-497 | A/G | G | 104 | A/G | 26.59% |
| rs572169 | GHSR | CDS | hsa-miR-510 | A/G | A | 82 | A/G | −9.50% |
| rs572169 | GHSR | CDS | hsa-miR-512- | A/G | A | 123 | A/G | −60.06% |
| | | | 5p | | | | | |
| rs572169 | GHSR | CDS | hsa-miR-524- | A/G | G | 97 | A/G | 10.30% |
| | | | 3p | | | | | |
| rs572169 | GHSR | CDS | hsa-miR- | A/G | G | 86 | A/G | 15.46% |
| | | | 541* | | | | | |
| rs572169 | GHSR | CDS | hsa-miR-572 | A/G | G | 145 | A/G | 23.61% |
| rs572169 | GHSR | CDS | hsa-miR-589 | A/G | G | 100 | A/G | 52.59% |
| rs572169 | GHSR | CDS | hsa-miR-596 | A/G | G | 116 | A/G | 16.04% |
| rs572169 | GHSR | CDS | hsa-miR-608 | A/G | G | 94 | A/G | 14.75% |
| rs572169 | GHSR | CDS | hsa-miR-611 | A/G | A | 105 | A/G | −12.69% |
| rs572169 | GHSR | CDS | hsa-miR-614 | A/G | G | 113 | A/G | 26.70% |
| rs572169 | GHSR | CDS | hsa-miR-615- | A/G | G | 127 | A/G | 18.21% |
| | | | 3p | | | | | |
| rs572169 | GHSR | CDS | hsa-miR-615- | A/G | G | 81 | A/G | 31.18% |
| | | | 5p | | | | | |
| rs572169 | GHSR | CDS | hsa-miR- | A/G | G | 125 | A/G | 38.02% |
| | | | 629* | | | | | |
| rs572169 | GHSR | CDS | hsa-miR-631 | A/G | G | 105 | A/G | 52.87% |
| rs572169 | GHSR | CDS | hsa-miR-634 | A/G | G | 139 | A/G | 31.13% |
| rs572169 | GHSR | CDS | hsa-miR-636 | A/G | A | 105 | A/G | −17.78% |
| rs572169 | GHSR | CDS | hsa-miR-638 | A/G | A | 92 | A/G | −18.06% |
| rs572169 | GHSR | CDS | hsa-miR-640 | A/G | G | 126 | A/G | 25.17% |
| rs572169 | GHSR | CDS | hsa-miR-642 | A/G | A | 99 | A/G | −19.93% |
| rs572169 | GHSR | CDS | hsa-miR-646 | A/G | G | 123 | A/G | 12.97% |
| rs572169 | GHSR | CDS | hsa-miR-657 | A/G | G | 119 | A/G | 39.82% |
| rs572169 | GHSR | CDS | hsa-miR-662 | A/G | G | 84 | A/G | 16.50% |
| rs572169 | GHSR | CDS | hsa-miR-663 | A/G | G | 110 | A/G | 12.76% |
| rs572169 | GHSR | CDS | hsa-miR- | A/G | G | 101 | A/G | 20.29% |
| | | | 663b | | | | | |
| rs572169 | GHSR | CDS | hsa-miR-671- | A/G | G | 112 | A/G | 39.36% |
| | | | 3p | | | | | |
| rs572169 | GHSR | CDS | hsa-miR- | A/G | A | 114 | A/G | −42.75% |
| | | | 744* | | | | | |
| rs572169 | GHSR | CDS | hsa-miR-769- | A/G | G | 67 | A/G | 16.31% |
| | | | 3p | | | | | |
| rs572169 | GHSR | CDS | hsa-miR-770- | A/G | G | 104 | A/G | 9.55% |
| | | | 5p | | | | | |
| rs572169 | GHSR | CDS | hsa-miR-874 | A/G | G | 109 | A/G | 22.78% |
| rs572169 | GHSR | CDS | hsa-miR- | A/G | G | 114 | A/G | 21.77% |
| | | | 877* | | | | | |
| rs572169 | GHSR | CDS | hsa-miR-885- | A/G | G | 93 | A/G | 8.25% |
| | | | 5p | | | | | |
| rs572169 | GHSR | CDS | hsa-miR-886- | A/G | G | 103 | A/G | 8.84% |
| | | | 3p | | | | | |
| rs572169 | GHSR | CDS | hsa-miR-887 | A/G | G | 110 | A/G | 16.08% |
| rs572169 | GHSR | CDS | hsa-miR- | A/G | A | 123 | A/G | −19.83% |
| | | | 892b | | | | | |
| rs572169 | GHSR | CDS | hsa-miR-93* | A/G | A | 94 | A/G | −10.81% |
| rs572169 | GHSR | CDS | hsa-miR-937 | A/G | G | 130 | A/G | 29.15% |
| rs572169 | GHSR | CDS | hsa-miR-940 | A/G | G | 92 | A/G | 24.03% |
| rs572169 | GHSR | CDS | hsa-miR-941 | A/G | G | 143 | A/G | 43.07% |
| rs572169 | GHSR | CDS | hsa-miR-943 | A/G | G | 93 | A/G | 17.09% |
| rs572169 | GHSR | CDS | hsa-miR-99a | A/G | G | 115 | A/G | 43.95% |
| rs572169 | GHSR | CDS | hsa-miR-99b | A/G | G | 124 | A/G | 53.06% |
| rs643788 | DPAGT1 | CDS | hsa-let-7e* | C/T | C | 110 | C/T | −43.12% |
| rs643788 | DPAGT1 | CDS | hsa-miR-103 | C/T | C | 91 | C/T | −155.86% |
| rs643788 | DPAGT1 | CDS | hsa-miR- | C/T | C | 148 | C/T | −17.85% |
| | | | 106b* | | | | | |
| rs643788 | DPAGT1 | CDS | hsa-miR- | C/T | C | 115 | C/T | −23.48% |
| | | | 1180 | | | | | |
| rs643788 | DPAGT1 | CDS | hsa-miR- | C/T | T | 94 | C/T | 31.39% |
| | | | 1202 | | | | | |
| rs643788 | DPAGT1 | CDS | hsa-miR- | C/T | C | 118 | C/T | −21.04% |
| | | | 1203 | | | | | |
| rs643788 | DPAGT1 | CDS | hsa-miR- | C/T | C | 141 | C/T | −41.43% |
| | | | 1234 | | | | | |
| rs643788 | DPAGT1 | CDS | hsa-miR- | C/T | C | 129 | C/T | −11.13% |
| | | | 1250 | | | | | |
| rs643788 | DPAGT1 | CDS | hsa-miR- | C/T | C | 118 | C/T | −26.99% |
| | | | 1275 | | | | | |
| rs643788 | DPAGT1 | CDS | hsa-miR- | C/T | C | 112 | C/T | −30.69% |
| | | | 1292 | | | | | |
| rs643788 | DPAGT1 | CDS | hsa-miR- | C/T | C | 83 | C/T | −34.96% |
| | | | 1298 | | | | | |
| rs643788 | DPAGT1 | CDS | hsa-miR- | C/T | T | 138 | C/T | 15.47% |
| | | | 1300 | | | | | |
| rs643788 | DPAGT1 | CDS | hsa-miR- | C/T | C | 115 | C/T | −21.40% |
| | | | 1307 | | | | | |
| rs643788 | DPAGT1 | CDS | hsa-miR-134 | C/T | C | 137 | C/T | −12.17% |
| rs643788 | DPAGT1 | CDS | hsa-miR- | C/T | C | 101 | C/T | −26.55% |
| | | | 135a* | | | | | |
| rs643788 | DPAGT1 | CDS | hsa-miR-138- | C/T | C | 81 | C/T | −26.64% |
| | | | 2* | | | | | |
| rs643788 | DPAGT1 | CDS | hsa-miR- | C/T | C | 93 | C/T | −22.33% |
| | | | 143* | | | | | |
| rs643788 | DPAGT1 | CDS | hsa-miR- | C/T | C | 141 | C/T | −24.23% |
| | | | 1469 | | | | | |
| rs643788 | DPAGT1 | CDS | hsa-miR- | C/T | C | 128 | C/T | −101.44% |
| | | | 147b | | | | | |
| rs643788 | DPAGT1 | CDS | hsa-miR- | C/T | C | 132 | C/T | −11.52% |
| | | | 149* | | | | | |
| rs643788 | DPAGT1 | CDS | hsa-miR-151- | C/T | C | 126 | C/T | −33.02% |
| | | | 5p | | | | | |
| rs643788 | DPAGT1 | CDS | hsa-miR-17* | C/T | T | 81 | C/T | 56.95% |
| rs643788 | DPAGT1 | CDS | hsa-miR-184 | C/T | C | 127 | C/T | −50.51% |
| rs643788 | DPAGT1 | CDS | hsa-miR- | C/T | T | 125 | C/T | 17.64% |
| | | | 185* | | | | | |
| rs643788 | DPAGT1 | CDS | hsa-miR-18a | C/T | C | 122 | C/T | −28.00% |
| rs643788 | DPAGT1 | CDS | hsa-miR-18b | C/T | C | 122 | C/T | −37.41% |
| rs643788 | DPAGT1 | CDS | hsa-miR- | C/T | C | 109 | C/T | −37.69% |
| | | | 1908 | | | | | |
| rs643788 | DPAGT1 | CDS | hsa-miR- | C/T | C | 116 | C/T | −13.55% |
| | | | 1909 | | | | | |
| rs643788 | DPAGT1 | CDS | hsa-miR- | C/T | C | 117 | C/T | −45.97% |
| | | | 1909* | | | | | |
| rs643788 | DPAGT1 | CDS | hsa-miR-191 | C/T | C | 121 | C/T | −45.71% |
| rs643788 | DPAGT1 | CDS | hsa-miR- | C/T | C | 96 | C/T | −35.76% |
| | | | 194* | | | | | |
| rs643788 | DPAGT1 | CDS | hsa-miR- | C/T | C | 94 | C/T | −24.09% |
| | | | 196a* | | | | | |
| rs643788 | DPAGT1 | CDS | hsa-miR-210 | C/T | T | 81 | C/T | 19.37% |
| rs643788 | DPAGT1 | CDS | hsa-miR- | C/T | C | 143 | C/T | −34.44% |
| | | | 2110 | | | | | |
| rs643788 | DPAGT1 | CDS | hsa-miR-219- | C/T | C | 92 | C/T | −10.86% |
| | | | 2-3p | | | | | |
| rs643788 | DPAGT1 | CDS | hsa-miR- | C/T | C | 92 | C/T | −69.97% |
| | | | 23b* | | | | | |
| rs643788 | DPAGT1 | CDS | hsa-miR-337- | C/T | C | 118 | C/T | −106.32% |
| | | | 5p | | | | | |
| rs643788 | DPAGT1 | CDS | hsa-miR- | C/T | T | 98 | C/T | 8.27% |
| | | | 378* | | | | | |
| rs643788 | DPAGT1 | CDS | hsa-miR-379 | C/T | T | 87 | C/T | 44.45% |
| rs643788 | DPAGT1 | CDS | hsa-miR-383 | C/T | T | 96 | C/T | 10.40% |
| rs643788 | DPAGT1 | CDS | hsa-miR-433 | C/T | T | 102 | C/T | 73.86% |
| rs643788 | DPAGT1 | CDS | hsa-miR-489 | C/T | C | 114 | C/T | −52.52% |
| rs643788 | DPAGT1 | CDS | hsa-miR-509- | C/T | T | 98 | C/T | 73.76% |
| | | | 3p | | | | | |
| rs643788 | DPAGT1 | CDS | hsa-miR- | C/T | C | 92 | C/T | −10.11% |
| | | | 518c* | | | | | |
| rs643788 | DPAGT1 | CDS | hsa-miR-523 | C/T | C | 103 | C/T | −13.16% |
| rs643788 | DPAGT1 | CDS | hsa-miR-567 | C/T | T | 81 | C/T | 25.00% |
| rs643788 | DPAGT1 | CDS | hsa-miR- | C/T | C | 83 | C/T | −13.25% |
| | | | 589* | | | | | |
| rs643788 | DPAGT1 | CDS | hsa-miR-615- | C/T | C | 100 | C/T | −41.68% |
| | | | 5p | | | | | |
| rs643788 | DPAGT1 | CDS | hsa-miR-638 | C/T | C | 134 | C/T | −24.34% |
| rs643788 | DPAGT1 | CDS | hsa-miR-646 | C/T | T | 88 | C/T | 17.40% |
| rs643788 | DPAGT1 | CDS | hsa-miR-647 | C/T | C | 121 | C/T | −56.57% |
| rs643788 | DPAGT1 | CDS | hsa-miR-650 | C/T | C | 122 | C/T | −9.02% |
| rs643788 | DPAGT1 | CDS | hsa-miR-663 | C/T | C | 121 | C/T | −16.23% |
| rs643788 | DPAGT1 | CDS | hsa-miR-668 | C/T | C | 100 | C/T | −24.95% |
| rs643788 | DPAGT1 | CDS | hsa-miR-671- | C/T | C | 110 | C/T | −14.18% |
| | | | 3p | | | | | |
| rs643788 | DPAGT1 | CDS | hsa-miR-675 | C/T | C | 143 | C/T | −28.04% |
| rs643788 | DPAGT1 | CDS | hsa-miR-760 | C/T | C | 146 | C/T | −8.36% |
| rs643788 | DPAGT1 | CDS | hsa-miR-885- | C/T | C | 92 | C/T | −41.35% |
| | | | 3p | | | | | |
| rs643788 | DPAGT1 | CDS | hsa-miR-886- | C/T | C | 118 | C/T | −18.69% |
| | | | 5p | | | | | |
| rs643788 | DPAGT1 | CDS | hsa-miR- | C/T | C | 157 | C/T | −35.59% |
| | | | 92b* | | | | | |
| rs643788 | DPAGT1 | CDS | hsa-miR- | C/T | C | 95 | C/T | −14.81% |
| | | | 99a* | | | | | |
| rs7759 | DPAGT1 | 3′ UTR | hsa-miR- | A/G | G | 92 | A/G | 14.18% |
| | | | 1183 | | | | | |
| rs7759 | DPAGT1 | 3′ UTR | hsa-miR- | A/G | G | 97 | A/G | 14.58% |
| | | | 1200 | | | | | |
| rs7759 | DPAGT1 | 3′ UTR | hsa-miR- | A/G | G | 103 | A/G | 58.25% |
| | | | 1244 | | | | | |
| rs7759 | DPAGT1 | 3′ UTR | hsa-miR- | A/G | G | 87 | A/G | 126.34% |
| | | | 1251 | | | | | |
| rs7759 | DPAGT1 | 3′ UTR | hsa-miR-17* | A/G | G | 110 | A/G | 16.97% |
| rs7759 | DPAGT1 | 3′ UTR | hsa-miR- | A/G | G | 106 | A/G | 11.44% |
| | | | 20a* | | | | | |
| rs7759 | DPAGT1 | 3′ UTR | hsa-miR-512- | A/G | G | 102 | A/G | 8.50% |
| | | | 5p | | | | | |
| rs7759 | DPAGT1 | 3′ UTR | hsa-miR-550 | A/G | G | 96 | A/G | 16.12% |
| rs7759 | DPAGT1 | 3′ UTR | hsa-miR- | A/G | G | 137 | A/G | 12.82% |
| | | | 550* | | | | | |
| rs7759 | DPAGT1 | 3′ UTR | hsa-miR-600 | A/G | G | 115 | A/G | 15.48% |
| rs7759 | DPAGT1 | 3′ UTR | hsa-miR-622 | A/G | G | 83 | A/G | 91.18% |
| rs7759 | DPAGT1 | 3′ UTR | hsa-miR-639 | A/G | G | 112 | A/G | 12.80% |
| rs7759 | DPAGT1 | 3′ UTR | hsa-miR-663 | A/G | G | 84 | A/G | 13.62% |
| rs7759 | DPAGT1 | 3′ UTR | hsa-miR-941 | A/G | G | 122 | A/G | 69.71% |
| rs8551 | DPAGT1 | 3′ UTR | hsa-let-7d | C/T | C | 92 | C/T | −32.63% |
| rs8551 | DPAGT1 | 3′ UTR | hsa-miR- | C/T | C | 82 | C/T | −9.21% |
| | | | 1180 | | | | | |
| rs8551 | DPAGT1 | 3′ UTR | hsa-miR- | C/T | C | 114 | C/T | −10.58% |
| | | | 1183 | | | | | |
| rs8551 | DPAGT1 | 3′ UTR | hsa-miR- | C/T | C | 85 | C/T | −31.05% |
| | | | 1208 | | | | | |
| rs8551 | DPAGT1 | 3′ UTR | hsa-miR- | C/T | C | 112 | C/T | −19.32% |
| | | | 1286 | | | | | |
| rs8551 | DPAGT1 | 3′ UTR | hsa-miR- | C/T | T | 85 | C/T | 25.67% |
| | | | 1307 | | | | | |
| rs8551 | DPAGT1 | 3′ UTR | hsa-miR-145 | C/T | C | 99 | C/T | −45.80% |
| rs8551 | DPAGT1 | 3′ UTR | hsa-miR- | C/T | C | 110 | C/T | −21.82% |
| | | | 1469 | | | | | |
| rs8551 | DPAGT1 | 3′ UTR | hsa-miR-16 | C/T | C | 119 | C/T | −118.36% |
| rs8551 | DPAGT1 | 3′ UTR | hsa-miR-17* | C/T | C | 107 | C/T | −63.37% |
| rs8551 | DPAGT1 | 3′ UTR | hsa-miR- | C/T | C | 114 | C/T | −9.60% |
| | | | 1908 | | | | | |
| rs8551 | DPAGT1 | 3′ UTR | hsa-miR- | C/T | C | 155 | C/T | −33.10% |
| | | | 1909 | | | | | |
| rs8551 | DPAGT1 | 3′ UTR | hsa-miR- | C/T | C | 106 | C/T | −55.92% |
| | | | 196a | | | | | |
| rs8551 | DPAGT1 | 3′ UTR | hsa-miR- | C/T | C | 90 | C/T | −35.06% |
| | | | 1977 | | | | | |
| rs8551 | DPAGT1 | 3′ UTR | hsa-miR-198 | C/T | C | 138 | C/T | −13.49% |
| rs8551 | DPAGT1 | 3′ UTR | hsa-miR- | C/T | C | 159 | C/T | −31.62% |
| | | | 199a-3p | | | | | |
| rs8551 | DPAGT1 | 3′ UTR | hsa-miR- | C/T | C | 159 | C/T | −31.62% |
| | | | 199b-3p | | | | | |
| rs8551 | DPAGT1 | 3′ UTR | hsa-miR-214 | C/T | C | 142 | C/T | −46.21% |
| rs8551 | DPAGT1 | 3′ UTR | hsa-miR- | C/T | T | 160 | C/T | 9.27% |
| | | | 222* | | | | | |
| rs8551 | DPAGT1 | 3′ UTR | hsa-miR-24 | C/T | C | 112 | C/T | −27.75% |
| rs8551 | DPAGT1 | 3′ UTR | hsa-miR-298 | C/T | C | 145 | C/T | −9.51% |
| rs8551 | DPAGT1 | 3′ UTR | hsa-miR- | C/T | C | 109 | C/T | −82.29% |
| | | | 30a* | | | | | |
| rs8551 | DPAGT1 | 3′ UTR | hsa-miR-30c- | C/T | C | 93 | C/T | −11.13% |
| | | | 1* | | | | | |
| rs8551 | DPAGT1 | 3′ UTR | hsa-miR- | C/T | T | 91 | C/T | 8.90% |
| | | | 320a | | | | | |
| rs8551 | DPAGT1 | 3′ UTR | hsa-miR- | C/T | T | 95 | C/T | 8.87% |
| | | | 320b | | | | | |
| rs8551 | DPAGT1 | 3′ UTR | hsa-miR-34a | C/T | C | 103 | C/T | −149.38% |
| rs8551 | DPAGT1 | 3′ UTR | hsa-miR-34c- | C/T | C | 109 | C/T | −50.86% |
| | | | 5p | | | | | |
| rs8551 | DPAGT1 | 3′ UTR | hsa-miR-361- | C/T | C | 114 | C/T | −32.44% |
| | | | 5p | | | | | |
| rs8551 | DPAGT1 | 3′ UTR | hsa-miR-432 | C/T | T | 85 | C/T | 49.48% |
| rs8551 | DPAGT1 | 3′ UTR | hsa-miR-498 | C/T | C | 100 | C/T | −18.83% |
| rs8551 | DPAGT1 | 3′ UTR | hsa-miR-503 | C/T | C | 136 | C/T | −17.17% |
| rs8551 | DPAGT1 | 3′ UTR | hsa-miR-508- | C/T | C | 105 | C/T | −112.49% |
| | | | 5p | | | | | |
| rs8551 | DPAGT1 | 3′ UTR | hsa-miR-510 | C/T | C | 114 | C/T | −11.62% |
| rs8551 | DPAGT1 | 3′ UTR | hsa-miR-512- | C/T | C | 133 | C/T | −17.53% |
| | | | 5p | | | | | |
| rs8551 | DPAGT1 | 3′ UTR | hsa-miR- | C/T | C | 112 | C/T | −17.13% |
| | | | 518a-5p | | | | | |
| rs8551 | DPAGT1 | 3′ UTR | hsa-miR- | C/T | C | 124 | C/T | −30.19% |
| | | | 520a-5p | | | | | |
| rs8551 | DPAGT1 | 3′ UTR | hsa-miR-525- | C/T | C | 117 | C/T | −23.86% |
| | | | 5p | | | | | |
| rs8551 | DPAGT1 | 3′ UTR | hsa-miR-527 | C/T | C | 112 | C/T | −17.13% |
| rs8551 | DPAGT1 | 3′ UTR | hsa-miR-541 | C/T | T | 131 | C/T | 14.85% |
| rs8551 | DPAGT1 | 3′ UTR | hsa-miR- | C/T | C | 103 | C/T | −11.51% |
| | | | 551b* | | | | | |
| rs8551 | DPAGT1 | 3′ UTR | hsa-miR- | C/T | C | 117 | C/T | −27.36% |
| | | | 593* | | | | | |
| rs8551 | DPAGT1 | 3′ UTR | hsa-miR-596 | C/T | C | 93 | C/T | −88.74% |
| rs8551 | DPAGT1 | 3′ UTR | hsa-miR-622 | C/T | C | 120 | C/T | −20.31% |
| rs8551 | DPAGT1 | 3′ UTR | hsa-miR-639 | C/T | C | 86 | C/T | −49.74% |
| rs8551 | DPAGT1 | 3′ UTR | hsa-miR-658 | C/T | C | 162 | C/T | −26.72% |
| rs8551 | DPAGT1 | 3′ UTR | hsa-miR-675 | C/T | C | 102 | C/T | −18.95% |
| rs8551 | DPAGT1 | 3′ UTR | hsa-miR-765 | C/T | T | 107 | C/T | 15.83% |
| rs8551 | DPAGT1 | 3′ UTR | hsa-miR-922 | C/T | C | 169 | C/T | −30.59% |
| rs8551 | DPAGT1 | 3′ UTR | hsa-miR-93 | C/T | C | 98 | C/T | −47.88% |
| rs11575213 | IGFBP5 | 3′ UTR | hsa-miR- | C/T | T | 88 | C/T | 9.62% |
| | | | 101* | | | | | |
| rs11575213 | IGFBP5 | 3′ UTR | hsa-miR- | C/T | T | 100 | C/T | 31.23% |
| | | | 1179 | | | | | |
| rs11575213 | IGFBP5 | 3′ UTR | hsa-miR- | C/T | C | 87 | C/T | −18.05% |
| | | | 1184 | | | | | |
| rs11575213 | IGFBP5 | 3′ UTR | hsa-miR- | C/T | T | 89 | C/T | 28.56% |
| | | | 1203 | | | | | |
| rs11575213 | IGFBP5 | 3′ UTR | hsa-miR- | C/T | T | 160 | C/T | 14.87% |
| | | | 1204 | | | | | |
| rs11575213 | IGFBP5 | 3′ UTR | hsa-miR- | C/T | C | 107 | C/T | −16.50% |
| | | | 1236 | | | | | |
| rs11575213 | IGFBP5 | 3′ UTR | hsa-miR- | C/T | C | 144 | C/T | −14.80% |
| | | | 1238 | | | | | |
| rs11575213 | IGFBP5 | 3′ UTR | hsa-miR- | C/T | C | 87 | C/T | −23.99% |
| | | | 124* | | | | | |
| rs11575213 | IGFBP5 | 3′ UTR | hsa-miR- | C/T | C | 125 | C/T | −14.49% |
| | | | 1247 | | | | | |
| rs11575213 | IGFBP5 | 3′ UTR | hsa-miR- | C/T | C | 82 | C/T | −38.69% |
| | | | 125b-2* | | | | | |
| rs11575213 | IGFBP5 | 3′ UTR | hsa-miR- | C/T | C | 142 | C/T | −9.38% |
| | | | 1274a | | | | | |
| rs11575213 | IGFBP5 | 3′ UTR | hsa-miR- | C/T | C | 132 | C/T | −11.27% |
| | | | 1274b | | | | | |
| rs11575213 | IGFBP5 | 3′ UTR | hsa-miR- | C/T | C | 93 | C/T | −13.96% |
| | | | 1294 | | | | | |
| rs11575213 | IGFBP5 | 3′ UTR | hsa-miR- | C/T | C | 110 | C/T | −26.47% |
| | | | 1295 | | | | | |
| rs11575213 | IGFBP5 | 3′ UTR | hsa-miR- | C/T | T | 117 | C/T | 33.76% |
| | | | 1307 | | | | | |
| rs11575213 | IGFBP5 | 3′ UTR | hsa-miR- | C/T | C | 118 | C/T | −10.75% |
| | | | 130b* | | | | | |
| rs11575213 | IGFBP5 | 3′ UTR | hsa-miR- | C/T | C | 121 | C/T | −14.79% |
| | | | 132* | | | | | |
| rs11575213 | IGFBP5 | 3′ UTR | hsa-miR- | C/T | T | 124 | C/T | 10.02% |
| | | | 133a | | | | | |
| rs11575213 | IGFBP5 | 3′ UTR | hsa-miR-139- | C/T | C | 113 | C/T | −50.99% |
| | | | 3p | | | | | |
| rs11575213 | IGFBP5 | 3′ UTR | hsa-miR- | C/T | T | 108 | C/T | 8.70% |
| | | | 1469 | | | | | |
| rs11575213 | IGFBP5 | 3′ UTR | hsa-miR- | C/T | T | 111 | C/T | 13.63% |
| | | | 146b-3p | | | | | |
| rs11575213 | IGFBP5 | 3′ UTR | hsa-miR- | C/T | C | 150 | C/T | −10.45% |
| | | | 1471 | | | | | |
| rs11575213 | IGFBP5 | 3′ UTR | hsa-miR- | C/T | C | 125 | C/T | −8.33% |
| | | | 1538 | | | | | |
| rs11575213 | IGFBP5 | 3′ UTR | hsa-miR- | C/T | T | 108 | C/T | 23.37% |
| | | | 15a* | | | | | |
| rs11575213 | IGFBP5 | 3′ UTR | hsa-miR-17* | C/T | C | 77 | C/T | −31.11% |
| rs11575213 | IGFBP5 | 3′ UTR | hsa-miR- | C/T | T | 119 | C/T | 25.67% |
| | | | 181b | | | | | |
| rs11575213 | IGFBP5 | 3′ UTR | hsa-miR- | C/T | T | 113 | C/T | 44.78% |
| | | | 181d | | | | | |
| rs11575213 | IGFBP5 | 3′ UTR | hsa-miR-187 | C/T | C | 120 | C/T | −16.34% |
| rs11575213 | IGFBP5 | 3′ UTR | hsa-miR-188- | C/T | T | 142 | C/T | 16.77% |
| | | | 3p | | | | | |
| rs11575213 | IGFBP5 | 3′ UTR | hsa-miR- | C/T | C | 95 | C/T | −22.76% |
| | | | 1914* | | | | | |
| rs11575213 | IGFBP5 | 3′ UTR | hsa-miR- | C/T | C | 99 | C/T | −37.48% |
| | | | 1915* | | | | | |
| rs11575213 | IGFBP5 | 3′ UTR | hsa-miR- | C/T | C | 88 | C/T | −21.52% |
| | | | 196a | | | | | |
| rs11575213 | IGFBP5 | 3′ UTR | hsa-miR- | C/T | C | 88 | C/T | −15.39% |
| | | | 196b | | | | | |
| rs11575213 | IGFBP5 | 3′ UTR | hsa-miR- | C/T | T | 100 | C/T | 10.56% |
| | | | 1976 | | | | | |
| rs11575213 | IGFBP5 | 3′ UTR | hsa-miR-205 | C/T | T | 144 | C/T | 36.12% |
| rs11575213 | IGFBP5 | 3′ UTR | hsa-miR-20b | C/T | T | 103 | C/T | 9.30% |
| rs11575213 | IGFBP5 | 3′ UTR | hsa-miR-222 | C/T | T | 88 | C/T | 75.87% |
| rs11575213 | IGFBP5 | 3′ UTR | hsa-miR- | C/T | C | 121 | C/T | −13.14% |
| | | | 27a* | | | | | |
| rs11575213 | IGFBP5 | 3′ UTR | hsa-miR-296- | C/T | T | 159 | C/T | 10.69% |
| | | | 5p | | | | | |
| rs11575213 | IGFBP5 | 3′ UTR | hsa-miR-299- | C/T | T | 92 | C/T | 17.00% |
| | | | 5p | | | | | |
| rs11575213 | IGFBP5 | 3′ UTR | hsa-miR- | C/T | C | 121 | C/T | −10.37% |
| | | | 30a* | | | | | |
| rs11575213 | IGFBP5 | 3′ UTR | hsa-miR-339- | C/T | C | 109 | C/T | −34.13% |
| | | | 3p | | | | | |
| rs11575213 | IGFBP5 | 3′ UTR | hsa-miR- | C/T | T | 121 | C/T | 24.46% |
| | | | 340* | | | | | |
| rs11575213 | IGFBP5 | 3′ UTR | hsa-miR-34c- | C/T | T | 90 | C/T | 27.29% |
| | | | 5p | | | | | |
| rs11575213 | IGFBP5 | 3′ UTR | hsa-miR-373 | C/T | C | 100 | C/T | −13.39% |
| rs11575213 | IGFBP5 | 3′ UTR | hsa-miR-423- | C/T | C | 116 | C/T | −20.85% |
| | | | 3p | | | | | |
| rs11575213 | IGFBP5 | 3′ UTR | hsa-miR-425 | C/T | C | 114 | C/T | −13.42% |
| rs11575213 | IGFBP5 | 3′ UTR | hsa-miR- | C/T | T | 104 | C/T | 12.75% |
| | | | 431* | | | | | |
| rs11575213 | IGFBP5 | 3′ UTR | hsa-miR- | C/T | C | 102 | C/T | −41.15% |
| | | | 449b | | | | | |
| rs11575213 | IGFBP5 | 3′ UTR | hsa-miR-501- | C/T | C | 110 | C/T | −24.11% |
| | | | 5p | | | | | |
| rs11575213 | IGFBP5 | 3′ UTR | hsa-miR- | C/T | T | 88 | C/T | 18.25% |
| | | | 505* | | | | | |
| rs11575213 | IGFBP5 | 3′ UTR | hsa-miR-508- | C/T | T | 112 | C/T | 16.78% |
| | | | 5p | | | | | |
| rs11575213 | IGFBP5 | 3′ UTR | hsa-miR-512- | C/T | C | 117 | C/T | −9.85% |
| | | | 5p | | | | | |
| rs11575213 | IGFBP5 | 3′ UTR | hsa-miR-532- | C/T | C | 95 | C/T | −17.24% |
| | | | 5p | | | | | |
| rs11575213 | IGFBP5 | 3′ UTR | hsa-miR-541 | C/T | C | 148 | C/T | −10.13% |
| rs11575213 | IGFBP5 | 3′ UTR | hsa-miR-542- | C/T | C | 99 | C/T | −14.05% |
| | | | 5p | | | | | |
| rs11575213 | IGFBP5 | 3′ UTR | hsa-miR- | C/T | C | 92 | C/T | −13.97% |
| | | | 548b-3p | | | | | |
| rs11575213 | IGFBP5 | 3′ UTR | hsa-miR-589 | C/T | C | 92 | C/T | −14.57% |
| rs11575213 | IGFBP5 | 3′ UTR | hsa-miR-591 | C/T | T | 114 | C/T | 30.56% |
| rs11575213 | IGFBP5 | 3′ UTR | hsa-miR- | C/T | T | 118 | C/T | 23.23% |
| | | | 593* | | | | | |
| rs11575213 | IGFBP5 | 3′ UTR | hsa-miR-596 | C/T | C | 101 | C/T | −12.05% |
| rs11575213 | IGFBP5 | 3′ UTR | hsa-miR-601 | C/T | C | 117 | C/T | −24.89% |
| rs11575213 | IGFBP5 | 3′ UTR | hsa-miR-608 | C/T | C | 101 | C/T | −8.19% |
| rs11575213 | IGFBP5 | 3′ UTR | hsa-miR-609 | C/T | T | 100 | C/T | 17.10% |
| rs11575213 | IGFBP5 | 3′ UTR | hsa-miR-611 | C/T | C | 123 | C/T | −11.24% |
| rs11575213 | IGFBP5 | 3′ UTR | hsa-miR-616 | C/T | T | 138 | C/T | 13.43% |
| rs11575213 | IGFBP5 | 3′ UTR | hsa-miR-619 | C/T | C | 81 | C/T | −17.15% |
| rs11575213 | IGFBP5 | 3′ UTR | hsa-miR-623 | C/T | C | 155 | C/T | −10.75% |
| rs11575213 | IGFBP5 | 3′ UTR | hsa-miR-630 | C/T | C | 90 | C/T | −9.43% |
| rs11575213 | IGFBP5 | 3′ UTR | hsa-miR-636 | C/T | C | 94 | C/T | −9.09% |
| rs11575213 | IGFBP5 | 3′ UTR | hsa-miR-637 | C/T | C | 131 | C/T | −8.94% |
| rs11575213 | IGFBP5 | 3′ UTR | hsa-miR-658 | C/T | C | 72 | C/T | −10.65% |
| rs11575213 | IGFBP5 | 3′ UTR | hsa-miR-659 | C/T | T | 97 | C/T | 9.67% |
| rs11575213 | IGFBP5 | 3′ UTR | hsa-miR-661 | C/T | C | 137 | C/T | −10.75% |
| rs11575213 | IGFBP5 | 3′ UTR | hsa-miR-662 | C/T | C | 150 | C/T | −10.20% |
| rs11575213 | IGFBP5 | 3′ UTR | hsa-miR- | C/T | C | 143 | C/T | −30.42% |
| | | | 663b | | | | | |
| rs11575213 | IGFBP5 | 3′ UTR | hsa-miR-675 | C/T | C | 119 | C/T | −20.43% |
| rs11575213 | IGFBP5 | 3′ UTR | hsa-miR-766 | C/T | T | 96 | C/T | 17.40% |
| rs11575213 | IGFBP5 | 3′ UTR | hsa-miR-769- | C/T | C | 104 | C/T | −16.69% |
| | | | 3p | | | | | |
| rs11575213 | IGFBP5 | 3′ UTR | hsa-miR-770- | C/T | C | 127 | C/T | −11.23% |
| | | | 5p | | | | | |
| rs11575213 | IGFBP5 | 3′ UTR | hsa-miR-874 | C/T | C | 95 | C/T | −21.90% |
| rs11575213 | IGFBP5 | 3′ UTR | hsa-miR-92a- | C/T | C | 115 | C/T | −30.03% |
| | | | 2* | | | | | |
| rs11575213 | IGFBP5 | 3′ UTR | hsa-miR-93 | C/T | C | 101 | C/T | −11.74% |
| rs11575213 | IGFBP5 | 3′ UTR | hsa-miR-933 | C/T | C | 101 | C/T | −9.39% |
| rs2069849 | IL6 | CDS | hsa-let-7c | C/T | T | 88 | C/T | 29.65% |
| rs2069849 | IL6 | CDS | hsa-miR- | C/T | C | 118 | C/T | −25.17% |
| | | | 106a | | | | | |
| rs2069849 | IL6 | CDS | hsa-miR- | C/T | C | 125 | C/T | −9.79% |
| | | | 106b* | | | | | |
| rs2069849 | IL6 | CDS | hsa-miR- | C/T | C | 148 | C/T | −11.43% |
| | | | 1183 | | | | | |
| rs2069849 | IL6 | CDS | hsa-miR- | C/T | C | 89 | C/T | −18.09% |
| | | | 1202 | | | | | |
| rs2069849 | IL6 | CDS | hsa-miR- | C/T | C | 171 | C/T | −15.18% |
| | | | 1205 | | | | | |
| rs2069849 | IL6 | CDS | hsa-miR- | C/T | C | 127 | C/T | −16.58% |
| | | | 1207-5p | | | | | |
| rs2069849 | IL6 | CDS | hsa-miR- | C/T | C | 107 | C/T | −17.60% |
| | | | 1224-5p | | | | | |
| rs2069849 | IL6 | CDS | hsa-miR- | C/T | C | 110 | C/T | −20.39% |
| | | | 1228* | | | | | |
| rs2069849 | IL6 | CDS | hsa-miR- | C/T | C | 141 | C/T | −15.61% |
| | | | 1254 | | | | | |
| rs2069849 | IL6 | CDS | hsa-miR- | C/T | C | 101 | C/T | −19.30% |
| | | | 125a-3p | | | | | |
| rs2069849 | IL6 | CDS | hsa-miR- | C/T | C | 107 | C/T | −15.68% |
| | | | 125b-1* | | | | | |
| rs2069849 | IL6 | CDS | hsa-miR- | C/T | C | 100 | C/T | −9.91% |
| | | | 1268 | | | | | |
| rs2069849 | IL6 | CDS | hsa-miR-127- | C/T | C | 91 | C/T | −10.44% |
| | | | 5p | | | | | |
| rs2069849 | IL6 | CDS | hsa-miR- | C/T | T | 99 | C/T | 12.26% |
| | | | 1270 | | | | | |
| rs2069849 | IL6 | CDS | hsa-miR- | C/T | C | 85 | C/T | −11.15% |
| | | | 1272 | | | | | |
| rs2069849 | IL6 | CDS | hsa-miR- | C/T | T | 117 | C/T | 10.90% |
| | | | 1273 | | | | | |
| rs2069849 | IL6 | CDS | hsa-miR- | C/T | C | 163 | C/T | −10.43% |
| | | | 1288 | | | | | |
| rs2069849 | IL6 | CDS | hsa-miR-134 | C/T | C | 109 | C/T | −32.68% |
| rs2069849 | IL6 | CDS | hsa-miR- | C/T | C | 106 | C/T | −14.43% |
| | | | 1471 | | | | | |
| rs2069849 | IL6 | CDS | hsa-miR- | C/T | T | 107 | C/T | 17.65% |
| | | | 147b | | | | | |
| rs2069849 | IL6 | CDS | hsa-miR- | C/T | C | 91 | C/T | −12.66% |
| | | | 149* | | | | | |
| rs2069849 | IL6 | CDS | hsa-miR- | C/T | C | 92 | C/T | −10.42% |
| | | | 150* | | | | | |
| rs2069849 | IL6 | CDS | hsa-miR-17 | C/T | C | 118 | C/T | −25.17% |
| rs2069849 | IL6 | CDS | hsa-miR-17* | C/T | C | 133 | C/T | −17.93% |
| rs2069849 | IL6 | CDS | hsa-miR-185 | C/T | T | 106 | C/T | 20.23% |
| rs2069849 | IL6 | CDS | hsa-miR- | C/T | C | 107 | C/T | −17.06% |
| | | | 186* | | | | | |
| rs2069849 | IL6 | CDS | hsa-miR-188- | C/T | C | 115 | C/T | −11.96% |
| | | | 5p | | | | | |
| rs2069849 | IL6 | CDS | hsa-miR- | C/T | C | 126 | C/T | −12.59% |
| | | | 1909 | | | | | |
| rs2069849 | IL6 | CDS | hsa-miR- | C/T | C | 88 | C/T | −13.49% |
| | | | 193b* | | | | | |
| rs2069849 | IL6 | CDS | hsa-miR- | C/T | C | 84 | C/T | −62.64% |
| | | | 196b | | | | | |
| rs2069849 | IL6 | CDS | hsa-miR-198 | C/T | C | 86 | C/T | −8.04% |
| rs2069849 | IL6 | CDS | hsa-miR- | C/T | C | 81 | C/T | −12.37% |
| | | | 200b* | | | | | |
| rs2069849 | IL6 | CDS | hsa-miR- | C/T | C | 118 | C/T | −15.58% |
| | | | 20a* | | | | | |
| rs2069849 | IL6 | CDS | hsa-miR-20b | C/T | C | 118 | C/T | −23.95% |
| rs2069849 | IL6 | CDS | hsa-miR- | C/T | T | 111 | C/T | 13.78% |
| | | | 2110 | | | | | |
| rs2069849 | IL6 | CDS | hsa-miR-217 | C/T | C | 138 | C/T | −18.72% |
| rs2069849 | IL6 | CDS | hsa-miR-219- | C/T | T | 87 | C/T | 94.65% |
| | | | 2-3p | | | | | |
| rs2069849 | IL6 | CDS | hsa-miR- | C/T | T | 105 | C/T | 18.70% |
| | | | 220c | | | | | |
| rs2069849 | IL6 | CDS | hsa-miR- | C/T | C | 104 | C/T | −94.95% |
| | | | 23b* | | | | | |
| rs2069849 | IL6 | CDS | hsa-miR-24 | C/T | C | 133 | C/T | −16.84% |
| rs2069849 | IL6 | CDS | hsa-miR-28- | C/T | C | 105 | C/T | −8.22% |
| | | | 3p | | | | | |
| rs2069849 | IL6 | CDS | hsa-miR-296- | C/T | C | 125 | C/T | −8.23% |
| | | | 3p | | | | | |
| rs2069849 | IL6 | CDS | hsa-miR-298 | C/T | C | 94 | C/T | −21.06% |
| rs2069849 | IL6 | CDS | hsa-miR- | C/T | T | 87 | C/T | 9.93% |
| | | | 30b* | | | | | |
| rs2069849 | IL6 | CDS | hsa-miR-330- | C/T | T | 101 | C/T | 14.63% |
| | | | 3p | | | | | |
| rs2069849 | IL6 | CDS | hsa-miR-330- | C/T | C | 113 | C/T | −18.58% |
| | | | 5p | | | | | |
| rs2069849 | IL6 | CDS | hsa-miR-331- | C/T | T | 115 | C/T | 10.91% |
| | | | 3p | | | | | |
| rs2069849 | IL6 | CDS | hsa-miR-331- | C/T | C | 86 | C/T | −15.47% |
| | | | 5p | | | | | |
| rs2069849 | IL6 | CDS | hsa-miR-339- | C/T | T | 129 | C/T | 15.62% |
| | | | 3p | | | | | |
| rs2069849 | IL6 | CDS | hsa-miR-339- | C/T | C | 119 | C/T | −13.34% |
| | | | 5p | | | | | |
| rs2069849 | IL6 | CDS | hsa-miR-370 | C/T | C | 125 | C/T | −15.63% |
| rs2069849 | IL6 | CDS | hsa-miR-371- | C/T | C | 108 | C/T | −18.84% |
| | | | 5p | | | | | |
| rs2069849 | IL6 | CDS | hsa-miR- | C/T | T | 91 | C/T | 114.05% |
| | | | 376a* | | | | | |
| rs2069849 | IL6 | CDS | hsa-miR-378 | C/T | C | 151 | C/T | −14.95% |
| rs2069849 | IL6 | CDS | hsa-miR- | C/T | C | 108 | C/T | −55.47% |
| | | | 380* | | | | | |
| rs2069849 | IL6 | CDS | hsa-miR- | C/T | C | 160 | C/T | −13.51% |
| | | | 422a | | | | | |
| rs2069849 | IL6 | CDS | hsa-miR-423- | C/T | C | 102 | C/T | −12.81% |
| | | | 3p | | | | | |
| rs2069849 | IL6 | CDS | hsa-miR-431 | C/T | T | 92 | C/T | 21.12% |
| rs2069849 | IL6 | CDS | hsa-miR- | C/T | C | 111 | C/T | −11.56% |
| | | | 431* | | | | | |
| rs2069849 | IL6 | CDS | hsa-miR-453 | C/T | C | 93 | C/T | −19.87% |
| rs2069849 | IL6 | CDS | hsa-miR-483- | C/T | C | 99 | C/T | −18.23% |
| | | | 5p | | | | | |
| rs2069849 | IL6 | CDS | hsa-miR-485- | C/T | C | 156 | C/T | −13.63% |
| | | | 5p | | | | | |
| rs2069849 | IL6 | CDS | hsa-miR-490- | C/T | C | 117 | C/T | −10.44% |
| | | | 3p | | | | | |
| rs2069849 | IL6 | CDS | hsa-miR-492 | C/T | C | 122 | C/T | −10.61% |
| rs2069849 | IL6 | CDS | hsa-miR-505 | C/T | C | 84 | C/T | −51.89% |
| rs2069849 | IL6 | CDS | hsa-miR-506 | C/T | C | 123 | C/T | −11.89% |
| rs2069849 | IL6 | CDS | hsa-miR-510 | C/T | C | 135 | C/T | −13.90% |
| rs2069849 | IL6 | CDS | hsa-miR- | C/T | T | 94 | C/T | 25.24% |
| | | | 516a-5p | | | | | |
| rs2069849 | IL6 | CDS | hsa-miR- | C/T | T | 118 | C/T | 34.40% |
| | | | 516b | | | | | |
| rs2069849 | IL6 | CDS | hsa-miR- | C/T | T | 81 | C/T | 42.75% |
| | | | 526b | | | | | |
| rs2069849 | IL6 | CDS | hsa-miR-539 | C/T | T | 109 | C/T | 14.69% |
| rs2069849 | IL6 | CDS | hsa-miR-541 | C/T | T | 124 | C/T | 11.26% |
| rs2069849 | IL6 | CDS | hsa-miR-543 | C/T | C | 82 | C/T | −92.59% |
| rs2069849 | IL6 | CDS | hsa-miR- | C/T | C | 90 | C/T | −39.30% |
| | | | 548o | | | | | |
| rs2069849 | IL6 | CDS | hsa-miR-564 | C/T | C | 98 | C/T | −21.64% |
| rs2069849 | IL6 | CDS | hsa-miR-575 | C/T | C | 106 | C/T | −15.92% |
| rs2069849 | IL6 | CDS | hsa-miR-593 | C/T | C | 97 | C/T | −22.93% |
| rs2069849 | IL6 | CDS | hsa-miR-604 | C/T | C | 128 | C/T | −17.27% |
| rs2069849 | IL6 | CDS | hsa-miR-612 | C/T | C | 147 | C/T | −10.65% |
| rs2069849 | IL6 | CDS | hsa-miR-614 | C/T | C | 123 | C/T | −15.53% |
| rs2069849 | IL6 | CDS | hsa-miR-615- | C/T | C | 120 | C/T | −13.15% |
| | | | 3p | | | | | |
| rs2069849 | IL6 | CDS | hsa-miR-619 | C/T | C | 119 | C/T | −9.55% |
| rs2069849 | IL6 | CDS | hsa-miR-622 | C/T | C | 136 | C/T | −8.05% |
| rs2069849 | IL6 | CDS | hsa-miR-623 | C/T | C | 110 | C/T | −13.83% |
| rs2069849 | IL6 | CDS | hsa-miR-637 | C/T | C | 126 | C/T | −11.61% |
| rs2069849 | IL6 | CDS | hsa-miR-638 | C/T | T | 95 | C/T | 40.45% |
| rs2069849 | IL6 | CDS | hsa-miR-644 | C/T | C | 82 | C/T | −11.56% |
| rs2069849 | IL6 | CDS | hsa-miR-657 | C/T | C | 112 | C/T | −20.38% |
| rs2069849 | IL6 | CDS | hsa-miR-661 | C/T | C | 119 | C/T | −11.43% |
| rs2069849 | IL6 | CDS | hsa-miR-663 | C/T | C | 139 | C/T | −14.70% |
| rs2069849 | IL6 | CDS | hsa-miR- | C/T | C | 124 | C/T | −17.17% |
| | | | 663b | | | | | |
| rs2069849 | IL6 | CDS | hsa-miR- | C/T | C | 104 | C/T | −8.55% |
| | | | 708* | | | | | |
| rs2069849 | IL6 | CDS | hsa-miR-744 | C/T | C | 128 | C/T | −10.31% |
| rs2069849 | IL6 | CDS | hsa-miR-760 | C/T | C | 111 | C/T | −16.16% |
| rs2069849 | IL6 | CDS | hsa-miR-769- | C/T | C | 98 | C/T | −14.85% |
| | | | 3p | | | | | |
| rs2069849 | IL6 | CDS | hsa-miR-873 | C/T | C | 161 | C/T | −17.47% |
| rs2069849 | IL6 | CDS | hsa-miR-875- | C/T | T | 125 | C/T | 33.54% |
| | | | 3p | | | | | |
| rs2069849 | IL6 | CDS | hsa-miR-877 | C/T | C | 91 | C/T | −21.64% |
| rs2069849 | IL6 | CDS | hsa-miR-885- | C/T | C | 91 | C/T | −15.92% |
| | | | 3p | | | | | |
| rs2069849 | IL6 | CDS | hsa-miR-921 | C/T | C | 85 | C/T | −20.69% |
| rs2069849 | IL6 | CDS | hsa-miR-93 | C/T | C | 112 | C/T | −53.29% |
| rs2069849 | IL6 | CDS | hsa-miR-939 | C/T | C | 142 | C/T | −8.83% |
| rs2675 | ITGB5 | 3′ UTR | hsa-let-7f-2* | A/C | A | 119 | A/C | −14.77% |
| rs2675 | ITGB5 | 3′ UTR | hsa-miR-103- | A/C | C | 130 | A/C | 13.54% |
| | | | 2* | | | | | |
| rs2675 | ITGB5 | 3′ UTR | hsa-miR- | A/C | A | 102 | A/C | −8.57% |
| | | | 1182 | | | | | |
| rs2675 | ITGB5 | 3′ UTR | hsa-miR- | A/C | C | 128 | A/C | 11.93% |
| | | | 1184 | | | | | |
| rs2675 | ITGB5 | 3′ UTR | hsa-miR- | A/C | C | 124 | A/C | 47.49% |
| | | | 1200 | | | | | |
| rs2675 | ITGB5 | 3′ UTR | hsa-miR- | A/C | C | 90 | A/C | 18.19% |
| | | | 1208 | | | | | |
| rs2675 | ITGB5 | 3′ UTR | hsa-miR- | A/C | C | 101 | A/C | 42.73% |
| | | | 1225-3p | | | | | |
| rs2675 | ITGB5 | 3′ UTR | hsa-miR- | A/C | A | 123 | A/C | −14.76% |
| | | | 1237 | | | | | |
| rs2675 | ITGB5 | 3′ UTR | hsa-miR- | A/C | C | 119 | A/C | 77.88% |
| | | | 1238 | | | | | |
| rs2675 | ITGB5 | 3′ UTR | hsa-miR- | A/C | C | 103 | A/C | 15.19% |
| | | | 1244 | | | | | |
| rs2675 | ITGB5 | 3′ UTR | hsa-miR- | A/C | A | 142 | A/C | −9.23% |
| | | | 1247 | | | | | |
| rs2675 | ITGB5 | 3′ UTR | hsa-miR- | A/C | A | 147 | A/C | −19.62% |
| | | | 1249 | | | | | |
| rs2675 | ITGB5 | 3′ UTR | hsa-miR- | A/C | C | 80 | A/C | 12.66% |
| | | | 1292 | | | | | |
| rs2675 | ITGB5 | 3′ UTR | hsa-miR- | A/C | C | 97 | A/C | 29.84% |
| | | | 1307 | | | | | |
| rs2675 | ITGB5 | 3′ UTR | hsa-miR-132 | A/C | A | 139 | A/C | −13.43% |
| rs2675 | ITGB5 | 3′ UTR | hsa-miR-136 | A/C | C | 95 | A/C | 12.66% |
| rs2675 | ITGB5 | 3′ UTR | hsa-miR-138 | A/C | C | 85 | A/C | 29.27% |
| rs2675 | ITGB5 | 3′ UTR | hsa-miR- | A/C | C | 110 | A/C | 17.62% |
| | | | 143* | | | | | |
| rs2675 | ITGB5 | 3′ UTR | hsa-miR-145 | A/C | A | 100 | A/C | −20.33% |
| rs2675 | ITGB5 | 3′ UTR | hsa-miR- | A/C | C | 87 | A/C | 143.05% |
| | | | 1468 | | | | | |
| rs2675 | ITGB5 | 3′ UTR | hsa-miR- | A/C | C | 93 | A/C | 25.84% |
| | | | 1471 | | | | | |
| rs2675 | ITGB5 | 3′ UTR | hsa-miR- | A/C | C | 93 | A/C | 32.73% |
| | | | 1537 | | | | | |
| rs2675 | ITGB5 | 3′ UTR | hsa-miR- | A/C | A | 104 | A/C | −29.23% |
| | | | 181a-2* | | | | | |
| rs2675 | ITGB5 | 3′ UTR | hsa-miR- | A/C | C | 121 | A/C | 9.67% |
| | | | 18a* | | | | | |
| rs2675 | ITGB5 | 3′ UTR | hsa-miR- | A/C | C | 130 | A/C | 8.41% |
| | | | 1913 | | | | | |
| rs2675 | ITGB5 | 3′ UTR | hsa-miR- | A/C | C | 129 | A/C | 9.35% |
| | | | 199a-3p | | | | | |
| rs2675 | ITGB5 | 3′ UTR | hsa-miR- | A/C | C | 129 | A/C | 9.35% |
| | | | 199b-3p | | | | | |
| rs2675 | ITGB5 | 3′ UTR | hsa-miR- | A/C | A | 103 | A/C | −10.46% |
| | | | 200a* | | | | | |
| rs2675 | ITGB5 | 3′ UTR | hsa-miR- | A/C | C | 88 | A/C | 22.73% |
| | | | 200b* | | | | | |
| rs2675 | ITGB5 | 3′ UTR | hsa-miR-205 | A/C | C | 123 | A/C | 38.28% |
| rs2675 | ITGB5 | 3′ UTR | hsa-miR-221 | A/C | A | 88 | A/C | −8.65% |
| rs2675 | ITGB5 | 3′ UTR | hsa-miR-222 | A/C | C | 78 | A/C | 9.84% |
| rs2675 | ITGB5 | 3′ UTR | hsa-miR-296- | A/C | C | 77 | A/C | 10.98% |
| | | | 3p | | | | | |
| rs2675 | ITGB5 | 3′ UTR | hsa-miR- | A/C | C | 108 | A/C | 22.71% |
| | | | 30a* | | | | | |
| rs2675 | ITGB5 | 3′ UTR | hsa-miR- | A/C | C | 99 | A/C | 21.89% |
| | | | 30d* | | | | | |
| rs2675 | ITGB5 | 3′ UTR | hsa-miR- | A/C | C | 99 | A/C | 24.91% |
| | | | 30e* | | | | | |
| rs2675 | ITGB5 | 3′ UTR | hsa-miR-330- | A/C | C | 99 | A/C | 42.29% |
| | | | 5p | | | | | |
| rs2675 | ITGB5 | 3′ UTR | hsa-miR-34a | A/C | C | 134 | A/C | 12.81% |
| rs2675 | ITGB5 | 3′ UTR | hsa-miR-369- | A/C | C | 108 | A/C | 34.36% |
| | | | 5p | | | | | |
| rs2675 | ITGB5 | 3′ UTR | hsa-miR- | A/C | C | 160 | A/C | 10.73% |
| | | | 378* | | | | | |
| rs2675 | ITGB5 | 3′ UTR | hsa-miR-412 | A/C | C | 120 | A/C | 18.24% |
| rs2675 | ITGB5 | 3′ UTR | hsa-miR- | A/C | C | 134 | A/C | 42.48% |
| | | | 431* | | | | | |
| rs2675 | ITGB5 | 3′ UTR | hsa-miR-432 | A/C | C | 101 | A/C | 10.79% |
| rs2675 | ITGB5 | 3′ UTR | hsa-miR- | A/C | C | 130 | A/C | 8.77% |
| | | | 449a | | | | | |
| rs2675 | ITGB5 | 3′ UTR | hsa-miR- | A/C | C | 131 | A/C | 9.75% |
| | | | 449b | | | | | |
| rs2675 | ITGB5 | 3′ UTR | hsa-miR- | A/C | C | 121 | A/C | 24.34% |
| | | | 454* | | | | | |
| rs2675 | ITGB5 | 3′ UTR | hsa-miR-483- | A/C | C | 119 | A/C | 12.86% |
| | | | 3p | | | | | |
| rs2675 | ITGB5 | 3′ UTR | hsa-miR-486- | A/C | C | 108 | A/C | 8.52% |
| | | | 5p | | | | | |
| rs2675 | ITGB5 | 3′ UTR | hsa-miR-493 | A/C | A | 136 | A/C | −11.71% |
| rs2675 | ITGB5 | 3′ UTR | hsa-miR-501- | A/C | C | 104 | A/C | 8.38% |
| | | | 5p | | | | | |
| rs2675 | ITGB5 | 3′ UTR | hsa-miR-504 | A/C | A | 139 | A/C | −13.96% |
| rs2675 | ITGB5 | 3′ UTR | hsa-miR-525- | A/C | C | 94 | A/C | 21.86% |
| | | | 3p | | | | | |
| rs2675 | ITGB5 | 3′ UTR | hsa-miR- | A/C | C | 100 | A/C | 60.04% |
| | | | 550* | | | | | |
| rs2675 | ITGB5 | 3′ UTR | hsa-miR- | A/C | A | 141 | A/C | −10.22% |
| | | | 551b | | | | | |
| rs2675 | ITGB5 | 3′ UTR | hsa-miR-564 | A/C | C | 91 | A/C | 31.36% |
| rs2675 | ITGB5 | 3′ UTR | hsa-miR-578 | A/C | A | 152 | A/C | −30.53% |
| rs2675 | ITGB5 | 3′ UTR | hsa-miR-617 | A/C | C | 117 | A/C | 15.35% |
| rs2675 | ITGB5 | 3′ UTR | hsa-miR-622 | A/C | A | 135 | A/C | −21.61% |
| rs2675 | ITGB5 | 3′ UTR | hsa-miR-630 | A/C | A | 122 | A/C | −11.03% |
| rs2675 | ITGB5 | 3′ UTR | hsa-miR-635 | A/C | C | 92 | A/C | 19.49% |
| rs2675 | ITGB5 | 3′ UTR | hsa-miR-642 | A/C | C | 114 | A/C | 24.71% |
| rs2675 | ITGB5 | 3′ UTR | hsa-miR-654- | A/C | C | 82 | A/C | 14.75% |
| | | | 3p | | | | | |
| rs2675 | ITGB5 | 3′ UTR | hsa-miR-671- | A/C | C | 97 | A/C | 12.61% |
| | | | 5p | | | | | |
| rs2675 | ITGB5 | 3′ UTR | hsa-miR-769- | A/C | C | 88 | A/C | 10.39% |
| | | | 3p | | | | | |
| rs2675 | ITGB5 | 3′ UTR | hsa-miR-885- | A/C | C | 115 | A/C | 12.28% |
| | | | 3p | | | | | |
| rs2675 | ITGB5 | 3′ UTR | hsa-miR-887 | A/C | A | 83 | A/C | −9.30% |
| rs2675 | ITGB5 | 3′ UTR | hsa-miR-937 | A/C | C | 119 | A/C | 10.96% |
| rs2675 | ITGB5 | 3′ UTR | hsa-miR-943 | A/C | C | 137 | A/C | 15.08% |
| rs11902171 | ITGAV | 3′ UTR | hsa-let-7c* | C/G | C | 116 | C/G | −24.57% |
| rs11902171 | ITGAV | 3′ UTR | hsa-miR- | C/G | C | 110 | C/G | −23.00% |
| | | | 1182 | | | | | |
| rs11902171 | ITGAV | 3′ UTR | hsa-miR- | C/G | C | 93 | C/G | −15.70% |
| | | | 1183 | | | | | |
| rs11902171 | ITGAV | 3′ UTR | hsa-miR- | C/G | G | 98 | C/G | 45.20% |
| | | | 1207-5p | | | | | |
| rs11902171 | ITGAV | 3′ UTR | hsa-miR- | C/G | C | 139 | C/G | −32.92% |
| | | | 1226* | | | | | |
| rs11902171 | ITGAV | 3′ UTR | hsa-miR- | C/G | G | 91 | C/G | 38.85% |
| | | | 124* | | | | | |
| rs11902171 | ITGAV | 3′ UTR | hsa-miR- | C/G | C | 94 | C/G | −23.93% |
| | | | 125a-3p | | | | | |
| rs11902171 | ITGAV | 3′ UTR | hsa-miR- | C/G | C | 96 | C/G | −24.56% |
| | | | 1270 | | | | | |
| rs11902171 | ITGAV | 3′ UTR | hsa-miR- | C/G | G | 86 | C/G | 47.52% |
| | | | 1272 | | | | | |
| rs11902171 | ITGAV | 3′ UTR | hsa-miR- | C/G | C | 100 | C/G | −77.85% |
| | | | 1275 | | | | | |
| rs11902171 | ITGAV | 3′ UTR | hsa-miR- | C/G | G | 118 | C/G | 26.80% |
| | | | 1288 | | | | | |
| rs11902171 | ITGAV | 3′ UTR | hsa-miR- | C/G | G | 83 | C/G | 15.33% |
| | | | 1292 | | | | | |
| rs11902171 | ITGAV | 3′ UTR | hsa-miR- | C/G | C | 114 | C/G | −45.02% |
| | | | 1300 | | | | | |
| rs11902171 | ITGAV | 3′ UTR | hsa-miR- | C/G | G | 83 | C/G | 26.82% |
| | | | 1469 | | | | | |
| rs11902171 | ITGAV | 3′ UTR | hsa-miR- | C/G | C | 109 | C/G | −36.64% |
| | | | 148a* | | | | | |
| rs11902171 | ITGAV | 3′ UTR | hsa-miR- | C/G | C | 111 | C/G | −29.82% |
| | | | 149* | | | | | |
| rs11902171 | ITGAV | 3′ UTR | hsa-miR- | C/G | G | 109 | C/G | 30.28% |
| | | | 1538 | | | | | |
| rs11902171 | ITGAV | 3′ UTR | hsa-miR-17* | C/G | G | 106 | C/G | 20.25% |
| rs11902171 | ITGAV | 3′ UTR | hsa-miR- | C/G | C | 117 | C/G | −14.66% |
| | | | 1827 | | | | | |
| rs11902171 | ITGAV | 3′ UTR | hsa-miR-184 | C/G | G | 98 | C/G | 61.43% |
| rs11902171 | ITGAV | 3′ UTR | hsa-miR- | C/G | G | 105 | C/G | 19.78% |
| | | | 1908 | | | | | |
| rs11902171 | ITGAV | 3′ UTR | hsa-miR- | C/G | C | 145 | C/G | −27.08% |
| | | | 1909* | | | | | |
| rs11902171 | ITGAV | 3′ UTR | hsa-miR-192 | C/G | G | 100 | C/G | 54.80% |
| rs11902171 | ITGAV | 3′ UTR | hsa-miR- | C/G | G | 83 | C/G | 10.89% |
| | | | 193a-5p | | | | | |
| rs11902171 | ITGAV | 3′ UTR | hsa-miR- | C/G | C | 106 | C/G | −24.99% |
| | | | 193b* | | | | | |
| rs11902171 | ITGAV | 3′ UTR | hsa-miR- | C/G | G | 155 | C/G | 41.66% |
| | | | 196b* | | | | | |
| rs11902171 | ITGAV | 3′ UTR | hsa-miR-202 | C/G | C | 100 | C/G | −11.10% |
| rs11902171 | ITGAV | 3′ UTR | hsa-miR-219- | C/G | C | 127 | C/G | −11.54% |
| | | | 1-3p | | | | | |
| rs11902171 | ITGAV | 3′ UTR | hsa-miR-22 | C/G | G | 106 | C/G | 99.68% |
| rs11902171 | ITGAV | 3′ UTR | hsa-miR- | C/G | G | 108 | C/G | 30.39% |
| | | | 223* | | | | | |
| rs11902171 | ITGAV | 3′ UTR | hsa-miR- | C/G | C | 122 | C/G | −34.63% |
| | | | 27b* | | | | | |
| rs11902171 | ITGAV | 3′ UTR | hsa-miR-296- | C/G | C | 100 | C/G | −20.07% |
| | | | 3p | | | | | |
| rs11902171 | ITGAV | 3′ UTR | hsa-miR- | C/G | C | 120 | C/G | −77.90% |
| | | | 30b* | | | | | |
| rs11902171 | ITGAV | 3′ UTR | hsa-miR-30c- | C/G | C | 119 | C/G | −51.64% |
| | | | 1* | | | | | |
| rs11902171 | ITGAV | 3′ UTR | hsa-miR-30c- | C/G | C | 129 | C/G | −112.51% |
| | | | 2* | | | | | |
| rs11902171 | ITGAV | 3′ UTR | hsa-miR- | C/G | G | 96 | C/G | 36.67% |
| | | | 30e* | | | | | |
| rs11902171 | ITGAV | 3′ UTR | hsa-miR- | C/G | G | 108 | C/G | 69.30% |
| | | | 33b* | | | | | |
| rs11902171 | ITGAV | 3′ UTR | hsa-miR-382 | C/G | G | 130 | C/G | 11.07% |
| rs11902171 | ITGAV | 3′ UTR | hsa-miR- | C/G | C | 125 | C/G | −23.62% |
| | | | 505* | | | | | |
| rs11902171 | ITGAV | 3′ UTR | hsa-miR-511 | C/G | G | 103 | C/G | 131.03% |
| rs11902171 | ITGAV | 3′ UTR | hsa-miR-512- | C/G | G | 94 | C/G | 58.20% |
| | | | 3p | | | | | |
| rs11902171 | ITGAV | 3′ UTR | hsa-miR-542- | C/G | G | 135 | C/G | 118.18% |
| | | | 3p | | | | | |
| rs11902171 | ITGAV | 3′ UTR | hsa-miR-550 | C/G | C | 93 | C/G | −26.19% |
| rs11902171 | ITGAV | 3′ UTR | hsa-miR-552 | C/G | G | 118 | C/G | 36.23% |
| rs11902171 | ITGAV | 3′ UTR | hsa-miR-584 | C/G | G | 82 | C/G | 19.21% |
| rs11902171 | ITGAV | 3′ UTR | hsa-miR-602 | C/G | G | 98 | C/G | 60.80% |
| rs11902171 | ITGAV | 3′ UTR | hsa-miR-608 | C/G | G | 85 | C/G | 17.14% |
| rs11902171 | ITGAV | 3′ UTR | hsa-miR-628- | C/G | G | 93 | C/G | 51.20% |
| | | | 3p | | | | | |
| rs11902171 | ITGAV | 3′ UTR | hsa-miR-637 | C/G | C | 129 | C/G | −20.78% |
| rs11902171 | ITGAV | 3′ UTR | hsa-miR-639 | C/G | G | 82 | C/G | 11.16% |
| rs11902171 | ITGAV | 3′ UTR | hsa-miR-649 | C/G | C | 111 | C/G | −36.34% |
| rs11902171 | ITGAV | 3′ UTR | hsa-miR-654- | C/G | C | 102 | C/G | −29.67% |
| | | | 5p | | | | | |
| rs11902171 | ITGAV | 3′ UTR | hsa-miR-661 | C/G | G | 97 | C/G | 48.02% |
| rs11902171 | ITGAV | 3′ UTR | hsa-miR- | C/G | G | 87 | C/G | 21.00% |
| | | | 663b | | | | | |
| rs11902171 | ITGAV | 3′ UTR | hsa-miR-769- | C/G | C | 114 | C/G | −19.28% |
| | | | 3p | | | | | |
| rs11902171 | ITGAV | 3′ UTR | hsa-miR-769- | C/G | C | 107 | C/G | −46.30% |
| | | | 5p | | | | | |
| rs11902171 | ITGAV | 3′ UTR | hsa-miR-921 | C/G | G | 82 | C/G | 34.34% |
| rs11902171 | ITGAV | 3′ UTR | hsa-miR-92a- | C/G | C | 93 | C/G | −14.70% |
| | | | 2* | | | | | |
| rs11902171 | ITGAV | 3′ UTR | hsa-miR-939 | C/G | C | 138 | C/G | −17.12% |
| rs11902171 | ITGAV | 3′ UTR | hsa-miR-941 | C/G | G | 97 | C/G | 18.36% |
| rs7702523 | JMY | 3′ UTR | hsa-miR- | C/T | C | 89 | C/T | −27.99% |
| | | | 1183 | | | | | |
| rs7702523 | JMY | 3′ UTR | hsa-miR- | C/T | C | 117 | C/T | −11.47% |
| | | | 1205 | | | | | |
| rs7702523 | JMY | 3′ UTR | hsa-miR- | C/T | T | 105 | C/T | 9.29% |
| | | | 1225-5p | | | | | |
| rs7702523 | JMY | 3′ UTR | hsa-miR- | C/T | C | 126 | C/T | −15.93% |
| | | | 1231 | | | | | |
| rs7702523 | JMY | 3′ UTR | hsa-miR- | C/T | T | 115 | C/T | 19.12% |
| | | | 1245 | | | | | |
| rs7702523 | JMY | 3′ UTR | hsa-miR- | C/T | C | 99 | C/T | −8.55% |
| | | | 1250 | | | | | |
| rs7702523 | JMY | 3′ UTR | hsa-miR- | C/T | C | 80 | C/T | −26.59% |
| | | | 1287 | | | | | |
| rs7702523 | JMY | 3′ UTR | hsa-miR-142- | C/T | T | 134 | C/T | 14.96% |
| | | | 3p | | | | | |
| rs7702523 | JMY | 3′ UTR | hsa-miR- | C/T | C | 124 | C/T | −15.00% |
| | | | 148b* | | | | | |
| rs7702523 | JMY | 3′ UTR | hsa-miR-379 | C/T | T | 87 | C/T | 28.76% |
| rs7702523 | JMY | 3′ UTR | hsa-miR-409- | C/T | C | 96 | C/T | −9.61% |
| | | | 5p | | | | | |
| rs7702523 | JMY | 3′ UTR | hsa-miR-423- | C/T | C | 91 | C/T | −9.36% |
| | | | 3p | | | | | |
| rs7702523 | JMY | 3′ UTR | hsa-miR-431 | C/T | C | 122 | C/T | −9.42% |
| rs7702523 | JMY | 3′ UTR | hsa-miR- | C/T | C | 98 | C/T | −36.17% |
| | | | 432* | | | | | |
| rs7702523 | JMY | 3′ UTR | hsa-miR-493 | C/T | T | 116 | C/T | 25.89% |
| rs7702523 | JMY | 3′ UTR | hsa-miR- | C/T | C | 90 | C/T | −160.99% |
| | | | 541* | | | | | |
| rs7702523 | JMY | 3′ UTR | hsa-miR-550 | C/T | C | 131 | C/T | −10.34% |
| rs7702523 | JMY | 3′ UTR | hsa-miR-572 | C/T | C | 114 | C/T | −9.89% |
| rs7702523 | JMY | 3′ UTR | hsa-miR-574- | C/T | C | 112 | C/T | −11.31% |
| | | | 5p | | | | | |
| rs7702523 | JMY | 3′ UTR | hsa-miR-610 | C/T | T | 80 | C/T | 10.01% |
| rs7702523 | JMY | 3′ UTR | hsa-miR-614 | C/T | C | 84 | C/T | −30.19% |
| rs7702523 | JMY | 3′ UTR | hsa-miR-622 | C/T | C | 130 | C/T | −9.90% |
| rs7702523 | JMY | 3′ UTR | hsa-miR-636 | C/T | C | 112 | C/T | −18.31% |
| rs7702523 | JMY | 3′ UTR | hsa-miR-639 | C/T | C | 102 | C/T | −14.39% |
| rs7702523 | JMY | 3′ UTR | hsa-miR-643 | C/T | C | 106 | C/T | −11.61% |
| rs7702523 | JMY | 3′ UTR | hsa-miR-665 | C/T | C | 81 | C/T | −43.64% |
| rs7702523 | JMY | 3′ UTR | hsa-miR-720 | C/T | C | 97 | C/T | −13.69% |
| rs7702523 | JMY | 3′ UTR | hsa-miR-744 | C/T | C | 88 | C/T | −27.76% |
| rs7702523 | JMY | 3′ UTR | hsa-miR-767- | C/T | C | 120 | C/T | −13.36% |
| | | | 3p | | | | | |
| rs7702523 | JMY | 3′ UTR | hsa-miR-874 | C/T | C | 120 | C/T | −11.92% |
| rs7702523 | JMY | 3′ UTR | hsa-miR-93 | C/T | C | 100 | C/T | −15.92% |
| rs7702523 | JMY | 3′ UTR | hsa-miR-934 | C/T | T | 152 | C/T | 33.53% |
| rs1940475 | MMP8 | CDS | hsa-let-7a-2* | C/T | C | 103 | C/T | −11.66% |
| rs1940475 | MMP8 | CDS | hsa-miR- | C/T | C | 95 | C/T | −19.30% |
| | | | 1204 | | | | | |
| rs1940475 | MMP8 | CDS | hsa-miR- | C/T | C | 106 | C/T | −24.11% |
| | | | 1227 | | | | | |
| rs1940475 | MMP8 | CDS | hsa-miR- | C/T | C | 120 | C/T | −11.25% |
| | | | 1284 | | | | | |
| rs1940475 | MMP8 | CDS | hsa-miR- | C/T | C | 90 | C/T | −104.77% |
| | | | 1296 | | | | | |
| rs1940475 | MMP8 | CDS | hsa-miR- | C/T | C | 137 | C/T | −11.09% |
| | | | 1307 | | | | | |
| rs1940475 | MMP8 | CDS | hsa-miR-138- | C/T | C | 100 | C/T | −8.49% |
| | | | 1* | | | | | |
| rs1940475 | MMP8 | CDS | hsa-miR-139- | C/T | T | 124 | C/T | 25.23% |
| | | | 5p | | | | | |
| rs1940475 | MMP8 | CDS | hsa-miR- | C/T | C | 80 | C/T | −51.69% |
| | | | 1471 | | | | | |
| rs1940475 | MMP8 | CDS | hsa-miR- | C/T | C | 91 | C/T | −51.70% |
| | | | 1538 | | | | | |
| rs1940475 | MMP8 | CDS | hsa-miR- | C/T | T | 95 | C/T | 60.89% |
| | | | 1539 | | | | | |
| rs1940475 | MMP8 | CDS | hsa-miR- | C/T | C | 130 | C/T | −13.50% |
| | | | 185* | | | | | |
| rs1940475 | MMP8 | CDS | hsa-miR- | C/T | C | 127 | C/T | −16.34% |
| | | | 1908 | | | | | |
| rs1940475 | MMP8 | CDS | hsa-miR- | C/T | C | 93 | C/T | −22.42% |
| | | | 191* | | | | | |
| rs1940475 | MMP8 | CDS | hsa-miR- | C/T | C | 92 | C/T | −29.81% |
| | | | 1914* | | | | | |
| rs1940475 | MMP8 | CDS | hsa-miR-197 | C/T | C | 117 | C/T | −19.90% |
| rs1940475 | MMP8 | CDS | hsa-miR- | C/T | C | 95 | C/T | −15.47% |
| | | | 199b-5p | | | | | |
| rs1940475 | MMP8 | CDS | hsa-miR- | C/T | C | 89 | C/T | −71.47% |
| | | | 20b* | | | | | |
| rs1940475 | MMP8 | CDS | hsa-miR-224 | C/T | C | 106 | C/T | −51.74% |
| rs1940475 | MMP8 | CDS | hsa-miR- | C/T | T | 84 | C/T | 121.89% |
| | | | 26b* | | | | | |
| rs1940475 | MMP8 | CDS | hsa-miR-27a | C/T | C | 127 | C/T | −33.25% |
| rs1940475 | MMP8 | CDS | hsa-miR-298 | C/T | C | 81 | C/T | −15.22% |
| rs1940475 | MMP8 | CDS | hsa-miR- | C/T | C | 95 | C/T | −12.01% |
| | | | 30d* | | | | | |
| rs1940475 | MMP8 | CDS | hsa-miR-326 | C/T | C | 88 | C/T | −38.93% |
| rs1940475 | MMP8 | CDS | hsa-miR-346 | C/T | C | 83 | C/T | −37.38% |
| rs1940475 | MMP8 | CDS | hsa-miR-363 | C/T | C | 107 | C/T | −43.59% |
| rs1940475 | MMP8 | CDS | hsa-miR-423- | C/T | T | 80 | C/T | 17.49% |
| | | | 3p | | | | | |
| rs1940475 | MMP8 | CDS | hsa-miR- | C/T | C | 90 | C/T | −24.95% |
| | | | 432* | | | | | |
| rs1940475 | MMP8 | CDS | hsa-miR-453 | C/T | T | 96 | C/T | 19.68% |
| rs1940475 | MMP8 | CDS | hsa-miR-485- | C/T | C | 132 | C/T | −30.06% |
| | | | 5p | | | | | |
| rs1940475 | MMP8 | CDS | hsa-miR-491- | C/T | C | 116 | C/T | −31.14% |
| | | | 5p | | | | | |
| rs1940475 | MMP8 | CDS | hsa-miR- | C/T | C | 101 | C/T | −47.71% |
| | | | 518c | | | | | |
| rs1940475 | MMP8 | CDS | hsa-miR- | C/T | C | 97 | C/T | −14.83% |
| | | | 551a | | | | | |
| rs1940475 | MMP8 | CDS | hsa-miR-557 | C/T | C | 107 | C/T | −10.39% |
| rs1940475 | MMP8 | CDS | hsa-miR-611 | C/T | C | 128 | C/T | −13.30% |
| rs1940475 | MMP8 | CDS | hsa-miR-618 | C/T | T | 104 | C/T | 10.34% |
| rs1940475 | MMP8 | CDS | hsa-miR-659 | C/T | C | 76 | C/T | −13.51% |
| rs1940475 | MMP8 | CDS | hsa-miR-662 | C/T | C | 106 | C/T | −18.45% |
| rs1940475 | MMP8 | CDS | hsa-miR-663 | C/T | C | 133 | C/T | −24.65% |
| rs1940475 | MMP8 | CDS | hsa-miR-665 | C/T | C | 112 | C/T | −17.47% |
| rs1940475 | MMP8 | CDS | hsa-miR-671- | C/T | C | 101 | C/T | −9.16% |
| | | | 3p | | | | | |
| rs1940475 | MMP8 | CDS | hsa-miR-744 | C/T | C | 148 | C/T | −10.15% |
| rs1940475 | MMP8 | CDS | hsa-miR-766 | C/T | C | 121 | C/T | −41.44% |
| rs1940475 | MMP8 | CDS | hsa-miR-767- | C/T | C | 86 | C/T | −84.82% |
| | | | 3p | | | | | |
| rs1800566 | NQO1 | CDS | hsa-let-7b | C/T | C | 108 | C/T | −16.79% |
| rs1800566 | NQO1 | CDS | hsa-miR- | C/T | C | 129 | C/T | −10.48% |
| | | | 1182 | | | | | |
| rs1800566 | NQO1 | CDS | hsa-miR- | C/T | C | 103 | C/T | −15.19% |
| | | | 1202 | | | | | |
| rs1800566 | NQO1 | CDS | hsa-miR- | C/T | C | 95 | C/T | −21.13% |
| | | | 1207-5p | | | | | |
| rs1800566 | NQO1 | CDS | hsa-miR- | C/T | C | 113 | C/T | −49.81% |
| | | | 1226* | | | | | |
| rs1800566 | NQO1 | CDS | hsa-miR- | C/T | C | 158 | C/T | −8.28% |
| | | | 125a-3p | | | | | |
| rs1800566 | NQO1 | CDS | hsa-miR- | C/T | C | 117 | C/T | −13.22% |
| | | | 1287 | | | | | |
| rs1800566 | NQO1 | CDS | hsa-miR- | C/T | C | 84 | C/T | −94.06% |
| | | | 135a* | | | | | |
| rs1800566 | NQO1 | CDS | hsa-miR- | C/T | C | 124 | C/T | −10.16% |
| | | | 146b-3p | | | | | |
| rs1800566 | NQO1 | CDS | hsa-miR- | C/T | C | 95 | C/T | −66.80% |
| | | | 150* | | | | | |
| rs1800566 | NQO1 | CDS | hsa-miR-17 | C/T | C | 106 | C/T | −14.78% |
| rs1800566 | NQO1 | CDS | hsa-miR- | C/T | C | 163 | C/T | −13.29% |
| | | | 182* | | | | | |
| rs1800566 | NQO1 | CDS | hsa-miR- | C/T | C | 110 | C/T | −13.43% |
| | | | 1914* | | | | | |
| rs1800566 | NQO1 | CDS | hsa-miR- | C/T | C | 119 | C/T | −16.29% |
| | | | 193b* | | | | | |
| rs1800566 | NQO1 | CDS | hsa-miR-202 | C/T | C | 117 | C/T | −14.22% |
| rs1800566 | NQO1 | CDS | hsa-miR- | C/T | C | 115 | C/T | −12.97% |
| | | | 23a* | | | | | |
| rs1800566 | NQO1 | CDS | hsa-miR-25* | C/T | C | 100 | C/T | −26.66% |
| rs1800566 | NQO1 | CDS | hsa-miR-296- | C/T | C | 95 | C/T | −20.50% |
| | | | 3p | | | | | |
| rs1800566 | NQO1 | CDS | hsa-miR-379 | C/T | C | 114 | C/T | −13.86% |
| rs1800566 | NQO1 | CDS | hsa-miR-512- | C/T | C | 125 | C/T | −13.54% |
| | | | 3p | | | | | |
| rs1800566 | NQO1 | CDS | hsa-miR-616 | C/T | C | 127 | C/T | −12.14% |
| rs1800566 | NQO1 | CDS | hsa-miR-630 | C/T | T | 105 | C/T | 19.17% |
| rs1800566 | NQO1 | CDS | hsa-miR-659 | C/T | C | 121 | C/T | −16.50% |
| rs1800566 | NQO1 | CDS | hsa-miR- | C/T | C | 112 | C/T | −11.21% |
| | | | 663b | | | | | |
| rs1800566 | NQO1 | CDS | hsa-miR-671- | C/T | C | 124 | C/T | −15.88% |
| | | | 3p | | | | | |
| rs1800566 | NQO1 | CDS | hsa-miR-675 | C/T | C | 107 | C/T | −10.10% |
| rs1800566 | NQO1 | CDS | hsa-miR-744 | C/T | C | 89 | C/T | −53.35% |
| rs1800566 | NQO1 | CDS | hsa-miR-760 | C/T | C | 86 | C/T | −25.93% |
| rs1800566 | NQO1 | CDS | hsa-miR-769- | C/T | T | 109 | C/T | 22.82% |
| | | | 3p | | | | | |
| rs1800566 | NQO1 | CDS | hsa-miR-769- | C/T | T | 95 | C/T | 31.21% |
| | | | 5p | | | | | |
| rs1800566 | NQO1 | CDS | hsa-miR-922 | C/T | T | 106 | C/T | 11.75% |
| rs1800566 | NQO1 | CDS | hsa-miR-936 | C/T | C | 134 | C/T | −12.37% |
| rs1042438 | EHHADH | CDS | hsa-miR- | A/C | C | 132 | A/C | 12.86% |
| | | | 1225-5p | | | | | |
| rs1042438 | EHHADH | CDS | hsa-miR- | A/C | A | 110 | A/C | −14.84% |
| | | | 1257 | | | | | |
| rs1042438 | EHHADH | CDS | hsa-miR- | A/C | C | 128 | A/C | 16.16% |
| | | | 1268 | | | | | |
| rs1042438 | EHHADH | CDS | hsa-miR-134 | A/C | C | 101 | A/C | 16.79% |
| rs1042438 | EHHADH | CDS | hsa-miR- | A/C | C | 123 | A/C | 45.90% |
| | | | 150* | | | | | |
| rs1042438 | EHHADH | CDS | hsa-miR- | A/C | C | 132 | A/C | 20.45% |
| | | | 1914* | | | | | |
| rs1042438 | EHHADH | CDS | hsa-miR- | A/C | C | 158 | A/C | 12.65% |
| | | | 193b* | | | | | |
| rs1042438 | EHHADH | CDS | hsa-miR- | A/C | C | 112 | A/C | 18.41% |
| | | | 2110 | | | | | |
| rs1042438 | EHHADH | CDS | hsa-miR-342- | A/C | C | 121 | A/C | 161.68% |
| | | | 5p | | | | | |
| rs1042438 | EHHADH | CDS | hsa-miR-373 | A/C | C | 114 | A/C | 30.79% |
| rs1042438 | EHHADH | CDS | hsa-miR-432 | A/C | A | 81 | A/C | −105.10% |
| rs1042438 | EHHADH | CDS | hsa-miR-608 | A/C | C | 114 | A/C | 16.85% |
| rs1042438 | EHHADH | CDS | hsa-miR-615- | A/C | C | 112 | A/C | 63.05% |
| | | | 5p | | | | | |
| rs1042438 | EHHADH | CDS | hsa-miR-659 | A/C | A | 141 | A/C | −9.62% |
| rs1042438 | EHHADH | CDS | hsa-miR-663 | A/C | C | 113 | A/C | 36.59% |
| rs1042438 | EHHADH | CDS | hsa-miR- | A/C | C | 108 | A/C | 11.38% |
| | | | 92b* | | | | | |
| rs1042438 | EHHADH | CDS | hsa-miR-939 | A/C | C | 100 | A/C | 15.45% |
| rs1042838 | PGR | CDS | hsa-let-7g* | G/T | G | 82 | G/T | −48.44% |
| rs1042838 | PGR | CDS | hsa-miR-105 | G/T | T | 109 | G/T | 9.08% |
| rs1042838 | PGR | CDS | hsa-miR- | G/T | G | 126 | G/T | −15.09% |
| | | | 106b* | | | | | |
| rs1042838 | PGR | CDS | hsa-miR- | G/T | G | 110 | G/T | −22.82% |
| | | | 1179 | | | | | |
| rs1042838 | PGR | CDS | hsa-miR- | G/T | T | 102 | G/T | 27.17% |
| | | | 1184 | | | | | |
| rs1042838 | PGR | CDS | hsa-miR-122 | G/T | T | 117 | G/T | 23.16% |
| rs1042838 | PGR | CDS | hsa-miR- | G/T | T | 122 | G/T | 8.77% |
| | | | 1225-5p | | | | | |
| rs1042838 | PGR | CDS | hsa-miR- | G/T | G | 99 | G/T | −9.10% |
| | | | 1226* | | | | | |
| rs1042838 | PGR | CDS | hsa-miR- | G/T | G | 83 | G/T | −64.17% |
| | | | 1250 | | | | | |
| rs1042838 | PGR | CDS | hsa-miR- | G/T | G | 103 | G/T | −20.04% |
| | | | 1254 | | | | | |
| rs1042838 | PGR | CDS | hsa-miR- | G/T | G | 101 | G/T | −17.30% |
| | | | 1263 | | | | | |
| rs1042838 | PGR | CDS | hsa-miR- | G/T | T | 113 | G/T | 37.52% |
| | | | 1266 | | | | | |
| rs1042838 | PGR | CDS | hsa-miR-127- | G/T | G | 89 | G/T | −10.75% |
| | | | 3p | | | | | |
| rs1042838 | PGR | CDS | hsa-miR- | G/T | G | 129 | G/T | −8.24% |
| | | | 1288 | | | | | |
| rs1042838 | PGR | CDS | hsa-miR- | G/T | G | 131 | G/T | −18.85% |
| | | | 1291 | | | | | |
| rs1042838 | PGR | CDS | hsa-miR- | G/T | G | 147 | G/T | −8.26% |
| | | | 1292 | | | | | |
| rs1042838 | PGR | CDS | hsa-miR- | G/T | G | 122 | G/T | −19.96% |
| | | | 1299 | | | | | |
| rs1042838 | PGR | CDS | hsa-miR- | G/T | G | 94 | G/T | −50.68% |
| | | | 1305 | | | | | |
| rs1042838 | PGR | CDS | hsa-miR- | G/T | G | 135 | G/T | −22.78% |
| | | | 1306 | | | | | |
| rs1042838 | PGR | CDS | hsa-miR- | G/T | G | 105 | G/T | −19.17% |
| | | | 1307 | | | | | |
| rs1042838 | PGR | CDS | hsa-miR- | G/T | G | 95 | G/T | −46.74% |
| | | | 130a* | | | | | |
| rs1042838 | PGR | CDS | hsa-miR-134 | G/T | G | 102 | G/T | −35.62% |
| rs1042838 | PGR | CDS | hsa-miR- | G/T | G | 129 | G/T | −43.79% |
| | | | 135a | | | | | |
| rs1042838 | PGR | CDS | hsa-miR-139- | G/T | G | 127 | G/T | −9.89% |
| | | | 3p | | | | | |
| rs1042838 | PGR | CDS | hsa-miR-140- | G/T | G | 124 | G/T | −179.67% |
| | | | 3p | | | | | |
| rs1042838 | PGR | CDS | hsa-miR- | G/T | G | 110 | G/T | −13.68% |
| | | | 1471 | | | | | |
| rs1042838 | PGR | CDS | hsa-miR- | G/T | G | 113 | G/T | −16.21% |
| | | | 150* | | | | | |
| rs1042838 | PGR | CDS | hsa-miR- | G/T | G | 112 | G/T | −35.19% |
| | | | 181b | | | | | |
| rs1042838 | PGR | CDS | hsa-miR- | G/T | G | 106 | G/T | −21.01% |
| | | | 181d | | | | | |
| rs1042838 | PGR | CDS | hsa-miR-182 | G/T | G | 112 | G/T | −36.46% |
| rs1042838 | PGR | CDS | hsa-miR- | G/T | G | 105 | G/T | −12.38% |
| | | | 186* | | | | | |
| rs1042838 | PGR | CDS | hsa-miR-188- | G/T | G | 121 | G/T | −28.52% |
| | | | 3p | | | | | |
| rs1042838 | PGR | CDS | hsa-miR-188- | G/T | G | 88 | G/T | −31.38% |
| | | | 5p | | | | | |
| rs1042838 | PGR | CDS | hsa-miR- | G/T | G | 96 | G/T | −35.11% |
| | | | 18b* | | | | | |
| rs1042838 | PGR | CDS | hsa-miR- | G/T | T | 127 | G/T | 9.58% |
| | | | 1909* | | | | | |
| rs1042838 | PGR | CDS | hsa-miR- | G/T | G | 82 | G/T | −42.36% |
| | | | 1914 | | | | | |
| rs1042838 | PGR | CDS | hsa-miR- | G/T | T | 80 | G/T | 17.08% |
| | | | 193a-5p | | | | | |
| rs1042838 | PGR | CDS | hsa-miR-194 | G/T | G | 90 | G/T | −11.95% |
| rs1042838 | PGR | CDS | hsa-miR-198 | G/T | T | 98 | G/T | 20.46% |
| rs1042838 | PGR | CDS | hsa-miR- | G/T | G | 90 | G/T | −19.32% |
| | | | 200a* | | | | | |
| rs1042838 | PGR | CDS | hsa-miR- | G/T | G | 113 | G/T | −25.61% |
| | | | 2110 | | | | | |
| rs1042838 | PGR | CDS | hsa-miR- | G/T | G | 92 | G/T | −14.90% |
| | | | 216a | | | | | |
| rs1042838 | PGR | CDS | hsa-miR-221 | G/T | G | 98 | G/T | −42.16% |
| rs1042838 | PGR | CDS | hsa-miR-222 | G/T | G | 106 | G/T | −42.86% |
| rs1042838 | PGR | CDS | hsa-miR- | G/T | G | 106 | G/T | −19.82% |
| | | | 23b* | | | | | |
| rs1042838 | PGR | CDS | hsa-miR-24- | G/T | G | 148 | G/T | −38.57% |
| | | | 1* | | | | | |
| rs1042838 | PGR | CDS | hsa-miR-24- | G/T | G | 149 | G/T | −38.57% |
| | | | 2* | | | | | |
| rs1042838 | PGR | CDS | hsa-miR- | G/T | G | 82 | G/T | −16.52% |
| | | | 302c* | | | | | |
| rs1042838 | PGR | CDS | hsa-miR- | G/T | G | 118 | G/T | −15.06% |
| | | | 30a* | | | | | |
| rs1042838 | PGR | CDS | hsa-miR- | G/T | G | 86 | G/T | −19.69% |
| | | | 30b* | | | | | |
| rs1042838 | PGR | CDS | hsa-miR- | G/T | G | 116 | G/T | −10.38% |
| | | | 30d* | | | | | |
| rs1042838 | PGR | CDS | hsa-miR- | G/T | G | 122 | G/T | −28.40% |
| | | | 30e* | | | | | |
| rs1042838 | PGR | CDS | hsa-miR- | G/T | G | 115 | G/T | −12.64% |
| | | | 320a | | | | | |
| rs1042838 | PGR | CDS | hsa-miR- | G/T | G | 119 | G/T | −12.59% |
| | | | 320b | | | | | |
| rs1042838 | PGR | CDS | hsa-miR-323- | G/T | G | 87 | G/T | −8.82% |
| | | | 5p | | | | | |
| rs1042838 | PGR | CDS | hsa-miR-337- | G/T | G | 105 | G/T | −48.58% |
| | | | 5p | | | | | |
| rs1042838 | PGR | CDS | hsa-miR-346 | G/T | G | 99 | G/T | −11.97% |
| rs1042838 | PGR | CDS | hsa-miR-362- | G/T | G | 93 | G/T | −11.62% |
| | | | 5p | | | | | |
| rs1042838 | PGR | CDS | hsa-miR-370 | G/T | G | 136 | G/T | −54.46% |
| rs1042838 | PGR | CDS | hsa-miR-378 | G/T | G | 116 | G/T | −45.31% |
| rs1042838 | PGR | CDS | hsa-miR-412 | G/T | G | 92 | G/T | −53.38% |
| rs1042838 | PGR | CDS | hsa-miR- | G/T | G | 120 | G/T | −53.44% |
| | | | 422a | | | | | |
| rs1042838 | PGR | CDS | hsa-miR-423- | G/T | G | 112 | G/T | −24.55% |
| | | | 3p | | | | | |
| rs1042838 | PGR | CDS | hsa-miR-432 | G/T | G | 129 | G/T | −23.46% |
| rs1042838 | PGR | CDS | hsa-miR-453 | G/T | G | 103 | G/T | −8.92% |
| rs1042838 | PGR | CDS | hsa-miR-493 | G/T | G | 126 | G/T | −48.70% |
| rs1042838 | PGR | CDS | hsa-miR- | G/T | G | 109 | G/T | −60.71% |
| | | | 497* | | | | | |
| rs1042838 | PGR | CDS | hsa-miR- | G/T | G | 85 | G/T | −38.10% |
| | | | 500* | | | | | |
| rs1042838 | PGR | CDS | hsa-miR-501- | G/T | G | 105 | G/T | −42.76% |
| | | | 5p | | | | | |
| rs1042838 | PGR | CDS | hsa-miR-509- | G/T | G | 104 | G/T | −14.22% |
| | | | 3p | | | | | |
| rs1042838 | PGR | CDS | hsa-miR- | G/T | G | 110 | G/T | −27.34% |
| | | | 518c* | | | | | |
| rs1042838 | PGR | CDS | hsa-miR- | G/T | G | 106 | G/T | −26.18% |
| | | | 518d-3p | | | | | |
| rs1042838 | PGR | CDS | hsa-miR-523 | G/T | G | 134 | G/T | −17.74% |
| rs1042838 | PGR | CDS | hsa-miR-524- | G/T | G | 101 | G/T | −20.46% |
| | | | 3p | | | | | |
| rs1042838 | PGR | CDS | hsa-miR-525- | G/T | G | 101 | G/T | −17.97% |
| | | | 3p | | | | | |
| rs1042838 | PGR | CDS | hsa-miR- | G/T | G | 90 | G/T | −36.11% |
| | | | 526b | | | | | |
| rs1042838 | PGR | CDS | hsa-miR-532- | G/T | T | 103 | G/T | 9.91% |
| | | | 3p | | | | | |
| rs1042838 | PGR | CDS | hsa-miR-550 | G/T | T | 134 | G/T | 11.82% |
| rs1042838 | PGR | CDS | hsa-miR- | G/T | G | 122 | G/T | −22.63% |
| | | | 551a | | | | | |
| rs1042838 | PGR | CDS | hsa-miR- | G/T | T | 91 | G/T | 131.17% |
| | | | 551b* | | | | | |
| rs1042838 | PGR | CDS | hsa-miR-557 | G/T | T | 84 | G/T | 12.72% |
| rs1042838 | PGR | CDS | hsa-miR-572 | G/T | G | 121 | G/T | −29.44% |
| rs1042838 | PGR | CDS | hsa-miR-579 | G/T | G | 143 | G/T | −13.95% |
| rs1042838 | PGR | CDS | hsa-miR-588 | G/T | G | 112 | G/T | −19.81% |
| rs1042838 | PGR | CDS | hsa-miR-593 | G/T | G | 98 | G/T | −80.35% |
| rs1042838 | PGR | CDS | hsa-miR-596 | G/T | G | 110 | G/T | −64.20% |
| rs1042838 | PGR | CDS | hsa-miR-600 | G/T | G | 86 | G/T | −101.62% |
| rs1042838 | PGR | CDS | hsa-miR-602 | G/T | G | 130 | G/T | −41.38% |
| rs1042838 | PGR | CDS | hsa-miR-614 | G/T | G | 152 | G/T | −18.08% |
| rs1042838 | PGR | CDS | hsa-miR-616 | G/T | G | 102 | G/T | −41.96% |
| rs1042838 | PGR | CDS | hsa-miR-619 | G/T | G | 93 | G/T | −20.48% |
| rs1042838 | PGR | CDS | hsa-miR-632 | G/T | G | 112 | G/T | −90.02% |
| rs1042838 | PGR | CDS | hsa-miR-636 | G/T | G | 122 | G/T | −19.53% |
| rs1042838 | PGR | CDS | hsa-miR-638 | G/T | T | 126 | G/T | 11.34% |
| rs1042838 | PGR | CDS | hsa-miR-660 | G/T | G | 128 | G/T | −35.13% |
| rs1042838 | PGR | CDS | hsa-miR-662 | G/T | G | 133 | G/T | −12.95% |
| rs1042838 | PGR | CDS | hsa-miR- | G/T | G | 144 | G/T | −46.68% |
| | | | 664* | | | | | |
| rs1042838 | PGR | CDS | hsa-miR-668 | G/T | G | 90 | G/T | −42.26% |
| rs1042838 | PGR | CDS | hsa-miR-766 | G/T | G | 83 | G/T | −26.99% |
| rs1042838 | PGR | CDS | hsa-miR-875- | G/T | G | 114 | G/T | −8.70% |
| | | | 3p | | | | | |
| rs1042838 | PGR | CDS | hsa-miR-887 | G/T | G | 107 | G/T | −8.84% |
| rs1042838 | PGR | CDS | hsa-miR- | G/T | G | 159 | G/T | −45.12% |
| | | | 892b | | | | | |
| rs1042838 | PGR | CDS | hsa-miR-92a | G/T | G | 86 | G/T | −15.11% |
| rs1042838 | PGR | CDS | hsa-miR- | G/T | G | 121 | G/T | −16.51% |
| | | | 92b* | | | | | |
| rs1042838 | PGR | CDS | hsa-miR-934 | G/T | G | 122 | G/T | −43.63% |
| rs1042838 | PGR | CDS | hsa-miR-942 | G/T | G | 91 | G/T | −20.13% |
| rs1042838 | PGR | CDS | hsa-miR- | G/T | G | 104 | G/T | −31.90% |
| | | | 99a* | | | | | |
| rs3180 | SPOCK2 | 3′ UTR | hsa-let-7b | A/G | A | 121 | A/G | −17.77% |
| rs3180 | SPOCK2 | 3′ UTR | hsa-let-7c | A/G | A | 119 | A/G | −24.00% |
| rs3180 | SPOCK2 | 3′ UTR | hsa-miR- | A/G | G | 131 | A/G | 12.75% |
| | | | 1182 | | | | | |
| rs3180 | SPOCK2 | 3′ UTR | hsa-miR- | A/G | G | 103 | A/G | 16.44% |
| | | | 1183 | | | | | |
| rs3180 | SPOCK2 | 3′ UTR | hsa-miR- | A/G | G | 105 | A/G | 10.33% |
| | | | 1184 | | | | | |
| rs3180 | SPOCK2 | 3′ UTR | hsa-miR- | A/G | A | 124 | A/G | −39.73% |
| | | | 1203 | | | | | |
| rs3180 | SPOCK2 | 3′ UTR | hsa-miR- | A/G | G | 117 | A/G | 14.21% |
| | | | 1224-5p | | | | | |
| rs3180 | SPOCK2 | 3′ UTR | hsa-miR- | A/G | A | 94 | A/G | −50.39% |
| | | | 1231 | | | | | |
| rs3180 | SPOCK2 | 3′ UTR | hsa-miR- | A/G | A | 148 | A/G | −15.75% |
| | | | 125a-3p | | | | | |
| rs3180 | SPOCK2 | 3′ UTR | hsa-miR- | A/G | G | 124 | A/G | 15.86% |
| | | | 125b-1* | | | | | |
| rs3180 | SPOCK2 | 3′ UTR | hsa-miR- | A/G | A | 123 | A/G | −15.33% |
| | | | 1262 | | | | | |
| rs3180 | SPOCK2 | 3′ UTR | hsa-miR- | A/G | A | 133 | A/G | −39.46% |
| | | | 1265 | | | | | |
| rs3180 | SPOCK2 | 3′ UTR | hsa-miR- | A/G | A | 147 | A/G | −9.73% |
| | | | 1273 | | | | | |
| rs3180 | SPOCK2 | 3′ UTR | hsa-miR- | A/G | G | 101 | A/G | 14.60% |
| | | | 1303 | | | | | |
| rs3180 | SPOCK2 | 3′ UTR | hsa-miR-139- | A/G | G | 112 | A/G | 31.16% |
| | | | 3p | | | | | |
| rs3180 | SPOCK2 | 3′ UTR | hsa-miR- | A/G | G | 155 | A/G | 13.48% |
| | | | 149* | | | | | |
| rs3180 | SPOCK2 | 3′ UTR | hsa-miR- | A/G | G | 126 | A/G | 15.56% |
| | | | 1538 | | | | | |
| rs3180 | SPOCK2 | 3′ UTR | hsa-miR- | A/G | A | 130 | A/G | −13.81% |
| | | | 1909 | | | | | |
| rs3180 | SPOCK2 | 3′ UTR | hsa-miR- | A/G | G | 121 | A/G | 52.34% |
| | | | 194* | | | | | |
| rs3180 | SPOCK2 | 3′ UTR | hsa-miR-202 | A/G | G | 98 | A/G | 18.05% |
| rs3180 | SPOCK2 | 3′ UTR | hsa-miR- | A/G | G | 127 | A/G | 17.10% |
| | | | 2110 | | | | | |
| rs3180 | SPOCK2 | 3′ UTR | hsa-miR-296- | A/G | G | 75 | A/G | 14.07% |
| | | | 3p | | | | | |
| rs3180 | SPOCK2 | 3′ UTR | hsa-miR-298 | A/G | A | 141 | A/G | −13.67% |
| rs3180 | SPOCK2 | 3′ UTR | hsa-miR- | A/G | A | 114 | A/G | −19.33% |
| | | | 30b* | | | | | |
| rs3180 | SPOCK2 | 3′ UTR | hsa-miR-30c- | A/G | G | 120 | A/G | 40.17% |
| | | | 1* | | | | | |
| rs3180 | SPOCK2 | 3′ UTR | hsa-miR-30c- | A/G | G | 116 | A/G | 21.93% |
| | | | 2* | | | | | |
| rs3180 | SPOCK2 | 3′ UTR | hsa-miR- | A/G | G | 72 | A/G | 33.19% |
| | | | 365* | | | | | |
| rs3180 | SPOCK2 | 3′ UTR | hsa-miR-381 | A/G | A | 105 | A/G | −9.09% |
| rs3180 | SPOCK2 | 3′ UTR | hsa-miR-423- | A/G | G | 106 | A/G | 35.68% |
| | | | 3p | | | | | |
| rs3180 | SPOCK2 | 3′ UTR | hsa-miR-423- | A/G | A | 199 | A/G | −13.24% |
| | | | 5p | | | | | |
| rs3180 | SPOCK2 | 3′ UTR | hsa-miR- | A/G | G | 87 | A/G | 43.34% |
| | | | 424* | | | | | |
| rs3180 | SPOCK2 | 3′ UTR | hsa-miR-433 | A/G | A | 124 | A/G | −16.65% |
| rs3180 | SPOCK2 | 3′ UTR | hsa-miR-483- | A/G | G | 163 | A/G | 18.65% |
| | | | 5p | | | | | |
| rs3180 | SPOCK2 | 3′ UTR | hsa-miR-486- | A/G | A | 176 | A/G | −33.17% |
| | | | 3p | | | | | |
| rs3180 | SPOCK2 | 3′ UTR | hsa-miR-492 | A/G | G | 103 | A/G | 15.94% |
| rs3180 | SPOCK2 | 3′ UTR | hsa-miR-503 | A/G | G | 136 | A/G | 16.03% |
| rs3180 | SPOCK2 | 3′ UTR | hsa-miR-541 | A/G | A | 135 | A/G | −23.73% |
| rs3180 | SPOCK2 | 3′ UTR | hsa-miR-549 | A/G | A | 94 | A/G | −16.90% |
| rs3180 | SPOCK2 | 3′ UTR | hsa-miR-564 | A/G | A | 123 | A/G | −44.38% |
| rs3180 | SPOCK2 | 3′ UTR | hsa-miR- | A/G | A | 104 | A/G | −45.69% |
| | | | 593* | | | | | |
| rs3180 | SPOCK2 | 3′ UTR | hsa-miR-602 | A/G | A | 117 | A/G | −60.34% |
| rs3180 | SPOCK2 | 3′ UTR | hsa-miR-608 | A/G | G | 140 | A/G | 23.90% |
| rs3180 | SPOCK2 | 3′ UTR | hsa-miR-615- | A/G | G | 96 | A/G | 13.60% |
| | | | 5p | | | | | |
| rs3180 | SPOCK2 | 3′ UTR | hsa-miR-623 | A/G | G | 92 | A/G | 24.26% |
| rs3180 | SPOCK2 | 3′ UTR | hsa-miR-628- | A/G | G | 93 | A/G | 12.93% |
| | | | 3p | | | | | |
| rs3180 | SPOCK2 | 3′ UTR | hsa-miR-637 | A/G | G | 132 | A/G | 12.41% |
| rs3180 | SPOCK2 | 3′ UTR | hsa-miR-638 | A/G | A | 99 | A/G | −72.66% |
| rs3180 | SPOCK2 | 3′ UTR | hsa-miR-648 | A/G | G | 110 | A/G | 9.16% |
| rs3180 | SPOCK2 | 3′ UTR | hsa-miR-658 | A/G | G | 142 | A/G | 29.88% |
| rs3180 | SPOCK2 | 3′ UTR | hsa-miR-663 | A/G | G | 137 | A/G | 14.85% |
| rs3180 | SPOCK2 | 3′ UTR | hsa-miR-675 | A/G | A | 126 | A/G | −13.44% |
| rs3180 | SPOCK2 | 3′ UTR | hsa-miR-744 | A/G | G | 142 | A/G | 10.06% |
| rs3180 | SPOCK2 | 3′ UTR | hsa-miR-769- | A/G | G | 112 | A/G | 16.64% |
| | | | 3p | | | | | |
| rs3180 | SPOCK2 | 3′ UTR | hsa-miR-873 | A/G | G | 96 | A/G | 11.79% |
| rs3180 | SPOCK2 | 3′ UTR | hsa-miR-885- | A/G | G | 152 | A/G | 10.11% |
| | | | 3p | | | | | |
| rs3180 | SPOCK2 | 3′ UTR | hsa-miR-886- | A/G | G | 122 | A/G | 58.84% |
| | | | 5p | | | | | |
| rs3180 | SPOCK2 | 3′ UTR | hsa-miR-887 | A/G | A | 103 | A/G | −32.07% |
| rs3180 | SPOCK2 | 3′ UTR | hsa-miR-920 | A/G | G | 80 | A/G | 38.07% |
| rs3180 | SPOCK2 | 3′ UTR | hsa-miR-921 | A/G | A | 169 | A/G | −16.00% |
| rs3180 | SPOCK2 | 3′ UTR | hsa-miR-922 | A/G | A | 120 | A/G | −34.52% |
| rs3180 | SPOCK2 | 3′ UTR | hsa-miR-939 | A/G | A | 136 | A/G | −18.52% |
| rs3180 | SPOCK2 | 3′ UTR | hsa-miR-940 | A/G | A | 130 | A/G | −37.81% |
| rs1982073 | TGFB1 | CDS | hsa-let-7b | C/T | C | 129 | C/T | −18.89% |
| rs1982073 | TGFB1 | CDS | hsa-let-7f | C/T | T | 127 | C/T | 11.28% |
| rs1982073 | TGFB1 | CDS | hsa-miR- | C/T | C | 91 | C/T | −26.81% |
| | | | 106a* | | | | | |
| rs1982073 | TGFB1 | CDS | hsa-miR- | C/T | C | 125 | C/T | −15.62% |
| | | | 1183 | | | | | |
| rs1982073 | TGFB1 | CDS | hsa-miR- | C/T | C | 135 | C/T | −63.12% |
| | | | 1207-5p | | | | | |
| rs1982073 | TGFB1 | CDS | hsa-miR- | C/T | C | 83 | C/T | −12.21% |
| | | | 1208 | | | | | |
| rs1982073 | TGFB1 | CDS | hsa-miR- | C/T | C | 102 | C/T | −20.37% |
| | | | 1225-5p | | | | | |
| rs1982073 | TGFB1 | CDS | hsa-miR- | C/T | C | 115 | C/T | −16.55% |
| | | | 1250 | | | | | |
| rs1982073 | TGFB1 | CDS | hsa-miR- | C/T | C | 82 | C/T | −13.80% |
| | | | 1255b | | | | | |
| rs1982073 | TGFB1 | CDS | hsa-miR- | C/T | C | 83 | C/T | −31.05% |
| | | | 125b-1* | | | | | |
| rs1982073 | TGFB1 | CDS | hsa-miR- | C/T | C | 116 | C/T | −14.37% |
| | | | 1266 | | | | | |
| rs1982073 | TGFB1 | CDS | hsa-miR- | C/T | C | 84 | C/T | −52.97% |
| | | | 1269 | | | | | |
| rs1982073 | TGFB1 | CDS | hsa-miR- | C/T | C | 99 | C/T | −26.75% |
| | | | 1271 | | | | | |
| rs1982073 | TGFB1 | CDS | hsa-miR- | C/T | C | 104 | C/T | −15.30% |
| | | | 1285 | | | | | |
| rs1982073 | TGFB1 | CDS | hsa-miR- | C/T | C | 113 | C/T | −13.52% |
| | | | 1286 | | | | | |
| rs1982073 | TGFB1 | CDS | hsa-miR- | C/T | C | 77 | C/T | −16.34% |
| | | | 1287 | | | | | |
| rs1982073 | TGFB1 | CDS | hsa-miR- | C/T | C | 96 | C/T | −19.33% |
| | | | 1288 | | | | | |
| rs1982073 | TGFB1 | CDS | hsa-miR- | C/T | T | 109 | C/T | 9.79% |
| | | | 1291 | | | | | |
| rs1982073 | TGFB1 | CDS | hsa-miR- | C/T | C | 106 | C/T | −12.45% |
| | | | 1292 | | | | | |
| rs1982073 | TGFB1 | CDS | hsa-miR- | C/T | C | 93 | C/T | −63.51% |
| | | | 1296 | | | | | |
| rs1982073 | TGFB1 | CDS | hsa-miR- | C/T | C | 117 | C/T | −10.52% |
| | | | 1307 | | | | | |
| rs1982073 | TGFB1 | CDS | hsa-miR- | C/T | C | 109 | C/T | −48.69% |
| | | | 1308 | | | | | |
| rs1982073 | TGFB1 | CDS | hsa-miR- | C/T | C | 82 | C/T | −18.25% |
| | | | 1321 | | | | | |
| rs1982073 | TGFB1 | CDS | hsa-miR-134 | C/T | C | 99 | C/T | −26.22% |
| rs1982073 | TGFB1 | CDS | hsa-miR- | C/T | C | 96 | C/T | −35.28% |
| | | | 136* | | | | | |
| rs1982073 | TGFB1 | CDS | hsa-miR-140- | C/T | C | 158 | C/T | −14.43% |
| | | | 3p | | | | | |
| rs1982073 | TGFB1 | CDS | hsa-miR-140- | C/T | C | 111 | C/T | −13.23% |
| | | | 5p | | | | | |
| rs1982073 | TGFB1 | CDS | hsa-miR-142- | C/T | C | 88 | C/T | −116.60% |
| | | | 3p | | | | | |
| rs1982073 | TGFB1 | CDS | hsa-miR- | C/T | C | 89 | C/T | −57.59% |
| | | | 146b-3p | | | | | |
| rs1982073 | TGFB1 | CDS | hsa-miR-151- | C/T | C | 102 | C/T | −12.24% |
| | | | 5p | | | | | |
| rs1982073 | TGFB1 | CDS | hsa-miR-152 | C/T | C | 107 | C/T | −10.13% |
| rs1982073 | TGFB1 | CDS | hsa-miR-15a | C/T | C | 148 | C/T | −10.98% |
| rs1982073 | TGFB1 | CDS | hsa-miR-15b | C/T | C | 151 | C/T | −15.86% |
| rs1982073 | TGFB1 | CDS | hsa-miR-16 | C/T | C | 157 | C/T | −28.91% |
| rs1982073 | TGFB1 | CDS | hsa-miR-16- | C/T | C | 90 | C/T | −13.27% |
| | | | 1* | | | | | |
| rs1982073 | TGFB1 | CDS | hsa-miR-17* | C/T | C | 129 | C/T | −13.58% |
| rs1982073 | TGFB1 | CDS | hsa-miR- | C/T | C | 82 | C/T | −75.86% |
| | | | 181a* | | | | | |
| rs1982073 | TGFB1 | CDS | hsa-miR-182 | C/T | T | 86 | C/T | 17.79% |
| rs1982073 | TGFB1 | CDS | hsa-miR- | C/T | C | 111 | C/T | −17.48% |
| | | | 185* | | | | | |
| rs1982073 | TGFB1 | CDS | hsa-miR-187 | C/T | C | 83 | C/T | −41.90% |
| rs1982073 | TGFB1 | CDS | hsa-miR- | C/T | C | 91 | C/T | −18.37% |
| | | | 1908 | | | | | |
| rs1982073 | TGFB1 | CDS | hsa-miR- | C/T | C | 150 | C/T | −24.10% |
| | | | 1909 | | | | | |
| rs1982073 | TGFB1 | CDS | hsa-miR- | C/T | C | 106 | C/T | −12.35% |
| | | | 1909* | | | | | |
| rs1982073 | TGFB1 | CDS | hsa-miR-191 | C/T | T | 117 | C/T | 28.59% |
| rs1982073 | TGFB1 | CDS | hsa-miR- | C/T | C | 108 | C/T | −8.71% |
| | | | 1915 | | | | | |
| rs1982073 | TGFB1 | CDS | hsa-miR- | C/T | C | 125 | C/T | −53.51% |
| | | | 194* | | | | | |
| rs1982073 | TGFB1 | CDS | hsa-miR- | C/T | C | 147 | C/T | −12.91% |
| | | | 196b* | | | | | |
| rs1982073 | TGFB1 | CDS | hsa-miR- | C/T | C | 110 | C/T | −20.11% |
| | | | 1974 | | | | | |
| rs1982073 | TGFB1 | CDS | hsa-miR- | C/T | C | 91 | C/T | −85.13% |
| | | | 199a-5p | | | | | |
| rs1982073 | TGFB1 | CDS | hsa-miR-19b- | C/T | C | 81 | C/T | −17.57% |
| | | | 1* | | | | | |
| rs1982073 | TGFB1 | CDS | hsa-miR- | C/T | C | 83 | C/T | −17.28% |
| | | | 205* | | | | | |
| rs1982073 | TGFB1 | CDS | hsa-miR- | C/T | C | 110 | C/T | −43.74% |
| | | | 20b* | | | | | |
| rs1982073 | TGFB1 | CDS | hsa-miR-21* | C/T | T | 93 | C/T | 15.53% |
| rs1982073 | TGFB1 | CDS | hsa-miR- | C/T | C | 100 | C/T | −71.06% |
| | | | 2110 | | | | | |
| rs1982073 | TGFB1 | CDS | hsa-miR-214 | C/T | C | 138 | C/T | −30.34% |
| rs1982073 | TGFB1 | CDS | hsa-miR-218- | C/T | C | 82 | C/T | −48.51% |
| | | | 1* | | | | | |
| rs1982073 | TGFB1 | CDS | hsa-miR- | C/T | T | 128 | C/T | 12.84% |
| | | | 222* | | | | | |
| rs1982073 | TGFB1 | CDS | hsa-miR-24 | C/T | C | 111 | C/T | −32.44% |
| rs1982073 | TGFB1 | CDS | hsa-miR-296- | C/T | C | 88 | C/T | −79.05% |
| | | | 5p | | | | | |
| rs1982073 | TGFB1 | CDS | hsa-miR-298 | C/T | C | 139 | C/T | −10.55% |
| rs1982073 | TGFB1 | CDS | hsa-miR-29a | C/T | T | 125 | C/T | 42.61% |
| rs1982073 | TGFB1 | CDS | hsa-miR-29b- | C/T | C | 86 | C/T | −38.79% |
| | | | 2* | | | | | |
| rs1982073 | TGFB1 | CDS | hsa-miR-29c | C/T | T | 119 | C/T | 57.98% |
| rs1982073 | TGFB1 | CDS | hsa-miR- | C/T | T | 92 | C/T | 20.24% |
| | | | 301a | | | | | |
| rs1982073 | TGFB1 | CDS | hsa-miR- | C/T | C | 83 | C/T | −14.48% |
| | | | 30d* | | | | | |
| rs1982073 | TGFB1 | CDS | hsa-miR-323- | C/T | C | 112 | C/T | −22.47% |
| | | | 5p | | | | | |
| rs1982073 | TGFB1 | CDS | hsa-miR-325 | C/T | C | 107 | C/T | −50.28% |
| rs1982073 | TGFB1 | CDS | hsa-miR-330- | C/T | C | 113 | C/T | −8.01% |
| | | | 3p | | | | | |
| rs1982073 | TGFB1 | CDS | hsa-miR- | C/T | C | 120 | C/T | −28.74% |
| | | | 33b* | | | | | |
| rs1982073 | TGFB1 | CDS | hsa-miR-34a | C/T | C | 127 | C/T | −72.14% |
| rs1982073 | TGFB1 | CDS | hsa-miR- | C/T | C | 108 | C/T | −14.70% |
| | | | 34a* | | | | | |
| rs1982073 | TGFB1 | CDS | hsa-miR- | C/T | T | 118 | C/T | 15.11% |
| | | | 34b* | | | | | |
| rs1982073 | TGFB1 | CDS | hsa-miR-379 | C/T | C | 104 | C/T | −22.28% |
| rs1982073 | TGFB1 | CDS | hsa-miR-423- | C/T | C | 115 | C/T | −8.27% |
| | | | 3p | | | | | |
| rs1982073 | TGFB1 | CDS | hsa-miR-423- | C/T | C | 118 | C/T | −11.90% |
| | | | 5p | | | | | |
| rs1982073 | TGFB1 | CDS | hsa-miR- | C/T | C | 90 | C/T | −103.83% |
| | | | 425* | | | | | |
| rs1982073 | TGFB1 | CDS | hsa-miR- | C/T | C | 126 | C/T | −18.62% |
| | | | 449a | | | | | |
| rs1982073 | TGFB1 | CDS | hsa-miR- | C/T | C | 132 | C/T | −13.70% |
| | | | 449b | | | | | |
| rs1982073 | TGFB1 | CDS | hsa-miR-453 | C/T | C | 95 | C/T | −12.41% |
| rs1982073 | TGFB1 | CDS | hsa-miR-485- | C/T | C | 114 | C/T | −23.88% |
| | | | 5p | | | | | |
| rs1982073 | TGFB1 | CDS | hsa-miR-491- | C/T | C | 93 | C/T | −81.46% |
| | | | 5p | | | | | |
| rs1982073 | TGFB1 | CDS | hsa-miR-497 | C/T | T | 168 | C/T | 20.71% |
| rs1982073 | TGFB1 | CDS | hsa-miR-509- | C/T | C | 94 | C/T | −36.61% |
| | | | 3p | | | | | |
| rs1982073 | TGFB1 | CDS | hsa-miR-512- | C/T | T | 102 | C/T | 14.81% |
| | | | 3p | | | | | |
| rs1982073 | TGFB1 | CDS | hsa-miR- | C/T | C | 96 | C/T | −37.97% |
| | | | 518a-3p | | | | | |
| rs1982073 | TGFB1 | CDS | hsa-miR- | C/T | C | 85 | C/T | −14.30% |
| | | | 518a-5p | | | | | |
| rs1982073 | TGFB1 | CDS | hsa-miR- | C/T | C | 83 | C/T | −32.68% |
| | | | 518e* | | | | | |
| rs1982073 | TGFB1 | CDS | hsa-miR- | C/T | C | 83 | C/T | −32.68% |
| | | | 519a* | | | | | |
| rs1982073 | TGFB1 | CDS | hsa-miR- | C/T | C | 83 | C/T | −32.68% |
| | | | 519b-5p | | | | | |
| rs1982073 | TGFB1 | CDS | hsa-miR- | C/T | C | 83 | C/T | −32.68% |
| | | | 519c-5p | | | | | |
| rs1982073 | TGFB1 | CDS | hsa-miR- | C/T | C | 83 | C/T | −32.68% |
| | | | 522* | | | | | |
| rs1982073 | TGFB1 | CDS | hsa-miR- | C/T | C | 83 | C/T | −32.68% |
| | | | 523* | | | | | |
| rs1982073 | TGFB1 | CDS | hsa-miR-527 | C/T | C | 85 | C/T | −14.30% |
| rs1982073 | TGFB1 | CDS | hsa-miR-541 | C/T | C | 113 | C/T | −27.79% |
| rs1982073 | TGFB1 | CDS | hsa-miR-550 | C/T | C | 89 | C/T | −11.86% |
| rs1982073 | TGFB1 | CDS | hsa-miR- | C/T | T | 107 | C/T | 18.18% |
| | | | 589* | | | | | |
| rs1982073 | TGFB1 | CDS | hsa-miR-602 | C/T | C | 125 | C/T | −43.79% |
| rs1982073 | TGFB1 | CDS | hsa-miR-608 | C/T | C | 134 | C/T | −9.01% |
| rs1982073 | TGFB1 | CDS | hsa-miR-621 | C/T | C | 133 | C/T | −8.85% |
| rs1982073 | TGFB1 | CDS | hsa-miR-623 | C/T | C | 80 | C/T | −38.66% |
| rs1982073 | TGFB1 | CDS | hsa-miR-628- | C/T | C | 98 | C/T | −30.94% |
| | | | 3p | | | | | |
| rs1982073 | TGFB1 | CDS | hsa-miR-647 | C/T | C | 130 | C/T | −16.04% |
| rs1982073 | TGFB1 | CDS | hsa-miR-648 | C/T | C | 100 | C/T | −26.44% |
| rs1982073 | TGFB1 | CDS | hsa-miR-650 | C/T | C | 129 | C/T | −18.11% |
| rs1982073 | TGFB1 | CDS | hsa-miR-654- | C/T | C | 117 | C/T | −11.41% |
| | | | 5p | | | | | |
| rs1982073 | TGFB1 | CDS | hsa-miR-658 | C/T | T | 128 | C/T | 13.00% |
| rs1982073 | TGFB1 | CDS | hsa-miR-661 | C/T | C | 89 | C/T | −9.50% |
| rs1982073 | TGFB1 | CDS | hsa-miR-663 | C/T | C | 130 | C/T | −10.75% |
| rs1982073 | TGFB1 | CDS | hsa-miR-744 | C/T | C | 137 | C/T | −13.55% |
| rs1982073 | TGFB1 | CDS | hsa-miR-767- | C/T | C | 100 | C/T | −63.31% |
| | | | 5p | | | | | |
| rs1982073 | TGFB1 | CDS | hsa-miR-769- | C/T | C | 84 | C/T | −100.97% |
| | | | 3p | | | | | |
| rs1982073 | TGFB1 | CDS | hsa-miR-877 | C/T | C | 104 | C/T | −26.75% |
| rs1982073 | TGFB1 | CDS | hsa-miR-885- | C/T | C | 160 | C/T | −18.97% |
| | | | 3p | | | | | |
| rs1982073 | TGFB1 | CDS | hsa-miR-887 | C/T | C | 111 | C/T | −22.31% |
| rs1982073 | TGFB1 | CDS | hsa-miR-921 | C/T | C | 85 | C/T | −44.09% |
| rs1982073 | TGFB1 | CDS | hsa-miR-922 | C/T | T | 134 | C/T | 21.97% |
| rs1982073 | TGFB1 | CDS | hsa-miR-92a | C/T | C | 88 | C/T | −11.91% |
| rs1982073 | TGFB1 | CDS | hsa-miR-93* | C/T | C | 93 | C/T | −37.57% |
| rs1982073 | TGFB1 | CDS | hsa-miR-936 | C/T | C | 137 | C/T | −37.76% |
| rs1982073 | TGFB1 | CDS | hsa-miR-939 | C/T | C | 136 | C/T | −9.57% |
| rs1982073 | TGFB1 | CDS | hsa-miR-940 | C/T | T | 110 | C/T | 9.74% |
| rs1982073 | TGFB1 | CDS | hsa-miR-941 | C/T | C | 107 | C/T | −13.61% |
| rs10735810 | VDR | CDS | hsa-let-7i* | A/G | G | 107 | A/G | 9.98% |
| rs10735810 | VDR | CDS | hsa-miR-103- | A/G | G | 129 | A/G | 33.20% |
| | | | as | | | | | |
| rs10735810 | VDR | CDS | hsa-miR- | A/G | G | 118 | A/G | 143.04 |
| | | | 1180 | | | | | |
| rs10735810 | VDR | CDS | hsa-miR- | A/G | G | 139 | A/G | 8.00% |
| | | | 1181 | | | | | |
| rs10735810 | VDR | CDS | hsa-miR- | A/G | A | 135 | A/G | −25.35% |
| | | | 1204 | | | | | |
| rs10735810 | VDR | CDS | hsa-miR- | A/G | G | 97 | A/G | 17.94% |
| | | | 1208 | | | | | |
| rs10735810 | VDR | CDS | hsa-miR-122 | A/G | G | 109 | A/G | 20.54% |
| rs10735810 | VDR | CDS | hsa-miR- | A/G | G | 87 | A/G | 55.53% |
| | | | 1225-5p | | | | | |
| rs10735810 | VDR | CDS | hsa-miR- | A/G | G | 108 | A/G | 39.07% |
| | | | 1226 | | | | | |
| rs10735810 | VDR | CDS | hsa-miR- | A/G | A | 111 | A/G | −23.20% |
| | | | 1234 | | | | | |
| rs10735810 | VDR | CDS | hsa-miR- | A/G | G | 102 | A/G | 9.85% |
| | | | 1237 | | | | | |
| rs10735810 | VDR | CDS | hsa-miR- | A/G | G | 143 | A/G | 12.79% |
| | | | 1247 | | | | | |
| rs10735810 | VDR | CDS | hsa-miR- | A/G | G | 105 | A/G | 24.90% |
| | | | 1269 | | | | | |
| rs10735810 | VDR | CDS | hsa-miR-127- | A/G | G | 93 | A/G | 13.50% |
| | | | 3p | | | | | |
| rs10735810 | VDR | CDS | hsa-miR- | A/G | G | 140 | A/G | 12.73% |
| | | | 1274a | | | | | |
| rs10735810 | VDR | CDS | hsa-miR- | A/G | G | 128 | A/G | 19.95% |
| | | | 1295 | | | | | |
| rs10735810 | VDR | CDS | hsa-miR- | A/G | A | 131 | A/G | −24.14% |
| | | | 1296 | | | | | |
| rs10735810 | VDR | CDS | hsa-miR- | A/G | G | 98 | A/G | 38.65% |
| | | | 1301 | | | | | |
| rs10735810 | VDR | CDS | hsa-miR- | A/G | A | 95 | A/G | −12.93% |
| | | | 1305 | | | | | |
| rs10735810 | VDR | CDS | hsa-miR- | A/G | G | 109 | A/G | 14.48% |
| | | | 1307 | | | | | |
| rs10735810 | VDR | CDS | hsa-miR- | A/G | G | 94 | A/G | 13.19% |
| | | | 130a* | | | | | |
| rs10735810 | VDR | CDS | hsa-miR- | A/G | G | 116 | A/G | 10.53% |
| | | | 132* | | | | | |
| rs10735810 | VDR | CDS | hsa-miR- | A/G | A | 105 | A/G | −48.04% |
| | | | 133a | | | | | |
| rs10735810 | VDR | CDS | hsa-miR- | A/G | A | 102 | A/G | −45.14% |
| | | | 133b | | | | | |
| rs10735810 | VDR | CDS | hsa-miR-134 | A/G | G | 97 | A/G | 22.49% |
| rs10735810 | VDR | CDS | hsa-miR- | A/G | G | 101 | A/G | 12.70% |
| | | | 135b | | | | | |
| rs10735810 | VDR | CDS | hsa-miR-138- | A/G | A | 99 | A/G | −10.27% |
| | | | 1* | | | | | |
| rs10735810 | VDR | CDS | hsa-miR- | A/G | A | 110 | A/G | −14.23% |
| | | | 143* | | | | | |
| rs10735810 | VDR | CDS | hsa-miR- | A/G | A | 103 | A/G | −11.45% |
| | | | 1468 | | | | | |
| rs10735810 | VDR | CDS | hsa-miR-147 | A/G | G | 112 | A/G | 10.17% |
| rs10735810 | VDR | CDS | hsa-miR- | A/G | G | 153 | A/G | 10.31% |
| | | | 1470 | | | | | |
| rs10735810 | VDR | CDS | hsa-miR- | A/G | G | 114 | A/G | 41.38% |
| | | | 1471 | | | | | |
| rs10735810 | VDR | CDS | hsa-miR- | A/G | G | 123 | A/G | 16.10% |
| | | | 148a* | | | | | |
| rs10735810 | VDR | CDS | hsa-miR-149 | A/G | A | 146 | A/G | −9.66% |
| rs10735810 | VDR | CDS | hsa-miR-150 | A/G | A | 96 | A/G | −22.15% |
| rs10735810 | VDR | CDS | hsa-miR- | A/G | G | 118 | A/G | 20.52% |
| | | | 1538 | | | | | |
| rs10735810 | VDR | CDS | hsa-miR- | A/G | G | 98 | A/G | 72.72% |
| | | | 181a-2* | | | | | |
| rs10735810 | VDR | CDS | hsa-miR- | A/G | A | 90 | A/G | −26.19% |
| | | | 181d | | | | | |
| rs10735810 | VDR | CDS | hsa-miR- | A/G | G | 136 | A/G | 18.11% |
| | | | 185* | | | | | |
| rs10735810 | VDR | CDS | hsa-miR-187 | A/G | G | 114 | A/G | 18.24% |
| rs10735810 | VDR | CDS | hsa-miR-188- | A/G | A | 142 | A/G | −26.19% |
| | | | 5p | | | | | |
| rs10735810 | VDR | CDS | hsa-miR- | A/G | G | 130 | A/G | 12.59% |
| | | | 1909* | | | | | |
| rs10735810 | VDR | CDS | hsa-miR- | A/G | A | 92 | A/G | −36.29% |
| | | | 1911 | | | | | |
| rs10735810 | VDR | CDS | hsa-miR- | A/G | A | 113 | A/G | −14.39% |
| | | | 192* | | | | | |
| rs10735810 | VDR | CDS | hsa-miR- | A/G | G | 159 | A/G | 11.16% |
| | | | 193b | | | | | |
| rs10735810 | VDR | CDS | hsa-miR-197 | A/G | A | 97 | A/G | −14.95% |
| rs10735810 | VDR | CDS | hsa-miR- | A/G | G | 110 | A/G | 39.69% |
| | | | 1973 | | | | | |
| rs10735810 | VDR | CDS | hsa-miR- | A/G | G | 83 | A/G | 58.10% |
| | | | 1975 | | | | | |
| rs10735810 | VDR | CDS | hsa-miR-205 | A/G | A | 138 | A/G | −14.29% |
| rs10735810 | VDR | CDS | hsa-miR-21* | A/G | G | 84 | A/G | 19.99% |
| rs10735810 | VDR | CDS | hsa-miR- | A/G | G | 110 | A/G | 25.58% |
| | | | 2113 | | | | | |
| rs10735810 | VDR | CDS | hsa-miR- | A/G | A | 116 | A/G | −28.06% |
| | | | 214* | | | | | |
| rs10735810 | VDR | CDS | hsa-miR-218- | A/G | G | 125 | A/G | 11.97% |
| | | | 1* | | | | | |
| rs10735810 | VDR | CDS | hsa-miR-219- | A/G | G | 88 | A/G | 61.00% |
| | | | 1-3p | | | | | |
| rs10735810 | VDR | CDS | hsa-miR-22 | A/G | G | 130 | A/G | 23.58% |
| rs10735810 | VDR | CDS | hsa-miR- | A/G | G | 111 | A/G | 11.66% |
| | | | 220b | | | | | |
| rs10735810 | VDR | CDS | hsa-miR-222 | A/G | A | 98 | A/G | −110.03 |
| rs10735810 | VDR | CDS | hsa-miR-224 | A/G | G | 94 | A/G | 12.94% |
| rs10735810 | VDR | CDS | hsa-miR- | A/G | G | 111 | A/G | 12.14% |
| | | | 23a* | | | | | |
| rs10735810 | VDR | CDS | hsa-miR-24 | A/G | G | 127 | A/G | 10.51% |
| rs10735810 | VDR | CDS | hsa-miR-24- | A/G | G | 107 | A/G | 23.47% |
| | | | 1* | | | | | |
| rs10735810 | VDR | CDS | hsa-miR-24- | A/G | G | 113 | A/G | 21.26% |
| | | | 2* | | | | | |
| rs10735810 | VDR | CDS | hsa-miR-25 | A/G | G | 116 | A/G | 26.32% |
| rs10735810 | VDR | CDS | hsa-miR- | A/G | G | 148 | A/G | 10.29% |
| | | | 302a | | | | | |
| rs10735810 | VDR | CDS | hsa-miR- | A/G | G | 145 | A/G | 18.58% |
| | | | 302d | | | | | |
| rs10735810 | VDR | CDS | hsa-miR-302f | A/G | A | 121 | A/G | −39.63% |
| rs10735810 | VDR | CDS | hsa-miR-31* | A/G | A | 110 | A/G | −30.72% |
| rs10735810 | VDR | CDS | hsa-miR-323- | A/G | G | 118 | A/G | 14.62% |
| | | | 5p | | | | | |
| rs10735810 | VDR | CDS | hsa-miR-324- | A/G | G | 97 | A/G | 22.45% |
| | | | 3p | | | | | |
| rs10735810 | VDR | CDS | hsa-miR-324- | A/G | A | 144 | A/G | −25.30% |
| | | | 5p | | | | | |
| rs10735810 | VDR | CDS | hsa-miR-331- | A/G | A | 85 | A/G | −34.59% |
| | | | 3p | | | | | |
| rs10735810 | VDR | CDS | hsa-miR-339- | A/G | A | 108 | A/G | −9.96% |
| | | | 3p | | | | | |
| rs10735810 | VDR | CDS | hsa-miR-339- | A/G | G | 132 | A/G | 21.28% |
| | | | 5p | | | | | |
| rs10735810 | VDR | CDS | hsa-miR- | A/G | G | 143 | A/G | 15.45% |
| | | | 340* | | | | | |
| rs10735810 | VDR | CDS | hsa-miR-362- | A/G | A | 137 | A/G | −9.08% |
| | | | 5p | | | | | |
| rs10735810 | VDR | CDS | hsa-miR- | A/G | A | 96 | A/G | −14.12% |
| | | | 377* | | | | | |
| rs10735810 | VDR | CDS | hsa-miR-409- | A/G | A | 96 | A/G | −11.42% |
| | | | 3p | | | | | |
| rs10735810 | VDR | CDS | hsa-miR-423- | A/G | G | 108 | A/G | 41.32% |
| | | | 3p | | | | | |
| rs10735810 | VDR | CDS | hsa-miR- | A/G | A | 105 | A/G | −55.08% |
| | | | 432* | | | | | |
| rs10735810 | VDR | CDS | hsa-miR- | A/G | A | 82 | A/G | −27.69% |
| | | | 450b-3p | | | | | |
| rs10735810 | VDR | CDS | hsa-miR- | A/G | G | 88 | A/G | 33.61% |
| | | | 450b-5p | | | | | |
| rs10735810 | VDR | CDS | hsa-miR-451 | A/G | A | 102 | A/G | −51.29% |
| rs10735810 | VDR | CDS | hsa-miR-453 | A/G | G | 103 | A/G | 16.84% |
| rs10735810 | VDR | CDS | hsa-miR-489 | A/G | A | 94 | A/G | −10.30% |
| rs10735810 | VDR | CDS | hsa-miR-490- | A/G | A | 85 | A/G | −11.83% |
| | | | 3p | | | | | |
| rs10735810 | VDR | CDS | hsa-miR-501- | A/G | G | 115 | A/G | 14.20% |
| | | | 5p | | | | | |
| rs10735810 | VDR | CDS | hsa-miR-515- | A/G | A | 104 | A/G | −9.94% |
| | | | 5p | | | | | |
| rs10735810 | VDR | CDS | hsa-miR- | A/G | A | 100 | A/G | −116.08 |
| | | | 517b | | | | | |
| rs10735810 | VDR | CDS | hsa-miR- | A/G | G | 149 | A/G | 10.29% |
| | | | 520a-3p | | | | | |
| rs10735810 | VDR | CDS | hsa-miR-524- | A/G | A | 102 | A/G | −21.99% |
| | | | 3p | | | | | |
| rs10735810 | VDR | CDS | hsa-miR-550 | A/G | G | 155 | A/G | 9.30% |
| rs10735810 | VDR | CDS | hsa-miR-574- | A/G | A | 99 | A/G | −15.51% |
| | | | 3p | | | | | |
| rs10735810 | VDR | CDS | hsa-miR-593 | A/G | G | 140 | A/G | 27.74% |
| rs10735810 | VDR | CDS | hsa-miR- | A/G | A | 109 | A/G | −10.99% |
| | | | 593* | | | | | |
| rs10735810 | VDR | CDS | hsa-miR-598 | A/G | G | 127 | A/G | 9.37% |
| rs10735810 | VDR | CDS | hsa-miR-602 | A/G | G | 93 | A/G | 10.54% |
| rs10735810 | VDR | CDS | hsa-miR-608 | A/G | G | 110 | A/G | 10.81% |
| rs10735810 | VDR | CDS | hsa-miR-612 | A/G | G | 116 | A/G | 29.52% |
| rs10735810 | VDR | CDS | hsa-miR-615- | A/G | G | 97 | A/G | 15.28% |
| | | | 5p | | | | | |
| rs10735810 | VDR | CDS | hsa-miR-623 | A/G | A | 136 | A/G | −8.12% |
| rs10735810 | VDR | CDS | hsa-miR-634 | A/G | A | 89 | A/G | −13.28% |
| rs10735810 | VDR | CDS | hsa-miR-636 | A/G | G | 140 | A/G | 21.23% |
| rs10735810 | VDR | CDS | hsa-miR-637 | A/G | G | 112 | A/G | 8.03% |
| rs10735810 | VDR | CDS | hsa-miR-646 | A/G | A | 102 | A/G | −11.34% |
| rs10735810 | VDR | CDS | hsa-miR-652 | A/G | G | 115 | A/G | 34.69% |
| rs10735810 | VDR | CDS | hsa-miR-660 | A/G | A | 120 | A/G | −20.24% |
| rs10735810 | VDR | CDS | hsa-miR-661 | A/G | G | 119 | A/G | 24.90% |
| rs10735810 | VDR | CDS | hsa-miR- | A/G | G | 134 | A/G | 33.51% |
| | | | 663b | | | | | |
| rs10735810 | VDR | CDS | hsa-miR-671- | A/G | G | 105 | A/G | 13.62% |
| | | | 3p | | | | | |
| rs10735810 | VDR | CDS | hsa-miR-671- | A/G | A | 120 | A/G | −8.16% |
| | | | 5p | | | | | |
| rs10735810 | VDR | CDS | hsa-miR- | A/G | G | 90 | A/G | 32.51% |
| | | | 675* | | | | | |
| rs10735810 | VDR | CDS | hsa-miR-769- | A/G | G | 70 | A/G | 25.92% |
| | | | 3p | | | | | |
| rs10735810 | VDR | CDS | hsa-miR-769- | A/G | G | 85 | A/G | 29.40% |
| | | | 5p | | | | | |
| rs10735810 | VDR | CDS | hsa-miR-770- | A/G | G | 105 | A/G | 13.69% |
| | | | 5p | | | | | |
| rs10735810 | VDR | CDS | hsa-miR-874 | A/G | G | 140 | A/G | 55.97% |
| rs10735810 | VDR | CDS | hsa-miR-885- | A/G | A | 128 | A/G | −9.07% |
| | | | 5p | | | | | |
| rs10735810 | VDR | CDS | hsa-miR-886- | A/G | G | 103 | A/G | 24.19% |
| | | | 3p | | | | | |
| rs10735810 | VDR | CDS | hsa-miR- | A/G | G | 125 | A/G | 29.61% |
| | | | 892b | | | | | |
| rs10735810 | VDR | CDS | hsa-miR-92a | A/G | G | 115 | A/G | 24.25% |
| rs10735810 | VDR | CDS | hsa-miR-92a- | A/G | G | 94 | A/G | 18.37% |
| | | | 1* | | | | | |
| rs10735810 | VDR | CDS | hsa-miR-92b | A/G | G | 121 | A/G | 9.45% |
| rs10735810 | VDR | CDS | hsa-miR-937 | A/G | G | 115 | A/G | 16.24% |
| rs10735810 | VDR | CDS | hsa-miR-939 | A/G | G | 98 | A/G | 40.03% |
| rs10735810 | VDR | CDS | hsa-miR-940 | A/G | G | 121 | A/G | 30.45% |
| rs731236 | VDR | CDS | hsa-miR- | C/T | C | 113 | C/T | −21.52% |
| | | | 105* | | | | | |
| rs731236 | VDR | CDS | hsa-miR- | C/T | C | 115 | C/T | −14.75% |
| | | | 1182 | | | | | |
| rs731236 | VDR | CDS | hsa-miR- | C/T | C | 92 | C/T | −26.91% |
| | | | 1183 | | | | | |
| rs731236 | VDR | CDS | hsa-miR- | C/T | C | 122 | C/T | −17.82% |
| | | | 1201 | | | | | |
| rs731236 | VDR | CDS | hsa-miR- | C/T | C | 121 | C/T | −11.01% |
| | | | 1203 | | | | | |
| rs731236 | VDR | CDS | hsa-miR- | C/T | T | 124 | C/T | 26.55% |
| | | | 1204 | | | | | |
| rs731236 | VDR | CDS | hsa-miR-122 | C/T | C | 79 | C/T | −8.80% |
| rs731236 | VDR | CDS | hsa-miR- | C/T | C | 113 | C/T | −18.40% |
| | | | 1231 | | | | | |
| rs731236 | VDR | CDS | hsa-miR- | C/T | C | 128 | C/T | −10.74% |
| | | | 1238 | | | | | |
| rs731236 | VDR | CDS | hsa-miR- | C/T | C | 90 | C/T | −29.83% |
| | | | 124* | | | | | |
| rs731236 | VDR | CDS | hsa-miR- | C/T | T | 92 | C/T | 12.57% |
| | | | 1263 | | | | | |
| rs731236 | VDR | CDS | hsa-miR- | C/T | C | 106 | C/T | −46.17% |
| | | | 1265 | | | | | |
| rs731236 | VDR | CDS | hsa-miR- | C/T | C | 119 | C/T | −15.77% |
| | | | 1288 | | | | | |
| rs731236 | VDR | CDS | hsa-miR- | C/T | C | 106 | C/T | −14.02% |
| | | | 1289 | | | | | |
| rs731236 | VDR | CDS | hsa-miR- | C/T | C | 86 | C/T | −26.02% |
| | | | 1290 | | | | | |
| rs731236 | VDR | CDS | hsa-miR- | C/T | C | 139 | C/T | −8.27% |
| | | | 1291 | | | | | |
| rs731236 | VDR | CDS | hsa-miR- | C/T | C | 142 | C/T | −9.66% |
| | | | 1293 | | | | | |
| rs731236 | VDR | CDS | hsa-miR- | C/T | T | 106 | C/T | 13.49% |
| | | | 1304 | | | | | |
| rs731236 | VDR | CDS | hsa-miR- | C/T | C | 114 | C/T | −15.66% |
| | | | 1308 | | | | | |
| rs731236 | VDR | CDS | hsa-miR- | C/T | T | 134 | C/T | 23.18% |
| | | | 133a | | | | | |
| rs731236 | VDR | CDS | hsa-miR- | C/T | T | 134 | C/T | 18.55% |
| | | | 133b | | | | | |
| rs731236 | VDR | CDS | hsa-miR-138 | C/T | T | 126 | C/T | 8.69% |
| rs731236 | VDR | CDS | hsa-miR-140- | C/T | C | 89 | C/T | −56.27% |
| | | | 3p | | | | | |
| rs731236 | VDR | CDS | hsa-miR- | C/T | T | 112 | C/T | 15.93% |
| | | | 146b-3p | | | | | |
| rs731236 | VDR | CDS | hsa-miR-149 | C/T | C | 118 | C/T | −9.80% |
| rs731236 | VDR | CDS | hsa-miR-150 | C/T | T | 81 | C/T | 66.80% |
| rs731236 | VDR | CDS | hsa-miR-154 | C/T | C | 94 | C/T | −8.59% |
| rs731236 | VDR | CDS | hsa-miR- | C/T | C | 110 | C/T | −10.75% |
| | | | 181a* | | | | | |
| rs731236 | VDR | CDS | hsa-miR- | C/T | C | 88 | C/T | −13.58% |
| | | | 181a-2* | | | | | |
| rs731236 | VDR | CDS | hsa-miR- | C/T | C | 132 | C/T | −32.39% |
| | | | 1826 | | | | | |
| rs731236 | VDR | CDS | hsa-miR- | C/T | C | 140 | C/T | −9.46% |
| | | | 1908 | | | | | |
| rs731236 | VDR | CDS | hsa-miR- | C/T | T | 108 | C/T | 29.07% |
| | | | 1909* | | | | | |
| rs731236 | VDR | CDS | hsa-miR- | C/T | C | 98 | C/T | −14.03% |
| | | | 1914 | | | | | |
| rs731236 | VDR | CDS | hsa-miR- | C/T | C | 102 | C/T | −11.77% |
| | | | 193b* | | | | | |
| rs731236 | VDR | CDS | hsa-miR- | C/T | C | 149 | C/T | −11.71% |
| | | | 196b* | | | | | |
| rs731236 | VDR | CDS | hsa-miR- | C/T | T | 87 | C/T | 10.85% |
| | | | 1974 | | | | | |
| rs731236 | VDR | CDS | hsa-miR- | C/T | T | 86 | C/T | 81.52% |
| | | | 1975 | | | | | |
| rs731236 | VDR | CDS | hsa-miR-19b- | C/T | T | 98 | C/T | 15.62% |
| | | | 1* | | | | | |
| rs731236 | VDR | CDS | hsa-miR-218- | C/T | T | 79 | C/T | 27.13% |
| | | | 1* | | | | | |
| rs731236 | VDR | CDS | hsa-miR- | C/T | T | 106 | C/T | 76.51% |
| | | | 222* | | | | | |
| rs731236 | VDR | CDS | hsa-miR-224 | C/T | T | 80 | C/T | 28.82% |
| rs731236 | VDR | CDS | hsa-miR- | C/T | C | 120 | C/T | −16.29% |
| | | | 23b* | | | | | |
| rs731236 | VDR | CDS | hsa-miR-24 | C/T | T | 119 | C/T | 52.73% |
| rs731236 | VDR | CDS | hsa-miR-296- | C/T | C | 113 | C/T | −12.37% |
| | | | 5p | | | | | |
| rs731236 | VDR | CDS | hsa-miR-29b- | C/T | T | 97 | C/T | 8.94% |
| | | | 1* | | | | | |
| rs731236 | VDR | CDS | hsa-miR-29b- | C/T | T | 127 | C/T | 8.98% |
| | | | 2* | | | | | |
| rs731236 | VDR | CDS | hsa-miR- | C/T | T | 107 | C/T | 11.54% |
| | | | 302a* | | | | | |
| rs731236 | VDR | CDS | hsa-miR-325 | C/T | T | 84 | C/T | 16.06% |
| rs731236 | VDR | CDS | hsa-miR-339- | C/T | C | 123 | C/T | −34.67% |
| | | | 3p | | | | | |
| rs731236 | VDR | CDS | hsa-miR- | C/T | C | 131 | C/T | −11.42% |
| | | | 33b* | | | | | |
| rs731236 | VDR | CDS | hsa-miR-342- | C/T | T | 101 | C/T | 47.37% |
| | | | 3p | | | | | |
| rs731236 | VDR | CDS | hsa-miR- | C/T | T | 158 | C/T | 25.13% |
| | | | 34a* | | | | | |
| rs731236 | VDR | CDS | hsa-miR- | C/T | T | 114 | C/T | 26.55% |
| | | | 363* | | | | | |
| rs731236 | VDR | CDS | hsa-miR-370 | C/T | C | 98 | C/T | −12.81% |
| rs731236 | VDR | CDS | hsa-miR-371- | C/T | T | 130 | C/T | 14.43% |
| | | | 5p | | | | | |
| rs731236 | VDR | CDS | hsa-miR- | C/T | T | 99 | C/T | 23.62% |
| | | | 373* | | | | | |
| rs731236 | VDR | CDS | hsa-miR-375 | C/T | C | 100 | C/T | −8.64% |
| rs731236 | VDR | CDS | hsa-miR-382 | C/T | C | 91 | C/T | −8.75% |
| rs731236 | VDR | CDS | hsa-miR-383 | C/T | C | 120 | C/T | −16.62% |
| rs731236 | VDR | CDS | hsa-miR-432 | C/T | C | 122 | C/T | −13.58% |
| rs731236 | VDR | CDS | hsa-miR- | C/T | C | 106 | C/T | −69.61% |
| | | | 450b-3p | | | | | |
| rs731236 | VDR | CDS | hsa-miR-453 | C/T | C | 130 | C/T | −12.40% |
| rs731236 | VDR | CDS | hsa-miR-498 | C/T | T | 116 | C/T | 41.92% |
| rs731236 | VDR | CDS | hsa-miR-505 | C/T | T | 102 | C/T | 46.27% |
| rs731236 | VDR | CDS | hsa-miR-509- | C/T | T | 82 | C/T | 17.66% |
| | | | 5p | | | | | |
| rs731236 | VDR | CDS | hsa-miR-510 | C/T | T | 102 | C/T | 17.95% |
| rs731236 | VDR | CDS | hsa-miR- | C/T | T | 100 | C/T | 17.78% |
| | | | 513a-5p | | | | | |
| rs731236 | VDR | CDS | hsa-miR- | C/T | T | 108 | C/T | 21.68% |
| | | | 513c | | | | | |
| rs731236 | VDR | CDS | hsa-miR- | C/T | T | 81 | C/T | 12.11% |
| | | | 518b | | | | | |
| rs731236 | VDR | CDS | hsa-miR- | C/T | T | 80 | C/T | 32.58% |
| | | | 518c | | | | | |
| rs731236 | VDR | CDS | hsa-miR- | C/T | C | 118 | C/T | −12.81% |
| | | | 518c* | | | | | |
| rs731236 | VDR | CDS | hsa-miR- | C/T | C | 85 | C/T | −40.09% |
| | | | 518e* | | | | | |
| rs731236 | VDR | CDS | hsa-miR- | C/T | C | 85 | C/T | −40.09% |
| | | | 519a* | | | | | |
| rs731236 | VDR | CDS | hsa-miR- | C/T | C | 85 | C/T | −40.09% |
| | | | 519b-5p | | | | | |
| rs731236 | VDR | CDS | hsa-miR- | C/T | C | 85 | C/T | −40.09% |
| | | | 519c-5p | | | | | |
| rs731236 | VDR | CDS | hsa-miR- | C/T | C | 85 | C/T | −40.09% |
| | | | 522* | | | | | |
| rs731236 | VDR | CDS | hsa-miR- | C/T | C | 85 | C/T | −40.09% |
| | | | 523* | | | | | |
| rs731236 | VDR | CDS | hsa-miR-532- | C/T | C | 108 | C/T | −21.43% |
| | | | 5p | | | | | |
| rs731236 | VDR | CDS | hsa-miR-543 | C/T | T | 96 | C/T | 26.00% |
| rs731236 | VDR | CDS | hsa-miR- | C/T | T | 80 | C/T | 14.20% |
| | | | 551b | | | | | |
| rs731236 | VDR | CDS | hsa-miR-554 | C/T | T | 113 | C/T | 23.84% |
| rs731236 | VDR | CDS | hsa-miR-574- | C/T | C | 104 | C/T | −42.10% |
| | | | 3p | | | | | |
| rs731236 | VDR | CDS | hsa-miR-581 | C/T | C | 103 | C/T | −11.34% |
| rs731236 | VDR | CDS | hsa-miR- | C/T | C | 105 | C/T | −21.72% |
| | | | 593* | | | | | |
| rs731236 | VDR | CDS | hsa-miR-596 | C/T | C | 90 | C/T | −11.23% |
| rs731236 | VDR | CDS | hsa-miR-600 | C/T | C | 96 | C/T | −65.08% |
| rs731236 | VDR | CDS | hsa-miR-608 | C/T | C | 139 | C/T | −25.06% |
| rs731236 | VDR | CDS | hsa-miR-614 | C/T | T | 87 | C/T | 10.22% |
| rs731236 | VDR | CDS | hsa-miR-615- | C/T | C | 158 | C/T | −10.23% |
| | | | 5p | | | | | |
| rs731236 | VDR | CDS | hsa-miR-616 | C/T | C | 113 | C/T | −18.90% |
| rs731236 | VDR | CDS | hsa-miR-621 | C/T | T | 80 | C/T | 19.66% |
| rs731236 | VDR | CDS | hsa-miR-623 | C/T | C | 105 | C/T | −37.15% |
| rs731236 | VDR | CDS | hsa-miR-650 | C/T | C | 111 | C/T | −10.50% |
| rs731236 | VDR | CDS | hsa-miR-658 | C/T | C | 104 | C/T | −31.30% |
| rs731236 | VDR | CDS | hsa-miR-662 | C/T | T | 74 | C/T | 12.72% |
| rs731236 | VDR | CDS | hsa-miR-668 | C/T | T | 123 | C/T | 10.30% |
| rs731236 | VDR | CDS | hsa-miR-671- | C/T | C | 119 | C/T | −9.14% |
| | | | 5p | | | | | |
| rs731236 | VDR | CDS | hsa-miR- | C/T | T | 112 | C/T | 12.76% |
| | | | 675* | | | | | |
| rs731236 | VDR | CDS | hsa-miR-708 | C/T | T | 106 | C/T | 34.02% |
| rs731236 | VDR | CDS | hsa-miR-765 | C/T | C | 97 | C/T | −21.38% |
| rs731236 | VDR | CDS | hsa-miR-769- | C/T | C | 116 | C/T | −8.21% |
| | | | 3p | | | | | |
| rs731236 | VDR | CDS | hsa-miR-769- | C/T | T | 101 | C/T | 94.42% |
| | | | 5p | | | | | |
| rs731236 | VDR | CDS | hsa-miR-770- | C/T | C | 80 | C/T | −17.55% |
| | | | 5p | | | | | |
| rs731236 | VDR | CDS | hsa-miR-874 | C/T | T | 110 | C/T | 14.63% |
| rs731236 | VDR | CDS | hsa-miR-924 | C/T | C | 106 | C/T | −25.82% |
| rs731236 | VDR | CDS | hsa-miR-92a- | C/T | C | 114 | C/T | −17.31% |
| | | | 1* | | | | | |
| rs731236 | VDR | CDS | hsa-miR-92b | C/T | C | 103 | C/T | −13.21% |
| rs731236 | VDR | CDS | hsa-miR-935 | C/T | T | 94 | C/T | 26.05% |
| rs731236 | VDR | CDS | hsa-miR-938 | C/T | T | 109 | C/T | 105.00% |
| rs4588 | GC | CDS | hsa-miR- | A/C | C | 132 | A/C | 24.43% |
| | | | 1180 | | | | | |
| rs4588 | GC | CDS | hsa-miR- | A/C | C | 111 | A/C | 16.36% |
| | | | 1183 | | | | | |
| rs4588 | GC | CDS | hsa-miR- | A/C | C | 149 | A/C | 8.49% |
| | | | 1202 | | | | | |
| rs4588 | GC | CDS | hsa-miR- | A/C | A | 94 | A/C | −27.51% |
| | | | 125b-2* | | | | | |
| rs4588 | GC | CDS | hsa-miR-127- | A/C | C | 125 | A/C | 31.08% |
| | | | 3p | | | | | |
| rs4588 | GC | CDS | hsa-miR- | A/C | A | 105 | A/C | −9.31% |
| | | | 1307 | | | | | |
| rs4588 | GC | CDS | hsa-miR-134 | A/C | C | 77 | A/C | 22.31% |
| rs4588 | GC | CDS | hsa-miR- | A/C | C | 107 | A/C | 25.51% |
| | | | 1468 | | | | | |
| rs4588 | GC | CDS | hsa-miR- | A/C | C | 98 | A/C | 25.19% |
| | | | 146b-3p | | | | | |
| rs4588 | GC | CDS | hsa-miR- | A/C | C | 106 | A/C | 25.04% |
| | | | 1471 | | | | | |
| rs4588 | GC | CDS | hsa-miR- | A/C | C | 140 | A/C | 30.98% |
| | | | 148b* | | | | | |
| rs4588 | GC | CDS | hsa-miR-152 | A/C | A | 88 | A/C | −33.00% |
| rs4588 | GC | CDS | hsa-miR- | A/C | A | 101 | A/C | −9.65% |
| | | | 1538 | | | | | |
| rs4588 | GC | CDS | hsa-miR- | A/C | C | 81 | A/C | 58.69% |
| | | | 181b | | | | | |
| rs4588 | GC | CDS | hsa-miR- | A/C | C | 105 | A/C | 51.72% |
| | | | 181c* | | | | | |
| rs4588 | GC | CDS | hsa-miR-187 | A/C | C | 106 | A/C | 18.38% |
| rs4588 | GC | CDS | hsa-miR-188- | A/C | A | 104 | A/C | −21.83% |
| | | | 5p | | | | | |
| rs4588 | GC | CDS | hsa-miR- | A/C | C | 125 | A/C | 19.14% |
| | | | 1910 | | | | | |
| rs4588 | GC | CDS | hsa-miR- | A/C | C | 101 | A/C | 36.36% |
| | | | 193b | | | | | |
| rs4588 | GC | CDS | hsa-miR- | A/C | A | 110 | A/C | −8.60% |
| | | | 194* | | | | | |
| rs4588 | GC | CDS | hsa-miR-21* | A/C | C | 96 | A/C | 13.28% |
| rs4588 | GC | CDS | hsa-miR-210 | A/C | C | 134 | A/C | 40.73% |
| rs4588 | GC | CDS | hsa-miR- | A/C | A | 82 | A/C | −209.07 |
| | | | 222* | | | | | |
| rs4588 | GC | CDS | hsa-miR-27a | A/C | C | 91 | A/C | 91.82% |
| rs4588 | GC | CDS | hsa-miR- | A/C | C | 100 | A/C | 26.74% |
| | | | 302c | | | | | |
| rs4588 | GC | CDS | hsa-miR-328 | A/C | C | 93 | A/C | 24.45% |
| rs4588 | GC | CDS | hsa-miR-362- | A/C | A | 132 | A/C | −41.73% |
| | | | 5p | | | | | |
| rs4588 | GC | CDS | hsa-miR-383 | A/C | C | 88 | A/C | 82.34% |
| rs4588 | GC | CDS | hsa-miR-423- | A/C | A | 92 | A/C | −37.68% |
| | | | 3p | | | | | |
| rs4588 | GC | CDS | hsa-miR-432 | A/C | A | 123 | A/C | −16.81% |
| rs4588 | GC | CDS | hsa-miR-497 | A/C | C | 95 | A/C | 66.84% |
| rs4588 | GC | CDS | hsa-miR-502- | A/C | C | 118 | A/C | 22.80% |
| | | | 5p | | | | | |
| rs4588 | GC | CDS | hsa-miR-503 | A/C | C | 104 | A/C | 26.10% |
| rs4588 | GC | CDS | hsa-miR-507 | A/C | A | 110 | A/C | −21.16% |
| rs4588 | GC | CDS | hsa-miR-509- | A/C | C | 90 | A/C | 105.97% |
| | | | 3p | | | | | |
| rs4588 | GC | CDS | hsa-miR-532- | A/C | A | 114 | A/C | −35.95% |
| | | | 5p | | | | | |
| rs4588 | GC | CDS | hsa-miR- | A/C | C | 82 | A/C | 42.32% |
| | | | 551b* | | | | | |
| rs4588 | GC | CDS | hsa-miR-557 | A/C | C | 110 | A/C | 14.36% |
| rs4588 | GC | CDS | hsa-miR-572 | A/C | C | 92 | A/C | 21.64% |
| rs4588 | GC | CDS | hsa-miR-596 | A/C | A | 136 | A/C | −11.71% |
| rs4588 | GC | CDS | hsa-miR-622 | A/C | C | 114 | A/C | 21.21% |
| rs4588 | GC | CDS | hsa-miR-623 | A/C | A | 124 | A/C | −10.89% |
| rs4588 | GC | CDS | hsa-miR- | A/C | A | 108 | A/C | −27.70% |
| | | | 624* | | | | | |
| rs4588 | GC | CDS | hsa-miR-632 | A/C | C | 96 | A/C | 12.26% |
| rs4588 | GC | CDS | hsa-miR-634 | A/C | A | 135 | A/C | −22.50% |
| rs4588 | GC | CDS | hsa-miR-638 | A/C | C | 57 | A/C | 54.15% |
| rs4588 | GC | CDS | hsa-miR-760 | A/C | C | 127 | A/C | 33.47% |
| rs4588 | GC | CDS | hsa-miR-770- | A/C | C | 135 | A/C | 10.24% |
| | | | 5p | | | | | |
| rs4588 | GC | CDS | hsa-miR-874 | A/C | C | 195 | A/C | 102.12% |
| rs4588 | GC | CDS | hsa-miR- | A/C | A | 108 | A/C | −26.95% |
| | | | 888* | | | | | |
| rs4588 | GC | CDS | hsa-miR-890 | A/C | A | 120 | A/C | −24.13% |
| rs4588 | GC | CDS | hsa-miR-93 | A/C | C | 96 | A/C | 14.22% |
| rs4588 | GC | CDS | hsa-miR-935 | A/C | C | 114 | A/C | 37.48% |
| rs4588 | GC | CDS | hsa-miR-939 | A/C | A | 94 | A/C | −9.08% |
| rs4588 | GC | CDS | hsa-miR-941 | A/C | C | 117 | A/C | 17.64% |
| rs4588 | GC | CDS | hsa-miR- | A/C | C | 98 | A/C | 45.56% |
| | | | 99b* | | | | | |
| rs7041 | GC | CDS | hsa-miR- | G/T | G | 128 | G/T | −26.66% |
| | | | 1205 | | | | | |
| rs7041 | GC | CDS | hsa-miR- | G/T | G | 117 | G/T | −25.21% |
| | | | 1224-5p | | | | | |
| rs7041 | GC | CDS | hsa-miR- | G/T | G | 118 | G/T | −28.27% |
| | | | 1225-5p | | | | | |
| rs7041 | GC | CDS | hsa-miR- | G/T | T | 110 | G/T | 15.50% |
| | | | 1226* | | | | | |
| rs7041 | GC | CDS | hsa-miR- | G/T | T | 118 | G/T | 8.68% |
| | | | 1228* | | | | | |
| rs7041 | GC | CDS | hsa-miR- | G/T | G | 117 | G/T | −33.44% |
| | | | 1250 | | | | | |
| rs7041 | GC | CDS | hsa-miR- | G/T | T | 131 | G/T | 17.26% |
| | | | 1262 | | | | | |
| rs7041 | GC | CDS | hsa-miR- | G/T | G | 134 | G/T | −28.30% |
| | | | 1266 | | | | | |
| rs7041 | GC | CDS | hsa-miR- | G/T | G | 96 | G/T | −14.91% |
| | | | 1292 | | | | | |
| rs7041 | GC | CDS | hsa-miR- | G/T | G | 128 | G/T | −21.04% |
| | | | 1301 | | | | | |
| rs7041 | GC | CDS | hsa-miR- | G/T | T | 105 | G/T | 11.41% |
| | | | 1307 | | | | | |
| rs7041 | GC | CDS | hsa-miR- | G/T | G | 130 | G/T | −8.97% |
| | | | 1308 | | | | | |
| rs7041 | GC | CDS | hsa-miR- | G/T | G | 85 | G/T | −19.97% |
| | | | 135a* | | | | | |
| rs7041 | GC | CDS | hsa-miR- | G/T | G | 102 | G/T | −20.86% |
| | | | 146b-3p | | | | | |
| rs7041 | GC | CDS | hsa-miR- | G/T | G | 146 | G/T | −21.72% |
| | | | 148b* | | | | | |
| rs7041 | GC | CDS | hsa-miR- | G/T | G | 137 | G/T | −38.05% |
| | | | 1538 | | | | | |
| rs7041 | GC | CDS | hsa-miR- | G/T | G | 91 | G/T | −32.43% |
| | | | 15a* | | | | | |
| rs7041 | GC | CDS | hsa-miR- | G/T | G | 111 | G/T | −27.33% |
| | | | 185* | | | | | |
| rs7041 | GC | CDS | hsa-miR-18b | G/T | T | 96 | G/T | 11.30% |
| rs7041 | GC | CDS | hsa-miR- | G/T | T | 109 | G/T | 25.96% |
| | | | 1909* | | | | | |
| rs7041 | GC | CDS | hsa-miR- | G/T | G | 134 | G/T | −27.18% |
| | | | 1910 | | | | | |
| rs7041 | GC | CDS | hsa-miR- | G/T | G | 91 | G/T | −28.22% |
| | | | 1911* | | | | | |
| rs7041 | GC | CDS | hsa-miR- | G/T | T | 120 | G/T | 8.52% |
| | | | 1915 | | | | | |
| rs7041 | GC | CDS | hsa-miR-19b- | G/T | T | 137 | G/T | 51.99% |
| | | | 1* | | | | | |
| rs7041 | GC | CDS | hsa-miR-219- | G/T | T | 81 | G/T | 64.93% |
| | | | 2-3p | | | | | |
| rs7041 | GC | CDS | hsa-miR- | G/T | T | 123 | G/T | 13.26% |
| | | | 23b* | | | | | |
| rs7041 | GC | CDS | hsa-miR-25* | G/T | G | 101 | G/T | −26.30% |
| rs7041 | GC | CDS | hsa-miR-296- | G/T | G | 115 | G/T | −18.28% |
| | | | 3p | | | | | |
| rs7041 | GC | CDS | hsa-miR-323- | G/T | G | 132 | G/T | −36.14% |
| | | | 5p | | | | | |
| rs7041 | GC | CDS | hsa-miR-330- | G/T | G | 138 | G/T | −21.16% |
| | | | 5p | | | | | |
| rs7041 | GC | CDS | hsa-miR-331- | G/T | G | 110 | G/T | −21.64% |
| | | | 5p | | | | | |
| rs7041 | GC | CDS | hsa-miR-338- | G/T | T | 118 | G/T | 41.82% |
| | | | 3p | | | | | |
| rs7041 | GC | CDS | hsa-miR- | G/T | G | 123 | G/T | −49.71% |
| | | | 33b* | | | | | |
| rs7041 | GC | CDS | hsa-miR-346 | G/T | G | 110 | G/T | −74.35% |
| rs7041 | GC | CDS | hsa-miR-370 | G/T | G | 103 | G/T | −14.26% |
| rs7041 | GC | CDS | hsa-miR-382 | G/T | G | 97 | G/T | −14.19% |
| rs7041 | GC | CDS | hsa-miR-423- | G/T | G | 112 | G/T | −18.98% |
| | | | 3p | | | | | |
| rs7041 | GC | CDS | hsa-miR- | G/T | G | 95 | G/T | −33.31% |
| | | | 431* | | | | | |
| rs7041 | GC | CDS | hsa-miR-453 | G/T | T | 106 | G/T | 38.62% |
| rs7041 | GC | CDS | hsa-miR-508- | G/T | T | 103 | G/T | 16.82% |
| | | | 5p | | | | | |
| rs7041 | GC | CDS | hsa-miR-541 | G/T | T | 148 | G/T | 9.05% |
| rs7041 | GC | CDS | hsa-miR- | G/T | G | 88 | G/T | −15.14% |
| | | | 548b-3p | | | | | |
| rs7041 | GC | CDS | hsa-miR-554 | G/T | G | 118 | G/T | −25.45% |
| rs7041 | GC | CDS | hsa-miR-557 | G/T | G | 119 | G/T | −28.37% |
| rs7041 | GC | CDS | hsa-miR-572 | G/T | G | 100 | G/T | −25.35% |
| rs7041 | GC | CDS | hsa-miR-596 | G/T | G | 99 | G/T | −33.15% |
| rs7041 | GC | CDS | hsa-miR-611 | G/T | G | 84 | G/T | −32.14% |
| rs7041 | GC | CDS | hsa-miR-614 | G/T | T | 92 | G/T | 34.84% |
| rs7041 | GC | CDS | hsa-miR-615- | G/T | G | 94 | G/T | −64.41% |
| | | | 3p | | | | | |
| rs7041 | GC | CDS | hsa-miR-638 | G/T | G | 82 | G/T | −39.36% |
| rs7041 | GC | CDS | hsa-miR-644 | G/T | G | 164 | G/T | −16.72% |
| rs7041 | GC | CDS | hsa-miR-654- | G/T | G | 153 | G/T | −20.35% |
| | | | 5p | | | | | |
| rs7041 | GC | CDS | hsa-miR-661 | G/T | G | 128 | G/T | −31.83% |
| rs7041 | GC | CDS | hsa-miR-662 | G/T | G | 108 | G/T | −33.29% |
| rs7041 | GC | CDS | hsa-miR-663 | G/T | G | 113 | G/T | −9.31% |
| rs7041 | GC | CDS | hsa-miR- | G/T | G | 140 | G/T | −38.80% |
| | | | 663b | | | | | |
| rs7041 | GC | CDS | hsa-miR-665 | G/T | G | 105 | G/T | −31.06% |
| rs7041 | GC | CDS | hsa-miR-766 | G/T | G | 96 | G/T | −184.03 |
| rs7041 | GC | CDS | hsa-miR-770- | G/T | G | 141 | G/T | −24.93% |
| | | | 5p | | | | | |
| rs7041 | GC | CDS | hsa-miR-874 | G/T | T | 95 | G/T | 47.85% |
| rs7041 | GC | CDS | hsa-miR-885- | G/T | G | 103 | G/T | −37.67% |
| | | | 3p | | | | | |
| rs7041 | GC | CDS | hsa-miR-890 | G/T | T | 92 | G/T | 42.55% |
| rs7041 | GC | CDS | hsa-miR-92a- | G/T | T | 122 | G/T | 15.68% |
| | | | 1* | | | | | |
| rs7041 | GC | CDS | hsa-miR-92a- | G/T | T | 140 | G/T | 21.47% |
| | | | 2* | | | | | |
| rs7041 | GC | CDS | hsa-miR-92b | G/T | G | 92 | G/T | −79.74% |
| rs1799782 | XRCC1 | CDS | hsa-let-7a-2* | C/T | T | 83 | C/T | 18.21% |
| rs1799782 | XRCC1 | CDS | hsa-miR- | C/T | T | 143 | C/T | 30.16% |
| | | | 1202 | | | | | |
| rs1799782 | XRCC1 | CDS | hsa-miR- | C/T | C | 96 | C/T | −32.74% |
| | | | 1207-5p | | | | | |
| rs1799782 | XRCC1 | CDS | hsa-miR- | C/T | T | 98 | C/T | 9.51% |
| | | | 1225-5p | | | | | |
| rs1799782 | XRCC1 | CDS | hsa-miR- | C/T | C | 86 | C/T | −53.37% |
| | | | 1228* | | | | | |
| rs1799782 | XRCC1 | CDS | hsa-miR- | C/T | T | 136 | C/T | 27.02% |
| | | | 1231 | | | | | |
| rs1799782 | XRCC1 | CDS | hsa-miR- | C/T | T | 105 | C/T | 12.57% |
| | | | 1254 | | | | | |
| rs1799782 | XRCC1 | CDS | hsa-miR- | C/T | T | 130 | C/T | 18.70% |
| | | | 125a-3p | | | | | |
| rs1799782 | XRCC1 | CDS | hsa-miR- | C/T | C | 96 | C/T | −47.67% |
| | | | 125b-1* | | | | | |
| rs1799782 | XRCC1 | CDS | hsa-miR- | C/T | C | 96 | C/T | −101.14 |
| | | | 1269 | | | | | |
| rs1799782 | XRCC1 | CDS | hsa-miR-127- | C/T | C | 127 | C/T | −9.80% |
| | | | 3p | | | | | |
| rs1799782 | XRCC1 | CDS | hsa-miR-128 | C/T | T | 92 | C/T | 26.05% |
| rs1799782 | XRCC1 | CDS | hsa-miR- | C/T | C | 121 | C/T | −11.18% |
| | | | 1292 | | | | | |
| rs1799782 | XRCC1 | CDS | hsa-miR- | C/T | C | 114 | C/T | −27.94% |
| | | | 1293 | | | | | |
| rs1799782 | XRCC1 | CDS | hsa-miR- | C/T | C | 114 | C/T | −20.50% |
| | | | 1298 | | | | | |
| rs1799782 | XRCC1 | CDS | hsa-miR- | C/T | T | 133 | C/T | 32.23% |
| | | | 1301 | | | | | |
| rs1799782 | XRCC1 | CDS | hsa-miR-138 | C/T | T | 125 | C/T | 9.58% |
| rs1799782 | XRCC1 | CDS | hsa-miR- | C/T | T | 81 | C/T | 10.81% |
| | | | 143* | | | | | |
| rs1799782 | XRCC1 | CDS | hsa-miR-145 | C/T | T | 132 | C/T | 35.62% |
| rs1799782 | XRCC1 | CDS | hsa-miR- | C/T | C | 108 | C/T | −17.47% |
| | | | 1469 | | | | | |
| rs1799782 | XRCC1 | CDS | hsa-miR-149 | C/T | C | 116 | C/T | −48.14% |
| rs1799782 | XRCC1 | CDS | hsa-miR-151- | C/T | T | 87 | C/T | 18.93% |
| | | | 3p | | | | | |
| rs1799782 | XRCC1 | CDS | hsa-miR- | C/T | C | 101 | C/T | −17.20% |
| | | | 187* | | | | | |
| rs1799782 | XRCC1 | CDS | hsa-miR- | C/T | C | 113 | C/T | −13.85% |
| | | | 1908 | | | | | |
| rs1799782 | XRCC1 | CDS | hsa-miR- | C/T | C | 95 | C/T | −26.32% |
| | | | 191* | | | | | |
| rs1799782 | XRCC1 | CDS | hsa-miR- | C/T | C | 125 | C/T | −15.77% |
| | | | 194* | | | | | |
| rs1799782 | XRCC1 | CDS | hsa-miR- | C/T | C | 101 | C/T | −25.92% |
| | | | 196a* | | | | | |
| rs1799782 | XRCC1 | CDS | hsa-miR-198 | C/T | T | 121 | C/T | 22.56% |
| rs1799782 | XRCC1 | CDS | hsa-miR-22 | C/T | T | 104 | C/T | 102.69 |
| rs1799782 | XRCC1 | CDS | hsa-miR-25* | C/T | C | 117 | C/T | −22.17% |
| rs1799782 | XRCC1 | CDS | hsa-miR-296- | C/T | C | 115 | C/T | −54.18% |
| | | | 3p | | | | | |
| rs1799782 | XRCC1 | CDS | hsa-miR-298 | C/T | T | 155 | C/T | 8.02% |
| rs1799782 | XRCC1 | CDS | hsa-miR- | C/T | T | 125 | C/T | 8.34% |
| | | | 320a | | | | | |
| rs1799782 | XRCC1 | CDS | hsa-miR- | C/T | T | 125 | C/T | 8.18% |
| | | | 320b | | | | | |
| rs1799782 | XRCC1 | CDS | hsa-miR- | C/T | T | 122 | C/T | 9.51% |
| | | | 320c | | | | | |
| rs1799782 | XRCC1 | CDS | hsa-miR- | C/T | T | 122 | C/T | 10.03% |
| | | | 320d | | | | | |
| rs1799782 | XRCC1 | CDS | hsa-miR-323- | C/T | C | 109 | C/T | −13.35% |
| | | | 5p | | | | | |
| rs1799782 | XRCC1 | CDS | hsa-miR-34a | C/T | C | 101 | C/T | −16.83% |
| rs1799782 | XRCC1 | CDS | hsa-miR- | C/T | T | 119 | C/T | 41.91% |
| | | | 365* | | | | | |
| rs1799782 | XRCC1 | CDS | hsa-miR-375 | C/T | C | 108 | C/T | −18.25% |
| rs1799782 | XRCC1 | CDS | hsa-miR-409- | C/T | C | 119 | C/T | −16.39% |
| | | | 3p | | | | | |
| rs1799782 | XRCC1 | CDS | hsa-miR- | C/T | C | 114 | C/T | −29.52% |
| | | | 425* | | | | | |
| rs1799782 | XRCC1 | CDS | hsa-miR-431 | C/T | C | 131 | C/T | −10.02% |
| rs1799782 | XRCC1 | CDS | hsa-miR- | C/T | C | 104 | C/T | −24.08% |
| | | | 449b | | | | | |
| rs1799782 | XRCC1 | CDS | hsa-miR-484 | C/T | C | 110 | C/T | −19.54% |
| rs1799782 | XRCC1 | CDS | hsa-miR-512- | C/T | T | 104 | C/T | 59.04% |
| | | | 3p | | | | | |
| rs1799782 | XRCC1 | CDS | hsa-miR-550 | C/T | C | 124 | C/T | −9.60% |
| rs1799782 | XRCC1 | CDS | hsa-miR-575 | C/T | T | 129 | C/T | 32.71% |
| rs1799782 | XRCC1 | CDS | hsa-miR-601 | C/T | T | 126 | C/T | 14.74% |
| rs1799782 | XRCC1 | CDS | hsa-miR-602 | C/T | T | 105 | C/T | 16.28% |
| rs1799782 | XRCC1 | CDS | hsa-miR-608 | C/T | T | 134 | C/T | 23.39% |
| rs1799782 | XRCC1 | CDS | hsa-miR-619 | C/T | T | 110 | C/T | 31.09% |
| rs1799782 | XRCC1 | CDS | hsa-miR-658 | C/T | C | 132 | C/T | −12.37% |
| rs1799782 | XRCC1 | CDS | hsa-miR-659 | C/T | T | 132 | C/T | 8.98% |
| rs1799782 | XRCC1 | CDS | hsa-miR-665 | C/T | T | 126 | C/T | 25.28% |
| rs1982073 | TGFB1 | CDS | hsa-miR-15a | C/T | C | 148 | C/T | −10.98% |
| rs1982073 | TGFB1 | CDS | hsa-miR-15b | C/T | C | 151 | C/T | −15.86% |
| rs1982073 | TGFB1 | CDS | hsa-miR-16 | C/T | C | 157 | C/T | −28.91% |
| rs1982073 | TGFB1 | CDS | hsa-miR-16- | C/T | C | 90 | C/T | −13.27% |
| | | | 1* | | | | | |
| rs1982073 | TGFB1 | CDS | hsa-miR-17* | C/T | C | 129 | C/T | −13.58% |
| rs1982073 | TGFB1 | CDS | hsa-miR- | C/T | C | 82 | C/T | −75.86% |
| | | | 181a* | | | | | |
| rs1982073 | TGFB1 | CDS | hsa-miR-182 | C/T | T | 86 | C/T | 17.79% |
| rs1982073 | TGFB1 | CDS | hsa-miR- | C/T | C | 111 | C/T | −17.48% |
| | | | 185* | | | | | |
| rs1982073 | TGFB1 | CDS | hsa-miR-187 | C/T | C | 83 | C/T | −41.90% |
| rs1982073 | TGFB1 | CDS | hsa-miR- | C/T | C | 91 | C/T | −18.37% |
| | | | 1908 | | | | | |
| rs1982073 | TGFB1 | CDS | hsa-miR- | C/T | C | 150 | C/T | −24.10% |
| | | | 1909 | | | | | |
| rs1982073 | TGFB1 | CDS | hsa-miR- | C/T | C | 106 | C/T | −12.35% |
| | | | 1909* | | | | | |
| rs1982073 | TGFB1 | CDS | hsa-miR-191 | C/T | T | 117 | C/T | 28.59% |
| rs1982073 | TGFB1 | CDS | hsa-miR- | C/T | C | 108 | C/T | −8.71% |
| | | | 1915 | | | | | |
| rs1982073 | TGFB1 | CDS | hsa-miR- | C/T | C | 125 | C/T | −53.51% |
| | | | 194* | | | | | |
| rs1982073 | TGFB1 | CDS | hsa-miR- | C/T | C | 147 | C/T | −12.91% |
| | | | 196b* | | | | | |
| rs1982073 | TGFB1 | CDS | hsa-miR- | C/T | C | 110 | C/T | −20.11% |
| | | | 1974 | | | | | |
| rs1982073 | TGFB1 | CDS | hsa-miR- | C/T | C | 91 | C/T | −85.13% |
| | | | 199a-5p | | | | | |
| rs1982073 | TGFB1 | CDS | hsa-miR-19b- | C/T | C | 81 | C/T | −17.57% |
| | | | 1* | | | | | |
| rs1982073 | TGFB1 | CDS | hsa-miR- | C/T | C | 83 | C/T | −17.28% |
| | | | 205* | | | | | |
| rs1982073 | TGFB1 | CDS | hsa-miR- | C/T | C | 110 | C/T | −43.74% |
| | | | 20b* | | | | | |
| rs1982073 | TGFB1 | CDS | hsa-miR-21* | C/T | T | 93 | C/T | 15.53% |
| rs1982073 | TGFB1 | CDS | hsa-miR- | C/T | C | 100 | C/T | −71.06% |
| | | | 2110 | | | | | |
| rs1982073 | TGFB1 | CDS | hsa-miR-214 | C/T | C | 138 | C/T | −30.34% |
| rs1982073 | TGFB1 | CDS | hsa-miR-218- | C/T | C | 82 | C/T | −48.51% |
| | | | 1* | | | | | |
| rs1982073 | TGFB1 | CDS | hsa-miR- | C/T | T | 128 | C/T | 12.84% |
| | | | 222* | | | | | |
| rs1982073 | TGFB1 | CDS | hsa-miR-24 | C/T | C | 111 | C/T | −32.44% |
| rs1982073 | TGFB1 | CDS | hsa-miR-296- | C/T | C | 88 | C/T | −79.05% |
| | | | 5p | | | | | |
| rs1982073 | TGFB1 | CDS | hsa-miR-298 | C/T | C | 139 | C/T | −10.55% |
| rs1982073 | TGFB1 | CDS | hsa-miR-29a | C/T | T | 125 | C/T | 42.61% |
| rs1982073 | TGFB1 | CDS | hsa-miR-29b- | C/T | C | 86 | C/T | −38.79% |
| | | | 2* | | | | | |
| rs1982073 | TGFB1 | CDS | hsa-miR-29c | C/T | T | 119 | C/T | 57.98% |
| rs1982073 | TGFB1 | CDS | hsa-miR- | C/T | T | 92 | C/T | 20.24% |
| | | | 301a | | | | | |
| rs1982073 | TGFB1 | CDS | hsa-miR- | C/T | C | 83 | C/T | −14.48% |
| | | | 30d* | | | | | |
| rs1982073 | TGFB1 | CDS | hsa-miR-323- | C/T | C | 112 | C/T | −22.47% |
| | | | 5p | | | | | |
| rs1982073 | TGFB1 | CDS | hsa-miR-325 | C/T | C | 107 | C/T | −50.28% |
| rs1982073 | TGFB1 | CDS | hsa-miR-330- | C/T | C | 113 | C/T | −8.01% |
| | | | 3p | | | | | |
| rs1982073 | TGFB1 | CDS | hsa-miR- | C/T | C | 120 | C/T | −28.74% |
| | | | 33b* | | | | | |
| rs1982073 | TGFB1 | CDS | hsa-miR-34a | C/T | C | 127 | C/T | −72.14% |
| rs1982073 | TGFB1 | CDS | hsa-miR- | C/T | C | 108 | C/T | −14.70% |
| | | | 34a* | | | | | |
| rs1982073 | TGFB1 | CDS | hsa-miR- | C/T | T | 118 | C/T | 15.11% |
| | | | 34b* | | | | | |
| rs1982073 | TGFB1 | CDS | hsa-miR-379 | C/T | C | 104 | C/T | −22.28% |
| rs1982073 | TGFB1 | CDS | hsa-miR-423- | C/T | C | 115 | C/T | −8.27% |
| | | | 3p | | | | | |
| rs1982073 | TGFB1 | CDS | hsa-miR-423- | C/T | C | 118 | C/T | −11.90% |
| | | | 5p | | | | | |
| rs1982073 | TGFB1 | CDS | hsa-miR- | C/T | C | 90 | C/T | −103.83 |
| | | | 425* | | | | | |
| rs1982073 | TGFB1 | CDS | hsa-miR- | C/T | C | 126 | C/T | −18.62% |
| | | | 449a | | | | | |
| rs1982073 | TGFB1 | CDS | hsa-miR- | C/T | C | 132 | C/T | −13.70% |
| | | | 449b | | | | | |
| rs1982073 | TGFB1 | CDS | hsa-miR-453 | C/T | C | 95 | C/T | −12.41% |
| rs1982073 | TGFB1 | CDS | hsa-miR-485- | C/T | C | 114 | C/T | −23.88% |
| | | | 5p | | | | | |
| rs1982073 | TGFB1 | CDS | hsa-miR-491- | C/T | C | 93 | C/T | −81.46% |
| | | | 5p | | | | | |
| rs1982073 | TGFB1 | CDS | hsa-miR-497 | C/T | T | 168 | C/T | 20.71% |
| rs1982073 | TGFB1 | CDS | hsa-miR-509- | C/T | C | 94 | C/T | −36.61% |
| | | | 3p | | | | | |
| rs1982073 | TGFB1 | CDS | hsa-miR-512- | C/T | T | 102 | C/T | 14.81% |
| | | | 3p | | | | | |
| rs1982073 | TGFB1 | CDS | hsa-miR- | C/T | C | 96 | C/T | −37.97% |
| | | | 518a-3p | | | | | |
| rs1982073 | TGFB1 | CDS | hsa-miR- | C/T | C | 85 | C/T | −14.30% |
| | | | 518a-5p | | | | | |
| rs1982073 | TGFB1 | CDS | hsa-miR- | C/T | C | 83 | C/T | −32.68% |
| | | | 518e* | | | | | |
| rs1982073 | TGFB1 | CDS | hsa-miR- | C/T | C | 83 | C/T | −32.68% |
| | | | 519a* | | | | | |
| rs1982073 | TGFB1 | CDS | hsa-miR- | C/T | C | 83 | C/T | −32.68% |
| | | | 519b-5p | | | | | |
| rs1982073 | TGFB1 | CDS | hsa-miR- | C/T | C | 83 | C/T | −32.68% |
| | | | 519c-5p | | | | | |
| rs1982073 | TGFB1 | CDS | hsa-miR- | C/T | C | 83 | C/T | −32.68% |
| | | | 522* | | | | | |
| rs1982073 | TGFB1 | CDS | hsa-miR- | C/T | C | 83 | C/T | −32.68% |
| | | | 523* | | | | | |
| rs1982073 | TGFB1 | CDS | hsa-miR-527 | C/T | C | 85 | C/T | −14.30% |
| rs1982073 | TGFB1 | CDS | hsa-miR-541 | C/T | C | 113 | C/T | −27.79% |
| rs1982073 | TGFB1 | CDS | hsa-miR-550 | C/T | C | 89 | C/T | −11.86% |
| rs1982073 | TGFB1 | CDS | hsa-miR- | C/T | T | 107 | C/T | 18.18% |
| | | | 589* | | | | | |
| rs1982073 | TGFB1 | CDS | hsa-miR-602 | C/T | C | 125 | C/T | −43.79% |
| rs1982073 | TGFB1 | CDS | hsa-miR-608 | C/T | C | 134 | C/T | −9.01% |
| rs1982073 | TGFB1 | CDS | hsa-miR-621 | C/T | C | 133 | C/T | −8.85% |
| rs1982073 | TGFB1 | CDS | hsa-miR-623 | C/T | C | 80 | C/T | −38.66% |
| rs1982073 | TGFB1 | CDS | hsa-miR-628- | C/T | C | 98 | C/T | −30.94% |
| | | | 3p | | | | | |
| rs1982073 | TGFB1 | CDS | hsa-miR-647 | C/T | C | 130 | C/T | −16.04% |
| rs1982073 | TGFB1 | CDS | hsa-miR-648 | C/T | C | 100 | C/T | −26.44% |
| rs1982073 | TGFB1 | CDS | hsa-miR-650 | C/T | C | 129 | C/T | −18.11% |
| rs1982073 | TGFB1 | CDS | hsa-miR-654- | C/T | C | 117 | C/T | −11.41% |
| | | | 5p | | | | | |
| rs1982073 | TGFB1 | CDS | hsa-miR-658 | C/T | T | 128 | C/T | 13.00% |
| rs1982073 | TGFB1 | CDS | hsa-miR-661 | C/T | C | 89 | C/T | −9.50% |
| rs1982073 | TGFB1 | CDS | hsa-miR-663 | C/T | C | 130 | C/T | −10.75% |
| rs1982073 | TGFB1 | CDS | hsa-miR-744 | C/T | C | 137 | C/T | −13.55% |
| rs1982073 | TGFB1 | CDS | hsa-miR-767- | C/T | C | 100 | C/T | −63.31% |
| | | | 5p | | | | | |
| rs1982073 | TGFB1 | CDS | hsa-miR-769- | C/T | C | 84 | C/T | −100.97 |
| | | | 3p | | | | | |
| rs1982073 | TGFB1 | CDS | hsa-miR-877 | C/T | C | 104 | C/T | −26.75% |
| rs1982073 | TGFB1 | CDS | hsa-miR-885- | C/T | C | 160 | C/T | −18.97% |
| | | | 3p | | | | | |
| rs1982073 | TGFB1 | CDS | hsa-miR-887 | C/T | C | 111 | C/T | −22.31% |
| rs1982073 | TGFB1 | CDS | hsa-miR-921 | C/T | C | 85 | C/T | −44.09% |
| rs1982073 | TGFB1 | CDS | hsa-miR-922 | C/T | T | 134 | C/T | 21.97% |
| rs1982073 | TGFB1 | CDS | hsa-miR-92a | C/T | C | 88 | C/T | −11.91% |
| rs1982073 | TGFB1 | CDS | hsa-miR-93* | C/T | C | 93 | C/T | −37.57% |
| rs1982073 | TGFB1 | CDS | hsa-miR-936 | C/T | C | 137 | C/T | −37.76% |
| rs1982073 | TGFB1 | CDS | hsa-miR-939 | C/T | C | 136 | C/T | −9.57% |
| rs1982073 | TGFB1 | CDS | hsa-miR-940 | C/T | T | 110 | C/T | 9.74% |
| rs1982073 | TGFB1 | CDS | hsa-miR-941 | C/T | C | 107 | C/T | −13.61% |
| rs10735810 | VDR | CDS | hsa-let-7i* | A/G | G | 107 | A/G | 9.98% |
| rs10735810 | VDR | CDS | hsa-miR-103- | A/G | G | 129 | A/G | 33.20% |
| | | | as | | | | | |
| rs10735810 | VDR | CDS | hsa-miR- | A/G | G | 118 | A/G | 143.04 |
| | | | 1180 | | | | | |
| rs10735810 | VDR | CDS | hsa-miR- | A/G | G | 139 | A/G | 8.00% |
| | | | 1181 | | | | | |
| rs10735810 | VDR | CDS | hsa-miR- | A/G | A | 135 | A/G | −25.35% |
| | | | 1204 | | | | | |
| rs10735810 | VDR | CDS | hsa-miR- | A/G | G | 97 | A/G | 17.94% |
| | | | 1208 | | | | | |
| rs10735810 | VDR | CDS | hsa-miR-122 | A/G | G | 109 | A/G | 20.54% |
| rs10735810 | VDR | CDS | hsa-miR- | A/G | G | 87 | A/G | 55.53% |
| | | | 1225-5p | | | | | |
| rs10735810 | VDR | CDS | hsa-miR- | A/G | G | 108 | A/G | 39.07% |
| | | | 1226 | | | | | |
| rs10735810 | VDR | CDS | hsa-miR- | A/G | A | 111 | A/G | −23.20% |
| | | | 1234 | | | | | |
| rs10735810 | VDR | CDS | hsa-miR- | A/G | G | 102 | A/G | 9.85% |
| | | | 1237 | | | | | |
| rs10735810 | VDR | CDS | hsa-miR- | A/G | G | 143 | A/G | 12.79% |
| | | | 1247 | | | | | |
| rs10735810 | VDR | CDS | hsa-miR- | A/G | G | 105 | A/G | 24.90% |
| | | | 1269 | | | | | |
| rs10735810 | VDR | CDS | hsa-miR-127- | A/G | G | 93 | A/G | 13.50% |
| | | | 3p | | | | | |
| rs10735810 | VDR | CDS | hsa-miR- | A/G | G | 140 | A/G | 12.73% |
| | | | 1274a | | | | | |
| rs10735810 | VDR | CDS | hsa-miR- | A/G | G | 128 | A/G | 19.95% |
| | | | 1295 | | | | | |
| rs10735810 | VDR | CDS | hsa-miR- | A/G | A | 131 | A/G | −24.14% |
| | | | 1296 | | | | | |
| rs10735810 | VDR | CDS | hsa-miR- | A/G | G | 98 | A/G | 38.65% |
| | | | 1301 | | | | | |
| rs10735810 | VDR | CDS | hsa-miR- | A/G | A | 95 | A/G | −12.93% |
| | | | 1305 | | | | | |
| rs10735810 | VDR | CDS | hsa-miR- | A/G | G | 109 | A/G | 14.48% |
| | | | 1307 | | | | | |
| rs10735810 | VDR | CDS | hsa-miR- | A/G | G | 94 | A/G | 13.19% |
| | | | 130a* | | | | | |
| rs10735810 | VDR | CDS | hsa-miR- | A/G | G | 116 | A/G | 10.53% |
| | | | 132* | | | | | |
| rs10735810 | VDR | CDS | hsa-miR- | A/G | A | 105 | A/G | −48.04% |
| | | | 133a | | | | | |
| rs10735810 | VDR | CDS | hsa-miR- | A/G | A | 102 | A/G | −45.14% |
| | | | 133b | | | | | |
| rs10735810 | VDR | CDS | hsa-miR-134 | A/G | G | 97 | A/G | 22.49% |
| rs10735810 | VDR | CDS | hsa-miR- | A/G | G | 101 | A/G | 12.70% |
| | | | 135b | | | | | |
| rs10735810 | VDR | CDS | hsa-miR-138- | A/G | A | 99 | A/G | −10.27% |
| | | | 1* | | | | | |
| rs10735810 | VDR | CDS | hsa-miR- | A/G | A | 110 | A/G | −14.23% |
| | | | 143* | | | | | |
| rs10735810 | VDR | CDS | hsa-miR- | A/G | A | 103 | A/G | −11.45% |
| | | | 1468 | | | | | |
| rs10735810 | VDR | CDS | hsa-miR-147 | A/G | G | 112 | A/G | 10.17% |
| rs10735810 | VDR | CDS | hsa-miR- | A/G | G | 153 | A/G | 10.31% |
| | | | 1470 | | | | | |
| rs10735810 | VDR | CDS | hsa-miR- | A/G | G | 114 | A/G | 41.38% |
| | | | 1471 | | | | | |
| rs10735810 | VDR | CDS | hsa-miR- | A/G | G | 123 | A/G | 16.10% |
| | | | 148a* | | | | | |
| rs10735810 | VDR | CDS | hsa-miR-149 | A/G | A | 146 | A/G | −9.66% |
| rs10735810 | VDR | CDS | hsa-miR-150 | A/G | A | 96 | A/G | −22.15% |
| rs10735810 | VDR | CDS | hsa-miR- | A/G | G | 118 | A/G | 20.52% |
| | | | 1538 | | | | | |
| rs10735810 | VDR | CDS | hsa-miR- | A/G | G | 98 | A/G | 72.72% |
| | | | 181a-2* | | | | | |
| rs10735810 | VDR | CDS | hsa-miR- | A/G | A | 90 | A/G | −26.19% |
| | | | 181d | | | | | |
| rs10735810 | VDR | CDS | hsa-miR- | A/G | G | 136 | A/G | 18.11% |
| | | | 185* | | | | | |
| rs10735810 | VDR | CDS | hsa-miR-187 | A/G | G | 114 | A/G | 18.24% |
| rs10735810 | VDR | CDS | hsa-miR-188- | A/G | A | 142 | A/G | −26.19% |
| | | | 5p | | | | | |
| rs10735810 | VDR | CDS | hsa-miR- | A/G | G | 130 | A/G | 12.59% |
| | | | 1909* | | | | | |
| rs10735810 | VDR | CDS | hsa-miR- | A/G | A | 92 | A/G | −36.29% |
| | | | 1911 | | | | | |
| rs10735810 | VDR | CDS | hsa-miR- | A/G | A | 113 | A/G | −14.39% |
| | | | 192* | | | | | |
| rs10735810 | VDR | CDS | hsa-miR- | A/G | G | 159 | A/G | 11.16% |
| | | | 193b | | | | | |
| rs10735810 | VDR | CDS | hsa-miR-197 | A/G | A | 97 | A/G | −14.95% |
| rs10735810 | VDR | CDS | hsa-miR- | A/G | G | 110 | A/G | 39.69% |
| | | | 1973 | | | | | |
| rs10735810 | VDR | CDS | hsa-miR- | A/G | G | 83 | A/G | 58.10% |
| | | | 1975 | | | | | |
| rs10735810 | VDR | CDS | hsa-miR-205 | A/G | A | 138 | A/G | −14.29% |
| rs10735810 | VDR | CDS | hsa-miR-21* | A/G | G | 84 | A/G | 19.99% |
| rs10735810 | VDR | CDS | hsa-miR- | A/G | G | 110 | A/G | 25.58% |
| | | | 2113 | | | | | |
| rs10735810 | VDR | CDS | hsa-miR- | A/G | A | 116 | A/G | −28.06% |
| | | | 214* | | | | | |
| rs10735810 | VDR | CDS | hsa-miR-218- | A/G | G | 125 | A/G | 11.97% |
| | | | 1* | | | | | |
| rs10735810 | VDR | CDS | hsa-miR-219- | A/G | G | 88 | A/G | 61.00% |
| | | | 1-3p | | | | | |
| rs10735810 | VDR | CDS | hsa-miR-22 | A/G | G | 130 | A/G | 23.58% |
| rs10735810 | VDR | CDS | hsa-miR- | A/G | G | 111 | A/G | 11.66% |
| | | | 220b | | | | | |
| rs10735810 | VDR | CDS | hsa-miR-222 | A/G | A | 98 | A/G | −110.03 |
| rs10735810 | VDR | CDS | hsa-miR-224 | A/G | G | 94 | A/G | 12.94% |
| rs10735810 | VDR | CDS | hsa-miR- | A/G | G | 111 | A/G | 12.14% |
| | | | 23a* | | | | | |
| rs10735810 | VDR | CDS | hsa-miR-24 | A/G | G | 127 | A/G | 10.51% |
| rs10735810 | VDR | CDS | hsa-miR-24- | A/G | G | 107 | A/G | 23.47% |
| | | | 1* | | | | | |
| rs10735810 | VDR | CDS | hsa-miR-24- | A/G | G | 113 | A/G | 21.26% |
| | | | 2* | | | | | |
| rs10735810 | VDR | CDS | hsa-miR-25 | A/G | G | 116 | A/G | 26.32% |
| rs10735810 | VDR | CDS | hsa-miR- | A/G | G | 148 | A/G | 10.29% |
| | | | 302a | | | | | |
| rs10735810 | VDR | CDS | hsa-miR- | A/G | G | 145 | A/G | 18.58% |
| | | | 302d | | | | | |
| rs10735810 | VDR | CDS | hsa-miR-302f | A/G | A | 121 | A/G | −39.63% |
| rs10735810 | VDR | CDS | hsa-miR-31* | A/G | A | 110 | A/G | −30.72% |
| rs10735810 | VDR | CDS | hsa-miR-323- | A/G | G | 118 | A/G | 14.62% |
| | | | 5p | | | | | |
| rs10735810 | VDR | CDS | hsa-miR-324- | A/G | G | 97 | A/G | 22.45% |
| | | | 3p | | | | | |
| rs10735810 | VDR | CDS | hsa-miR-324- | A/G | A | 144 | A/G | −25.30% |
| | | | 5p | | | | | |
| rs10735810 | VDR | CDS | hsa-miR-331- | A/G | A | 85 | A/G | −34.59% |
| | | | 3p | | | | | |
| rs10735810 | VDR | CDS | hsa-miR-339- | A/G | A | 108 | A/G | −9.96% |
| | | | 3p | | | | | |
| rs10735810 | VDR | CDS | hsa-miR-339- | A/G | G | 132 | A/G | 21.28% |
| | | | 5p | | | | | |
| rs10735810 | VDR | CDS | hsa-miR- | A/G | G | 143 | A/G | 15.45% |
| | | | 340* | | | | | |
| rs10735810 | VDR | CDS | hsa-miR-362- | A/G | A | 137 | A/G | −9.08% |
| | | | 5p | | | | | |
| rs10735810 | VDR | CDS | hsa-miR- | A/G | A | 96 | A/G | −14.12% |
| | | | 377* | | | | | |
| rs10735810 | VDR | CDS | hsa-miR-409- | A/G | A | 96 | A/G | −11.42% |
| | | | 3p | | | | | |
| rs10735810 | VDR | CDS | hsa-miR-423- | A/G | G | 108 | A/G | 41.32% |
| | | | 3p | | | | | |
| rs10735810 | VDR | CDS | hsa-miR- | A/G | A | 105 | A/G | −55.08% |
| | | | 432* | | | | | |
| rs10735810 | VDR | CDS | hsa-miR- | A/G | A | 82 | A/G | −27.69% |
| | | | 450b-3p | | | | | |
| rs10735810 | VDR | CDS | hsa-miR- | A/G | G | 88 | A/G | 33.61% |
| | | | 450b-5p | | | | | |
| rs10735810 | VDR | CDS | hsa-miR-451 | A/G | A | 102 | A/G | −51.29% |
| rs10735810 | VDR | CDS | hsa-miR-453 | A/G | G | 103 | A/G | 16.84% |
| rs10735810 | VDR | CDS | hsa-miR-489 | A/G | A | 94 | A/G | −10.30% |
| rs10735810 | VDR | CDS | hsa-miR-490- | A/G | A | 85 | A/G | −11.83% |
| | | | 3p | | | | | |
| rs10735810 | VDR | CDS | hsa-miR-501- | A/G | G | 115 | A/G | 14.20% |
| | | | 5p | | | | | |
| rs10735810 | VDR | CDS | hsa-miR-515- | A/G | A | 104 | A/G | −9.94% |
| | | | 5p | | | | | |
| rs10735810 | VDR | CDS | hsa-miR- | A/G | A | 100 | A/G | −116.08 |
| | | | 517b | | | | | |
| rs10735810 | VDR | CDS | hsa-miR- | A/G | G | 149 | A/G | 10.29% |
| | | | 520a-3p | | | | | |
| rs10735810 | VDR | CDS | hsa-miR-524- | A/G | A | 102 | A/G | −21.99% |
| | | | 3p | | | | | |
| rs10735810 | VDR | CDS | hsa-miR-550 | A/G | G | 155 | A/G | 9.30% |
| rs10735810 | VDR | CDS | hsa-miR-574- | A/G | A | 99 | A/G | −15.51% |
| | | | 3p | | | | | |
| rs10735810 | VDR | CDS | hsa-miR-593 | A/G | G | 140 | A/G | 27.74% |
| rs10735810 | VDR | CDS | hsa-miR- | A/G | A | 109 | A/G | −10.99% |
| | | | 593* | | | | | |
| rs10735810 | VDR | CDS | hsa-miR-598 | A/G | G | 127 | A/G | 9.37% |
| rs10735810 | VDR | CDS | hsa-miR-602 | A/G | G | 93 | A/G | 10.54% |
| rs10735810 | VDR | CDS | hsa-miR-608 | A/G | G | 110 | A/G | 10.81% |
| rs10735810 | VDR | CDS | hsa-miR-612 | A/G | G | 116 | A/G | 29.52% |
| rs10735810 | VDR | CDS | hsa-miR-615- | A/G | G | 97 | A/G | 15.28% |
| | | | 5p | | | | | |
| rs10735810 | VDR | CDS | hsa-miR-623 | A/G | A | 136 | A/G | −8.12% |
| rs10735810 | VDR | CDS | hsa-miR-634 | A/G | A | 89 | A/G | −13.28% |
| rs10735810 | VDR | CDS | hsa-miR-636 | A/G | G | 140 | A/G | 21.23% |
| rs10735810 | VDR | CDS | hsa-miR-637 | A/G | G | 112 | A/G | 8.03% |
| rs10735810 | VDR | CDS | hsa-miR-646 | A/G | A | 102 | A/G | −11.34% |
| rs10735810 | VDR | CDS | hsa-miR-652 | A/G | G | 115 | A/G | 34.69% |
| rs10735810 | VDR | CDS | hsa-miR-660 | A/G | A | 120 | A/G | −20.24% |
| rs10735810 | VDR | CDS | hsa-miR-661 | A/G | G | 119 | A/G | 24.90% |
| rs10735810 | VDR | CDS | hsa-miR- | A/G | G | 134 | A/G | 33.51% |
| | | | 663b | | | | | |
| rs10735810 | VDR | CDS | hsa-miR-671- | A/G | G | 105 | A/G | 13.62% |
| | | | 3p | | | | | |
| rs10735810 | VDR | CDS | hsa-miR-671- | A/G | A | 120 | A/G | −8.16% |
| | | | 5p | | | | | |
| rs10735810 | VDR | CDS | hsa-miR- | A/G | G | 90 | A/G | 32.51% |
| | | | 675* | | | | | |
| rs10735810 | VDR | CDS | hsa-miR-769- | A/G | G | 70 | A/G | 25.92% |
| | | | 3p | | | | | |
| rs10735810 | VDR | CDS | hsa-miR-769- | A/G | G | 85 | A/G | 29.40% |
| | | | 5p | | | | | |
| rs10735810 | VDR | CDS | hsa-miR-770- | A/G | G | 105 | A/G | 13.69% |
| | | | 5p | | | | | |
| rs10735810 | VDR | CDS | hsa-miR-874 | A/G | G | 140 | A/G | 55.97% |
| rs10735810 | VDR | CDS | hsa-miR-885- | A/G | A | 128 | A/G | −9.07% |
| | | | 5p | | | | | |
| rs10735810 | VDR | CDS | hsa-miR-886- | A/G | G | 103 | A/G | 24.19% |
| | | | 3p | | | | | |
| rs10735810 | VDR | CDS | hsa-miR- | A/G | G | 125 | A/G | 29.61% |
| | | | 892b | | | | | |
| rs10735810 | VDR | CDS | hsa-miR-92a | A/G | G | 115 | A/G | 24.25% |
| rs10735810 | VDR | CDS | hsa-miR-92a- | A/G | G | 94 | A/G | 18.37% |
| | | | 1* | | | | | |
| rs10735810 | VDR | CDS | hsa-miR-92b | A/G | G | 121 | A/G | 9.45% |
| rs10735810 | VDR | CDS | hsa-miR-937 | A/G | G | 115 | A/G | 16.24% |
| rs10735810 | VDR | CDS | hsa-miR-939 | A/G | G | 98 | A/G | 40.03% |
| rs10735810 | VDR | CDS | hsa-miR-940 | A/G | G | 121 | A/G | 30.45% |
| rs731236 | VDR | CDS | hsa-miR- | C/T | C | 113 | C/T | −21.52% |
| | | | 105* | | | | | |
| rs731236 | VDR | CDS | hsa-miR- | C/T | C | 115 | C/T | −14.75% |
| | | | 1182 | | | | | |
| rs731236 | VDR | CDS | hsa-miR- | C/T | C | 92 | C/T | −26.91% |
| | | | 1183 | | | | | |
| rs731236 | VDR | CDS | hsa-miR- | C/T | C | 122 | C/T | −17.82% |
| | | | 1201 | | | | | |
| rs731236 | VDR | CDS | hsa-miR- | C/T | C | 121 | C/T | −11.01% |
| | | | 1203 | | | | | |
| rs731236 | VDR | CDS | hsa-miR- | C/T | T | 124 | C/T | 26.55% |
| | | | 1204 | | | | | |
| rs731236 | VDR | CDS | hsa-miR-122 | C/T | C | 79 | C/T | −8.80% |
| rs731236 | VDR | CDS | hsa-miR- | C/T | C | 113 | C/T | −18.40% |
| | | | 1231 | | | | | |
| rs731236 | VDR | CDS | hsa-miR- | C/T | C | 128 | C/T | −10.74% |
| | | | 1238 | | | | | |
| rs731236 | VDR | CDS | hsa-miR- | C/T | C | 90 | C/T | −29.83% |
| | | | 124* | | | | | |
| rs731236 | VDR | CDS | hsa-miR- | C/T | T | 92 | C/T | 12.57% |
| | | | 1263 | | | | | |
| rs731236 | VDR | CDS | hsa-miR- | C/T | C | 106 | C/T | −46.17% |
| | | | 1265 | | | | | |
| rs731236 | VDR | CDS | hsa-miR- | C/T | C | 119 | C/T | −15.77% |
| | | | 1288 | | | | | |
| rs731236 | VDR | CDS | hsa-miR- | C/T | C | 106 | C/T | −14.02% |
| | | | 1289 | | | | | |
| rs731236 | VDR | CDS | hsa-miR- | C/T | C | 86 | C/T | −26.02% |
| | | | 1290 | | | | | |
| rs731236 | VDR | CDS | hsa-miR- | C/T | C | 139 | C/T | −8.27% |
| | | | 1291 | | | | | |
| rs731236 | VDR | CDS | hsa-miR- | C/T | C | 142 | C/T | −9.66% |
| | | | 1293 | | | | | |
| rs731236 | VDR | CDS | hsa-miR- | C/T | T | 106 | C/T | 13.49% |
| | | | 1304 | | | | | |
| rs731236 | VDR | CDS | hsa-miR- | C/T | C | 114 | C/T | −15.66% |
| | | | 1308 | | | | | |
| rs731236 | VDR | CDS | hsa-miR- | C/T | T | 134 | C/T | 23.18% |
| | | | 133a | | | | | |
| rs731236 | VDR | CDS | hsa-miR- | C/T | T | 134 | C/T | 18.55% |
| | | | 133b | | | | | |
| rs731236 | VDR | CDS | hsa-miR-138 | C/T | T | 126 | C/T | 8.69% |
| rs731236 | VDR | CDS | hsa-miR-140- | C/T | C | 89 | C/T | −56.27% |
| | | | 3p | | | | | |
| rs731236 | VDR | CDS | hsa-miR- | C/T | T | 112 | C/T | 15.93% |
| | | | 146b-3p | | | | | |
| rs731236 | VDR | CDS | hsa-miR-149 | C/T | C | 118 | C/T | −9.80% |
| rs731236 | VDR | CDS | hsa-miR-150 | C/T | T | 81 | C/T | 66.80% |
| rs731236 | VDR | CDS | hsa-miR-154 | C/T | C | 94 | C/T | −8.59% |
| rs731236 | VDR | CDS | hsa-miR- | C/T | C | 110 | C/T | −10.75% |
| | | | 181a* | | | | | |
| rs731236 | VDR | CDS | hsa-miR- | C/T | C | 88 | C/T | −13.58% |
| | | | 181a-2* | | | | | |
| rs731236 | VDR | CDS | hsa-miR- | C/T | C | 132 | C/T | −32.39% |
| | | | 1826 | | | | | |
| rs731236 | VDR | CDS | hsa-miR- | C/T | C | 140 | C/T | −9.46% |
| | | | 1908 | | | | | |
| rs731236 | VDR | CDS | hsa-miR- | C/T | T | 108 | C/T | 29.07% |
| | | | 1909* | | | | | |
| rs731236 | VDR | CDS | hsa-miR- | C/T | C | 98 | C/T | −14.03% |
| | | | 1914 | | | | | |
| rs731236 | VDR | CDS | hsa-miR- | C/T | C | 102 | C/T | −11.77% |
| | | | 193b* | | | | | |
| rs731236 | VDR | CDS | hsa-miR- | C/T | C | 149 | C/T | −11.71% |
| | | | 196b* | | | | | |
| rs731236 | VDR | CDS | hsa-miR- | C/T | T | 87 | C/T | 10.85% |
| | | | 1974 | | | | | |
| rs731236 | VDR | CDS | hsa-miR- | C/T | T | 86 | C/T | 81.52% |
| | | | 1975 | | | | | |
| rs731236 | VDR | CDS | hsa-miR-19b- | C/T | T | 98 | C/T | 15.62% |
| | | | 1* | | | | | |
| rs731236 | VDR | CDS | hsa-miR-218- | C/T | T | 79 | C/T | 27.13% |
| | | | 1* | | | | | |
| rs731236 | VDR | CDS | hsa-miR- | C/T | T | 106 | C/T | 76.51% |
| | | | 222* | | | | | |
| rs731236 | VDR | CDS | hsa-miR-224 | C/T | T | 80 | C/T | 28.82% |
| rs731236 | VDR | CDS | hsa-miR- | C/T | C | 120 | C/T | −16.29% |
| | | | 23b* | | | | | |
| rs731236 | VDR | CDS | hsa-miR-24 | C/T | T | 119 | C/T | 52.73% |
| rs731236 | VDR | CDS | hsa-miR-296- | C/T | C | 113 | C/T | −12.37% |
| | | | 5p | | | | | |
| rs731236 | VDR | CDS | hsa-miR-29b- | C/T | T | 97 | C/T | 8.94% |
| | | | 1* | | | | | |
| rs731236 | VDR | CDS | hsa-miR-29b- | C/T | T | 127 | C/T | 8.98% |
| | | | 2* | | | | | |
| rs731236 | VDR | CDS | hsa-miR- | C/T | T | 107 | C/T | 11.54% |
| | | | 302a* | | | | | |
| rs731236 | VDR | CDS | hsa-miR-325 | C/T | T | 84 | C/T | 16.06% |
| rs731236 | VDR | CDS | hsa-miR-339- | C/T | C | 123 | C/T | −34.67% |
| | | | 3p | | | | | |
| rs731236 | VDR | CDS | hsa-miR- | C/T | C | 131 | C/T | −11.42% |
| | | | 33b* | | | | | |
| rs731236 | VDR | CDS | hsa-miR-342- | C/T | T | 101 | C/T | 47.37% |
| | | | 3p | | | | | |
| rs731236 | VDR | CDS | hsa-miR- | C/T | T | 158 | C/T | 25.13% |
| | | | 34a* | | | | | |
| rs731236 | VDR | CDS | hsa-miR- | C/T | T | 114 | C/T | 26.55% |
| | | | 363* | | | | | |
| rs731236 | VDR | CDS | hsa-miR-370 | C/T | C | 98 | C/T | −12.81% |
| rs731236 | VDR | CDS | hsa-miR-371- | C/T | T | 130 | C/T | 14.43% |
| | | | 5p | | | | | |
| rs731236 | VDR | CDS | hsa-miR- | C/T | T | 99 | C/T | 23.62% |
| | | | 373* | | | | | |
| rs731236 | VDR | CDS | hsa-miR-375 | C/T | C | 100 | C/T | −8.64% |
| rs731236 | VDR | CDS | hsa-miR-382 | C/T | C | 91 | C/T | −8.75% |
| rs731236 | VDR | CDS | hsa-miR-383 | C/T | C | 120 | C/T | −16.62% |
| rs731236 | VDR | CDS | hsa-miR-432 | C/T | C | 122 | C/T | −13.58% |
| rs731236 | VDR | CDS | hsa-miR- | C/T | C | 106 | C/T | −69.61% |
| | | | 450b-3p | | | | | |
| rs731236 | VDR | CDS | hsa-miR-453 | C/T | C | 130 | C/T | −12.40% |
| rs731236 | VDR | CDS | hsa-miR-498 | C/T | T | 116 | C/T | 41.92% |
| rs731236 | VDR | CDS | hsa-miR-505 | C/T | T | 102 | C/T | 46.27% |
| rs731236 | VDR | CDS | hsa-miR-509- | C/T | T | 82 | C/T | 17.66% |
| | | | 5p | | | | | |
| rs731236 | VDR | CDS | hsa-miR-510 | C/T | T | 102 | C/T | 17.95% |
| rs731236 | VDR | CDS | hsa-miR- | C/T | T | 100 | C/T | 17.78% |
| | | | 513a-5p | | | | | |
| rs731236 | VDR | CDS | hsa-miR- | C/T | T | 108 | C/T | 21.68% |
| | | | 513c | | | | | |
| rs731236 | VDR | CDS | hsa-miR- | C/T | T | 81 | C/T | 12.11% |
| | | | 518b | | | | | |
| rs731236 | VDR | CDS | hsa-miR- | C/T | T | 80 | C/T | 32.58% |
| | | | 518c | | | | | |
| rs731236 | VDR | CDS | hsa-miR- | C/T | C | 118 | C/T | −12.81% |
| | | | 518c* | | | | | |
| rs731236 | VDR | CDS | hsa-miR- | C/T | C | 85 | C/T | −40.09% |
| | | | 518e* | | | | | |
| rs731236 | VDR | CDS | hsa-miR- | C/T | C | 85 | C/T | −40.09% |
| | | | 519a* | | | | | |
| rs731236 | VDR | CDS | hsa-miR- | C/T | C | 85 | C/T | −40.09% |
| | | | 519b-5p | | | | | |
| rs731236 | VDR | CDS | hsa-miR- | C/T | C | 85 | C/T | −40.09% |
| | | | 519c-5p | | | | | |
| rs731236 | VDR | CDS | hsa-miR- | C/T | C | 85 | C/T | −40.09% |
| | | | 522* | | | | | |
| rs731236 | VDR | CDS | hsa-miR- | C/T | C | 85 | C/T | −40.09% |
| | | | 523* | | | | | |
| rs731236 | VDR | CDS | hsa-miR-532- | C/T | C | 108 | C/T | −21.43% |
| | | | 5p | | | | | |
| rs731236 | VDR | CDS | hsa-miR-543 | C/T | T | 96 | C/T | 26.00% |
| rs731236 | VDR | CDS | hsa-miR- | C/T | T | 80 | C/T | 14.20% |
| | | | 551b | | | | | |
| rs731236 | VDR | CDS | hsa-miR-554 | C/T | T | 113 | C/T | 23.84% |
| rs731236 | VDR | CDS | hsa-miR-574- | C/T | C | 104 | C/T | −42.10% |
| | | | 3p | | | | | |
| rs731236 | VDR | CDS | hsa-miR-581 | C/T | C | 103 | C/T | −11.34% |
| rs731236 | VDR | CDS | hsa-miR- | C/T | C | 105 | C/T | −21.72% |
| | | | 593* | | | | | |
| rs731236 | VDR | CDS | hsa-miR-596 | C/T | C | 90 | C/T | −11.23% |
| rs731236 | VDR | CDS | hsa-miR-600 | C/T | C | 96 | C/T | −65.08% |
| rs731236 | VDR | CDS | hsa-miR-608 | C/T | C | 139 | C/T | −25.06% |
| rs731236 | VDR | CDS | hsa-miR-614 | C/T | T | 87 | C/T | 10.22% |
| rs731236 | VDR | CDS | hsa-miR-615- | C/T | C | 158 | C/T | −10.23% |
| | | | 5p | | | | | |
| rs731236 | VDR | CDS | hsa-miR-616 | C/T | C | 113 | C/T | −18.90% |
| rs731236 | VDR | CDS | hsa-miR-621 | C/T | T | 80 | C/T | 19.66% |
| rs731236 | VDR | CDS | hsa-miR-623 | C/T | C | 105 | C/T | −37.15% |
| rs731236 | VDR | CDS | hsa-miR-650 | C/T | C | 111 | C/T | −10.50% |
| rs731236 | VDR | CDS | hsa-miR-658 | C/T | C | 104 | C/T | −31.30% |
| rs731236 | VDR | CDS | hsa-miR-662 | C/T | T | 74 | C/T | 12.72% |
| rs731236 | VDR | CDS | hsa-miR-668 | C/T | T | 123 | C/T | 10.30% |
| rs731236 | VDR | CDS | hsa-miR-671- | C/T | C | 119 | C/T | −9.14% |
| | | | 5p | | | | | |
| rs731236 | VDR | CDS | hsa-miR- | C/T | T | 112 | C/T | 12.76% |
| | | | 675* | | | | | |
| rs731236 | VDR | CDS | hsa-miR-708 | C/T | T | 106 | C/T | 34.02% |
| rs731236 | VDR | CDS | hsa-miR-765 | C/T | C | 97 | C/T | −21.38% |
| rs731236 | VDR | CDS | hsa-miR-769- | C/T | C | 116 | C/T | −8.21% |
| | | | 3p | | | | | |
| rs731236 | VDR | CDS | hsa-miR-769- | C/T | T | 101 | C/T | 94.42% |
| | | | 5p | | | | | |
| rs731236 | VDR | CDS | hsa-miR-770- | C/T | C | 80 | C/T | −17.55% |
| | | | 5p | | | | | |
| rs731236 | VDR | CDS | hsa-miR-874 | C/T | T | 110 | C/T | 14.63% |
| rs731236 | VDR | CDS | hsa-miR-924 | C/T | C | 106 | C/T | −25.82% |
| rs731236 | VDR | CDS | hsa-miR-92a- | C/T | C | 114 | C/T | −17.31% |
| | | | 1* | | | | | |
| rs731236 | VDR | CDS | hsa-miR-92b | C/T | C | 103 | C/T | −13.21% |
| rs731236 | VDR | CDS | hsa-miR-935 | C/T | T | 94 | C/T | 26.05% |
| rs731236 | VDR | CDS | hsa-miR-938 | C/T | T | 109 | C/T | 105.00 |
| rs4588 | GC | CDS | hsa-miR- | A/C | C | 132 | A/C | 24.43% |
| | | | 1180 | | | | | |
| rs4588 | GC | CDS | hsa-miR- | A/C | C | 111 | A/C | 16.36% |
| | | | 1183 | | | | | |
| rs4588 | GC | CDS | hsa-miR- | A/C | C | 149 | A/C | 8.49% |
| | | | 1202 | | | | | |
| rs4588 | GC | CDS | hsa-miR- | A/C | A | 94 | A/C | −27.51% |
| | | | 125b-2* | | | | | |
| rs4588 | GC | CDS | hsa-miR-127- | A/C | C | 125 | A/C | 31.08% |
| | | | 3p | | | | | |
| rs4588 | GC | CDS | hsa-miR- | A/C | A | 105 | A/C | −9.31% |
| | | | 1307 | | | | | |
| rs4588 | GC | CDS | hsa-miR-134 | A/C | C | 77 | A/C | 22.31% |
| rs4588 | GC | CDS | hsa-miR- | A/C | C | 107 | A/C | 25.51% |
| | | | 1468 | | | | | |
| rs4588 | GC | CDS | hsa-miR- | A/C | C | 98 | A/C | 25.19% |
| | | | 146b-3p | | | | | |
| rs4588 | GC | CDS | hsa-miR- | A/C | C | 106 | A/C | 25.04% |
| | | | 1471 | | | | | |
| rs4588 | GC | CDS | hsa-miR- | A/C | C | 140 | A/C | 30.98% |
| | | | 148b* | | | | | |
| rs4588 | GC | CDS | hsa-miR-152 | A/C | A | 88 | A/C | −33.00% |
| rs4588 | GC | CDS | hsa-miR- | A/C | A | 101 | A/C | −9.65% |
| | | | 1538 | | | | | |
| rs4588 | GC | CDS | hsa-miR- | A/C | C | 81 | A/C | 58.69% |
| | | | 181b | | | | | |
| rs4588 | GC | CDS | hsa-miR- | A/C | C | 105 | A/C | 51.72% |
| | | | 181c* | | | | | |
| rs4588 | GC | CDS | hsa-miR-187 | A/C | C | 106 | A/C | 18.38% |
| rs4588 | GC | CDS | hsa-miR-188- | A/C | A | 104 | A/C | −21.83% |
| | | | 5p | | | | | |
| rs4588 | GC | CDS | hsa-miR- | A/C | C | 125 | A/C | 19.14% |
| | | | 1910 | | | | | |
| rs4588 | GC | CDS | hsa-miR- | A/C | C | 101 | A/C | 36.36% |
| | | | 193b | | | | | |
| rs4588 | GC | CDS | hsa-miR- | A/C | A | 110 | A/C | −8.60% |
| | | | 194* | | | | | |
| rs4588 | GC | CDS | hsa-miR-21* | A/C | C | 96 | A/C | 13.28% |
| rs4588 | GC | CDS | hsa-miR-210 | A/C | C | 134 | A/C | 40.73% |
| rs4588 | GC | CDS | hsa-miR- | A/C | A | 82 | A/C | −209.07 |
| | | | 222* | | | | | |
| rs4588 | GC | CDS | hsa-miR-27a | A/C | C | 91 | A/C | 91.82% |
| rs4588 | GC | CDS | hsa-miR- | A/C | C | 100 | A/C | 26.74% |
| | | | 302c | | | | | |
| rs4588 | GC | CDS | hsa-miR-328 | A/C | C | 93 | A/C | 24.45% |
| rs4588 | GC | CDS | hsa-miR-362- | A/C | A | 132 | A/C | −41.73% |
| | | | 5p | | | | | |
| rs4588 | GC | CDS | hsa-miR-383 | A/C | C | 88 | A/C | 82.34% |
| rs4588 | GC | CDS | hsa-miR-423- | A/C | A | 92 | A/C | −37.68% |
| | | | 3p | | | | | |
| rs4588 | GC | CDS | hsa-miR-432 | A/C | A | 123 | A/C | −16.81% |
| rs4588 | GC | CDS | hsa-miR-497 | A/C | C | 95 | A/C | 66.84% |
| rs4588 | GC | CDS | hsa-miR-502- | A/C | C | 118 | A/C | 22.80% |
| | | | 5p | | | | | |
| rs4588 | GC | CDS | hsa-miR-503 | A/C | C | 104 | A/C | 26.10% |
| rs4588 | GC | CDS | hsa-miR-507 | A/C | A | 110 | A/C | −21.16% |
| rs4588 | GC | CDS | hsa-miR-509- | A/C | C | 90 | A/C | 105.97 |
| | | | 3p | | | | | |
| rs4588 | GC | CDS | hsa-miR-532- | A/C | A | 114 | A/C | −35.95% |
| | | | 5p | | | | | |
| rs4588 | GC | CDS | hsa-miR- | A/C | C | 82 | A/C | 42.32% |
| | | | 551b* | | | | | |
| rs4588 | GC | CDS | hsa-miR-557 | A/C | C | 110 | A/C | 14.36% |
| rs4588 | GC | CDS | hsa-miR-572 | A/C | C | 92 | A/C | 21.64% |
| rs4588 | GC | CDS | hsa-miR-596 | A/C | A | 136 | A/C | −11.71% |
| rs4588 | GC | CDS | hsa-miR-622 | A/C | C | 114 | A/C | 21.21% |
| rs4588 | GC | CDS | hsa-miR-623 | A/C | A | 124 | A/C | −10.89% |
| rs4588 | GC | CDS | hsa-miR- | A/C | A | 108 | A/C | −27.70% |
| | | | 624* | | | | | |
| rs4588 | GC | CDS | hsa-miR-632 | A/C | C | 96 | A/C | 12.26% |
| rs4588 | GC | CDS | hsa-miR-634 | A/C | A | 135 | A/C | −22.50% |
| rs4588 | GC | CDS | hsa-miR-638 | A/C | C | 57 | A/C | 54.15% |
| rs4588 | GC | CDS | hsa-miR-760 | A/C | C | 127 | A/C | 33.47% |
| rs4588 | GC | CDS | hsa-miR-770- | A/C | C | 135 | A/C | 10.24% |
| | | | 5p | | | | | |
| rs4588 | GC | CDS | hsa-miR-874 | A/C | C | 195 | A/C | 102.12 |
| rs4588 | GC | CDS | hsa-miR- | A/C | A | 108 | A/C | −26.95% |
| | | | 888* | | | | | |
| rs4588 | GC | CDS | hsa-miR-890 | A/C | A | 120 | A/C | −24.13% |
| rs4588 | GC | CDS | hsa-miR-93 | A/C | C | 96 | A/C | 14.22% |
| rs4588 | GC | CDS | hsa-miR-935 | A/C | C | 114 | A/C | 37.48% |
| rs4588 | GC | CDS | hsa-miR-939 | A/C | A | 94 | A/C | −9.08% |
| rs4588 | GC | CDS | hsa-miR-941 | A/C | C | 117 | A/C | 17.64% |
| rs4588 | GC | CDS | hsa-miR- | A/C | C | 98 | A/C | 45.56% |
| | | | 99b* | | | | | |
| rs7041 | GC | CDS | hsa-miR- | G/T | G | 128 | G/T | −26.66% |
| | | | 1205 | | | | | |
| rs7041 | GC | CDS | hsa-miR- | G/T | G | 117 | G/T | −25.21% |
| | | | 1224-5p | | | | | |
| rs7041 | GC | CDS | hsa-miR- | G/T | G | 118 | G/T | −28.27% |
| | | | 1225-5p | | | | | |
| rs7041 | GC | CDS | hsa-miR- | G/T | T | 110 | G/T | 15.50% |
| | | | 1226* | | | | | |
| rs7041 | GC | CDS | hsa-miR- | G/T | T | 118 | G/T | 8.68% |
| | | | 1228* | | | | | |
| rs7041 | GC | CDS | hsa-miR- | G/T | G | 117 | G/T | −33.44% |
| | | | 1250 | | | | | |
| rs7041 | GC | CDS | hsa-miR- | G/T | T | 131 | G/T | 17.26% |
| | | | 1262 | | | | | |
| rs7041 | GC | CDS | hsa-miR- | G/T | G | 134 | G/T | −28.30% |
| | | | 1266 | | | | | |
| rs7041 | GC | CDS | hsa-miR- | G/T | G | 96 | G/T | −14.91% |
| | | | 1292 | | | | | |
| rs7041 | GC | CDS | hsa-miR- | G/T | G | 128 | G/T | −21.04% |
| | | | 1301 | | | | | |
| rs7041 | GC | CDS | hsa-miR- | G/T | T | 105 | G/T | 11.41% |
| | | | 1307 | | | | | |
| rs7041 | GC | CDS | hsa-miR- | G/T | G | 130 | G/T | −8.97% |
| | | | 1308 | | | | | |
| rs7041 | GC | CDS | hsa-miR- | G/T | G | 85 | G/T | −19.97% |
| | | | 135a* | | | | | |
| rs7041 | GC | CDS | hsa-miR- | G/T | G | 102 | G/T | −20.86% |
| | | | 146b-3p | | | | | |
| rs7041 | GC | CDS | hsa-miR- | G/T | G | 146 | G/T | −21.72% |
| | | | 148b* | | | | | |
| rs7041 | GC | CDS | hsa-miR- | G/T | G | 137 | G/T | −38.05% |
| | | | 1538 | | | | | |
| rs7041 | GC | CDS | hsa-miR- | G/T | G | 91 | G/T | −32.43% |
| | | | 15a* | | | | | |
| rs7041 | GC | CDS | hsa-miR- | G/T | G | 111 | G/T | −27.33% |
| | | | 185* | | | | | |
| rs7041 | GC | CDS | hsa-miR-18b | G/T | T | 96 | G/T | 11.30% |
| rs7041 | GC | CDS | hsa-miR- | G/T | T | 109 | G/T | 25.96% |
| | | | 1909* | | | | | |
| rs7041 | GC | CDS | hsa-miR- | G/T | G | 134 | G/T | −27.18% |
| | | | 1910 | | | | | |
| rs7041 | GC | CDS | hsa-miR- | G/T | G | 91 | G/T | −28.22% |
| | | | 1911* | | | | | |
| rs7041 | GC | CDS | hsa-miR- | G/T | T | 120 | G/T | 8.52% |
| | | | 1915 | | | | | |
| rs7041 | GC | CDS | hsa-miR-19b- | G/T | T | 137 | G/T | 51.99% |
| | | | 1* | | | | | |
| rs7041 | GC | CDS | hsa-miR-219- | G/T | T | 81 | G/T | 64.93% |
| | | | 2-3p | | | | | |
| rs7041 | GC | CDS | hsa-miR- | G/T | T | 123 | G/T | 13.26% |
| | | | 23b* | | | | | |
| rs7041 | GC | CDS | hsa-miR-25* | G/T | G | 101 | G/T | −26.30% |
| rs7041 | GC | CDS | hsa-miR-296- | G/T | G | 115 | G/T | −18.28% |
| | | | 3p | | | | | |
| rs7041 | GC | CDS | hsa-miR-323- | G/T | G | 132 | G/T | −36.14% |
| | | | 5p | | | | | |
| rs7041 | GC | CDS | hsa-miR-330- | G/T | G | 138 | G/T | −21.16% |
| | | | 5p | | | | | |
| rs7041 | GC | CDS | hsa-miR-331- | G/T | G | 110 | G/T | −21.64% |
| | | | 5p | | | | | |
| rs7041 | GC | CDS | hsa-miR-338- | G/T | T | 118 | G/T | 41.82% |
| | | | 3p | | | | | |
| rs7041 | GC | CDS | hsa-miR- | G/T | G | 123 | G/T | −49.71% |
| | | | 33b* | | | | | |
| rs7041 | GC | CDS | hsa-miR-346 | G/T | G | 110 | G/T | −74.35% |
| rs7041 | GC | CDS | hsa-miR-370 | G/T | G | 103 | G/T | −14.26% |
| rs7041 | GC | CDS | hsa-miR-382 | G/T | G | 97 | G/T | −14.19% |
| rs7041 | GC | CDS | hsa-miR-423- | G/T | G | 112 | G/T | −18.98% |
| | | | 3p | | | | | |
| rs7041 | GC | CDS | hsa-miR- | G/T | G | 95 | G/T | −33.31% |
| | | | 431* | | | | | |
| rs7041 | GC | CDS | hsa-miR-453 | G/T | T | 106 | G/T | 38.62% |
| rs7041 | GC | CDS | hsa-miR-508- | G/T | T | 103 | G/T | 16.82% |
| | | | 5p | | | | | |
| rs7041 | GC | CDS | hsa-miR-541 | G/T | T | 148 | G/T | 9.05% |
| rs7041 | GC | CDS | hsa-miR- | G/T | G | 88 | G/T | −15.14% |
| | | | 548b-3p | | | | | |
| rs7041 | GC | CDS | hsa-miR-554 | G/T | G | 118 | G/T | −25.45% |
| rs7041 | GC | CDS | hsa-miR-557 | G/T | G | 119 | G/T | −28.37% |
| rs7041 | GC | CDS | hsa-miR-572 | G/T | G | 100 | G/T | −25.35% |
| rs7041 | GC | CDS | hsa-miR-596 | G/T | G | 99 | G/T | −33.15% |
| rs7041 | GC | CDS | hsa-miR-611 | G/T | G | 84 | G/T | −32.14% |
| rs7041 | GC | CDS | hsa-miR-614 | G/T | T | 92 | G/T | 34.84% |
| rs7041 | GC | CDS | hsa-miR-615- | G/T | G | 94 | G/T | −64.41% |
| | | | 3p | | | | | |
| rs7041 | GC | CDS | hsa-miR-638 | G/T | G | 82 | G/T | −39.36% |
| rs7041 | GC | CDS | hsa-miR-644 | G/T | G | 164 | G/T | −16.72% |
| rs7041 | GC | CDS | hsa-miR-654- | G/T | G | 153 | G/T | −20.35% |
| | | | 5p | | | | | |
| rs7041 | GC | CDS | hsa-miR-661 | G/T | G | 128 | G/T | −31.83% |
| rs7041 | GC | CDS | hsa-miR-662 | G/T | G | 108 | G/T | −33.29% |
| rs7041 | GC | CDS | hsa-miR-663 | G/T | G | 113 | G/T | −9.31% |
| rs7041 | GC | CDS | hsa-miR- | G/T | G | 140 | G/T | −38.80% |
| | | | 663b | | | | | |
| rs7041 | GC | CDS | hsa-miR-665 | G/T | G | 105 | G/T | −31.06% |
| rs7041 | GC | CDS | hsa-miR-766 | G/T | G | 96 | G/T | −184.03 |
| rs7041 | GC | CDS | hsa-miR-770- | G/T | G | 141 | G/T | −24.93% |
| | | | 5p | | | | | |
| rs7041 | GC | CDS | hsa-miR-874 | G/T | T | 95 | G/T | 47.85% |
| rs7041 | GC | CDS | hsa-miR-885- | G/T | G | 103 | G/T | −37.67% |
| | | | 3p | | | | | |
| rs7041 | GC | CDS | hsa-miR-890 | G/T | T | 92 | G/T | 42.55% |
| rs7041 | GC | CDS | hsa-miR-92a- | G/T | T | 122 | G/T | 15.68% |
| | | | 1* | | | | | |
| rs7041 | GC | CDS | hsa-miR-92a- | G/T | T | 140 | G/T | 21.47% |
| | | | 2* | | | | | |
| rs7041 | GC | CDS | hsa-miR-92b | G/T | G | 92 | G/T | −79.74% |
| rs1799782 | XRCC1 | CDS | hsa-let-7a-2* | C/T | T | 83 | C/T | 18.21% |
| rs1799782 | XRCC1 | CDS | hsa-miR- | C/T | T | 143 | C/T | 30.16% |
| | | | 1202 | | | | | |
| rs1799782 | XRCC1 | CDS | hsa-miR- | C/T | C | 96 | C/T | −32.74% |
| | | | 1207-5p | | | | | |
| rs1799782 | XRCC1 | CDS | hsa-miR- | C/T | T | 98 | C/T | 9.51% |
| | | | 1225-5p | | | | | |
| rs1799782 | XRCC1 | CDS | hsa-miR- | C/T | C | 86 | C/T | −53.37% |
| | | | 1228* | | | | | |
| rs1799782 | XRCC1 | CDS | hsa-miR- | C/T | T | 136 | C/T | 27.02% |
| | | | 1231 | | | | | |
| rs1799782 | XRCC1 | CDS | hsa-miR- | C/T | T | 105 | C/T | 12.57% |
| | | | 1254 | | | | | |
| rs1799782 | XRCC1 | CDS | hsa-miR- | C/T | T | 130 | C/T | 18.70% |
| | | | 125a-3p | | | | | |
| rs1799782 | XRCC1 | CDS | hsa-miR- | C/T | C | 96 | C/T | −47.67% |
| | | | 125b-1* | | | | | |
| rs1799782 | XRCC1 | CDS | hsa-miR- | C/T | C | 96 | C/T | −101.14 |
| | | | 1269 | | | | | |
| rs1799782 | XRCC1 | CDS | hsa-miR-127- | C/T | C | 127 | C/T | −9.80% |
| | | | 3p | | | | | |
| rs1799782 | XRCC1 | CDS | hsa-miR-128 | C/T | T | 92 | C/T | 26.05% |
| rs1799782 | XRCC1 | CDS | hsa-miR- | C/T | C | 121 | C/T | −11.18% |
| | | | 1292 | | | | | |
| rs1799782 | XRCC1 | CDS | hsa-miR- | C/T | C | 114 | C/T | −27.94% |
| | | | 1293 | | | | | |
| rs1799782 | XRCC1 | CDS | hsa-miR- | C/T | C | 114 | C/T | −20.50% |
| | | | 1298 | | | | | |
| rs1799782 | XRCC1 | CDS | hsa-miR- | C/T | T | 133 | C/T | 32.23% |
| | | | 1301 | | | | | |
| rs1799782 | XRCC1 | CDS | hsa-miR-138 | C/T | T | 125 | C/T | 9.58% |
| rs1799782 | XRCC1 | CDS | hsa-miR- | C/T | T | 81 | C/T | 10.81% |
| | | | 143* | | | | | |
| rs1799782 | XRCC1 | CDS | hsa-miR-145 | C/T | T | 132 | C/T | 35.62% |
| rs1799782 | XRCC1 | CDS | hsa-miR- | C/T | C | 108 | C/T | −17.47% |
| | | | 1469 | | | | | |
| rs1799782 | XRCC1 | CDS | hsa-miR-149 | C/T | C | 116 | C/T | −48.14% |
| rs1799782 | XRCC1 | CDS | hsa-miR-151- | C/T | T | 87 | C/T | 18.93% |
| | | | 3p | | | | | |
| rs1799782 | XRCC1 | CDS | hsa-miR- | C/T | C | 101 | C/T | −17.20% |
| | | | 187* | | | | | |
| rs1799782 | XRCC1 | CDS | hsa-miR- | C/T | C | 113 | C/T | −13.85% |
| | | | 1908 | | | | | |
| rs1799782 | XRCC1 | CDS | hsa-miR- | C/T | C | 95 | C/T | −26.32% |
| | | | 191* | | | | | |
| rs1799782 | XRCC1 | CDS | hsa-miR- | C/T | C | 125 | C/T | −15.77% |
| | | | 194* | | | | | |
| rs1799782 | XRCC1 | CDS | hsa-miR- | C/T | C | 101 | C/T | −25.92% |
| | | | 196a* | | | | | |
| rs1799782 | XRCC1 | CDS | hsa-miR-198 | C/T | T | 121 | C/T | 22.56% |
| rs1799782 | XRCC1 | CDS | hsa-miR-22 | C/T | T | 104 | C/T | 102.69 |
| rs1799782 | XRCC1 | CDS | hsa-miR-25* | C/T | C | 117 | C/T | −22.17% |
| rs1799782 | XRCC1 | CDS | hsa-miR-296- | C/T | C | 115 | C/T | −54.18% |
| | | | 3p | | | | | |
| rs1799782 | XRCC1 | CDS | hsa-miR-298 | C/T | T | 155 | C/T | 8.02% |
| rs1799782 | XRCC1 | CDS | hsa-miR- | C/T | T | 125 | C/T | 8.34% |
| | | | 320a | | | | | |
| rs1799782 | XRCC1 | CDS | hsa-miR- | C/T | T | 125 | C/T | 8.18% |
| | | | 320b | | | | | |
| rs1799782 | XRCC1 | CDS | hsa-miR- | C/T | T | 122 | C/T | 9.51% |
| | | | 320c | | | | | |
| rs1799782 | XRCC1 | CDS | hsa-miR- | C/T | T | 122 | C/T | 10.03% |
| | | | 320d | | | | | |
| rs1799782 | XRCC1 | CDS | hsa-miR-323- | C/T | C | 109 | C/T | −13.35% |
| | | | 5p | | | | | |
| rs1799782 | XRCC1 | CDS | hsa-miR-34a | C/T | C | 101 | C/T | −16.83% |
| rs1799782 | XRCC1 | CDS | hsa-miR- | C/T | T | 119 | C/T | 41.91% |
| | | | 365* | | | | | |
| rs1799782 | XRCC1 | CDS | hsa-miR-375 | C/T | C | 108 | C/T | −18.25% |
| rs1799782 | XRCC1 | CDS | hsa-miR-409- | C/T | C | 119 | C/T | −16.39% |
| | | | 3p | | | | | |
| rs1799782 | XRCC1 | CDS | hsa-miR- | C/T | C | 114 | C/T | −29.52% |
| | | | 425* | | | | | |
| rs1799782 | XRCC1 | CDS | hsa-miR-431 | C/T | C | 131 | C/T | −10.02% |
| rs1799782 | XRCC1 | CDS | hsa-miR- | C/T | C | 104 | C/T | −24.08% |
| | | | 449b | | | | | |
| rs1799782 | XRCC1 | CDS | hsa-miR-484 | C/T | C | 110 | C/T | −19.54% |
| rs1799782 | XRCC1 | CDS | hsa-miR-512- | C/T | T | 104 | C/T | 59.04% |
| | | | 3p | | | | | |
| rs1799782 | XRCC1 | CDS | hsa-miR-550 | C/T | C | 124 | C/T | −9.60% |
| rs1799782 | XRCC1 | CDS | hsa-miR-575 | C/T | T | 129 | C/T | 32.71% |
| rs1799782 | XRCC1 | CDS | hsa-miR-601 | C/T | T | 126 | C/T | 14.74% |
| rs1799782 | XRCC1 | CDS | hsa-miR-602 | C/T | T | 105 | C/T | 16.28% |
| rs1799782 | XRCC1 | CDS | hsa-miR-608 | C/T | T | 134 | C/T | 23.39% |
| rs1799782 | XRCC1 | CDS | hsa-miR-619 | C/T | T | 110 | C/T | 31.09% |
| rs1799782 | XRCC1 | CDS | hsa-miR-658 | C/T | C | 132 | C/T | −12.37% |
| rs1799782 | XRCC1 | CDS | hsa-miR-659 | C/T | T | 132 | C/T | 8.98% |
| rs1799782 | XRCC1 | CDS | hsa-miR-665 | C/T | T | 126 | C/T | 25.28% |
| rs1799782 | XRCC1 | CDS | hsa-miR-671- | C/T | C | 119 | C/T | −14.48% |
| | | | 3p | | | | | |
| rs1799782 | XRCC1 | CDS | hsa-miR-675 | C/T | C | 134 | C/T | −11.48% |
| rs1799782 | XRCC1 | CDS | hsa-miR-708 | C/T | T | 91 | C/T | 24.48% |
| rs1799782 | XRCC1 | CDS | hsa-miR-760 | C/T | T | 92 | C/T | 11.03% |
| rs1799782 | XRCC1 | CDS | hsa-miR-766 | C/T | T | 144 | C/T | 25.74% |
| rs1799782 | XRCC1 | CDS | hsa-miR-767- | C/T | C | 97 | C/T | −9.65% |
| | | | 3p | | | | | |
| rs1799782 | XRCC1 | CDS | hsa-miR-770- | C/T | T | 92 | C/T | 71.59% |
| | | | 5p | | | | | |
| rs1799782 | XRCC1 | CDS | hsa-miR-886- | C/T | C | 95 | C/T | −67.82% |
| | | | 5p | | | | | |
| rs1799782 | XRCC1 | CDS | hsa-miR-9 | C/T | T | 100 | C/T | 27.38% |
| rs1799782 | XRCC1 | CDS | hsa-miR-920 | C/T | T | 113 | C/T | 25.51% |
| rs1799782 | XRCC1 | CDS | hsa-miR-921 | C/T | T | 106 | C/T | 74.28% |
| rs1799782 | XRCC1 | CDS | hsa-miR-935 | C/T | T | 86 | C/T | 33.22% |
| rs1799782 | XRCC1 | CDS | hsa-miR-936 | C/T | T | 119 | C/T | 39.25% |
| rs1799782 | XRCC1 | CDS | hsa-miR-937 | C/T | C | 123 | C/T | −16.42% |
| rs1799782 | XRCC1 | CDS | hsa-miR-939 | C/T | C | 121 | C/T | −19.68% |
| rs1799782 | XRCC1 | CDS | hsa-miR- | C/T | C | 91 | C/T | −9.09% |
| | | | 99b* | | | | | |
| rs25487 | XRCC1 | CDS | hsa-miR-10a | A/G | G | 114 | A/G | 26.48% |
| rs25487 | XRCC1 | CDS | hsa-miR- | A/G | G | 106 | A/G | 14.68% |
| | | | 1184 | | | | | |
| rs25487 | XRCC1 | CDS | hsa-miR- | A/G | A | 106 | A/G | −33.65% |
| | | | 1202 | | | | | |
| rs25487 | XRCC1 | CDS | hsa-miR- | A/G | G | 116 | A/G | 58.53% |
| | | | 1203 | | | | | |
| rs25487 | XRCC1 | CDS | hsa-miR- | A/G | G | 82 | A/G | 71.29% |
| | | | 1208 | | | | | |
| rs25487 | XRCC1 | CDS | hsa-miR- | A/G | G | 106 | A/G | 25.92% |
| | | | 1225-3p | | | | | |
| rs25487 | XRCC1 | CDS | hsa-miR- | A/G | G | 136 | A/G | 13.30% |
| | | | 1225-5p | | | | | |
| rs25487 | XRCC1 | CDS | hsa-miR- | A/G | A | 147 | A/G | −16.71% |
| | | | 1228* | | | | | |
| rs25487 | XRCC1 | CDS | hsa-miR- | A/G | G | 132 | A/G | 39.96% |
| | | | 1233 | | | | | |
| rs25487 | XRCC1 | CDS | hsa-miR- | A/G | G | 120 | A/G | 17.02% |
| | | | 1247 | | | | | |
| rs25487 | XRCC1 | CDS | hsa-miR- | A/G | A | 89 | A/G | −33.39% |
| | | | 1248 | | | | | |
| rs25487 | XRCC1 | CDS | hsa-miR- | A/G | A | 97 | A/G | −9.20% |
| | | | 125b-1* | | | | | |
| rs25487 | XRCC1 | CDS | hsa-miR-127- | A/G | A | 92 | A/G | −19.88% |
| | | | 3p | | | | | |
| rs25487 | XRCC1 | CDS | hsa-miR- | A/G | A | 71 | A/G | −18.55% |
| | | | 1274b | | | | | |
| rs25487 | XRCC1 | CDS | hsa-miR- | A/G | A | 127 | A/G | −18.11% |
| | | | 1275 | | | | | |
| rs25487 | XRCC1 | CDS | hsa-miR- | A/G | G | 122 | A/G | 26.36% |
| | | | 1296 | | | | | |
| rs25487 | XRCC1 | CDS | hsa-miR- | A/G | G | 96 | A/G | 49.92% |
| | | | 1298 | | | | | |
| rs25487 | XRCC1 | CDS | hsa-miR- | A/G | G | 113 | A/G | 9.78% |
| | | | 1307 | | | | | |
| rs25487 | XRCC1 | CDS | hsa-miR- | A/G | G | 160 | A/G | 24.16% |
| | | | 1470 | | | | | |
| rs25487 | XRCC1 | CDS | hsa-miR- | A/G | G | 84 | A/G | 16.45% |
| | | | 148a* | | | | | |
| rs25487 | XRCC1 | CDS | hsa-miR-149 | A/G | G | 151 | A/G | 34.50% |
| rs25487 | XRCC1 | CDS | hsa-miR-154 | A/G | G | 85 | A/G | 33.76% |
| rs25487 | XRCC1 | CDS | hsa-miR- | A/G | G | 80 | A/G | 11.55% |
| | | | 187* | | | | | |
| rs25487 | XRCC1 | CDS | hsa-miR- | A/G | A | 148 | A/G | −8.21% |
| | | | 18a* | | | | | |
| rs25487 | XRCC1 | CDS | hsa-miR- | A/G | G | 125 | A/G | 25.24% |
| | | | 1908 | | | | | |
| rs25487 | XRCC1 | CDS | hsa-miR- | A/G | G | 132 | A/G | 16.03% |
| | | | 1911 | | | | | |
| rs25487 | XRCC1 | CDS | hsa-miR- | A/G | G | 143 | A/G | 13.75% |
| | | | 1911* | | | | | |
| rs25487 | XRCC1 | CDS | hsa-miR- | A/G | G | 138 | A/G | 13.73% |
| | | | 1913 | | | | | |
| rs25487 | XRCC1 | CDS | hsa-miR- | A/G | G | 121 | A/G | 14.86% |
| | | | 1915 | | | | | |
| rs25487 | XRCC1 | CDS | hsa-miR- | A/G | A | 109 | A/G | −9.29% |
| | | | 194* | | | | | |
| rs25487 | XRCC1 | CDS | hsa-miR- | A/G | G | 95 | A/G | 20.40% |
| | | | 20b* | | | | | |
| rs25487 | XRCC1 | CDS | hsa-miR-211 | A/G | G | 90 | A/G | 30.99% |
| rs25487 | XRCC1 | CDS | hsa-miR- | A/G | A | 119 | A/G | −36.47% |
| | | | 214* | | | | | |
| rs25487 | XRCC1 | CDS | hsa-miR-218- | A/G | G | 118 | A/G | 8.61% |
| | | | 2* | | | | | |
| rs25487 | XRCC1 | CDS | hsa-miR-25* | A/G | G | 97 | A/G | 22.82% |
| rs25487 | XRCC1 | CDS | hsa-miR-296- | A/G | G | 69 | A/G | 11.30% |
| | | | 3p | | | | | |
| rs25487 | XRCC1 | CDS | hsa-miR- | A/G | A | 52 | A/G | −8.64% |
| | | | 30a* | | | | | |
| rs25487 | XRCC1 | CDS | hsa-miR- | A/G | A | 87 | A/G | −80.48% |
| | | | 320a | | | | | |
| rs25487 | XRCC1 | CDS | hsa-miR- | A/G | G | 87 | A/G | 31.80% |
| | | | 320b | | | | | |
| rs25487 | XRCC1 | CDS | hsa-miR-324- | A/G | A | 81 | A/G | −46.41% |
| | | | 5p | | | | | |
| rs25487 | XRCC1 | CDS | hsa-miR-328 | A/G | G | 173 | A/G | 27.92% |
| rs25487 | XRCC1 | CDS | hsa-miR-331- | A/G | G | 115 | A/G | 39.66% |
| | | | 5p | | | | | |
| rs25487 | XRCC1 | CDS | hsa-miR-339- | A/G | G | 159 | A/G | 35.71% |
| | | | 5p | | | | | |
| rs25487 | XRCC1 | CDS | hsa-miR-34a | A/G | A | 105 | A/G | −13.43% |
| rs25487 | XRCC1 | CDS | hsa-miR-361- | A/G | A | 112 | A/G | −48.02% |
| | | | 3p | | | | | |
| rs25487 | XRCC1 | CDS | hsa-miR-362- | A/G | G | 101 | A/G | 31.89% |
| | | | 5p | | | | | |
| rs25487 | XRCC1 | CDS | hsa-miR- | A/G | G | 120 | A/G | 19.40% |
| | | | 363* | | | | | |
| rs25487 | XRCC1 | CDS | hsa-miR- | A/G | G | 141 | A/G | 8.01% |
| | | | 365* | | | | | |
| rs25487 | XRCC1 | CDS | hsa-miR- | A/G | G | 124 | A/G | 19.70% |
| | | | 425* | | | | | |
| rs25487 | XRCC1 | CDS | hsa-miR-448 | A/G | G | 87 | A/G | 21.47% |
| rs25487 | XRCC1 | CDS | hsa-miR-453 | A/G | G | 90 | A/G | 17.53% |
| rs25487 | XRCC1 | CDS | hsa-miR-483- | A/G | G | 116 | A/G | 23.06% |
| | | | 5p | | | | | |
| rs25487 | XRCC1 | CDS | hsa-miR-484 | A/G | G | 125 | A/G | 79.16% |
| rs25487 | XRCC1 | CDS | hsa-miR-486- | A/G | G | 118 | A/G | 28.42% |
| | | | 5p | | | | | |
| rs25487 | XRCC1 | CDS | hsa-miR-489 | A/G | A | 93 | A/G | −63.65% |
| rs25487 | XRCC1 | CDS | hsa-miR-490- | A/G | G | 118 | A/G | 14.71% |
| | | | 5p | | | | | |
| rs25487 | XRCC1 | CDS | hsa-miR-491- | A/G | A | 93 | A/G | −10.67% |
| | | | 5p | | | | | |
| rs25487 | XRCC1 | CDS | hsa-miR-501- | A/G | G | 100 | A/G | 55.43% |
| | | | 3p | | | | | |
| rs25487 | XRCC1 | CDS | hsa-miR- | A/G | G | 110 | A/G | 11.49% |
| | | | 505* | | | | | |
| rs25487 | XRCC1 | CDS | hsa-miR-508- | A/G | G | 137 | A/G | 18.42% |
| | | | 5p | | | | | |
| rs25487 | XRCC1 | CDS | hsa-miR- | A/G | G | 94 | A/G | 8.31% |
| | | | 513a-5p | | | | | |
| rs25487 | XRCC1 | CDS | hsa-miR- | A/G | G | 92 | A/G | 9.40% |
| | | | 513c | | | | | |
| rs25487 | XRCC1 | CDS | hsa-miR-515- | A/G | G | 120 | A/G | 13.55% |
| | | | 3p | | | | | |
| rs25487 | XRCC1 | CDS | hsa-miR- | A/G | G | 102 | A/G | 8.30% |
| | | | 516a-3p | | | | | |
| rs25487 | XRCC1 | CDS | hsa-miR- | A/G | G | 102 | A/G | 8.30% |
| | | | 516b* | | | | | |
| rs25487 | XRCC1 | CDS | hsa-miR- | A/G | G | 91 | A/G | 19.40% |
| | | | 519e* | | | | | |
| rs25487 | XRCC1 | CDS | hsa-miR- | A/G | G | 94 | A/G | 23.05% |
| | | | 520b | | | | | |
| rs25487 | XRCC1 | CDS | hsa-miR- | A/G | G | 94 | A/G | 20.46% |
| | | | 520c-3p | | | | | |
| rs25487 | XRCC1 | CDS | hsa-miR- | A/G | G | 94 | A/G | 16.15% |
| | | | 520e | | | | | |
| rs25487 | XRCC1 | CDS | hsa-miR-520f | A/G | G | 102 | A/G | 16.68% |
| rs25487 | XRCC1 | CDS | hsa-miR-525- | A/G | G | 108 | A/G | 37.95% |
| | | | 5p | | | | | |
| rs25487 | XRCC1 | CDS | hsa-miR- | A/G | A | 107 | A/G | −64.78% |
| | | | 526b | | | | | |
| rs25487 | XRCC1 | CDS | hsa-miR-532- | A/G | G | 123 | A/G | 25.90% |
| | | | 5p | | | | | |
| rs25487 | XRCC1 | CDS | hsa-miR-542- | A/G | G | 107 | A/G | 23.48% |
| | | | 5p | | | | | |
| rs25487 | XRCC1 | CDS | hsa-miR- | A/G | A | 83 | A/G | −65.89% |
| | | | 548a-3p | | | | | |
| rs25487 | XRCC1 | CDS | hsa-miR-584 | A/G | A | 87 | A/G | −27.47% |
| rs25487 | XRCC1 | CDS | hsa-miR-601 | A/G | A | 98 | A/G | −26.92% |
| rs25487 | XRCC1 | CDS | hsa-miR-602 | A/G | G | 129 | A/G | 10.24% |
| rs25487 | XRCC1 | CDS | hsa-miR-611 | A/G | G | 127 | A/G | 9.02% |
| rs25487 | XRCC1 | CDS | hsa-miR-615- | A/G | G | 121 | A/G | 42.64% |
| | | | 5p | | | | | |
| rs25487 | XRCC1 | CDS | hsa-miR- | A/G | A | 95 | A/G | −35.65% |
| | | | 629* | | | | | |
| rs25487 | XRCC1 | CDS | hsa-miR-636 | A/G | A | 93 | A/G | −49.64% |
| rs25487 | XRCC1 | CDS | hsa-miR-638 | A/G | G | 126 | A/G | 10.08% |
| rs25487 | XRCC1 | CDS | hsa-miR-645 | A/G | A | 92 | A/G | −23.90% |
| rs25487 | XRCC1 | CDS | hsa-miR-671- | A/G | G | 103 | A/G | 34.21% |
| | | | 3p | | | | | |
| rs25487 | XRCC1 | CDS | hsa-miR-720 | A/G | G | 94 | A/G | 21.82% |
| rs25487 | XRCC1 | CDS | hsa-miR-760 | A/G | A | 153 | A/G | −15.09% |
| rs25487 | XRCC1 | CDS | hsa-miR-769- | A/G | G | 110 | A/G | 12.79% |
| | | | 3p | | | | | |
| rs25487 | XRCC1 | CDS | hsa-miR-886- | A/G | G | 119 | A/G | 27.74% |
| | | | 5p | | | | | |
| rs25487 | XRCC1 | CDS | hsa-miR-921 | A/G | A | 55 | A/G | −45.94% |
| rs25487 | XRCC1 | CDS | hsa-miR-937 | A/G | A | 95 | A/G | −50.18% |
| rs25487 | XRCC1 | CDS | hsa-miR-943 | A/G | G | 91 | A/G | 22.97% |
| rs132788 | XRCC6 | CDS | hsa-let-7g* | G/T | T | 104 | G/T | 16.25% |
| rs132788 | XRCC6 | CDS | hsa-miR- | G/T | T | 84 | G/T | 19.25% |
| | | | 106b* | | | | | |
| rs132788 | XRCC6 | CDS | hsa-miR- | G/T | G | 139 | G/T | −25.29% |
| | | | 1178 | | | | | |
| rs132788 | XRCC6 | CDS | hsa-miR- | G/T | G | 114 | G/T | −29.69% |
| | | | 1181 | | | | | |
| rs132788 | XRCC6 | CDS | hsa-miR- | G/T | G | 133 | G/T | −18.90% |
| | | | 1225-3p | | | | | |
| rs132788 | XRCC6 | CDS | hsa-miR- | G/T | T | 111 | G/T | 9.09% |
| | | | 1227 | | | | | |
| rs132788 | XRCC6 | CDS | hsa-miR- | G/T | G | 116 | G/T | −12.24% |
| | | | 1228 | | | | | |
| rs132788 | XRCC6 | CDS | hsa-miR- | G/T | G | 121 | G/T | −32.52% |
| | | | 1233 | | | | | |
| rs132788 | XRCC6 | CDS | hsa-miR- | G/T | T | 83 | G/T | 15.75% |
| | | | 125b-2* | | | | | |
| rs132788 | XRCC6 | CDS | hsa-miR- | G/T | G | 121 | G/T | −35.05% |
| | | | 1260 | | | | | |
| rs132788 | XRCC6 | CDS | hsa-miR- | G/T | T | 80 | G/T | 15.45% |
| | | | 1269 | | | | | |
| rs132788 | XRCC6 | CDS | hsa-miR-127- | G/T | T | 100 | G/T | 27.51% |
| | | | 5p | | | | | |
| rs132788 | XRCC6 | CDS | hsa-miR- | G/T | T | 90 | G/T | 37.05% |
| | | | 1276 | | | | | |
| rs132788 | XRCC6 | CDS | hsa-miR- | G/T | T | 94 | G/T | 16.57% |
| | | | 1286 | | | | | |
| rs132788 | XRCC6 | CDS | hsa-miR- | G/T | T | 125 | G/T | 28.93% |
| | | | 1291 | | | | | |
| rs132788 | XRCC6 | CDS | hsa-miR- | G/T | G | 92 | G/T | −23.94% |
| | | | 1307 | | | | | |
| rs132788 | XRCC6 | CDS | hsa-miR- | G/T | G | 118 | G/T | −30.00% |
| | | | 133a | | | | | |
| rs132788 | XRCC6 | CDS | hsa-miR- | G/T | G | 118 | G/T | −30.00% |
| | | | 133b | | | | | |
| rs132788 | XRCC6 | CDS | hsa-miR-138 | G/T | T | 105 | G/T | 13.63% |
| rs132788 | XRCC6 | CDS | hsa-miR-139- | G/T | G | 124 | G/T | −23.69% |
| | | | 5p | | | | | |
| rs132788 | XRCC6 | CDS | hsa-miR-140- | G/T | T | 99 | G/T | 47.12% |
| | | | 3p | | | | | |
| rs132788 | XRCC6 | CDS | hsa-miR-145 | G/T | G | 113 | G/T | −19.67% |
| rs132788 | XRCC6 | CDS | hsa-miR- | G/T | G | 81 | G/T | −30.91% |
| | | | 146a* | | | | | |
| rs132788 | XRCC6 | CDS | hsa-miR- | G/T | G | 118 | G/T | −10.81% |
| | | | 146b-3p | | | | | |
| rs132788 | XRCC6 | CDS | hsa-miR- | G/T | G | 111 | G/T | −17.39% |
| | | | 1470 | | | | | |
| rs132788 | XRCC6 | CDS | hsa-miR- | G/T | G | 106 | G/T | −58.56% |
| | | | 1471 | | | | | |
| rs132788 | XRCC6 | CDS | hsa-miR- | G/T | G | 110 | G/T | −20.77% |
| | | | 148a | | | | | |
| rs132788 | XRCC6 | CDS | hsa-miR-149 | G/T | G | 126 | G/T | −8.77% |
| rs132788 | XRCC6 | CDS | hsa-miR-152 | G/T | G | 100 | G/T | −23.62% |
| rs132788 | XRCC6 | CDS | hsa-miR- | G/T | G | 116 | G/T | −11.57% |
| | | | 1539 | | | | | |
| rs132788 | XRCC6 | CDS | hsa-miR- | G/T | T | 123 | G/T | 14.46% |
| | | | 15a* | | | | | |
| rs132788 | XRCC6 | CDS | hsa-miR- | G/T | G | 112 | G/T | −22.78% |
| | | | 191* | | | | | |
| rs132788 | XRCC6 | CDS | hsa-miR- | G/T | T | 92 | G/T | 33.41% |
| | | | 1911 | | | | | |
| rs132788 | XRCC6 | CDS | hsa-miR- | G/T | T | 96 | G/T | 41.39% |
| | | | 1912 | | | | | |
| rs132788 | XRCC6 | CDS | hsa-miR- | G/T | G | 127 | G/T | −43.43% |
| | | | 1913 | | | | | |
| rs132788 | XRCC6 | CDS | hsa-miR- | G/T | G | 120 | G/T | −11.71% |
| | | | 1914 | | | | | |
| rs132788 | XRCC6 | CDS | hsa-miR- | G/T | T | 104 | G/T | 19.69% |
| | | | 193b | | | | | |
| rs132788 | XRCC6 | CDS | hsa-miR- | G/T | T | 143 | G/T | 15.61% |
| | | | 1972 | | | | | |
| rs132788 | XRCC6 | CDS | hsa-miR- | G/T | G | 115 | G/T | −24.27% |
| | | | 1975 | | | | | |
| rs132788 | XRCC6 | CDS | hsa-miR- | G/T | G | 148 | G/T | −24.03% |
| | | | 1979 | | | | | |
| rs132788 | XRCC6 | CDS | hsa-miR- | G/T | G | 107 | G/T | −28.30% |
| | | | 199a-5p | | | | | |
| rs132788 | XRCC6 | CDS | hsa-miR- | G/T | G | 102 | G/T | −38.71% |
| | | | 199b-5p | | | | | |
| rs132788 | XRCC6 | CDS | hsa-miR- | G/T | G | 91 | G/T | −12.46% |
| | | | 200b* | | | | | |
| rs132788 | XRCC6 | CDS | hsa-miR-218- | G/T | G | 108 | G/T | −9.35% |
| | | | 2* | | | | | |
| rs132788 | XRCC6 | CDS | hsa-miR-24- | G/T | G | 136 | G/T | −57.97% |
| | | | 2* | | | | | |
| rs132788 | XRCC6 | CDS | hsa-miR-25 | G/T | G | 84 | G/T | −9.28% |
| rs132788 | XRCC6 | CDS | hsa-miR-26a- | G/T | G | 115 | G/T | −37.17% |
| | | | 1* | | | | | |
| rs132788 | XRCC6 | CDS | hsa-miR-26a- | G/T | G | 110 | G/T | −40.20% |
| | | | 2* | | | | | |
| rs132788 | XRCC6 | CDS | hsa-miR-27a | G/T | G | 93 | G/T | −16.85% |
| rs132788 | XRCC6 | CDS | hsa-miR- | G/T | G | 89 | G/T | −52.91% |
| | | | 27a* | | | | | |
| rs132788 | XRCC6 | CDS | hsa-miR-298 | G/T | G | 107 | G/T | −11.10% |
| rs132788 | XRCC6 | CDS | hsa-miR-29a | G/T | G | 111 | G/T | −10.58% |
| rs132788 | XRCC6 | CDS | hsa-miR-29b | G/T | G | 105 | G/T | −55.02% |
| rs132788 | XRCC6 | CDS | hsa-miR-29c | G/T | G | 105 | G/T | −16.41% |
| rs132788 | XRCC6 | CDS | hsa-miR- | G/T | T | 107 | G/T | 32.53% |
| | | | 29c* | | | | | |
| rs132788 | XRCC6 | CDS | hsa-miR- | G/T | T | 110 | G/T | 26.20% |
| | | | 30a* | | | | | |
| rs132788 | XRCC6 | CDS | hsa-miR- | G/T | T | 110 | G/T | 25.77% |
| | | | 30e* | | | | | |
| rs132788 | XRCC6 | CDS | hsa-miR-337- | G/T | G | 88 | G/T | −52.34% |
| | | | 3p | | | | | |
| rs132788 | XRCC6 | CDS | hsa-miR-339- | G/T | T | 120 | G/T | 22.98% |
| | | | 3p | | | | | |
| rs132788 | XRCC6 | CDS | hsa-miR- | G/T | T | 115 | G/T | 24.57% |
| | | | 33b* | | | | | |
| rs132788 | XRCC6 | CDS | hsa-miR-342- | G/T | G | 112 | G/T | −31.44% |
| | | | 3p | | | | | |
| rs132788 | XRCC6 | CDS | hsa-miR-345 | G/T | G | 120 | G/T | −18.86% |
| rs132788 | XRCC6 | CDS | hsa-miR- | G/T | T | 122 | G/T | 23.68% |
| | | | 34a* | | | | | |
| rs132788 | XRCC6 | CDS | hsa-miR-411 | G/T | T | 98 | G/T | 8.99% |
| rs132788 | XRCC6 | CDS | hsa-miR-412 | G/T | G | 135 | G/T | −18.88% |
| rs132788 | XRCC6 | CDS | hsa-miR-423- | G/T | G | 108 | G/T | −13.10% |
| | | | 3p | | | | | |
| rs132788 | XRCC6 | CDS | hsa-miR-425 | G/T | T | 89 | G/T | 29.88% |
| rs132788 | XRCC6 | CDS | hsa-miR- | G/T | G | 116 | G/T | −19.27% |
| | | | 449b* | | | | | |
| rs132788 | XRCC6 | CDS | hsa-miR- | G/T | T | 100 | G/T | 34.90% |
| | | | 450b-3p | | | | | |
| rs132788 | XRCC6 | CDS | hsa-miR-455- | G/T | T | 84 | G/T | 43.65% |
| | | | 5p | | | | | |
| rs132788 | XRCC6 | CDS | hsa-miR-484 | G/T | G | 121 | G/T | −22.07% |
| rs132788 | XRCC6 | CDS | hsa-miR-486- | G/T | T | 100 | G/T | 13.81% |
| | | | 5p | | | | | |
| rs132788 | XRCC6 | CDS | hsa-miR-492 | G/T | T | 104 | G/T | 15.98% |
| rs132788 | XRCC6 | CDS | hsa-miR-504 | G/T | T | 111 | G/T | 24.80% |
| rs132788 | XRCC6 | CDS | hsa-miR-512- | G/T | T | 110 | G/T | 19.71% |
| | | | 5p | | | | | |
| rs132788 | XRCC6 | CDS | hsa-miR-525- | G/T | T | 91 | G/T | 24.30% |
| | | | 3p | | | | | |
| rs132788 | XRCC6 | CDS | hsa-miR-532- | G/T | G | 126 | G/T | −20.98% |
| | | | 3p | | | | | |
| rs132788 | XRCC6 | CDS | hsa-miR- | G/T | G | 81 | G/T | −14.42% |
| | | | 541* | | | | | |
| rs132788 | XRCC6 | CDS | hsa-miR-544 | G/T | G | 124 | G/T | −58.70% |
| rs132788 | XRCC6 | CDS | hsa-miR- | G/T | T | 88 | G/T | 141.20 |
| | | | 548e | | | | | |
| rs132788 | XRCC6 | CDS | hsa-miR- | G/T | G | 124 | G/T | −19.46% |
| | | | 551a | | | | | |
| rs132788 | XRCC6 | CDS | hsa-miR-554 | G/T | G | 88 | G/T | −50.24% |
| rs132788 | XRCC6 | CDS | hsa-miR-572 | G/T | G | 136 | G/T | −17.26% |
| rs132788 | XRCC6 | CDS | hsa-miR-574- | G/T | G | 106 | G/T | −29.23% |
| | | | 3p | | | | | |
| rs132788 | XRCC6 | CDS | hsa-miR-581 | G/T | T | 96 | G/T | 30.41% |
| rs132788 | XRCC6 | CDS | hsa-miR-589 | G/T | T | 100 | G/T | 54.73% |
| rs132788 | XRCC6 | CDS | hsa-miR-596 | G/T | G | 132 | G/T | −10.48% |
| rs132788 | XRCC6 | CDS | hsa-miR-602 | G/T | T | 87 | G/T | 19.34% |
| rs132788 | XRCC6 | CDS | hsa-miR-611 | G/T | T | 87 | G/T | 20.82% |
| rs132788 | XRCC6 | CDS | hsa-miR-634 | G/T | T | 150 | G/T | 17.96% |
| rs132788 | XRCC6 | CDS | hsa-miR-638 | G/T | T | 81 | G/T | 10.92% |
| rs132788 | XRCC6 | CDS | hsa-miR-642 | G/T | G | 110 | G/T | −24.03% |
| rs132788 | XRCC6 | CDS | hsa-miR-654- | G/T | T | 86 | G/T | 33.20% |
| | | | 3p | | | | | |
| rs132788 | XRCC6 | CDS | hsa-miR-657 | G/T | G | 119 | G/T | −40.97% |
| rs132788 | XRCC6 | CDS | hsa-miR-660 | G/T | G | 109 | G/T | −36.72% |
| rs132788 | XRCC6 | CDS | hsa-miR-661 | G/T | G | 103 | G/T | −21.20% |
| rs132788 | XRCC6 | CDS | hsa-miR-662 | G/T | G | 129 | G/T | −24.99% |
| rs132788 | XRCC6 | CDS | hsa-miR-664 | G/T | T | 84 | G/T | 16.01% |
| rs132788 | XRCC6 | CDS | hsa-miR-665 | G/T | G | 125 | G/T | −20.00% |
| rs132788 | XRCC6 | CDS | hsa-miR-668 | G/T | G | 87 | G/T | −12.73% |
| rs132788 | XRCC6 | CDS | hsa-miR-769- | G/T | T | 111 | G/T | 13.42% |
| | | | 5p | | | | | |
| rs132788 | XRCC6 | CDS | hsa-miR-770- | G/T | T | 124 | G/T | 9.48% |
| | | | 5p | | | | | |
| rs132788 | XRCC6 | CDS | hsa-miR-886- | G/T | T | 87 | G/T | 122.83% |
| | | | 3p | | | | | |
| rs132788 | XRCC6 | CDS | hsa-miR-886- | G/T | T | 85 | G/T | 46.39% |
| | | | 5p | | | | | |
| rs132788 | XRCC6 | CDS | hsa-miR-934 | G/T | G | 113 | G/T | −27.42% |
| rs132788 | XRCC6 | CDS | hsa-miR-937 | G/T | G | 129 | G/T | −21.03% |
| rs132788 | XRCC6 | CDS | hsa-miR- | G/T | G | 117 | G/T | −11.66% |
| | | | 99b* | | | | | |

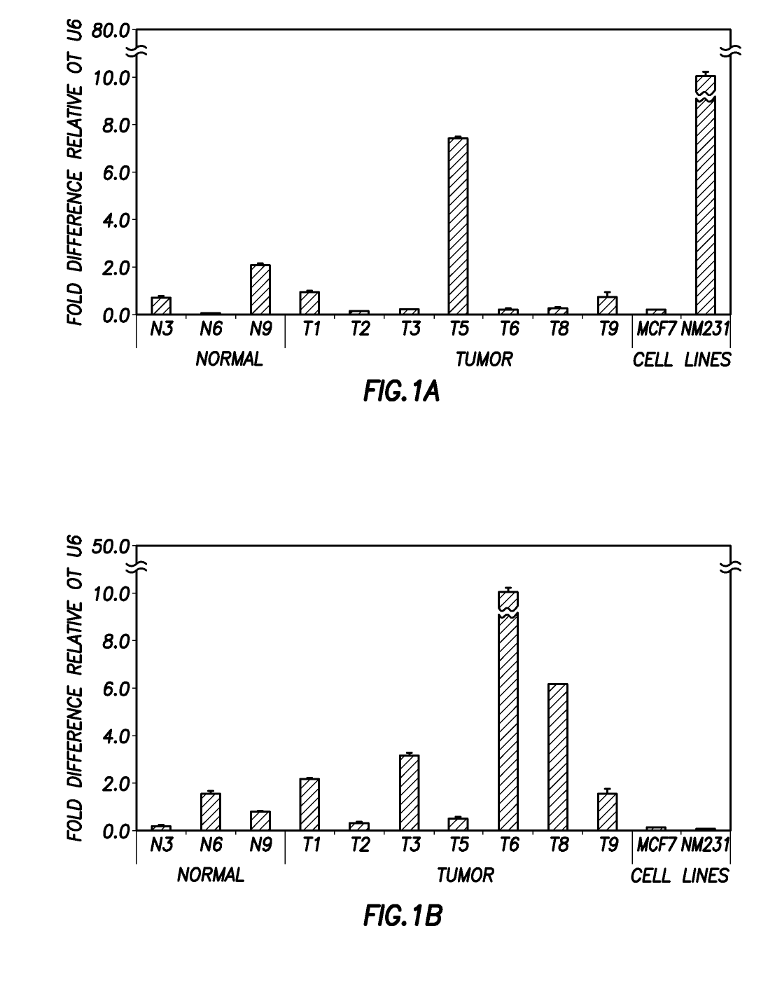

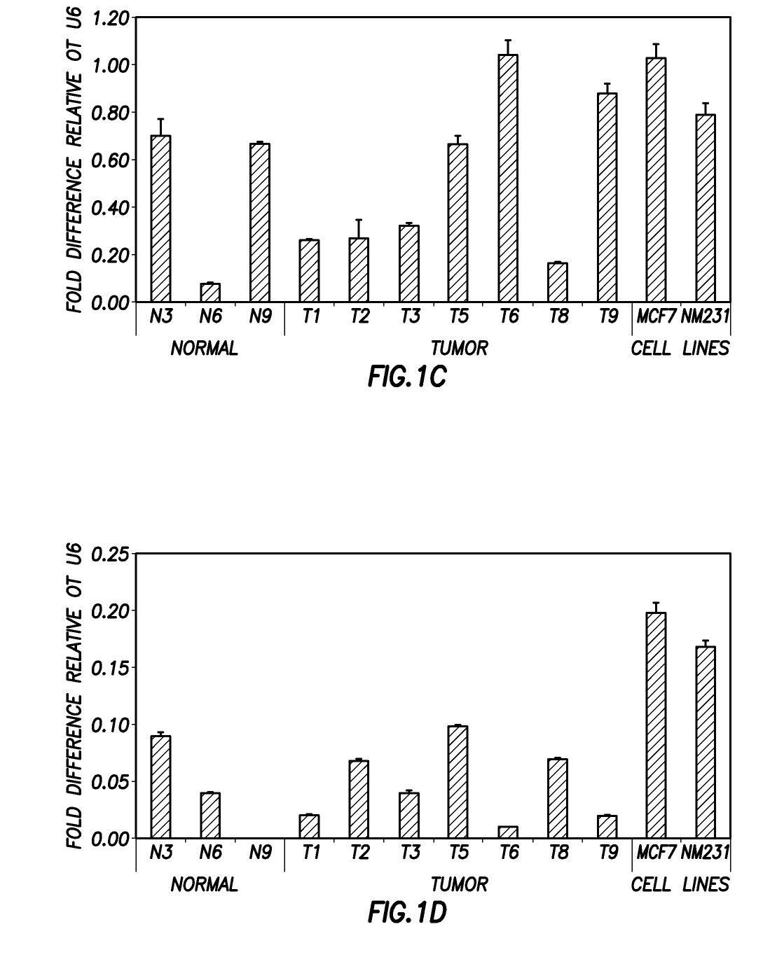

As shown in Table II, we observed that the allelic variants can either increase or decrease the MFE of the corresponding RNA duplexes, leading to either a stronger or weaker miRNA::mRNA binding, respectively. This mechanism can also lead to either creation of a new binding site or destruction of an existing target site. We focused only on those miRNAs (included in Table II) that were expressed in BC samples and cell lines (FIG. 1 and data not shown) and selected 3 candidate target SNPs for functional validation, for a total of 16 miRNA:: SNP interactions as follows.

TABLE III

| | | | Allelic | Active | | MFE |
|---|---|---|---|---|---|---|
| SNP_ID | GENE | miRNA | Variants | Allele | MFE | change |
| rs1799782 | XRCC1 | miR-127-5p | C/T | C | −24.15 | 0% |
| rs1799782 | XRCC1 | miR-125b-1* | C/T | C | −16.14 | −48% |
| rs1799782 | XRCC1 | miR-22 | C/T | T | −16.58 | 103% |
| rs1799782 | XRCC1 | miR-138 | C/T | T | −20.59 | 10% |
| rs1799782 | XRCC1 | miR-127-3p | C/T | C | −18.04 | −10% |
| rs1799782 | XRCC1 | miR-145 | C/T | T | −20.6 | 36% |
| rs1982073 | TGFB1 | miR-127-5p | C/T | C | −23.18 | 0% |
| rs1982073 | TGFB1 | miR-142-3p | C/T | C | −16.57 | −117% |
| rs1982073 | TGFB1 | miR-199a-5p | C/T | C | −17.31 | −85% |
| rs1982073 | TGFB1 | miR-187 | C/T | C | −21.98 | −42% |
| rs1982073 | TGFB1 | miR-214 | C/T | C | −27.28 | −30% |
| rs1982073 | TGFB1 | miR-34a | C/T | C | −24.1 | −72% |
| rs1982073 | TGFB1 | miR-194* | C/T | C | −24.73 | −54% |
| rs572169 | GHSR | miR-181b | A/G | A | −16.31 | −33% |
| rs572169 | GHSR | miR-222 | A/G | G | −21.55 | 31% |
| rs572169 | GHSR | miR-145 | A/G | G | −22.4 | 38% |

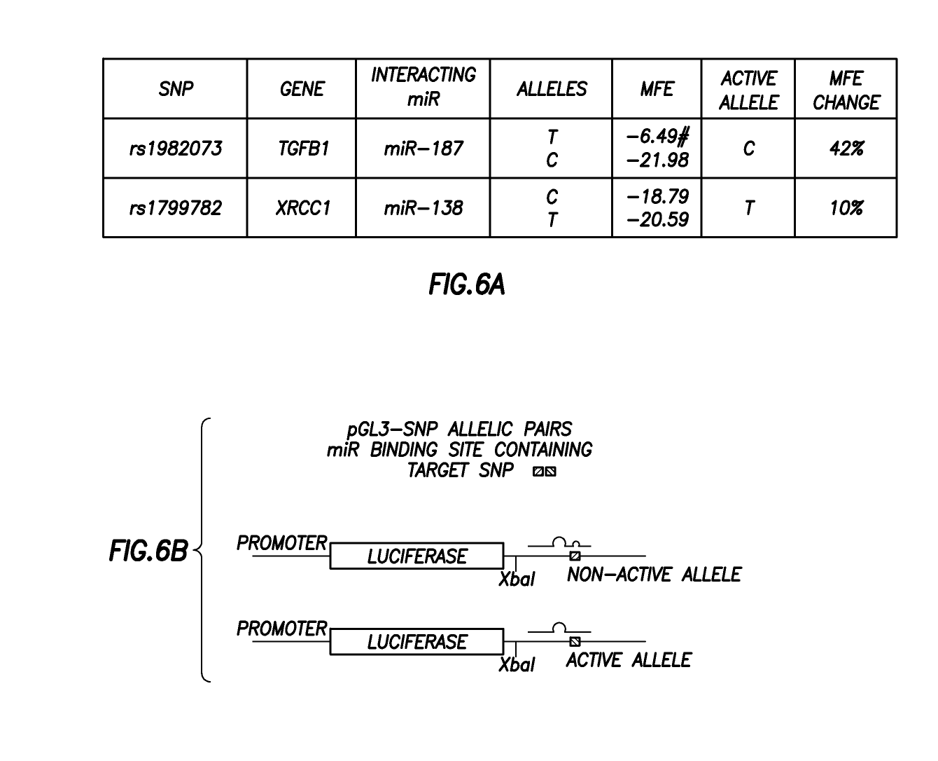

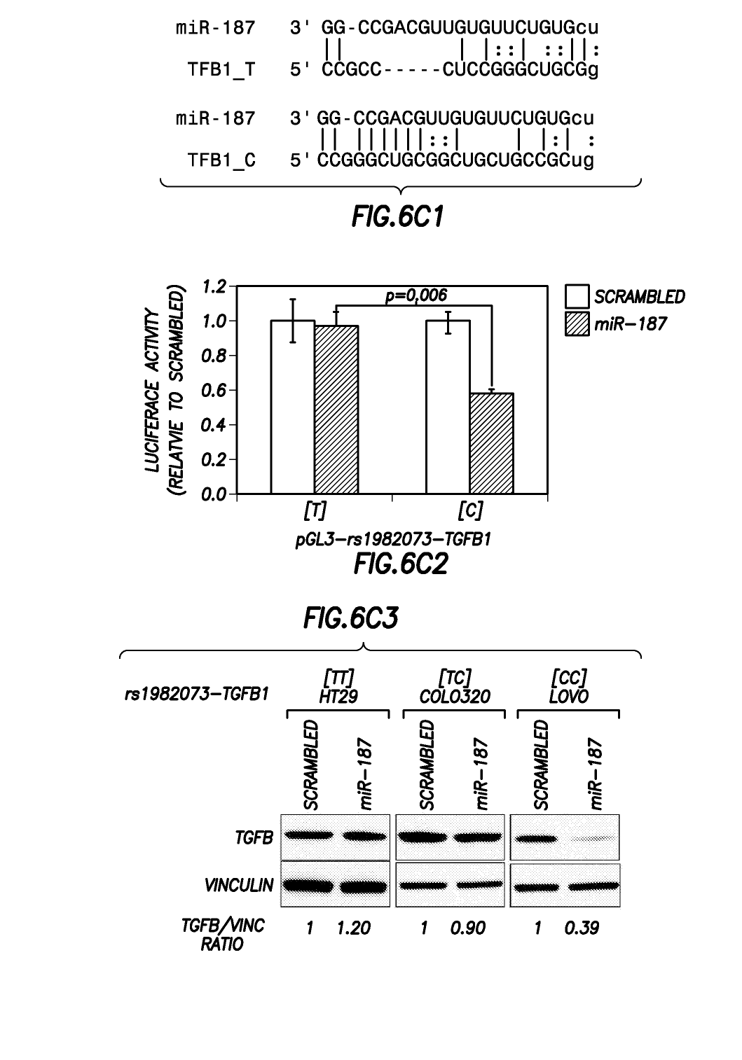

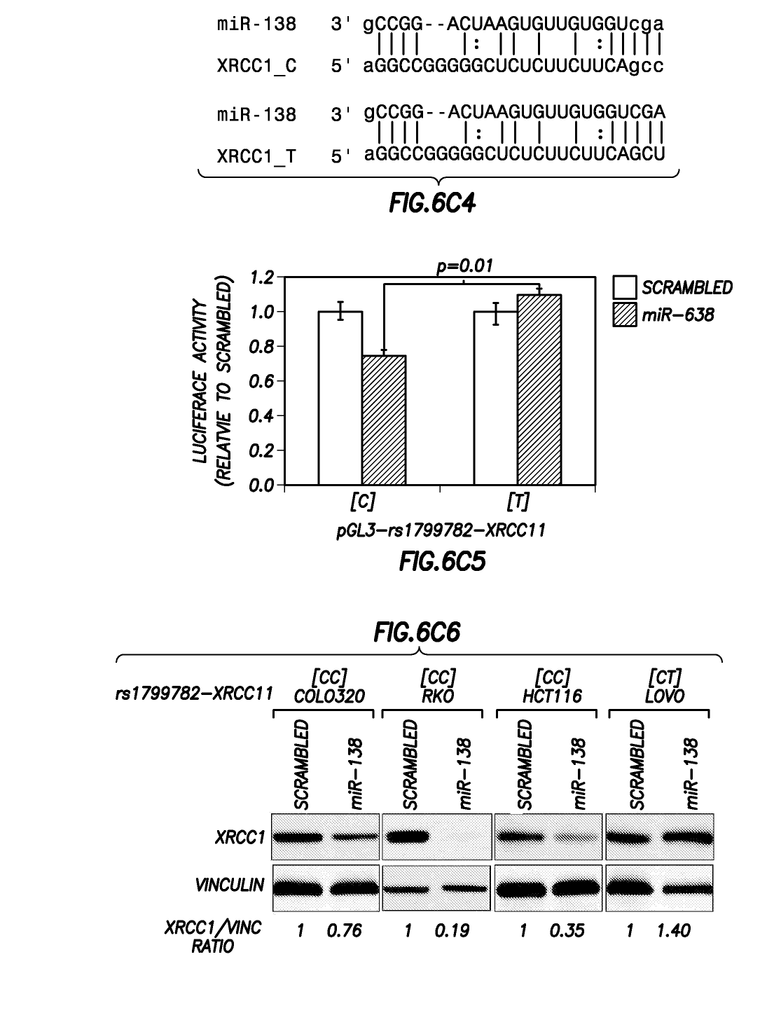

Two out of the 16 interactions chosen were negative controls, in which the interacting miRNA binding was not modified by the SNP allelic variants in DNA and miRNA expression in RNA preparation. To verify whether the selected BC-associated SNPs can actually affect miRNA binding, we performed in vitro luciferase reporter assays. For each SNP, we produced two pGL3-SNP constructs (FIG. 6b) carrying either the active or non-active allele, and co-transfected them in parallel with the predicted interacting miRNAs or the scrambled negative control. rs1982073 inside TGFB1 and rs1799782 inside XRCC1 showed statistically significant effect on miR-187 and miR-138 activity, respectively (FIG. 6a and FIG. 6c). TGFB1's rs1982073 [C] active allele was predicted by miRanda analysis to create a new target site for miR-187, which was absent with the more common [T] variant when stringent settings were used.

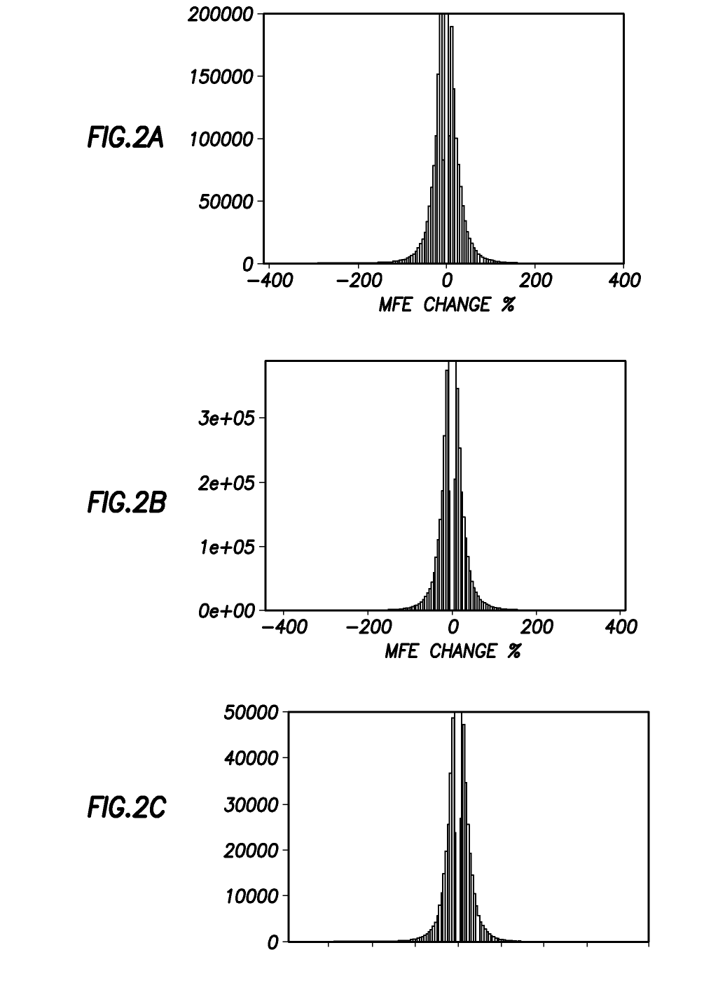

Luciferase assay showed statistically significant suppressive effect of miR-187 on the construct carrying the [C] active allele, which is completely absent with the [T] non-active allele (40% decrease in luciferase activity, p=0.006) (FIG. 6c, left panel). This is in agreement with the major line of evidence assigning an inhibitory role of miRNAs on gene expression. Conversely, according to miRanda prediction, the rs1799782-XRCC1 [T] was the active allele increasing the binding energy of miR-138 compared to the [C] variant. When miR-138 was assayed with pGL3-rs1799782-XRCC1 constructs, we observed a significant increase in the luciferase activity with the [T] allele compared to the [C] allele (34% increase, p=0.0003)(FIG. 2c, right panel). This clearly indicates a stabilizing role, rather than an inhibitory role, for miR-138 stronger binding with the rs1799782-XRCC1 [T] variant.

Subsequently, we tested the effect of these two target SNPs on endogenous TGFB1 and XRCC1 protein levels in cancer cell lines carrying different SNP genotypes by transfecting with the interacting miRNAs. miR-187, consistent with the luciferase results, preferentially down-regulated protein levels of the TGFB1 gene carrying the [CC] rs1982073 active alleles; whereas, miR-187 had an intermediate and opposite effect on the [TC] and [TT] rs1982073 genotypes, respectively (FIG. 6d, left panel). Similarly, miR-138 showed a stabilizing effect when over-expressed in a cell line that was heterozygous for rs1799782-XRCC1. While [CC] carriers displayed decreased XRCC1 protein levels, a cell line carrying the [CT] genotype displayed increased XRCC1 levels, in agreement with the luciferase results (FIG. 2d, right panel). Among the 25 different cancer cell lines we screened, we did not find any rs1799782-XRCC1 [TT] homozygous carrier, supporting the notion that [TT] carriers are protected from developing cancer. Hu Z, et al., XRCC1 Polymorphisms And Cancer Risk: A Meta-Analysis of 38 Case-Control Studies, 14:1810-1818 Cancer Epidemiol Biomarkers Prey (2005). Taken together, these results suggest that for approximately 15% of the investigated transcribed SNPs (2 out of 14 target SNP interactions predicted to be allele-dependent and tested in our analysis, FIG. 6, Table 3 and data not shown), their known association with BC can be biologically due to interference with miRNA binding and therefore miRNA gene regulation.

The fact that only one seventh of the target SNP interactions could be biologically confirmed is not surprising, given the high rate of false positives of all miRNA target prediction programs. While miRanda program is known to produce more false positive predictions than other existing programs that give preference to seed complementarity or inter-species target conservation, it has a higher sensitivity. We, therefore, choose miRanda to identify non-canonical miRNA targets (i.e. with low seed complementarity) like rs1982073-TGFB1 and rs1799782-XRCC1 that would have been otherwise overlooked by using prediction programs focusing only on 3′UTR and considering extensive 5′ complementarity (data not shown). Overall, from our in silico predictions, we validated target SNP interactions that were predicted to have a wide range of MFE change (ranging from 8% up to 42%) with no direct correlation between MFE and probability of a true miRNA::target SNP interaction. Therefore, for subsequent analyses, we filtered the predicted target interactions with a MFE change of at least 8%, at which, in our in vitro experiments, a significant biological effect was detected. Moreover, at this cut off, through a genome-wide target SNP analyses (see below), we noticed a clear peak in the MFE change distribution, further supporting a possible biological significance for MFE changes greater than 8% (FIG. 2).

Identification and Characterization of Target SNPs that Disrupt miRNA::mRNA Interaction.

Out of 3,839,363 SNPs comprised in the human HapMap database (Release 21a), we identified 90,985 SNPs that are located in mRNA regions (i.e. transcribed SNPs) and grouped them according to their genomic locations (5′ UTRs, CDS and 3′ UTRs). We identified candidate target SNPs that can potentially create, destroy or modify miRNA binding sites due to allele-specific MFE changes, through an integrated bioinformatics approach (FIG. 5 and Methods) that includes the predictions of miRanda.

In Table IV, Table V and Table VI (shown immediately below), the top 100 miRNA::SNP allele-specific MFE changes for SNPs located in: 3′ UTR, CDS, and 5′ UTR, respectively are presented. Highly similar bi-modal distribution of MFE change for 3′ UTR, 5′ UTR, and CDS (FIG. 2) were observed. We considered all SNPs that induce at least an 8% MFE change as being a target SNPs.

TABLE IV

| | | | | | Score | MFE | |
|---|---|---|---|---|---|---|---|
| | | | | | for | for | |
| | | | | Active | miRNA: | miRNA: | |
| | Gene | | Allelic | Allele | mRNA- | mRNA- | MFE |
| SNP ID¹ | Symbol | miRNA ID | Variants | (AA) | AA | AA | Change % |
| rs2257136 | ZNF638 | hsa-miR-518a-3p | G/T | G | 146 | −23.77 | −364% |
| rs2257136 | ZNF638 | hsa-miR-526b* | G/T | G | 127 | −21.95 | −233% |
| rs2257136 | ZNF638 | hsa-miR-520c-3p | G/T | G | 123 | −21.95 | −196% |
| rs10417148 | ZNF565 | hsa-miR-937 | C/G | C | 99 | −22.59 | −352% |
| rs4478433 | TULP4 | hsa-miR-520g | G/T | G | 95 | −24.53 | −350% |
| rs10860582 | SLC17A8 | hsa-miR-885-3p | C/T | C | 88 | −22.43 | −349% |
| rs10860582 | SLC17A8 | hsa-miR-518b | C/T | C | 90 | −20.4 | −189% |
| rs10860582 | SLC17A8 | hsa-miR-519d | C/T | C | 84 | −17.88 | −168% |
| rs3213944 | C2orf74 | hsa-miR-1914 | C/G | G | 83 | −21.88 | 338% |
| rs3811106 | STXBP5 | hsa-miR-191 | A/G | G | 96 | −21.92 | 338% |
| rs465736 | RFPL1 | hsa-miR-92b | A/G | G | 115 | −21.41 | 328% |
| rs13195465 | FAM65B | hsa-miR-28-3p | A/C | C | 93 | −27.54 | 326% |
| rs3826412 | C17orf65 | hsa-miR-486-5p | A/G | G | 86 | −24.46 | 322% |
| rs3826412 | C17orf65 | hsa-miR-196b* | A/G | G | 89 | −18.27 | 189% |
| rs11542126 | C10orf2 | hsa-miR-377* | A/G | G | 86 | −22.16 | 320% |
| rs11542126 | C10orf2 | hsa-miR-532-5p | A/G | G | 88 | −16.32 | 188% |
| rs1932 | SUPT5H | hsa-miR-766 | C/T | C | 112 | −21 | −320% |
| rs1950505 | LTB4R2 | hsa-miR-130b* | A/C | C | 108 | −20.88 | 318% |
| rs1950505 | LTB4R2 | hsa-miR-519e | A/C | C | 113 | −18.71 | 265% |
| rs1950505 | LTB4R2 | hsa-miR-664 | A/C | A | 105 | −16.7 | −234% |
| rs218719 | C17orf85 | hsa-miR-1468 | A/G | G | 102 | −20.84 | 317% |
| rs218719 | C17orf85 | hsa-miR-892b | A/G | G | 94 | −20.75 | 154% |
| rs218719 | C17orf85 | hsa-miR-28-5p | A/G | G | 92 | −16.48 | 146% |
| rs10949938 | ZNF138 | hsa-miR-33b | G/T | T | 99 | −22.76 | 313% |
| rs10949938 | ZNF138 | hsa-miR-517a | G/T | T | 109 | −22.68 | 313% |
| rs10949938 | ZNF138 | hsa-miR-517c | G/T | T | 103 | −20.1 | 272% |
| rs12724450 | ECM1 | hsa-miR-203 | A/G | G | 80 | −20.48 | 310% |
| rs12724450 | ECM1 | hsa-miR-342-5p | A/G | A | 89 | −16.53 | −175% |
| rs4252569 | FAM71E2 | hsa-miR-1301 | C/T | T | 88 | −20.42 | 308% |
| rs4252569 | FAM71E2 | hsa-miR-939 | C/T | T | 97 | −23.34 | 144% |
| rs2290595 | SKIV2L2 | hsa-miR-609 | C/T | C | 85 | −20.48 | −305% |
| rs12660274 | GCNT2 | hsa-miR-144* | G/T | T | 85 | −20.23 | 305% |
| rs2286758 | LGALS14 | hsa-miR-1237 | A/G | G | 107 | −20.22 | 304% |
| rs12936731 | NFE2L1 | hsa-miR-380* | C/G | C | 82 | −20.17 | −303% |
| rs12936731 | NFE2L1 | hsa-miR-193a-5p | C/G | C | 91 | −17.23 | −158% |
| rs12342165 | GAS1 | hsa-miR-135a | C/T | C | 109 | −20.02 | −300% |
| rs7906863 | TACR2 | hsa-miR-1914 | A/G | A | 87 | −19.93 | −299% |
| rs7640976 | VPS8 | hsa-miR-936 | C/G | C | 88 | −22.54 | −298% |
| rs7640976 | VPS8 | hsa-miR-1277 | C/G | C | 92 | −20.2 | −176% |
| rs7297130 | TPCN1 | hsa-let-7e* | C/T | T | 88 | −19.86 | 297% |
| rs12038009 | PDE4B | hsa-miR-519e | C/T | C | 93 | −21.56 | −296% |
| rs6690757 | CDC42 | hsa-miR-582-5p | G/T | G | 86 | −19.59 | −292% |
| rs6690757 | CDC42 | hsa-miR-605 | G/T | G | 80 | −19.34 | −264% |
| rs6690757 | CDC42 | hsa-miR-412 | G/T | G | 80 | −21.7 | −173% |
| rs2269728 | ZNF800 | hsa-miR-151-5p | A/G | G | 108 | −22.19 | 292% |
| rs11569363 | CD27 | hsa-miR-1227 | A/G | G | 93 | −19.56 | 291% |
| rs11569363 | CD27 | hsa-miR-517c | A/G | A | 80 | −17.66 | −155% |
| rs132734 | APOL4 | hsa-miR-766 | A/G | A | 87 | −21.29 | −290% |
| rs2071463 | PSMB8 | hsa-let-7f-1* | A/G | G | 90 | −19.5 | 290% |
| rs1802539 | AZIN1 | hsa-miR-16-1* | C/T | T | 100 | −23.38 | 289% |
| rs3134295 | SLC25A32 | hsa-miR-1226 | A/C | C | 118 | −19.44 | 289% |
| rs1043150 | RSL24D1 | hsa-miR-671-3p | C/T | C | 90 | −22.26 | −288% |
| rs12339493 | RGS3 | hsa-miR-548b-3p | A/G | A | 92 | −19.35 | −287% |
| rs3746619 | MC3R | hsa-miR-509-3p | A/C | C | 80 | −19.29 | 286% |
| rs132736 | APOL4 | hsa-miR-376a* | C/T | C | 86 | −19.47 | −286% |
| rs4150248 | ERCC5 | hsa-miR-409-3p | A/C | C | 80 | −19.27 | 285% |
| rs2302437 | STYXL1 | hsa-miR-520a-3p | A/G | A | 89 | −19.23 | −285% |
| rs1814763 | RPLP1 | hsa-miR-664 | G/T | T | 82 | −19.19 | 284% |
| rs1814763 | RPLP1 | hsa-miR-511 | G/T | G | 86 | −21.26 | −193% |
| rs3814026 | ANAPC1 | hsa-miR-320a | C/T | T | 96 | −19.16 | 283% |
| rs3814026 | ANAPC1 | hsa-miR-125a-5p | C/T | T | 92 | −18.01 | 155% |
| rs3737809 | SMPDL3B | hsa-miR-877 | A/G | G | 89 | −19.47 | 282% |
| rs632148 | SRD5A2 | hsa-miR-135a | C/G | C | 90 | −19.07 | −281% |
| rs632148 | SRD5A2 | hsa-miR-574-3p | C/G | C | 88 | −17.15 | −189% |
| rs4705668 | KCNN2 | hsa-miR-128 | A/G | G | 85 | −20.62 | 281% |
| rs2072750 | PPP1R8 | hsa-miR-199a-5p | C/T | C | 104 | −18.98 | −280% |
| rs2072750 | PPP1R8 | hsa-miR-618 | C/T | C | 83 | −20.37 | −153% |
| rs2285635 | OFD1 | hsa-miR-520g | G/T | T | 83 | −18.99 | 280% |
| rs2285635 | OFD1 | hsa-miR-542-3p | G/T | G | 93 | −16.75 | −208% |
| rs2285635 | OFD1 | hsa-miR-302a | G/T | T | 80 | −16.92 | 162% |
| rs3177269 | WBP11 | hsa-miR-92a | A/G | G | 95 | −20.39 | 279% |
| rs3177269 | WBP11 | hsa-miR-300 | A/G | G | 96 | −19.47 | 221% |
| rs3746569 | FITM2 | hsa-miR-517b | A/T | A | 105 | −21.32 | −279% |
| rs3746569 | FITM2 | hsa-miR-1182 | A/T | T | 81 | −16.23 | 199% |
| rs6033785 | TASP1 | hsa-miR-502-5p | C/T | C | 106 | −18.9 | −278% |
| rs6033785 | TASP1 | hsa-miR-891b | C/T | C | 82 | −20.49 | −180% |
| rs6033785 | TASP1 | hsa-miR-520e | C/T | C | 84 | −16.23 | −146% |
| rs3802587 | PHYH | hsa-miR-221 | A/G | A | 84 | −18.78 | −276% |
| rs13043797 | RTEL1 | hsa-miR-675* | A/C | A | 86 | −18.8 | −276% |
| rs13043797 | RTEL1 | hsa-miR-636 | A/C | A | 92 | −17.43 | −236% |
| rs13043797 | RTEL1 | hsa-miR-129-5p | A/C | C | 90 | −24.67 | 164% |
| rs2270819 | THG1L | hsa-miR-1236 | G/T | T | 136 | −24.12 | 276% |
| rs2270819 | THG1L | hsa-miR-1248 | G/T | T | 104 | −20.22 | 199% |
| rs2270819 | THG1L | hsa-miR-412 | G/T | T | 113 | −17.45 | 169% |
| rs11848386 | DCAF11 | hsa-miR-432* | A/C | C | 89 | −20.05 | 275% |
| rs5743565 | TLR1 | hsa-miR-492 | A/G | G | 88 | −18.77 | 275% |
| rs5743565 | TLR1 | hsa-miR-181b | A/G | A | 83 | −19.7 | −143% |
| rs10401707 | ZNF561 | hsa-miR-491-3p | A/G | A | 87 | −18.71 | −274% |
| rs2247216 | C19orf48 | hsa-miR-202* | C/T | C | 101 | −18.69 | −274% |
| rs3811436 | GPBP1L1 | hsa-miR-937 | C/T | C | 105 | −18.61 | −272% |
| rs529224 | CDC14A | hsa-miR-522 | C/G | G | 98 | −18.6 | 272% |
| rs529224 | CDC14A | hsa-miR-377* | C/G | G | 80 | −18.89 | 241% |
| rs529224 | CDC14A | hsa-miR-520g | C/G | G | 167 | −26.07 | 199% |
| rs10153729 | DDX1 | hsa-miR-9 | A/C | A | 115 | −21.05 | −272% |
| rs6000189 | APOL4 | hsa-miR-512-3p | A/G | G | 84 | −18.56 | 271% |
| rs594814 | PLEKHB1 | hsa-miR-1182 | C/G | C | 114 | −22.19 | −270% |
| rs594814 | PLEKHB1 | hsa-miR-500* | C/G | G | 102 | −17.1 | 217% |
| rs2294920 | SAMM50 | hsa-miR-302c | C/T | C | 90 | −21.07 | −270% |
| rs7690197 | ARSJ | hsa-miR-567 | C/T | C | 89 | −19.32 | −270% |
| rs1194182 | CD36 | hsa-miR-1200 | C/G | G | 108 | −18.46 | 269% |
| rs922483 | BLK | hsa-miR-509-3p | C/T | C | 85 | −21.39 | −269% |
| rs922483 | BLK | hsa-miR-30a | C/T | T | 91 | −18.35 | 267% |
| rs922483 | BLK | hsa-miR-377 | C/T | C | 101 | −17.48 | −146% |
| rs16884568 | CSMD3 | hsa-miR-744* | C/G | C | 91 | −23.67 | −269% |
| rs16884568 | CSMD3 | hsa-miR-337-5p | C/G | C | 115 | −19.34 | −168% |
| rs329379 | DBR1 | hsa-miR-1236 | G/T | T | 114 | −18.65 | 268% |
| rs329379 | DBR1 | hsa-miR-200c | G/T | G | 91 | −19.98 | −165% |
| rs329379 | DBR1 | hsa-miR-593 | G/T | G | 110 | −17.7 | −148% |
| rs12187908 | NNT | hsa-miR-150* | C/T | C | 86 | −23.98 | −268% |
| rs12187908 | NNT | hsa-miR-518b | C/T | C | 85 | −17.25 | −245% |
| rs1057454 | ZNF800 | hsa-miR-548p | G/T | G | 96 | −18.39 | −268% |
| rs1057454 | ZNF800 | hsa-miR-138 | G/T | T | 86 | −19.47 | 146% |
| rs12898092 | CPNE6 | hsa-miR-200b* | A/G | G | 91 | −19.06 | 267% |
| rs3752085 | MAPK4 | hsa-miR-574-3p | G/T | G | 103 | −18.37 | −267% |
| rs3752085 | MAPK4 | hsa-miR-210 | G/T | G | 92 | −18.87 | −180% |
| rs3752085 | MAPK4 | hsa-miR-504 | G/T | G | 89 | −20.42 | −142% |
| rs937652 | MCCC1 | hsa-miR-299-5p | C/G | G | 99 | −18.33 | 267% |
| rs2276786 | OXNAD1 | hsa-miR-186 | G/T | G | 90 | −18.32 | −266% |
| rs7358459 | BBOX1 | hsa-miR-146a* | C/T | C | 88 | −18.23 | −265% |
| rs11231696 | FLRT1 | hsa-miR-520g | G/T | T | 106 | −18.27 | 265% |
| rs11231696 | FLRT1 | hsa-miR-520h | G/T | T | 103 | −18.27 | 265% |
| rs11231696 | FLRT1 | hsa-miR-1324 | G/T | T | 81 | −19.28 | 167% |
| rs1455774 | C15orf32 | hsa-miR-1228 | A/C | C | 82 | −18.25 | 265% |
| rs1455774 | C15orf32 | hsa-miR-539 | A/C | A | 101 | −16.28 | −176% |
| rs1455774 | C15orf32 | hsa-let-7i* | A/C | C | 92 | −21.65 | 152% |
| rs3744165 | ZNF750 | hsa-miR-1249 | A/C | A | 95 | −18.25 | −265% |
| rs10176303 | HEATR5B | hsa-let-7e* | C/G | C | 110 | −20.89 | −265% |
| rs199738 | TRIM38 | hsa-miR-27b* | C/T | C | 100 | −18.25 | −265% |
| rs11546519 | CCNC | hsa-miR-629 | G/T | T | 87 | −18.26 | 265% |
| rs3751212 | WSCD2 | hsa-miR-122 | A/C | C | 97 | −18.85 | 264% |
| rs872556 | SPERT | hsa-miR-589 | C/G | C | 97 | −18.13 | −263% |
| rs2070609 | SERPINA1 | hsa-miR-21* | C/G | G | 104 | −20.81 | 263% |
| rs1077511 | TXNL4A | hsa-miR-662 | G/T | G | 92 | −18.41 | −263% |
| rs6917933 | DDX43 | hsa-miR-675* | G/T | G | 83 | −18.17 | −263% |
| rs6917933 | DDX43 | hsa-miR-1976 | G/T | G | 91 | −17.67 | −228% |
| rs6917933 | DDX43 | hsa-miR-1249 | G/T | G | 135 | −17.66 | −200% |
| rs1046188 | SCAMP3 | hsa-miR-101* | A/G | A | 80 | −18.12 | −262% |
| rs1046188 | SCAMP3 | hsa-miR-24-2* | A/G | G | 93 | −16.57 | 163% |
| rs3016326 | PICALM | hsa-miR-1182 | G/T | G | 114 | −22.32 | −262% |
| rs3016326 | PICALM | hsa-miR-135a* | G/T | G | 102 | −28.72 | −225% |
| rs3016326 | PICALM | hsa-miR-629 | G/T | G | 80 | −17.63 | −184% |
| rs6580940 | ESPL1 | hsa-miR-551b | A/T | A | 100 | −18.31 | −262% |
| rs6580940 | ESPL1 | hsa-miR-1286 | A/T | T | 124 | −24.05 | 177% |
| rs6580940 | ESPL1 | hsa-miR-551a | A/T | A | 97 | −16.76 | −158% |
| rs9556649 | OXGR1 | hsa-miR-16-1* | A/C | A | 93 | −21.67 | −261% |
| rs3742689 | KCNK10 | hsa-miR-671-3p | A/G | G | 116 | −19.6 | 261% |
| rs3742689 | KCNK10 | hsa-miR-154 | A/G | G | 99 | −19.04 | 244% |
| rs3813089 | MRO | hsa-miR-514 | G/T | T | 97 | −18.04 | 261% |
| rs3813089 | MRO | hsa-miR-1285 | G/T | G | 122 | −20.87 | −170% |
| rs17340169 | MCF2 | hsa-miR-548p | A/G | A | 84 | −18.03 | −261% |
| rs17340169 | MCF2 | hsa-miR-154 | A/G | A | 88 | −17.11 | −157% |
| rs3212379 | MC1R | hsa-miR-885-5p | C/T | T | 120 | −19.57 | 260% |
| rs3212379 | MC1R | hsa-miR-299-5p | C/T | T | 81 | −16.5 | 182% |
| rs12103435 | CARD14 | hsa-miR-1281 | C/T | C | 110 | −18.02 | −260% |
| rs12103435 | CARD14 | hsa-miR-103-as | C/T | T | 120 | −20.37 | 179% |
| rs1885311 | RASSF2 | hsa-miR-302d* | C/G | C | 80 | −19.52 | −260% |
| rs3112369 | SLC13A4 | hsa-miR-191 | C/T | C | 92 | −18.02 | −260% |
| rs3219467 | TOE1 | hsa-miR-100 | C/G | C | 92 | −21.03 | −259% |
| rs9578070 | TPTE2 | hsa-miR-1913 | C/T | C | 109 | −18.09 | −259% |
| rs9578070 | TPTE2 | hsa-miR-532-3p | C/T | C | 102 | −16.96 | −188% |
| rs12340011 | KLF4 | hsa-miR-548i | A/G | A | 83 | −17.95 | −259% |
| rs12340011 | KLF4 | hsa-miR-139-5p | A/G | G | 93 | −16.88 | 158% |
| rs4648257 | PTGS2 | hsa-miR-891a | A/T | A | 87 | −21.63 | −258% |
| rs4648257 | PTGS2 | hsa-miR-578 | A/T | T | 84 | −18.03 | 160% |
| rs1134669 | IFITM1 | hsa-miR-511 | A/G | G | 106 | −17.88 | 258% |

³Location of all SNPs identified in Table 4 is 5′ UTR.

TABLE 5

| | | | | Active | Score for | MFE for | |
|---|---|---|---|---|---|---|---|
| | | | Allelic | Allele | miRNA: | miRNA: | MFE |
| SNP ID² | Gene Symbol | miRNA ID | Variants | (AA) | mRNA-AA | mRNA-AA | Change % |
| rs8187758 | SLC28A1 | hsa-miR-675* | A/C | C | 108 | −25.61 | 412% |
| rs2306646 | XPO7 | hsa-miR-1182 | C/G | C | 121 | −25.31 | −406% |
| rs2306646 | XPO7 | hsa-miR-1280 | C/G | C | 89 | −17.48 | −236% |
| rs7944051 | SLC3A2 | hsa-miR-520g | C/T | T | 97 | −24.96 | 399% |
| rs7944051 | SLC3A2 | hsa-miR-520h | C/T | T | 97 | −23.94 | 379% |
| rs1177573 | PSMD9 | hsa-miR-1284 | A/G | A | 91 | −24.73 | −395% |
| rs9282553 | ABCA6 | hsa-miR-26a-1* | C/G | C | 87 | −24.26 | −385% |
| rs6907125 | GPR111 | hsa-miR-338-3p | A/G | G | 130 | −25.15 | 382% |
| rs3104230 | CDC20B | hsa-let-7c* | G/T | G | 81 | −24.25 | −380% |
| rs4823054 | ASCC2 | hsa-miR-567 | C/T | T | 99 | −24.92 | 377% |
| rs17122002 | MPZL2 | hsa-miR-1914 | C/T | T | 87 | −23.82 | 376% |
| rs7323 | GTF3A | hsa-miR-517b | C/G | G | 127 | −23.53 | 371% |
| rs3752497 | SUSD2 | hsa-miR-567 | A/G | G | 92 | −24.54 | 370% |
| rs2072650 | COL9A1 | hsa-miR-1181 | A/G | G | 98 | −23.47 | 369% |
| rs5911 | ITGA2B | hsa-miR-758 | A/C | C | 81 | −23.41 | 368% |
| rs11889464 | TEKT4 | hsa-miR-935 | C/T | C | 103 | −25.92 | −365% |
| rs17051488 | MAMDC2 | hsa-miR-625 | A/C | C | 93 | −22.99 | 360% |
| rs340151 | TMPRSS7 | hsa-miR-30c | A/T | T | 103 | −22.89 | 358% |
| rs340151 | TMPRSS7 | hsa-miR-30e | A/T | T | 98 | −18.6 | 258% |
| rs340151 | TMPRSS7 | hsa-miR-30d | A/T | T | 91 | −17.54 | 242% |
| rs730313 | ATP1A4 | hsa-miR-518b | C/T | C | 124 | −22.8 | −356% |
| rs730313 | ATP1A4 | hsa-miR-518d-3p | C/T | C | 122 | −21.22 | −240% |
| rs3177821 | CTSH | hsa-miR-125a-5p | C/T | C | 102 | −22.78 | −356% |
| rs3736422 | TMF1 | hsa-miR-885-5p | C/T | C | 105 | −22.74 | −355% |
| rs3792234 | STON1 | hsa-miR-1539 | G/T | G | 103 | −22.68 | −354% |
| rs11954456 | FGFR4 | hsa-miR-150 | C/G | G | 94 | −22.96 | 354% |
| rs12063768 | CCBL2 | hsa-miR-328 | C/T | C | 98 | −22.68 | −352% |
| rs5743513 | BPI | hsa-miR-491-3p | A/C | C | 120 | −22.59 | 352% |
| rs3827026 | ZBTB46 | hsa-miR-211 | C/T | C | 102 | −22.56 | −351% |
| rs17097226 | PCDHGA4 | hsa-miR-328 | G/T | G | 87 | −22.53 | −351% |
| rs2004074 | KIAA0649 | hsa-let-7b* | A/G | G | 80 | −23.6 | 350% |
| rs2004074 | KIAA0649 | hsa-miR-520g | A/G | A | 81 | −20.76 | −308% |
| rs12185800 | C20orf152 | hsa-miR-1978 | G/T | G | 80 | −22.4 | −348% |
| rs12185800 | C20orf152 | hsa-miR-1243 | G/T | G | 83 | −18.92 | −238% |
| rs4253219 | ERCC6 | hsa-miR-1911* | G/T | G | 128 | −25.67 | −347% |
| rs1053176 | NAB1 | hsa-miR-1825 | C/T | T | 84 | −22.28 | 346% |
| rs12337278 | C9orf144B | hsa-miR-1256 | A/G | G | 90 | −22.29 | 346% |
| rs6698807 | NASP | hsa-miR-103-as | A/G | G | 110 | −22.13 | 343% |
| rs7528067 | RPF1 | hsa-miR-572 | A/G | G | 111 | −27.58 | 343% |
| rs4727749 | IMMP2L | hsa-miR-197 | A/G | A | 113 | −22.03 | −341% |
| rs2072163 | JHDM1D | hsa-let-7g* | G/T | G | 83 | −23.62 | −340% |
| rs7359861 | COX6B1 | hsa-miR-365* | C/T | T | 102 | −21.94 | 339% |
| rs3783613 | VCAM1 | hsa-miR-524-5p | C/G | C | 95 | −21.89 | −338% |
| rs13330462 | CD2BP2 | hsa-miR-522 | C/T | C | 80 | −23.76 | −338% |
| rs6050797 | TMC2 | hsa-miR-376c | A/G | G | 88 | −21.85 | 337% |
| rs17277515 | OR6S1 | hsa-let-7f-1* | A/G | A | 84 | −22.29 | −335% |
| rs2561545 | NUMBL | hsa-miR-27b | C/T | T | 100 | −22.14 | 335% |
| rs12627708 | B3GALT5 | hsa-miR-23b* | G/T | G | 116 | −21.77 | −335% |
| rs2272662 | SLC39A4 | hsa-miR-518b | A/G | G | 99 | −21.73 | 335% |
| rs2272662 | SLC39A4 | hsa-miR-518e | A/G | G | 109 | −22.58 | 277% |
| rs2272662 | SLC39A4 | hsa-miR-187 | A/G | A | 109 | −22.07 | −244% |
| rs133072 | MCHR1 | hsa-miR-1267 | A/G | G | 87 | −21.71 | 334% |
| rs11961941 | TRIM15 | hsa-miR-1299 | G/T | T | 92 | −24.29 | 334% |
| rs17313892 | MCC | hsa-miR-30e* | A/T | A | 92 | −22.05 | −333% |
| rs17313892 | MCC | hsa-miR-30a* | A/T | A | 88 | −18.76 | −269% |
| rs1041073 | B3GALTL | hsa-miR-200c* | C/T | C | 82 | −22.76 | −330% |
| rs17501584 | OR4D9 | hsa-miR-671-3p | A/G | G | 88 | −21.42 | 328% |
| rs17040157 | C3orf20 | hsa-miR-1249 | A/G | G | 100 | −21.41 | 328% |
| rs1051264 | APPBP2 | hsa-miR-921 | C/G | C | 115 | −21.49 | −326% |
| rs1780316 | ALPL | hsa-miR-23a | C/T | T | 95 | −21.27 | 325% |
| rs1780316 | ALPL | hsa-miR-23b | C/T | T | 91 | −20.43 | 309% |
| rs6510758 | C19orf71 | hsa-miR-574-5p | C/T | C | 91 | −22.08 | −324% |
| rs6510758 | C19orf71 | hsa-miR-186 | C/T | T | 81 | −21.62 | 252% |
| rs17749904 | CLIP4 | hsa-miR-26a | C/G | G | 86 | −21.19 | 324% |
| rs2278017 | ERAP1 | hsa-miR-125b | C/G | G | 105 | −21.19 | 324% |
| rs2278017 | ERAP1 | hsa-miR-1207-3p | C/G | G | 88 | −18.59 | 272% |
| rs2250000 | FN3KRP | hsa-miR-135a* | C/G | C | 81 | −21.16 | −323% |
| rs1022477 | RIBC2 | hsa-miR-663b | A/G | G | 104 | −25.44 | 323% |
| rs2856655 | MYBPC3 | hsa-miR-1181 | A/G | G | 120 | −24.24 | 322% |
| rs7927388 | TSGA10IP | hsa-miR-520c-3p | A/C | C | 88 | −21.45 | 322% |
| rs7927388 | TSGA10IP | hsa-miR-520b | A/C | C | 88 | −20.79 | 316% |
| rs4757986 | OR52E4 | hsa-miR-329 | G/T | G | 109 | −22.67 | −321% |
| rs17158278 | WHAMM | hsa-miR-23b* | C/T | C | 101 | −25.22 | −321% |
| rs5912 | ITGA2B | hsa-miR-508-3p | A/G | G | 89 | −21.05 | 321% |
| rs386598 | C3orf18 | hsa-miR-548b-5p | A/C | C | 83 | −21.04 | 321% |
| rs11542126 | C10orf2 | hsa-miR-377* | A/G | G | 86 | −22.16 | 320% |
| rs11573591 | PTCH2 | hsa-miR-433 | A/G | A | 107 | −20.93 | −319% |
| rs6721024 | LY75 | hsa-miR-302b | C/T | C | 80 | −20.97 | −319% |
| rs6721024 | LY75 | hsa-miR-30c | C/T | T | 108 | −17.46 | 249% |
| rs3737277 | TRIM58 | hsa-miR-1323 | A/G | A | 80 | −20.85 | −317% |
| rs218719 | C17orf85 | hsa-miR-1468 | A/G | G | 102 | −20.84 | 317% |
| rs3103004 | EZR | hsa-miR-101* | A/G | A | 102 | −20.85 | −317% |
| rs7803499 | TMEM229A | hsa-miR-514 | C/T | C | 90 | −23.43 | −316% |
| rs7803499 | TMEM229A | hsa-miR-586 | C/T | C | 80 | −18.83 | −276% |
| rs7803499 | TMEM229A | hsa-miR-32 | C/T | C | 104 | −16.92 | −238% |
| rs1051375 | CACNA1C | hsa-miR-935 | A/G | G | 88 | −22.36 | 315% |
| rs2273952 | AKAP11 | hsa-miR-885-3p | C/T | C | 84 | −20.74 | −315% |
| rs7150973 | PLEKHH1 | hsa-miR-1250 | A/G | G | 85 | −20.76 | 315% |
| rs2277524 | KCNK10 | hsa-miR-371-3p | C/G | G | 82 | −20.74 | 315% |
| rs10250 | MAP2K2 | hsa-miR-561 | G/T | T | 89 | −20.75 | 315% |
| rs10947087 | MDC1 | hsa-miR-34a | A/G | A | 130 | −25.27 | −315% |
| rs2297236 | ZC3HAV1 | hsa-miR-373* | C/G | C | 80 | −20.75 | −315% |
| rs10817021 | SVEP1 | hsa-miR-125b | A/T | T | 99 | −20.76 | 315% |
| rs1815739 | ACTN3 | hsa-miR-129-5p | C/T | C | 86 | −20.71 | −314% |
| rs2305652 | PLCB2 | hsa-miR-203 | C/T | T | 90 | −21.59 | 314% |
| rs226788 | MKL2 | hsa-miR-571 | C/T | C | 118 | −22.24 | −314% |
| rs6566987 | ALPK2 | hsa-miR-575 | G/T | T | 81 | −20.69 | 314% |
| rs4927910 | COPG | hsa-miR-338-3p | A/G | A | 89 | −20.7 | −314% |
| rs13571 | MRPL37 | hsa-miR-106a | C/G | C | 85 | −20.63 | −313% |
| rs2275303 | SIPA1L2 | hsa-miR-1229 | C/T | C | 90 | −24.89 | −313% |
| rs7295376 | TAPBPL | hsa-miR-1224-3p | C/G | G | 119 | −20.66 | 313% |
| rs943992 | FLJ10357 | hsa-miR-518b | C/T | C | 104 | −26.2 | −313% |
| rs2248069 | CACNA1A | hsa-miR-766 | A/G | G | 111 | −20.64 | 313% |
| rs2248069 | CACNA1A | hsa-miR-449b* | A/G | G | 101 | −16.65 | 233% |
| rs2285744 | THSD7A | hsa-miR-518b | C/G | C | 104 | −20.67 | −313% |
| rs3748570 | NES | hsa-miR-708* | A/G | A | 82 | −20.62 | −312% |
| rs2774279 | ARHGAP30 | hsa-miR-629 | C/T | T | 89 | −20.58 | 312% |
| rs2466613 | OR8G2 | hsa-miR-30d | C/T | C | 95 | −20.6 | −312% |
| rs2466613 | OR8G2 | hsa-miR-30a | C/T | T | 93 | −19.4 | 288% |
| rs2075520 | ZP2 | hsa-miR-501-5p | A/C | C | 96 | −20.95 | 312% |
| rs9333548 | POLH | hsa-miR-125a-5p | C/T | C | 122 | −20.6 | −312% |
| rs2797492 | C10orf18 | hsa-miR-133b | A/G | G | 86 | −20.56 | 311% |
| rs10897533 | EHD1 | hsa-miR-374b* | C/G | C | 98 | −20.53 | −311% |
| rs3782886 | BRAP | hsa-miR-192* | A/G | G | 95 | −20.55 | 311% |
| rs7240666 | ALPK2 | hsa-miR-1284 | C/T | T | 86 | −20.54 | 311% |
| rs10419202 | OR7D4 | hsa-miR-1244 | A/G | G | 95 | −20.53 | 311% |
| rs6728999 | FN1 | hsa-miR-1181 | C/T | C | 112 | −24.55 | −311% |
| rs3130626 | BAT2 | hsa-miR-593* | A/G | G | 109 | −24.7 | 311% |
| rs3742002 | BRAP | hsa-miR-576-5p | C/T | T | 101 | −20.48 | 310% |

²Location for all SNPs identified in Table 5 is CDS

TABLE VI

| | | | | Active | Score for | MFE for | |
|---|---|---|---|---|---|---|---|
| | | | Allelic | Allele | miRNA: | miRNA: | MFE |
| SNP ID³ | Gene Symbol | miRNA ID | Variants | (AA) | mRNA-AA | mRNA-AA | Change % |
| rs2241056 | MACC1 | hsa-miR-937 | A/G | G | 95 | −25.32 | 406% |
| rs9995 | NBN | hsa-miR-223* | C/T | C | 128 | −24.02 | −380% |
| rs281437 | ICAM1 | hsa-miR-30d | C/T | T | 91 | −23.93 | 379% |
| rs281437 | ICAM1 | hsa-miR-30a | C/T | T | 88 | −20.86 | 289% |
| rs281437 | ICAM1 | hsa-miR-30e | C/T | T | 85 | −18.4 | 268% |
| rs3731754 | PLEKHA3 | hsa-miR-520h | C/G | G | 89 | −23.94 | 379% |
| rs3731754 | PLEKHA3 | hsa-miR-520g | C/G | G | 90 | −24.95 | 343% |
| rs1203 | ITSN2 | hsa-miR-450a | A/G | A | 127 | −23.77 | −375% |
| rs2289046 | IRS2 | hsa-miR-935 | A/G | G | 102 | −23.52 | 370% |
| rs2010604 | OAS3 | hsa-miR-541* | C/G | C | 94 | −23.26 | −365% |
| rs3814452 | JARID2 | hsa-miR-30d | G/T | T | 83 | −23.14 | 363% |
| rs7952784 | SVOP | hsa-miR-183* | A/G | A | 84 | −23.03 | −361% |
| rs10903032 | IL28RA | hsa-miR-1238 | A/G | G | 112 | −23.81 | 357% |
| rs5745933 | MAN2C1 | hsa-miR-659 | A/G | G | 97 | −24.07 | 357% |
| rs3209896 | AKR1C3 | hsa-miR-369-5p | A/G | G | 84 | −22.8 | 356% |
| rs6053666 | SRXN1 | hsa-miR-509-3p | C/T | C | 80 | −22.87 | −356% |
| rs7601998 | CAPN14 | hsa-miR-532-5p | A/C | C | 128 | −23.12 | 355% |
| rs12945 | FERMT1 | hsa-miR-520g | C/T | C | 101 | −22.66 | −353% |
| rs12945 | FERMT1 | hsa-miR-520h | C/T | C | 91 | −18.69 | −274% |
| rs17205908 | KIAA0408 | hsa-miR-302b | C/T | C | 92 | −22.46 | −349% |
| rs17392389 | PAP2D | hsa-miR-770-5p | C/T | C | 96 | −22.33 | −347% |
| rs3742134 | STK24 | hsa-miR-181a* | A/G | G | 94 | −22.34 | 347% |
| rs6091816 | RPRD1B | hsa-miR-491-3p | C/G | C | 120 | −22.36 | −347% |
| rs3735172 | C7orf29 | hsa-miR-518a-3p | C/T | C | 116 | −23.49 | −346% |
| rs3735172 | C7orf29 | hsa-miR-518c | C/T | C | 109 | −19.59 | −292% |
| rs3735172 | C7orf29 | hsa-miR-518b | C/T | C | 110 | −18.79 | −276% |
| rs7516087 | PADI2 | hsa-miR-137 | A/G | G | 83 | −22.26 | 345% |
| rs531393 | SNX3 | hsa-miR-194 | A/G | G | 82 | −22.24 | 345% |
| rs399535 | DHRS7 | hsa-miR-105 | A/G | G | 86 | −22.2 | 344% |
| rs1787474 | WDR7 | hsa-miR-885-5p | A/G | A | 101 | −24.04 | −342% |
| rs4804572 | KANK2 | hsa-miR-483-5p | C/T | C | 129 | −23.44 | −340% |
| rs10900867 | NUDT12 | hsa-let-7c* | A/G | G | 107 | −22 | 340% |
| rs9590958 | N4BP2L1 | hsa-miR-200c* | A/G | G | 86 | −21.93 | 339% |
| rs2072878 | MAPK11 | hsa-miR-19b-2* | A/G | G | 110 | −21.92 | 338% |
| rs10888263 | TRIM58 | hsa-miR-103 | G/T | G | 94 | −21.87 | −337% |
| rs9957261 | ST8SIA3 | hsa-miR-361-5p | C/T | C | 85 | −21.81 | −336% |
| rs3741133 | PPME1 | hsa-miR-181c | C/T | C | 85 | −24.81 | −335% |
| rs3741133 | PPME1 | hsa-miR-181a | C/T | C | 87 | −25.79 | −330% |
| rs10473 | MXRA7 | hsa-miR-30b* | A/C | C | 81 | −21.69 | 334% |
| rs2034475 | ZNF167 | hsa-miR-1233 | A/C | C | 95 | −21.66 | 333% |
| rs2034475 | ZNF167 | hsa-miR-625* | A/C | A | 95 | −21.19 | −324% |
| rs3743517 | C16orf63 | hsa-miR-1293 | A/G | G | 82 | −21.53 | 331% |
| rs890336 | CNDP2 | hsa-miR-582-5p | A/G | A | 98 | −21.49 | −330% |
| rs1436607 | UNC5D | hsa-miR-1281 | A/G | G | 117 | −21.51 | 330% |
| rs1406093 | RIC3 | hsa-miR-1184 | C/T | C | 93 | −21.43 | −329% |
| rs12459634 | TMEM149 | hsa-miR-1237 | C/T | C | 86 | −21.4 | −328% |
| rs9574 | PROCR | hsa-miR-551b* | C/G | C | 95 | −24.06 | −328% |
| rs9574 | PROCR | hsa-miR-1324 | C/G | C | 82 | −19.07 | −281% |
| rs9574 | PROCR | hsa-miR-520c-3p | C/G | C | 84 | −18.39 | −258% |
| rs3744801 | USP36 | hsa-miR-548o | C/T | T | 84 | −23.23 | 327% |
| rs1044045 | EFHA1 | hsa-miR-517c | A/G | G | 81 | −21.32 | 326% |
| rs13218274 | L3MBTL3 | hsa-miR-876-5p | A/C | C | 94 | −24.14 | 326% |
| rs4589703 | SH3BP4 | hsa-miR-1909 | C/G | C | 81 | −21.27 | −325% |
| rs6001161 | LOC646851 | hsa-miR-1183 | C/G | C | 122 | −21.23 | −325% |
| rs3921 | CXCL10 | hsa-miR-520g | C/G | G | 82 | −21.23 | 325% |
| rs3921 | CXCL10 | hsa-miR-1324 | C/G | C | 87 | −17.72 | −254% |
| rs6854393 | FHDC1 | hsa-let-7d* | A/G | G | 87 | −21.24 | 325% |
| rs480153 | RPUSD4 | hsa-miR-520g | C/T | T | 98 | −21.2 | 324% |
| rs480153 | RPUSD4 | hsa-miR-520h | C/T | T | 98 | −21.2 | 227% |
| rs3025529 | VAPA | hsa-miR-214* | A/C | C | 87 | −21.51 | 323% |
| rs16845388 | UTP3 | hsa-miR-151-3p | G/T | G | 103 | −21.1 | −322% |
| rs2781667 | MED23 | hsa-miR-1913 | C/T | C | 120 | −21.1 | −322% |
| rs11046536 | ETNK1 | hsa-miR-550* | A/G | G | 99 | −21.07 | 321% |
| rs3809422 | ATG2B | hsa-miR-892b | C/T | C | 97 | −21.07 | −321% |
| rs4729655 | MUC17 | hsa-miR-30a | C/T | T | 92 | −20.99 | 320% |
| rs4729655 | MUC17 | hsa-miR-30b | C/T | T | 93 | −19.12 | 282% |
| rs4729655 | MUC17 | hsa-miR-30c | C/T | T | 96 | −19.12 | 282% |
| rs4647153 | ERCC8 | hsa-miR-633 | C/T | C | 86 | −20.95 | −319% |
| rs4647153 | ERCC8 | hsa-miR-380 | C/T | T | 83 | −18.51 | 270% |
| rs3135824 | FGFR2 | hsa-miR-505 | C/T | C | 107 | −20.92 | −318% |
| rs1801833 | AATK | hsa-miR-34a | A/G | G | 116 | −23.48 | 317% |
| rs10404926 | ZNF681 | hsa-miR-139-3p | C/T | C | 81 | −20.87 | −317% |
| rs10404926 | ZNF681 | hsa-miR-425 | C/T | C | 93 | −17.18 | −244% |
| rs1150789 | TMEM217 | hsa-miR-1299 | A/G | A | 123 | −20.8 | −316% |
| rs3801062 | SDK1 | hsa-miR-590-5p | C/G | C | 81 | −22.19 | −316% |
| rs1042541 | BIRC5 | hsa-miR-187 | A/G | G | 82 | −23.26 | 314% |
| rs1128880 | INF2 | hsa-miR-1237 | G/T | G | 99 | −20.66 | −313% |
| rs16853105 | PLEKHA6 | hsa-miR-484 | C/T | C | 103 | −21.8 | −312% |
| rs11536888 | TLR4 | hsa-miR-486-5p | C/T | C | 95 | −24.61 | −312% |
| rs2181413 | MOBKL2C | hsa-miR-589* | C/T | T | 98 | −21.51 | 311% |
| rs1225891 | MACROD2 | hsa-miR-106a | C/T | C | 94 | −21.93 | −311% |
| rs1225891 | MACROD2 | hsa-miR-17 | C/T | C | 94 | −21.93 | −311% |
| rs1046662 | RCHY1 | hsa-miR-23a | A/C | A | 91 | −20.8 | −311% |
| rs1046662 | RCHY1 | hsa-miR-23b | A/C | A | 87 | −20.33 | −270% |
| rs11422 | MEOX1 | hsa-miR-1249 | A/G | G | 105 | −20.52 | 310% |
| rs6744 | PPP6C | hsa-miR-105 | A/G | G | 116 | −21.02 | 310% |
| rs10999326 | EIF4EBP2 | hsa-miR-1226 | C/G | G | 104 | −20.96 | 309% |
| rs10999326 | EIF4EBP2 | hsa-miR-130b* | C/G | G | 118 | −24.4 | 263% |
| rs2658557 | LDHAL6A | hsa-miR-18a | G/T | G | 102 | −20.47 | −309% |
| rs12510386 | GAB1 | hsa-miR-380 | A/C | C | 111 | −20.46 | 309% |
| rs10060444 | MTX3 | hsa-miR-1224-3p | A/T | T | 94 | −22.16 | 309% |
| rs3180249 | CPLX2 | hsa-miR-634 | A/G | G | 87 | −20.44 | 309% |
| rs4658 | SLC2A1 | hsa-miR-186 | C/G | G | 83 | −22.16 | 308% |
| rs2306124 | EFNA3 | hsa-miR-541* | A/G | G | 81 | −21.36 | 308% |
| rs3213588 | ADCY10 | hsa-miR-212 | A/G | G | 95 | −20.38 | 308% |
| rs11848973 | SEL1L | hsa-miR-675* | G/T | G | 95 | −20.42 | −308% |
| rs17754 | RFC1 | hsa-miR-29a | C/G | C | 123 | −20.41 | −308% |
| rs9373717 | FUT9 | hsa-miR-383 | A/C | A | 116 | −20.41 | −308% |
| rs4839688 | FHL5 | hsa-miR-492 | A/G | G | 80 | −21.29 | 308% |
| rs11578216 | RHOU | hsa-miR-30d* | A/T | T | 135 | −20.34 | 307% |
| rs9892296 | HELZ | hsa-miR-380* | C/T | C | 109 | −20.36 | −307% |
| rs1052278 | GLT8D4 | hsa-miR-890 | A/C | C | 120 | −20.39 | 307% |
| rs1996 | SAPS3 | hsa-miR-921 | A/G | G | 88 | −22.11 | 306% |
| rs1044630 | TMEM177 | hsa-miR-760 | A/G | G | 102 | −23.04 | 306% |
| rs1535381 | RASSF2 | hsa-miR-30c | C/T | C | 82 | −20.28 | −306% |
| rs14192 | ACPP | hsa-miR-342-3p | C/T | T | 87 | −20.31 | 306% |
| rs14192 | ACPP | hsa-miR-33a* | C/T | T | 87 | −18.91 | 227% |
| rs9842062 | TMEM43 | hsa-miR-138-2* | A/G | G | 87 | −20.24 | 305% |
| rs3654 | NTRK2 | hsa-miR-1182 | A/G | G | 88 | −20.25 | 305% |
| rs3654 | NTRK2 | hsa-miR-483-5p | A/G | G | 111 | −16.62 | 228% |
| rs12271969 | C11orf31 | hsa-miR-1 | C/T | T | 109 | −20.21 | 304% |
| rs12271969 | C11orf31 | hsa-miR-206 | C/T | T | 110 | −19.42 | 288% |
| rs535443 | TMEM133 | hsa-miR-488* | C/T | T | 84 | −20.21 | 304% |
| rs11658829 | ULK2 | hsa-miR-520g | A/C | C | 96 | −20.24 | 304% |
| rs11658829 | ULK2 | hsa-miR-520h | A/C | C | 96 | −18.58 | 272% |
| rs9833685 | SLC41A3 | hsa-miR-1914 | C/T | C | 106 | −21.57 | −304% |
| rs13147697 | EVC | hsa-miR-25* | A/G | G | 125 | −20.18 | 304% |
| rs3796308 | TMEM43 | hsa-miR-210 | A/C | C | 99 | −20.13 | 303% |
| rs9821047 | TBC1D5 | hsa-miR-636 | C/G | C | 119 | −20.16 | −303% |
| rs4728185 | KLHDC10 | hsa-miR-1290 | C/G | C | 92 | −20.16 | −303% |
| rs11999 | FXYD2 | hsa-miR-133a | A/C | C | 81 | −20.08 | 302% |
| rs1045670 | RPH3A | hsa-miR-452* | A/G | G | 97 | −20.08 | 302% |
| rs1045670 | RPH3A | hsa-miR-449b* | A/G | A | 98 | −18.87 | −244% |
| rs1045670 | RPH3A | hsa-miR-362-3p | A/G | G | 109 | −21.22 | 243% |
| rs2841 | DOK5 | hsa-miR-1914 | C/T | C | 116 | −20.1 | −302% |

³Location is 3′ UTR for each SNP described in Table 6.

Using our research strategy (FIG. 5) and stringent miRanda settings (see Methods), we found that 41%, 6% and 53% of target SNPs were located inside 5′ UTR, CDS and 3′ UTR, respectively. In the MFE change distribution of all transcribed SNPs, the lower 2.5 and the higher 97.5 percentile corresponded to minimum 94% MFE change. By using 94% cut-off, we found that approximately 64% of transcribed SNPs disrupt (increase/decrease) the MFE of putative miRNA::mRNA duplexes. In summary, through this genome-wide bioinformatics approach, we identified a set of putative target SNPs that modify the MFE of miRNA::mRNA binding and eventually influence the interaction and the regulatory function of miRNAs with their targets.

Additionally, we performed a similar analysis on the 17 SNPs that Saunders et al. identified inside experimentally verified miRNA target sites with the corresponding interacting miRNAs. Saunders M A, et al., Human polymorphism at microRNAs and microRNA Target Sites, 104:3300-3305, Proc Natl Acad Sci USA. (2007) as follows:

TABLE VII

| | Target | | |
|---|---|---|---|
| SNP | gene | miRNA | Target sequence |
| rs11583293 | LDLRAP1 | miR-124 | G[T/G]GCCTTT |
| rs2893808 | SLC16A9 | miR-1/-206 | TCATTCC[G/A] |
| rs11475489 | SDC4 | miR-1/-206 | TCATT [C/-]CT |
| rs4030414 | CD24 | miR-93/-302/ | |
| | | -372/-373 | AGCACT[T/A]A |
| rs11539178 | ATP6V0E | miR-124 | ATGCCT[T/C]A |
| rs11555067 | POLR2K | miR-1/-206 | ACATT[C/T]CA |
| rs1054280 | DVL2 | miR-124 | CT[G/A]CCTTT |
| rs1042538 | IQGAP1 | miR-124 | CTGCCTT[T/A] |
| rs8226 | MYH9 | miR-124 | GTGC[C/T]TTA |
| rs17620927 | MKRN1 | miR-93/-302/ | |
| | | -372/-373 | G[G/C]CACTTT |
| rs8829 | EZH2 | miR-101 | GTACTG[T/G]A |
| rs28635788 | TTC7A | miR-124 | GTGCCT[T/C]T |
| rs10196117 | TTC7A | miR-124 | GTGCCTT[T/C] |
| rs11553391 | CAV1 | miR-124 | AT[G/A]CCTTA |
| rs7233791 | ACAA2 | miR-124 | TTG[C/G]CTTA |
| rs1050288 | KLHDC5 | miR-1/-206 | A[C/T]ATTCCC |
| rs17168525 | MTPN | let-7/miR-98 | GTA[C/T]CTCA |

We observed that 78% of these SNPs were competent to change the miRNA MFE by at least 8%. Therefore, variant alleles inside experimentally verified miRNA target sites can change the status of miRNA::mRNA interactions and affect gene expression, consistent with our genome-wide bioinformatics predictions.

Target SNP Distribution in BC and Control Populations.

We sequenced a panel of BC patients (166 sporadic and 169 familial BRCA1 and BRCA2 negative probands) and controls (186) for the germline presence of selected target SNPs located inside cancer relevant genes. (Table 2).

Table VIII provides a summary of the distribution of target SNPs genotypes and their association with BC risk in Caucasian cases (sporadic and familial BC) with controls:

TABLE VIII

| | | | | | Fisher's Exact |
|---|---|---|---|---|---|
| SNP (gene) | Genotype | CONTROL | SPORADIC | FAMILIAL | Test P-value |
| rs17739 | CC | 111(35.2%) | 105(33.3%) | 99(31.4%) | 0.1134 |
| (TP53BP2) | CT | 67(38.5%) | 48(27.6%) | 59(33.9%) | |
| | TT | 7(31.8%) | 12(54.5%) | 3(13.6%) | |
| rs2832751 | GG | 109(35.6%) | 96(31.4%) | 101(33%) | 0.9115 |
| (BIRC4) | GA | 63(35.2%) | 60(33.5%) | 56(31.3%) | |
| | AA | 13(41.9%) | 10(32.3%) | 8(25.8%) | |
| rs2832752 | GG | 109(35.6%) | 96(31.4%) | 101(33%) | 0.9115 |
| (BIRC4) | GC | 63(35.2%) | 60(33.5%) | 56(31.3%) | |
| | CC | 13(41.9%) | 10(32.3%) | 8(25.8%) | |
| rs334348 | AA | 51(33.3%) | 54(35.3%) | 48(31.4%) | 0.0075 |
| (TGFBR1) | AG | 32(22.9%) | 39(27.9%) | 69(49.3%) | |
| | GG | 7(41.2%) | 7(41.2%) | 3(17.6%) | |
| rs769412 | AA | 70(39.3%) | 64(36%) | 44(24.7%) | 0.9336 |
| (MDM2) | AG | 11(34.4%) | 12(37.5%) | 9(28.1%) | |
| | GG | 1(100%) | 0(0%) | 0(0%) | |
| rs7760 | TT | 66(36.3%) | 73(40.1%) | 43(23.6%) | 0.6079 |
| (TP53INP1) | TG | 18(39.1%) | 14(30.4%) | 14(30.4%) | |
| | GG | 2(40%) | 1(20%) | 2(40%) | |
| rs799917 | CC | 90(50%) | 40(22.2%) | 50(27.8%) | 0.0177 |
| (BRCA1) | CT | 75(38.9%) | 66(34.2%) | 52(26.9%) | |
| | TT | 20(33.9%) | 25(42.4%) | 14(23.7%) | |

The logistic regression analysis indicated that rs799917-BRCA1 [T] carriers were more likely associated with BC (all cases vs controls:univariate analysis odds ratio (OR) for [CT] carriers 1.57, p=0.03 and still significant in multivariate analysis with OR=1.86 and p=0.046, and univariate analysis OR for [TT] carriers 1.95, p=0.03) and in particular with sporadic BC (sporadic cases vs controls: univariate analysis OR for CT carriers 1.98, p=0.007 and OR for [TT] carriers 2.81, p=0.003) (Table 3). Furthermore, the analysis suggested that rs334348-TGFBR1 [AG] carriers were more likely to have BC (all cases vs controls: univariate analysis OR 1.69, p=0.048) and in particular familiar BC (familiar vs control: univariate analysis OR 2.2, p=0.005; and familiar vs sporadic: univariate analysis OR 1.99, p=0.01) and this association was also significant in the multivariate analysis (familiar vs control: multivariate analysis OR 2.67, p=0.002; and familiar vs sporadic: multivariate analysis OR 2.17, p=0.02) Despite the small size of the case-control study population, this evidence suggests that specific target SNPs have differential distribution among familial BCs, sporadic BCs and controls.

Validation of Target SNP Predictions.

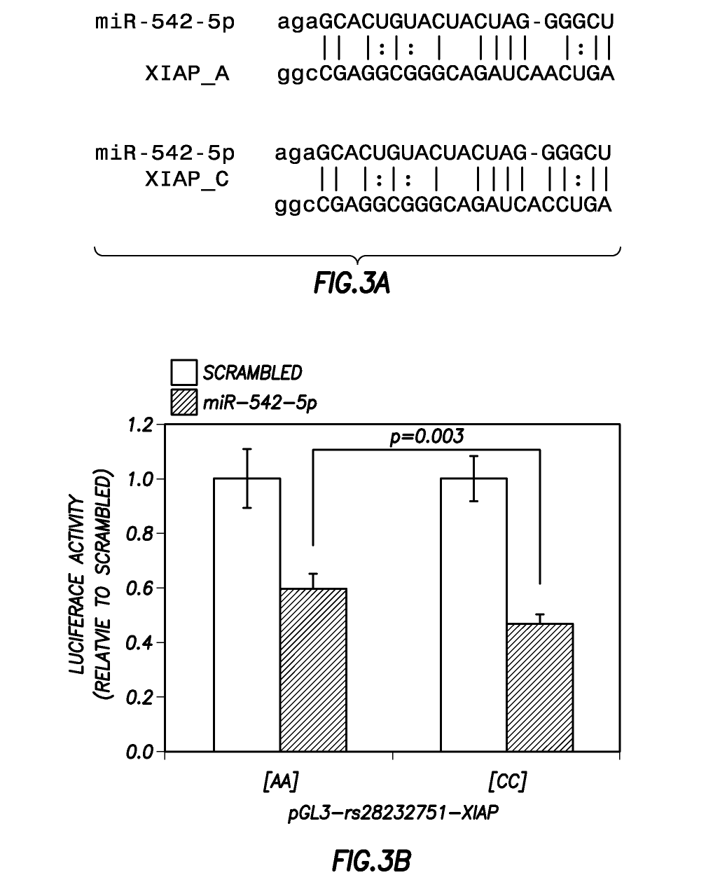

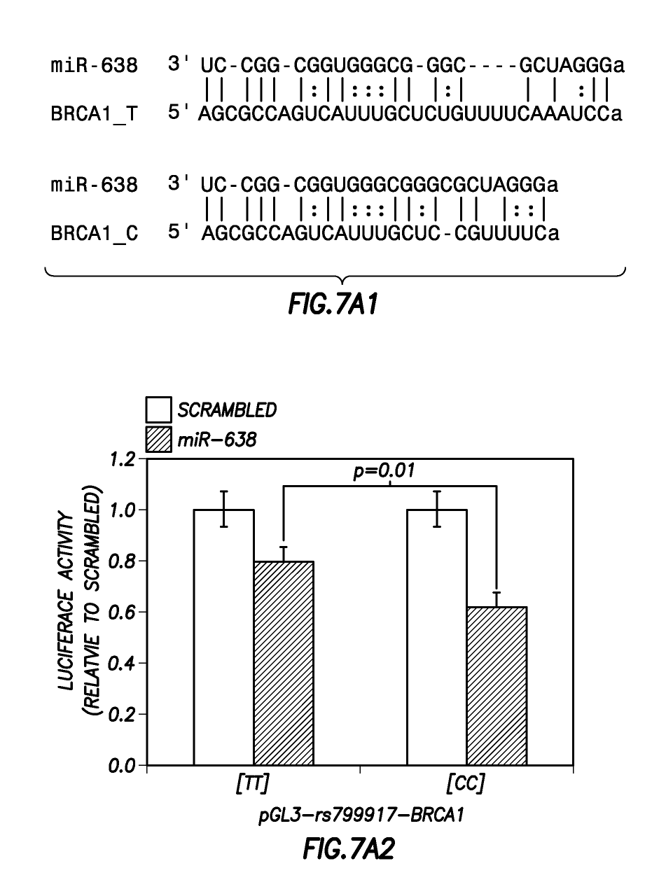

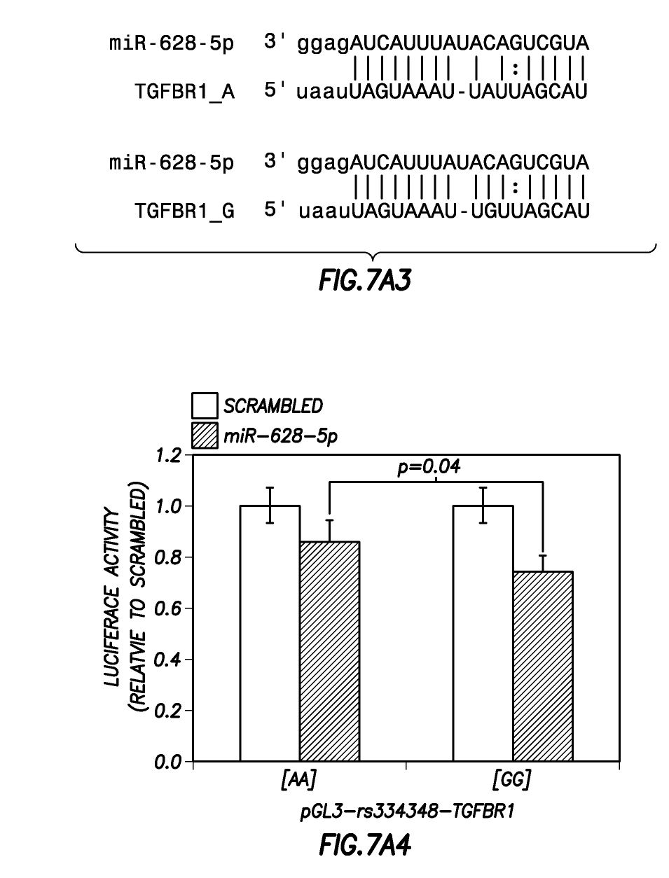

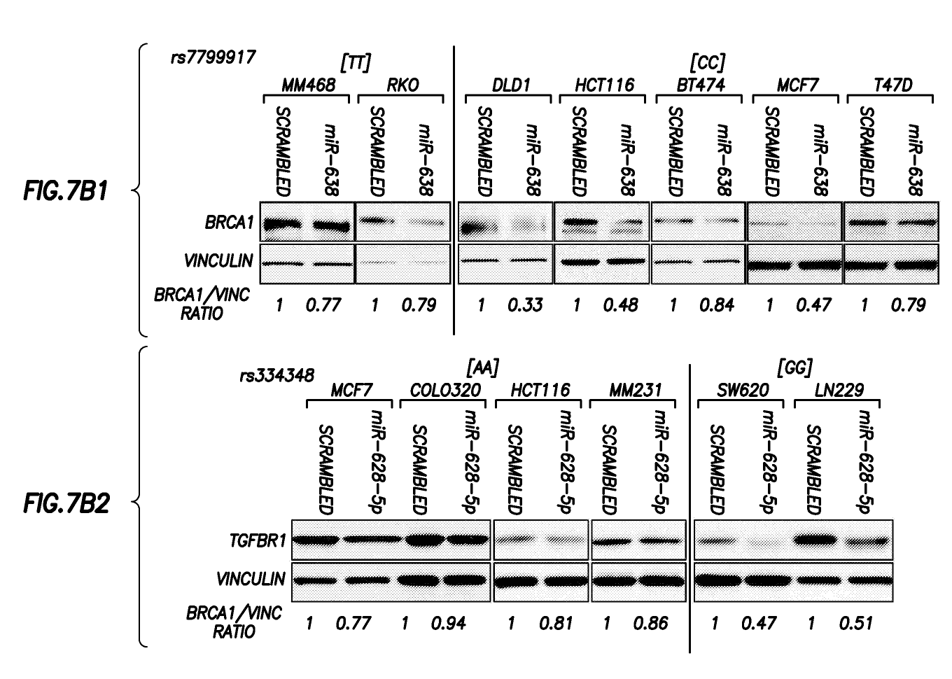

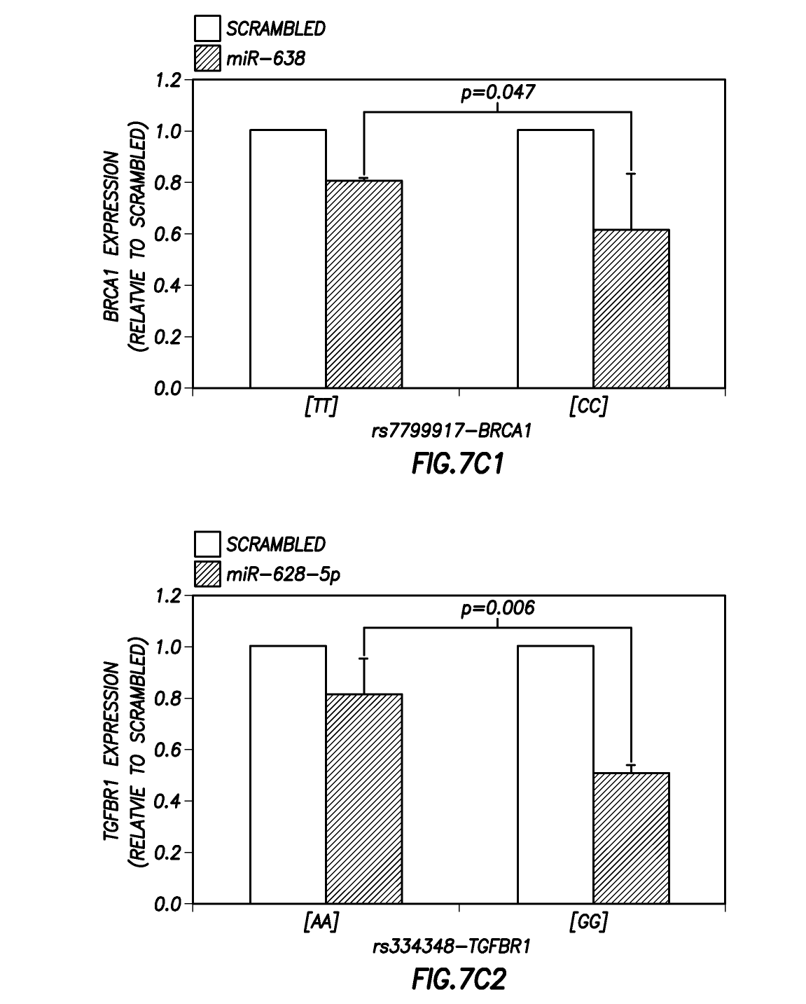

To validate the computational predictions and the biological relevance of target SNPs, first we carried out in vitro luciferase reporter assays. The identified target SNPs that are located in BRCA1, TGFBR1 and XIAP (Table 2) affected significantly (p<0.05) the pGL3-SNP luciferase activity by the predicted interacting miRNAs (FIG. 7a and FIG. 3). In all three cases tested, miRNAs displayed a higher repressive effect when interacting with the active allele. Next, we tested the effect of the two target SNPs that we found to be associated with BC, rs799917-BRCA1 and rs334348-TGFBR1, on endogenous BRCA1 and TGFBR1 protein levels.

We transfected the interacting miRNAs (miR-638 and miR-628-5p) in cancer cell lines carrying different target SNP genotypes. Following miR-638 over-expression, 7 out of 9 cell lines (in which BRCA1 was clearly detected in order to perform correct band quantification) showed a reduction in BRCA1 protein levels (FIG. 7b left panel and data not shown). By sub-grouping cell lines responsive to miR-638 induced BRCA1 reduction according to rs799917 genotype, we observed that the presence of the [CC] genotype was responsible for a stronger reduction of BRCA1 protein levels (61% reduction vs. 79%, p=0.047; FIG. 7c left panel).

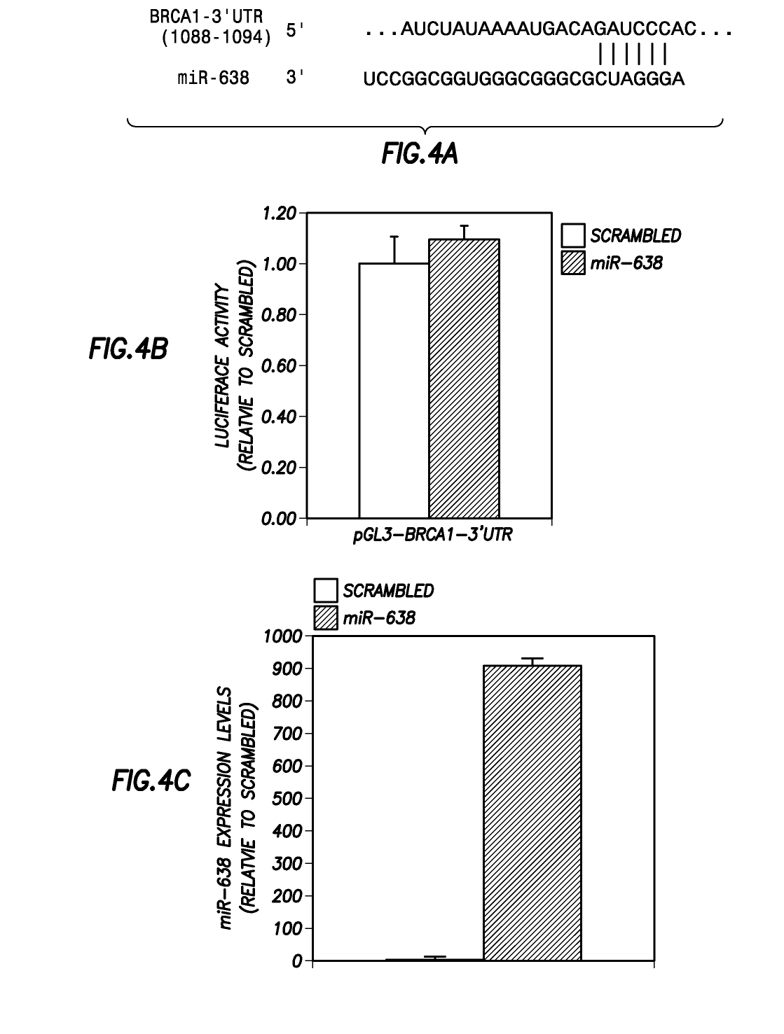

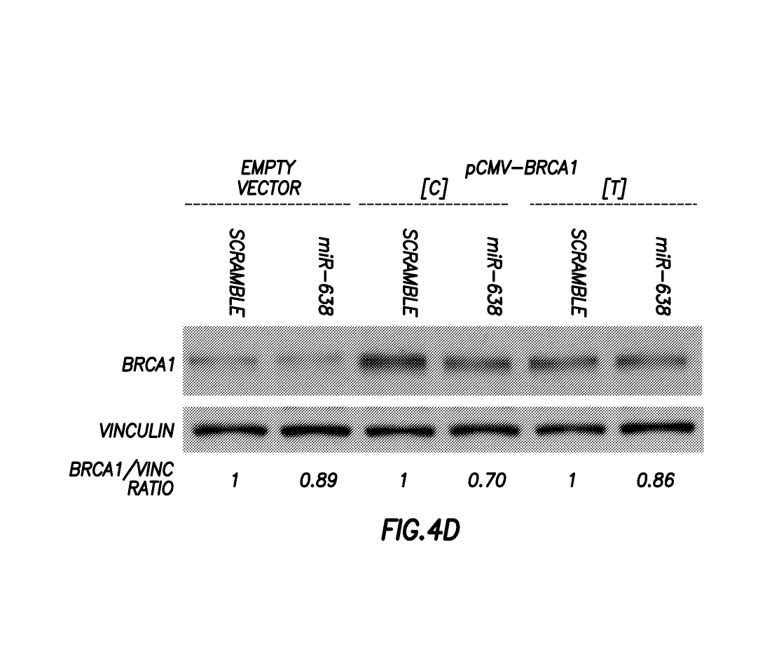

Additionally, we excluded the possibility that miR-638 dependent BRCA1 reduction was due to a conserved target site inside BRCA 1 3′ UTR predicted by TargetScan via luciferase and BRCA1 over-expression experiments (FIG. 4), further confirming the functional significance of the target SNP interaction site. Following similar approach, we evaluated the effect of rs334348 on miR-628-5p regulation of TGFBR1. By over-expressing miR-628-5p in different cell lines, we observed that miR-628-5p behaves as a true repressor of TGFBR1 (FIG. 7b right panel, and data not shown) in a cell specific manner and that its repressor activity on TGFBR1 protein levels is dependent on the rs334348 variant inside the TGFBR1 3′ UTR miRNA target sequence (80% vs 50% reduction with the [AA] and the [CC] genotypes respectively, p=0.006; FIG. 7c right panel). The observed significant effects on protein modulation by miRNA in the presence of the active or non-active alleles (FIG. 3, FIG. 6, and FIG. 7) support our hypothesis that genetic variants inside miRNA targets can actually disrupt miRNA gene regulation and affect protein expression, eventually leading to tumor susceptibility.

In order to detect the presence of a SNP-miRNA expression combination predisposing an individual to tumors or cancer, a biological sample such as blood is prepared and analyzed for the presence or absence of allelic variants and miRNA expression. In order to detect the presence of neoplasia, the progression toward malignancy of a precursor lesion, or as a prognostic indicator, a biological sample of the lesion is prepared and analyzed for the presence or absence of allelic variants of the SNPs described herein. Results of these tests and interpretive information are returned to the health care provider for communication to the tested individual. Such diagnoses may be performed by diagnostic laboratories, or, alternatively, diagnostic kits are manufactured and sold to health care providers or to private individuals for self-diagnosis.

Initially, the screening method may involve amplification of the relevant sequences. Alternatively, the screening method could involve a non-PCR based strategy. Such screening methods include two-step label amplification methodologies that are well known in the art. Both PCR and non-PCR based screening strategies can detect target sequences with a high level of sensitivity. When the probes are used to detect the presence of the target sequences (for example, in screening for cancer susceptibility), the biological sample to be analyzed, such as blood or serum, may be treated, if desired, to extract the nucleic acids. The sample nucleic acid may be prepared in various ways to facilitate detection of the target sequence; e.g. denaturation, restriction digestion, electrophoresis or dot blotting.

Methods for detecting the SNP-miRNA expression combination may include, but are not limited to, enzyme linked immunosorbent assays (ELISA), radioimmunoassays (RIA), immunoradiometric assays (IRMA) and immunoenzymatic assays (IEMA), including sandwich assays using monoclonal and/or polyclonal antibodies. Detection is often accomplished by the use of labeled probes. Suitable labels, and methods for labeling probes and ligands are known in the art, and include, for example, radioactive labels which may be incorporated by known methods (e.g., nick translation, random priming or kinasing), biotin, fluorescent groups, chemiluminescent groups (e.g., dioxetanes, particularly triggered dioxetanes), enzymes, antibodies and the like. Variations are known in the art, and include those variations that facilitate separation of the hybrids to be detected from extraneous materials and/or that amplify the signal from the labeled moiety. It is further contemplated that the nucleic acid probe assays of this invention may employ a cocktail of nucleic acid probes. Any number of probes can be used, and can include probes corresponding to the major gene mutations identified as predisposing an individual to cancer.

As taught above, the methods of determining the risk of breast cancer using the combination of SNP allelic variant and miRNA expression can be used alone or as a second step, after a negative diagnosis is made via the BCRA1 and BCRA2 tests are administered. The following is information provided by National Cancer Institute. http://www.cancer.gov/cancertopics/factsheet/Risk/BRCA#r14.

“BRCA1 and BRCA2 are human genes that belong to a class of genes known as tumor suppressors. In normal cells, BRCA1 and BRCA2 help ensure the stability of the cell's genetic material (DNA) and help prevent uncontrolled cell growth. Mutation of these genes has been linked to the development of hereditary breast and ovarian cancer. The names BRCA1 and BRCA2 stand for breast cancer susceptibility gene 1 and breast cancer susceptibility gene 2, respectively.

Not all gene changes, or mutations, are deleterious (harmful). Some mutations may be beneficial, whereas others may have no obvious effect (neutral). Harmful mutations can increase a person's risk of developing a disease, such as cancer. A woman's lifetime risk of developing breast and/or ovarian cancer is greatly increased if she inherits a harmful mutation in BRCA1 or BRCA2. Such a woman has an increased risk of developing breast and/or ovarian cancer at an early age (before menopause) and often has multiple, close family members who have been diagnosed with these diseases. Harmful BRCA1 mutations may also increase a woman's risk of developing cervical, uterine, pancreatic, and colon cancer. Harmful BRCA2 mutations may additionally increase the risk of pancreatic cancer, stomach cancer, gallbladder and bile duct cancer, and melanoma. Men with harmful BRCA1 mutations also have an increased risk of breast cancer and, possibly, of pancreatic cancer, testicular cancer, and early-onset prostate cancer. However, male breast cancer, pancreatic cancer, and prostate cancer appear to be more strongly associated with BRCA2 gene mutations.

The likelihood that a breast and/or ovarian cancer is associated with a harmful mutation in BRCA1 or BRCA2 is highest in families with a history of multiple cases of breast cancer, cases of both breast and ovarian cancer, one or more family members with two primary cancers (original tumors that develop at different sites in the body), or an Ashkenazi (Eastern European) Jewish background. However, not every woman in such families carries a harmful BRCA1 or BRCA2 mutation, and not every cancer in such families is linked to a harmful mutation in one of these genes. Furthermore, not every woman who has a harmful BRCA1 or BRCA2 mutation will develop breast and/or ovarian cancer.

According to estimates of lifetime risk, about 12.0 percent of women (120 out of 1,000) in the general population will develop breast cancer sometime during their lives compared with about 60 percent of women (600 out of 1,000) who have inherited a harmful mutation in BRCA1 or BRCA2. In other words, a woman who has inherited a harmful mutation in BRCA1 or BRCA2 is about five times more likely to develop breast cancer than a woman who does not have such a mutation.

Lifetime risk estimates for ovarian cancer among women in the general population indicate that 1.4 percent (14 out of 1,000) will be diagnosed with ovarian cancer compared with 15 to 40 percent of women (150-400 out of 1,000) who have a harmful BRCA1 or BRCA2 mutation. It is important to note, however, that most research related to BRCA1 and BRCA2 has been done on large families with many individuals affected by cancer. Estimates of breast and ovarian cancer risk associated with BRCA1 and BRCA2 mutations have been calculated from studies of these families. Because family members share a proportion of their genes and, often, their environment, it is possible that the large number of cancer cases seen in these families may be due in part to other genetic or environmental factors. Therefore, risk estimates that are based on families with many affected members may not accurately reflect the levels of risk for BRCA1 and BRCA2 mutation carriers in the general population. In addition, to date, no data are available from long-term studies of the general population comparing cancer risk in women who have harmful BRCA1 or BRCA2 mutations with women who do not have such mutations. Therefore, the percentages given above are estimates that may change as more data become available.

Several methods are available to test for BRCA1 and BRCA2 mutations. See e.g., Palma M, et al., BRCA1 and BRCA2: The Genetic Testing And The Current Management Options For Mutation Carriers, 57(1):1-23, Critical Reviews in Oncology/Hematology (2006), incorporated herein by reference. Most of these methods look for changes in BRCA1 and BRCA2 DNA. At least one method looks for changes in the proteins produced by these genes. Frequently, a combination of methods is used. A blood sample is needed for these tests. The blood is drawn in a laboratory, doctor's office, hospital, or clinic and then sent to a laboratory that specializes in the tests. It can take several weeks or longer to get the test results.”

We claim:

1. A method of determining tumor susceptibility in a patient, said method comprising the step of determining the amount of at least one SNP allelic variant that disrupts an miRNA::mRNA interaction as identified in Tables I through IX in a sample taken from the patient, wherein the presence of the SNP allelic variant and expression of the miRNA in the sample indicates an increase susceptibility of breast cancer in the patient.

2. A method for diagnosis in a patient of a cancerous or precancerous condition through detection of change in minimum free energy of the miRNA::mRNA interaction as induced by a SNP allelic variant and as classified as non-cancerous by pathology, the method comprising the step of: determining the minimum free energy change of at least 8 percent, wherein the change is indicative of breast cancer.

3. A method of determining the risk of breast cancer in a patient comprising the steps of:
detecting mutations in the BRCA1 gene or BRCA2 gene in the patient resulting in a susceptibility to breast and ovarian cancers, wherein, provided no such mutations are found, and detecting at least one SNP-miRNA expression pattern combination identified in Tables I through IX in the patient, wherein the presence of the combination of the SNP allelic variant and expression of miRNA indicates susceptibility to breast cancer.
